(19) United States
(12) Patent Application Publication
(10) Pub. No.: US 2025/0067741 A1
(43) Pub. Date: Feb. 27, 2025

(54) PROSTATE CANCER CLINICAL STATUS MARKERS

(71) Applicant: UNIVERSITÄT ZÜRICH, Zürich (CH)

(72) Inventors: Irina BANZOLA, Zürich (CH); Blaz PAVLOVIC, Zürich (CH); Nagjie Laila ALIJAJ, Zürich (CH); Daniel EBERLI, Zürich (CH)

(73) Assignee: UNIVERSITÄT ZÜRICH, Zürich (CH)

(21) Appl. No.: 18/720,675

(22) PCT Filed: Dec. 16, 2022
(86) PCT No.: PCT/EP2022/086491

§ 371 Date: Jun. 17, 2024

(30) Foreign Application Priority Data
Dec. 17, 2021 (EP) 21215742.4

(51) Int. Cl. G01N 33/574

(52) U.S. Cl. CPC G01N 33/57434

(57) ABSTRACT

The present invention relates to the field of assessment of the health status, particular with regard to prostate cancer risk, by measurement of certain proteins in human samples, in particular in human urine.

This application claims the right of priority of European Patent Application EP21215742.4 filed 17 Dec. 2021, incorporated by reference herein.

### FIELD

The present invention relates to the field of assessment of the health status, particular with regard to prostate cancer risk, by measurement of certain proteins in human samples, in particular in human urine.

### BACKGROUND OF THE INVENTION

The early detection and clinical management of prostate cancer (PCa) has become a controversial subject in the past decades. PCa is the second most frequently diagnosed cancer and the fourth leading cause of cancer deaths in men worldwide. The implementation of the serum biomarker Prostate Specific Antigen (PSA) as a standard for the screening of PCa in the early 1990s resulted in an increased diagnosis of early-stage tumors and the reduction of PCa-specific mortality rates. Additional refinements in the PCa screening procedure due to new biomarkers and technologies, such as magnetic resonance imaging (MRI), have further improved the predictive performances of PSA. Nevertheless, specificities of current diagnostic examinations remain low and still lead to a high number of false positives resulting in unnecessarily performed prostate biopsies. Therefore, overdiagnosis of healthy men and overtreatment of indolent PCa remains a clinical challenge with significant impact on the quality of life of patients due to possible severe side effects.

The use of multi-parameter magnetic resonance imaging (mpMRI) prior to the first prostate biopsy has been introduced in the EAU guidelines to increase the accuracy in the diagnosis of prostate cancer, due to its ability to identify and locate suspicious lesions (putative lesions). The introduction of upfront mpMRI has improved patient selection for biopsy and allowed the direct targeting of lesions. In this clinical scenario, mpMRI followed by an in-bore MRI-guided transrectal targeted prostate biopsy (MRGB), has a prominent role to play in selecting patients who can benefit from AS, thanks to its ability to identify lesions that are non-significant.

Prostate Imaging Reporting and Data System (PI-RADS) v2 is a standardized method to report and asses lesion characteristics, by categorizing lesions in a five-point scale based on the likelihood of harbouring a clinically significant tumor (1 has the lowest and 5 has the highest probability) (Weinreb, J. C., et al., Eur Urol, 2016. 69(1): p. 16-40; Esen, T., et al.; Biomed Res Int, 2014. 2014: p. 296810).

Despite its superior performance compared to ultrasound guided biopsy, the interpretation of equivocal lesions (PI-RADS 3) remains a major issue, with the consequent misdiagnosis of clinically significant tumors, which lead to the over-treatment (False positives: PI-RADS 3-5 resulting Gleason score 0) or the potentially harmful oversight of clinically relevant PCa (False negatives: PI-RADS 1-2 resulting Gleason score 6 to 9). mpMRI have been demonstrated to have a suboptimal sensitivity (74 to 86%) in detecting clinically important PCa, thus indicating that a relevant number of potentially harmful lesions are missed.

More specific risk stratification models that can complement PSA testing are urgently needed to discriminate clinically significant PCa and to reduce the number of unnecessary biopsies performed.

Based on the above-mentioned state of the art, the objective of the present invention is to provide means and methods to define a male individual's health status, particularly with respect to possible concerns regarding the individual's prostate cancer risk or status.

This objective is attained by the subject-matter of the independent claims of the present specification, with further advantageous embodiments described in the dependent claims, examples, figures and general description of this specification.

### SUMMARY OF THE INVENTION

The inventors aimed to identify novel biomarkers for the detection of PCa and investigate their potential for an improved diagnostic test. One particular objective underlying the present invention is the desire to increase the specificity of PSA screening and reduce the number of unnecessary prostate biopsies performed.

A mass spectrometry (MS) screening on subjects' samples was performed on a discovery cohort of 43 patients, which identified top potential biomarkers as well as control molecules for the detection of all PCa grades (Table 1), high-grade PCa (Table 2) and PI-RADS (Table 3). The three tables comprise statistics and diagnostic performance of the biomarkers based on MS data. The overall list of the best 60 candidates and three control molecules is shown in Table 4 and 5 (Table 4 is separated in the three conditions all PCa grades, high-grade PCa and PI-RADS; Table 5 is a summary of all biomarkers from the three conditions). The diagnostic performance of MS data from all biomarkers from Table 4 for the identification of all PCa grades (GS≥6) or high-grade PCa (GS≥7) are summarized in Table 6. The combinatory analysis of seven biomarkers (examples) with and without clinical variables Age and PI-RADS are shown in Table 7. These candidates were then validated by ELISA as single biomarkers (Table 8), and examples of a combinatory analysis (Table 9) predicted their performances as diagnostic test for PCa screening.

Accordingly, the present invention relates to a method for collecting information about the health status of a human subject, in particular for determining if a subject has prostate cancer or not, said method comprising the quantitative detection, in a subject's sample, in particular a urine or blood sample, of the concentration of at least one of the biomarkers selected from Table 5.1, wherein the differential expression in comparison to a healthy control of at least one of the biomarkers indicates whether the subject has prostate cancer or not. Optionally, the method further comprises the transmitting of the result to the subject or a third party, for example a physician or genetic counselor.

In some embodiments, additionally the quantitative detection of the concentration of at least one of the control biomarkers listed in Table 5.2 is performed.

The present invention further relates to a therapeutic agent for use in the treatment of PCa in a subject, wherein the subject to be treated has been diagnosed with the method of the present invention to have prostate cancer.

The present invention further relates to a kit comprising the components for performing the method of the present invention.

### TERMS AND DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term human subject in the context of the present specification relates to a patient.

The PI-RADS classification is based on the multiparametric MRI images and indicates the probability of the presence of clinically significant carcinoma for each lesion on a scale of 0 to 5.

### DETAILED DESCRIPTION OF THE INVENTION

With the help of the experiment performed in accordance with the present invention it was possible to develop a test based solely on biomarkers quantification as a feasible method to improve prostate biopsy eligibility and to detect the presence of PCa, independently from serum PSA. The clinical implementation of such a test represents an important way to fulfil the need of new screening methods that are urgently needed to reduce the number of unnecessary prostate biopsies and to accurately select patients benefitting from active treatment.

A key selection criterion for the best target molecules from the screening, was the ability to discriminate healthy patients, with high specificity and accuracy, resulting in a negligible number of false negatives. For this reason, all proteins that were not detected in more than three patients' samples were excluded from further analysis. Additionally, proteins with low diagnostic performances that display a receiver operating characteristic (ROC) area under the curve (AUC) and a specificity/sensitivity below a certain threshold, were removed. For the selection of biomarkers detecting all grades of PCa an AUC of bigger than 0.670 and a specificity of more than 10% at 100% sensitivity were chosen and resulted in 43 biomarkers of which the top 25 biomarkers were further selected as candidates (Table 4; column 1). For the selection of biomarkers detecting high-grade PCa (GS=7-9), all proteins with an AUC higher than 0.610 and a specificity of more than 25% at 100% sensitivity resulted in a list of 118 biomarkers of which the top 25 biomarkers were further selected as candidates (Table 4; column 2). For the selection of biomarkers detecting a PI-RADS score of 3-5, the selection criteria were an AUC of more than 0.670 and a specificity of at least 35% at 90% sensitivity. This resulted in a list of 25 candidates (Table 4; column 3).

A first aspect of the invention relates to a method for collecting information about the health status of a human subject, said method comprising
a. the quantitative detection, in a sample obtained from the subject, of the concentration of a biomarker selected from Table 5.1,
b. establishing the statistical significance of the concentration of the biomarker.

An alternative of the first aspect of the invention relates to a method for
determining whether a subject has prostate cancer, and/or
assessing the risk of a subject for developing prostate cancer; and/or
determining whether a subject has a high grade prostate tumor; and/or
determining whether a subject has a prostate tumor with PI-RADS score of 3-5;
   said method comprising
a. the quantitative detection, in a sample obtained from the subject, of the concentration of a biomarker selected from Table 5.1,
b. establishing the statistical significance of the concentration of the biomarker.

In certain embodiments, the statistical significance is established by a test selected from the group of the unpaired non-parametric Mann-Whitney U test, ROC curve analysis (for example: Wilson/Brown method), t-test, ANOVA test, or the Pearson correlation method.

In some embodiments, additionally the quantitative detection of the concentration of at least one of the control biomarkers shown in Table 5.2 is performed.

In certain embodiments, a likelihood that the subject has prostate cancer is increased if the expression of a biomarker selected from Table 5.1 is decreased compared to the mean expression of the biomarker in a healthy control cohort.

In certain embodiments, the method is an in-vitro method.

In certain embodiments, the sample obtained from the subject is a urine or blood sample. In certain embodiments, the sample obtained from the subject is a urine sample.

The above-mentioned ranking resulted in the top 25 candidates listed in Table 1, 2 and 3 for the detection of all PCa grades, high-grade PCa and PI-RADS score 3-5, respectively. In particular, MS results of the top 25 biomarkers of all three conditions, showed a significant decrease in signal intensity when a prostate tumor is present and can identify PCa patients with better performance compared to the standard of care PSA (Table 6).

In certain embodiments, a (at least one) biomarker of at least one of columns 1, 2 and/or 3 of Table 4 is determined. In certain embodiments, a (at least one) biomarker of Table 4 is determined.

Table 4: Column 1 is No Tumor vs. Tumor GS0 vs GS6-9; Column 2 is Low vs High Grade GS0-6 vs GS7-9 and Column 3 is PI-RADS 0-2 vs 3-5. PI-RADS 0 is used to classify patients who performed the MRI but got a negative result without score. Thus, in some embodiments, Column 3 may be considered as PI-RADS 1-2 vs. 3-5.

In certain embodiments, a (at least one) biomarker of column 1 is determined. In certain embodiments, a (at least one) biomarker of column 1 and a (at least one) biomarker of column 2 and/or 3 is determined. In certain embodiments, a (at least one) biomarker of column 2 is determined. In certain embodiments, a (at least one) biomarker of column 2 and a (at least one) biomarker of column 1 and/or 3 is determined. In certain embodiments, a (at least one) biomarker of column 3 is determined. In certain embodiments, a (at least one) biomarker of column 3 and a (at least one) biomarker of column 1 and/or 2 is determined.

The combination of the biomarkers from the same or different columns improves the diagnostic performance.

In certain embodiments, a (at least one) biomarker of column 1 is determined and it is determined whether the subject has a (prostate) tumor or has no (prostate) tumor. In certain embodiments, a (at least one) biomarker of column 2 is determined and it is determined whether the subject has a low grade tumor (Grade GS0-6) or a high grade tumor (GS7-9). In certain embodiments, a (at least one) biomarker of column 1 is determined and it is determined whether the subject has a PI-RADS score of 1-2 or a PI-RADS score of 3-5.

Accordingly, in a particular embodiment, the present invention relates to a method for determining if a subject has prostate cancer, said method comprising the quantitative detection, in a subject's sample, of the concentration of at least one of the biomarkers selected from Table 4, wherein the differential expression in comparison to a healthy control of at least one of the biomarkers indicates whether the subject has prostate cancer or not.

Among those 60 biomarkers of Table 5.1, respectively, PEDF, HPX, CD99, CANX, FCER2, HRNR and KRT13 showed remarkable diagnostic performance (Table 6). For example, PEDF showed the best performance as a single biomarker, with AUC of 0.8023 and specificity of 36.4% at 100% sensitivity.

Among those 60 biomarkers of Table 5.1, respectively, PEDF, HPX, CD99, CANX, FCER2, HRNR and KRT13 showed remarkable performance in predicting the PI-RADS score (Table 3). For example, TALDO1 showed the best performance as a single biomarker, with AUC of 0.7964 and specificity of 63.6% at 90% sensitivity.

Accordingly, in a further particular embodiment, the present invention relates to a method for determining if a subject has prostate cancer, said method comprising the quantitative detection, in a subject's sample, of the concentration of at least one of the biomarkers selected from the group consisting of: PEDF, HPX, CD99, CANX, FCER2, HRNR and KRT13, wherein the differential expression in comparison to a healthy control of at least one of the biomarkers indicates whether the subject has prostate cancer or not. In one embodiment, the method of the present invention comprises at least the quantitative detection of the biomarker PEDF.

In one embodiment, the method of the present invention comprises the determination of the concentration, i.e. quantification, of more than one biomarker. In particular, the method comprises the quantitative detection of two, three, four, five, six, seven, eight, nine, ten, elven, twelve, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or all 60 of the biomarkers listed in Table 5.1.

As regards the combination of two biomarkers, the following combinations are of particular interest: PEDF with CALR, PEDF with HPX, CALR with HPX, PEDF with PNP, etc. Particularly, at least biomarker PEDF is comprised.

As regards the combination of two biomarkers, the following combinations are of particular interest: KRT13 with FECR2, KRT13 with HPX, SPARCL1 with HPX, PEDF with KRT13, etc. Particularly, at least biomarker KRT13 is comprised.

As regards the combination of two biomarkers, the following combinations are of particular interest: CD99 with FECR2, CD99 with HPX, CD99 with HPX, CD99 with KRT13, etc. Particularly, at least biomarker CD99 is comprised.

As regards the combination of two biomarkers, the following combinations are of particular interest: SPARCL1 with FECR2, SPARCL1 with HPX, SPARCL1 with HPX, SPARCL1 with KRT13, etc. Particularly, at least biomarker SPARCL1 is comprised.

In certain embodiments, the method comprises the quantification of two, three, four, five, six, seven, eight, nine, ten, elven, twelve, 13, 14, 15, 16, 17, 18, 19, 20, 21, 22, 23, 24, or 25 of the biomarkers listed in Tables 1, 2 and 3. In certain embodiments, the method comprises the quantification of two, three, four, five, six or seven of the biomarkers selected from the following list: PEDF, HPX, CD99, CANX, FCER2, HRNR and KRT13. Particularly, at least biomarker PEDF is comprised.

In certain embodiments, the method comprises the quantification of two, three, four, five, six or seven, eight, nine, ten of the biomarkers selected from the following list: PEDF, HPX, CD99, CANX, FCER2, HRNR, KRT13, AMBP, LYVE1 and SPARCL1. Particularly, at least biomarker KRT13 is comprised.

In certain embodiments, the method comprises the quantification of two, three, four, five, six or seven, eight, nine, ten of the biomarkers selected from the following list: PEDF, HPX, CD99, CANX, FCER2, HRNR, KRT13, AMBP, LYVE1 and SPARCL1. Particularly, at least biomarker SPARCL1 is comprised.

In certain embodiments, the method comprises the quantification of two, three, four, five, six or seven, eight, nine, ten of the biomarkers selected from the following list: PEDF, HPX, CD99, CANX, FCER2, HRNR, KRT13, AMBP, LYVE1 and SPARCL1. Particularly, at least biomarker HPX is comprised.

The person skilled in the art knows about the 1770 possible combinations and all of them are disclosed herein. Similar regards to the combination of three of the biomarkers wherein 34220 combinations are possible, of four of the biomarkers, etc.

In Table 7, 9 and 10 possible combinations are shown and those combinations are also encompassed by the method of the present invention. In a particular embodiment, the method of the present invention comprises the quantitative detection of PEDF and FCER2, or of PEDF and CANX, or of HPX and KRT13, or of PEDF and FCER2 and CANX, or of PEDF and FCER2 and CANX and KRT13, or of PEDF and FCER2 and CANX and KRT13 and HPX, or of PEDF and FCER2 and CANX and KRT13 and HPX and HRNR, or of PEDF and FCER2 and CANX and KRT13 and HPX and HRNR and CD99. As can be further derived from the Examples, the best performing combination of two biomarkers is shown by PEDF and FCER2 and markedly increase the AUC in predicting PCa compared to each single marker and also to PSA. Specifically, this combination could spare 72.2% of unnecessary biopsies, without missing any patient affected by PCa (100% sensitivity). Accordingly, in a particular embodiment the method comprises at least the quantification of PEDF and FCER2.

In one embodiment, the method further comprises the transmitting of the result to the subject or a third party, for example a physician or genetic counselor.

The method of the present invention is also suitable for the detection of very early stages of prostate cancer, for example such early stage that might not be visible when examining the prostate tissue obtained for example by prostate biopsy.

The specificity at 100% sensitivity shows the ability of the single biomarkers to detect all PCa in comparison to the current standard of care, serum PSA (Table 6). Accordingly, the method of the present invention is useful for the detection of patients which have any grades of PCa, in particular grades 6 to 9.

Furthermore, the quantitative analysis by ELISA showed that the seven exemplarily biomarkers can detect high-grade PCa with high performance. Thus, the method of the present invention is suitable for detecting clinically significant tumors, i.e. high grade PCa (GS≥7). The detection of high grade PCa (GS≥7) has a relevant clinical impact, as it can discriminate between patients who would benefit from active surveillance and those who need active treatments, like prostatectomy and/or chemotherapy or radiotherapy or hormone depletion treatment.

The higher the quantitative difference between the biomarkers detected in the subject's sample and the biomarkers detected in the healthy control sample, the more severe is the prostate cancer. For example, a small difference point towards a low grade PCa (GS≤6) and a strong difference points towards a high grade PCa ((GS≥7).

Each of the seven exemplarily biomarkers had a superior performance compared to PSA and were able to correctly classify 100% of patients with PCa, while also identifying true negative patients that could be spared from performing an unnecessary prostate biopsy. Thus, the method of the present invention can be used for the detection of true negative patients, meaning that with the help of the present invention unnecessary prostate biopsy can be avoided. Accordingly, the method of the present invention is useful for identifying if a patient is likely to benefit from a prostate biopsy. Furthermore, the combination of uncorrelated analytes increases the overall performance of the single biomarkers. As model example, the ELISA quantification of PEDF, FCER2 and age shows a striking AUC of 0.8022 with a specificity of 39.1% at 100% sensitivity (Table 9). Thus, in one embodiment, the method of the present invention is combined with clinical data of the human subject, for example the age of the subject.

Three exemplarily biomarkers were able to predict the PI-RADS score. Thus, the method of the present invention can be used for the detection of patients that would not receive a useful PI-RADS score (1-2 compared to 3-5), thus these patients could avoid the mpMRI reading.

Furthermore, the combination of uncorrelated analytes increases the overall performance of the single biomarkers. As model example, the ELISA quantification of AMBP showed the best performance as a single biomarker, with AUC of 0.7493 and specificity of 23.1% at 100% sensitivity (when normalized to CD44 and RNASE2).

As model example, the ELISA quantification of SPARCL1 and age shows a striking AUC of 0.0766 with a specificity of 46.2% at 90% sensitivity (Table 10). Thus, in one embodiment, the method of the present invention is combined with clinical data of the human subject, for example the age of the subject. Thus, in one embodiment, the method of the present invention is combined with clinical data of the human subject, for example the age of the subject.

The present invention further relates to a therapeutic agent for use in treating PCa in a subject, wherein the subject has been diagnosed to have PCa with the method of the present invention. In other words, the present invention relates to a therapeutic agent for use in a method of treating PCs, wherein the method comprises the diagnosing of the subject to have PCa with the method of the present invention, and further comprises administering the therapeutic agent to said subject.

Furthermore, the present invention relates to a method for treating PCa, comprising determining if a subject has prostate cancer with the method of the present invention, and treating the patient that has prostate cancer with any therapeutic agent, i.e. administering the therapeutic agent to the subject.

The therapeutic agent can be an androgen receptor blocker (also called anti-androgen, e.g., bicalutamide, flutamide, nilutamide), a second-generation androgen blocker (e.g., enzalutamide, apalutamide and darolutamide, or PARP (poly-ADP-ribose polymerase) inhibitor like olaparib, or combinations thereof.

The PCa to be treated can be any grade of PCa, but particularly high grade PCa, i.e. clinically significant tumors (GS≥7).

In case a subject has been diagnosed to have PCa (any grade or especially high grade PCa), this subject is likely amendable to the treatment with an anti-PCa agent, for example anti-tumor agent. Furthermore, the subject which has been diagnosed to have PCa, in particular high grade PCa, is likely to benefit from a prostate biopsy, and/or from active treatment, and/or from active surveillance, and/or from prostatectomy, and/or from chemotherapy or radiotherapy or hormone depletion treatment.

Furthermore, the method of the present invention can be used to monitor treatment success or the therapeutic utility of a candidate anti-PCa drug.

In principle any biological material can be used as sample for the assay of the present invention. Particularly, any body fluid is used as sample for the assay of the present invention. Particularly, the sample can be taken easily and more particularly even non-invasively. In a particular embodiment, the sample is blood or urine.

The MS screening was performed on urine samples. Urine is an ideal clinical specimen for diagnostic tests. Its collection is completely non-invasive and allows the easy collection and processing of large volumes, compared to tissue, blood or other biological materials. This enables the detection of biomarkers at any time point during patient care and facilitates not only diagnosis, but also monitoring of diseases. The detection of biomarkers in urines has been studied for a wide range of cancers with ultrasensitive screening methods such as nuclear magnetic resonance (NMR) spectroscopy and mass spectrometry (MS). Specific metabolites were examined for their potential to screen for cancers of the urological system, but also for non-urological tumors such as lung, breast, colorectal, gastric, hepatic, pancreatic and renal cancer.

The prostate epithelium secretes cellular substances into the gland and prostate cancer cells can be shed into the prostatic fluids where they exude into the urine. Sensitive assays can then detect DNA, RNA, proteins and exosomes of tumor origin. Thus, particularly urine is used as sample in the method of the present invention.

Mass spectrometry (MS)-based proteomic analysis is a powerful tool for high-throughput identification of proteins in urine and can be used for the discovery of new biomarkers. Thus, in one embodiment the quantitative detection of the biomarkers in accordance with the method of the present invention is performed by MS.

The translation of such method into the clinic for standard diagnostic screening is elusive because of high instrument costs and the need of specifically instructed personnel. Therefore, validation studies of potential biomarkers are often performed on larger patient cohorts with immunological assays such as ELISA, or SIMOA which are well-established method for protein quantification. Thus, in one embodiment the quantitative detection of the biomarkers in accordance with the method of the present invention is performed by ELISA.

In another embodiment the quantitative detection of the biomarkers in accordance with the method of the present invention is performed by SIMOA.

In certain embodiments, the sample is a urine sample.

In certain embodiments, the concentration is determined by ELISA.

In certain embodiments, the concentration is determined by SIMOA.

In certain embodiments, the concentration is determined by mass spectrometry.

In certain embodiments, the concentration of the following biomarkers is determined:
a. PEDF and FCER2; or
b. PEDF and CANX; or
c. HPX and KRT13; or
d. PEDF and FCER2 and CANX; or
e. PEDF and FCER2 and CANX and KRT13; or
f. PEDF and FCER2 and CANX and KRT13 and HPX; or
g. PEDF and FCER2 and CANX and KRT13 and HPX and HRNR; or
h. PEDF and FCER2 and CANX and KRT13 and HPX and HRNR and CD99.

In certain embodiments, the concentration of the following biomarkers is determined:
a. PEDF and FCER2 and CANX and KRT13 and HPX and HRNR and CD99 and AMBP; or
b. PEDF and FCER2 and CANX and KRT13 and HPX and HRNR and CD99 and AMBP and LYVE1; or
c. PEDF and FCER2 and CANX and KRT13 and HPX and HRNR and CD99 and AMBP and LYVE1 and SPARCL1.

In certain embodiments, the concentration of the following biomarkers is determined:
a. KRT13 and FCER2; or
b. KRT13 and CANX; or
c. KRT13 and HPX; or
d. KRT13 and PEDF; or
e. KRT13 and FCER2 and CANX; or
f. KRT13 and FCER2 and CANX and PEDF; or
g. KRT13 and FCER2 and CANX and PEDF and HPX; or
h. KRT13 and FCER2 and CANX and PEDF and HPX and HRNR; or
i. KRT13 and FCER2 and CANX and PEDF and HPX and HRNR and CD99 and AMBP; or
j. KRT13 and FCER2 and CANX and PEDF and HPX and HRNR and CD99 and AMBP and LYVE1; or
k. KRT13 and FCER2 and CANX and PEDF and HPX and HRNR and CD99 and AMBP and LYVE1 and SPARCL1.

In certain embodiments, the concentration of the following biomarkers is determined:
a. CD99 and FCER2; or
b. CD99 and CANX; or
c. CD99 and HPX; or
d. CD99 and PEDF; or
e. CD99 and FCER2 and CANX; or
f. CD99 and FCER2 and CANX and PEDF; or
g. CD99 and FCER2 and CANX and PEDF and HPX; or
h. CD99 and FCER2 and CANX and PEDF and HPX and HRNR; or
i. CD99 and FCER2 and CANX and PEDF and HPX and HRNR and KRT13 and AMBP; or
j. CD99 and FCER2 and CANX and PEDF and HPX and HRNR and KRT13 and AMBP and LYVE1; or
k. CD99 and FCER2 and CANX and PEDF and HPX and HRNR and KRT13 and AMBP and LYVE1 and SPARCL1.

In certain embodiments, the concentration of the following biomarkers is determined:
a. SPARCL1 and FCER2; or
b. SPARCL1 and CANX; or
c. SPARCL1 and HPX; or
d. SPARCL1 and PEDF; or
e. SPARCL1 and FCER2 and CANX; or
f. SPARCL1 and FCER2 and CANX and PEDF; or
g. SPARCL1 and FCER2 and CANX and PEDF and HPX; or
h. SPARCL1 and FCER2 and CANX and PEDF and HPX and HRNR; or
i. SPARCL1 and FCER2 and CANX and PEDF and HPX and HRNR and KRT13 and AMBP; or
j. SPARCL1 and FCER2 and CANX and PEDF and HPX and HRNR and KRT13 and AMBP and LYVE1; or
k. SPARCL1 and FCER2 and CANX and PEDF and HPX and HRNR and KRT13 and AMBP and LYVE1 and CD99.

In certain embodiments, the biomarker is PEDF, and a concentration of PEDF is determined by mass spectrometry, and an intensity threshold score to detect men who should perform a prostate biopsy is below 100,000.

In certain embodiments, the concentration of the biomarkers is used to calculate a score value,
particularly wherein the score value is calculated by the following formula:

$$Score = {β}_{0} + {β}_{1} ⋆ {x}_{1} + {β}_{2} ⋆ {x}_{2} + … + {β}_{n} ⋆ {x}_{n}$$

wherein
Depending on the score, the subject has a high or low probability to have prostate cancer, particularly high-grade prostate cancer and/or a PI-RADS score of 3-5;
“β” values are the regression coefficients,
“x” values are the measured concentrations of the respective proteins in urine samples or the value of clinical data, particularly age and/or PI-RADS score.
β₀ is the intercept,
index “n” represents the number of variables used.

The logistic regression model used in all the results of combinatory analysis provides an estimate of the coefficients to be used in the equation. For example, the coefficients of Table 7, 9 and 10 can be used.

The regression coefficients are determined beforehand with an optimization (typically a maximization of the AUC in a ROC approach using experimental data).

The result is the probability for an observation with the given pattern of values of the independent variables to have the event. These results are the scores that are used to build the ROC curves.

The values for the shown examples are listed in Table 7, 9 and 10, see one particular example at the end.

In certain embodiments, the age and/or PI-RADS of the subject contributes to the calculation of the score value.

In certain embodiments, the biomarker concentration is determined via mass spectrometry, and the score value is calculated by the following formula:

$$Score = 5.075 + ( - 0.00005188 ⋆ {x}_{1} ) + ( - 0.000008438 ⋆ {x}_{2} )$$

wherein:
Depending on the score, the subject has a high or low probability to have prostate cancer, particularly high-grade prostate cancer and/or a PI-RADS score of 3-5;
x₁=MS intensity of patient x for PEDF
   x₂=MS intensity of patient x for FCER2.

In this example, a score of below −1.22 is a true negative (threshold for 100% sensitivity).

### Ranges for MS Formula:

In certain embodiments, β₀ is in the range of −10,000 to 10,000. In certain embodiments, β₀ is in the range of −1000 to 1000. In certain embodiments, β₀ is in the range of −10 to 10. In certain embodiments, β₀ is in the range of 4 to 6.

In certain embodiments, β₁ is in the range of −10,000 to 10,000. In certain embodiments, β₁ is in the range of −1000 to 1000. In certain embodiments, β₁ is in the range of −10 to 10. In certain embodiments, β₁ is in the range of −1 to 1.

In certain embodiments, β₂ is in the range of −10,000 to 10,000. In certain embodiments, β₂ is in the range of −1000 to 1000. In certain embodiments, β₂ is in the range of −10 to 10. In certain embodiments, β₂ is in the range of −1 to 1.

In certain embodiments, βₙ is in the range of −10,000 to 10,000. In certain embodiments, βₙ is in the range of −1000 to 1000. In certain embodiments, βₙ is in the range of −10 to 10. In certain embodiments, βₙ is in the range of −1 to 1.

In certain embodiments, Score is in the range of −100 to 1000.

In certain embodiments, the biomarker concentration is determined via ELISA, and the score value is calculated by the following formula:

$$Score = 1.931 + ( - 0.6994 ⋆ {x}_{1} ) + ( - 0.001579 ⋆ {x}_{2} )$$

wherein:
Depending on the score, the subject has a high or low probability to have prostate cancer, particularly high-grade prostate cancer and/or a PI-RADS score of 3-5;
x₁=ELISA quantification of patient x for PEDF concentration
   x₂=ELISA quantification of patient x for FCER2 concentration.

In this example, a score of below −1.3 is a true negative (threshold for 100% sensitivity).

In certain embodiments, the biomarker concentration is determined via ELISA, and the score value is calculated by the following formula:

$$Score = 0.4349 + ( - 6.045 ⋆ {x}_{1} ) + ( - 0.001971 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for KRT13 concentration
   x₂=ELISA quantification of patient x for FCER2 concentration.

In certain embodiments, the biomarker concentration is determined via ELISA, and the score value is calculated by the following formula:

$$Score = 0.3256 + ( - 38.53 ⋆ {x}_{1} ) + ( - 19.75 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for PEDF concentration
   x₂=ELISA quantification of patient x for CD99 concentration.

In certain embodiments, the biomarker concentration is determined via ELISA, and the score value is calculated by the following formula:

$$Score = 0.4546 + ( - 0.007238 ⋆ {x}_{1} ) + ( - 6.736 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for HPX concentration
   x₂=ELISA quantification of patient x for HRNR concentration.

In certain embodiments, the biomarker concentration is determined via ELISA, and the score value is calculated by the following formula:

$$Score = 1.071 + ( - 427.2 ⋆ {x}_{1} ) + ( - 2.875 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for CD99 concentration
   x₂=ELISA quantification of patient x for HRNR concentration.

In certain embodiments, the biomarker concentration is determined via ELISA, and the score value is calculated by the following formula:

$$Score = 1.237 + ( - 10.22 ⋆ {x}_{1} ) + ( 5605 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for CD99 concentration
   x₂=ELISA quantification of patient x for SPARCL1 concentration.

In certain embodiments, the biomarker concentration is determined via ELISA, and the score value is calculated by the following formula:

$$Score = 1.413 + ( - 1.307 ⋆ {x}_{1} ) + ( - 22.82 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for AMBP concentration
   x₂=ELISA quantification of patient x for SPARCL1 concentration.

In certain embodiments, the biomarker concentration is determined via ELISA, and the score value is calculated by the following formula:

$$Score = 1.434 + ( - 0.1478 ⋆ {x}_{1} ) + ( - 222.5 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for KRT13 concentration
   x₂=ELISA quantification of patient x for LYVE1 concentration.

### Ranges for ELISA Formula:

In certain embodiments, β₀ is in the range of −10,000 to 10,000. In certain embodiments, β₀ is in the range of −1000 to 1000. In certain embodiments, β₀ is in the range of −10 to 10. In certain embodiments, β₀ is in the range of 4 to 6.

In certain embodiments, β₁ is in the range of −10,000 to 10,000. In certain embodiments, β₁ is in the range of −1000 to 1000. In certain embodiments, β₁ is in the range of −10 to 10. In certain embodiments, β₁ is in the range of −1 to 1.

In certain embodiments, β₂ is in the range of −10,000 to 10,000. In certain embodiments, β₂ is in the range of −1000 to 1000. In certain embodiments, β₂ is in the range of −10 to 10. In certain embodiments, β₂ is in the range of −1 to 1.

In certain embodiments, βₙ is in the range of −10,000 to 10,000. In certain embodiments, βₙ is in the range of −1000 to 1000. In certain embodiments, βₙ is in the range of −10 to 10. In certain embodiments, βₙ is in the range of −1 to 1.

Score is in the range of −100 to 1000.

In certain embodiments, collecting information about the health status comprises determining whether the subject has, or is at risk of developing prostate cancer. In certain embodiments, collecting information about the health status comprises determining whether the subject has, or is at risk of having a high-grade prostate cancer. In certain embodiments, collecting information about the health status comprises determining whether the subject has, or is at risk of biochemical recurrence. In certain embodiments, collecting information about the health status comprises determining whether the subject has, or is at risk of relapsing. In certain embodiments, collecting information about the health status comprises determining whether the subject is likely to benefit from a biopsy. In certain embodiments, collecting information about the health status comprises determining whether the subject is likely to benefit from active treatment. In certain embodiments, collecting information about the health status comprises determining whether the subject is likely to benefit from active surveillance. In certain embodiments, collecting information about the health status comprises determining whether the subject is likely to benefit from prostatectomy. In certain embodiments, collecting information about the health status comprises determining whether the subject is likely to benefit from chemotherapy or radiotherapy or hormone depletion treatment.

The invention further encompasses the use of ELISA, SIMOA, and/or mass spectrometry for biomarker quantification as identified herein for use in the manufacture of a kit for the determination of the health status of a human subject, particularly for the assessment of the subject's likelihood to be diagnosed with prostate cancer or the need to undergo biopsy. Thus, the present invention also relates to a corresponding kit.

Wherever alternatives for single separable features are laid out herein as “embodiments”, it is to be understood that such alternatives may be combined freely to form discrete embodiments of the invention disclosed herein.

The specification further encompasses the following items:

### Items:

1. A method for collecting information about the health status of a human subject, said method comprising the quantitative detection, in a sample obtained from the subject, of the concentration of a biomarker selected from Table 5.1, establishing the statistical significance of the concentration of the biomarker.

2. The method according to item 1, wherein the concentration of more than one biomarker is determined, and optionally combined with clinical data of the human subject.

3. The method according to any one of the preceding items, wherein a biomarker of at least one, two or of each column of Table 4 is determined.

4. The method according to any one of the preceding items, wherein the sample is a urine or blood sample, particularly wherein the sample is a urine sample.

5. The method according to any one of the preceding items 1 to 4, wherein the concentration is determined by ELISA.

6. The method according to any one of the preceding items 1 to 4, wherein the concentration is determined by SIMOA.

7. The method according to any one of the preceding items 1 to 4, wherein the concentration is determined by mass spectrometry.

8. The method according to any one of the preceding items, wherein the concentration of the following biomarkers is determined:
a. PEDF and FCER2; or
b. PEDF and CANX; or
c. HPX and KRT13; or
d. PEDF and FCER2 and CANX; or
e. PEDF and FCER2 and CANX and KRT13; or
f. PEDF and FCER2 and CANX and KRT13 and HPX; or
g. PEDF and FCER2 and CANX and KRT13 and HPX and HRNR; or
h. PEDF and FCER2 and CANX and KRT13 and HPX and HRNR and CD99
i. PEDF and FCER2 and CANX and KRT13 and HPX and HRNR and CD99 and SPARCL1
j. PEDF and FCER2 and CANX and KRT13 and HPX and HRNR and CD99 and SPARCL1 and AMBP
k. PEDF and FCER2 and CANX and KRT13 and HPX and HRNR and CD99 and SPARCL1 and AMBP and LYVE1.

9. The method according to item 8, wherein the biomarker is PEDF, and a concentration of PEDF is determined by mass spectrometry, and an intensity threshold score to detect men who should perform a prostate biopsy is below 100,000.

10. The method according to any one of the preceding items, wherein the concentration of the biomarkers is used to calculate a score value, particularly wherein the score value is calculated by the following formula:

$$Score = {β}_{0} + {β}_{1} ⋆ {x}_{1} + {β}_{2} ⋆ {x}_{2} + … + {β}_{n} ⋆ {x}_{n}$$

wherein
Score is indicative for the probability of the subject to have prostate cancer, particularly high-grade prostate cancer and/or a PI-RADS score of 3-5;
“β” values are the regression coefficients,
“x” values are the measured concentrations of the respective proteins in urine samples or the value of clinical data, particularly age and/or PI-RADS score.
β₀ is the intercept,
index “n” represents the number of variables used.

11. The method according to item 11, wherein the biomarker concentration is determined via mass spectrometry, and
β₀ is in the range of −10,000 to 10,000, particularly β₀ is in the range of −10 to 10, more particularly β₀ is in the range of 4 to 6, and/or
β₁ is in the range of −10,000 to 10,000, particularly β₁ is in the range of −10 to 10, particularly β₁ is in the range of −1 to 1, and/or
β₂ is in the range of −10,000 to 10,000, particularly β₂ is in the range of −10 to 10, particularly β₂ is in the range of −1 to 1, and/or
βₙ is in the range of −10,000 to 10,000, particularly βₙ is in the range of −10 to 10, particularly βₙ is in the range of −1 to 1, and/or
Score is in the range of −100 to 1000.

12. The method according to item 11, wherein the biomarker concentration is determined via mass spectrometry, and the score value is calculated by the following formula:

$$Score = 5.075 + ( - 0.00005188 ⋆ {x}_{1} ) + ( - 0.000008438 ⋆ {x}_{2} )$$

wherein:
Score is indicative for the probability of the subject to have prostate cancer, particularly high-grade prostate cancer and/or a PI-RADS score of 3-5;
x₁=MS intensity of patient x for PEDF
   x₂=MS intensity of patient x for FCER2.

13. The method according to item 11, wherein the biomarker concentration is determined via ELISA, and
β₀ is in the range of −10,000 to 10,000, particularly β₀ is in the range of −10 to 10, more particularly β₀ is in the range of 4 to 6, and/or
β₁ is in the range of −10,000 to 10,000, particularly β₁ is in the range of −10 to 10, particularly β₁ is in the range of −1 to 1, and/or
β₂ is in the range of −10,000 to 10,000, particularly β₂ is in the range of −10 to 10, particularly β₂ is in the range of −1 to 1, and/or
βₙ is in the range of −10,000 to 10,000, particularly βₙ is in the range of −10 to 10, particularly βₙ is in the range of −1 to 1, and/or
Score is in the range of −100 to 1000.

14. The method according to item 11, wherein the biomarker concentration is determined via ELISA, and the score value is calculated by the following formula:

$$Score = 1.931 + ( - 0.6994 ⋆ {x}_{1} ) + ( - 0.001579 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for PEDF concentration
   x₂=ELISA quantification of patient x for FCER2 concentration.

15. The method according to item 11, wherein the biomarker concentration is determined via ELISA, and the score value is calculated by the following formula:

$$Score = 0.4349 + ( - 6.045 ⋆ {x}_{1} ) + ( - 0.001971 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for KRT13 concentration
   x₂=ELISA quantification of patient x for FCER2 concentration.

16. The method according to item 11, wherein the biomarker concentration is determined via ELISA, and the score value is calculated by the following formula:

$$Score = 0.3256 + ( - 38.53 ⋆ {x}_{1} ) + ( - 19.75 ⋆ {x}_{2} )$$

wherein:
Score is indicative for the probability of the subject to have prostate cancer, particularly high-grade prostate cancer and/or a PI-RADS score of 3-5;
x₁=ELISA quantification of patient x for PEDF concentration
   x₂=ELISA quantification of patient x for CD99 concentration.

17. The method according to item 11, wherein the biomarker concentration is determined via ELISA, and the score value is calculated by the following formula:

$$Score = 0.4546 + ( - {0.007238}^{*} {x}_{1} ) + ( - {6.736}^{*} {x}_{2} )$$

wherein:
Score is indicative for the probability of the subject to have prostate cancer, particularly high-grade prostate cancer and/or a PI-RADS score of 3-5;
x₁=ELISA quantification of patient x for HPX concentration
   x₂=ELISA quantification of patient x for HRNR concentration.

18. The method according to item 11, wherein the biomarker concentration is determined via ELISA, and the score value is calculated by the following formula:

$$Score = 1.071 + ( - {427.2}^{*} {x}_{1} ) + ( - {2.875}^{*} {x}_{2} )$$

wherein:
Score is indicative for the probability of the subject to have prostate cancer, particularly high-grade prostate cancer and/or a PI-RADS score of 3-5;
x₁=ELISA quantification of patient x for CD99 concentration
   x₂=ELISA quantification of patient x for HRNR concentration.

19. The method according to item 11, wherein the biomarker concentration is determined via ELISA, and the score value is calculated by the following formula:

$$Score = 1.237 + ( - {10.22}^{*} {x}_{1} ) + ( {5605}^{*} {x}_{2} )$$

wherein:
Score is indicative for the probability of the subject to have prostate cancer, particularly high-grade prostate cancer and/or a PI-RADS score of 3-5;
x₁=ELISA quantification of patient x for CD99 concentration
   x₂=ELISA quantification of patient x for SPARCL1 concentration.

20. The method according to item 11, wherein the biomarker concentration is determined via ELISA, and the score value is calculated by the following formula:

$$Score = 1.413 + ( - {1.307}^{*} {x}_{1} ) + ( - {22.82}^{*} {x}_{2} )$$

wherein:
Score is indicative for the probability of the subject to have prostate cancer, particularly high-grade prostate cancer and/or a PI-RADS score of 3-5;
x₁=ELISA quantification of patient x for AMBP concentration
   x₂=ELISA quantification of patient x for SPARCL1 concentration.

21. The method according to item 11, wherein the biomarker concentration is determined via ELISA, and the score value is calculated by the following formula:

$$Score = 1.434 + ( - {0.1478}^{*} {x}_{1} ) + ( - {222.5}^{*} {x}_{2} )$$

wherein:
Score is indicative for the probability of the subject to have prostate cancer, particularly high-grade prostate cancer and/or a PI-RADS score of 3-5;
x₁=ELISA quantification of patient x for KRT13 concentration
   x₂=ELISA quantification of patient x for LYVE1 concentration.

22. The method according to item 11, wherein the age and/or PI-RADS of the subject contributes to the calculation of the score value.

23. The method according to any one of the preceding items, wherein collecting information about the health status comprises determining whether the subject
a. has, or is at risk of developing prostate cancer; and/or
b. has, or is at risk of having a high-grade prostate cancer; and/or
c. has, or is at risk of biochemical recurrence; and/or
d. has, or is at risk of relapsing; and/or
e. is likely to benefit from a biopsy; and/or
f. is likely to benefit from active treatment; and/or
g. is likely to benefit from active surveillance; and/or
h. is likely to benefit from prostatectomy; and/or
i. is likely to benefit from chemotherapy or radiotherapy or hormone depletion treatment.

24. A therapeutic agent for use in treating prostate cancer in a subject, characterized in that the subject has been diagnosed to have prostate cancer with the method of any one of the preceding items.

25. A kit adapted to carry out the method of any one of the preceding items 1 to 23, comprising means for the quantitative detection of at least one of the biomarkers as defined in the preceding items in a sample from the subject and means for comparing the detected amount to a control.

The invention is further illustrated by the following examples and figures, from which further embodiments and advantages can be drawn. These examples are meant to illustrate the invention but not to limit its scope.

### DESCRIPTION OF THE FIGURES AND TABLES

FIG. 1: Examples of combinatory analysis of ELISA data via multiple logistic regression for the identification of all grades or high-grade prostate cancer. Example of biomarker combination PEDF and FCER2 with age and PIRADS for the detection of (A) all grades and (B) high-grade PCa. All data are shown as normalized and not normalized.

FIG. 2: Identification of candidate urine biomarkers by mass spectrometry. (A) Schematic workflow overview of urine biomarker screening via mass spectrometry and validation with ELISA; (B) 2.768 proteins, 23.059 peptides, and 38.454 precursors were quantified across all 43 urine samples. (C) Volcano plot of 2.768 proteins quantified by mass spectrometry. The 351 differently distributed protein candidates are shown in blue (decreased in tumors) and red (increased in tumors) and were defined by: q-value<0.05 and average fold change>1.75. The seven candidates PEDF, HPX, CD99, CANX, FCER2, HRNR, and KRT13 are indicated.

FIG. 3: Potential candidate biomarkers for the detection of healthy men. Mass-spectrometry based quantification of the biomarkers (A) PEDF, HPX, CD99, CANX, FCER2, HRNR, and KRT13 in patients with and without PCa. Results are expressed as box-plots (from the 25th to the 75th percentile and median) with whiskers representing the minimum and the maximum values. Statistical difference was assessed by the unpaired non-parametric Mann-Whitney U test with p≤0.05 defined as statistically significant (ns p>0.05; *p≤0.05; **p≤0.01; ***p≤0.001) (B) Diagnostic performances of the selected biomarkers assessed with the receiver operating characteristic (ROC). Each single biomarker (red curve) has a higher performance compared to serum PSA (black curve, AUC=0.6020). (C) Correlation matrix assessed with the Pearson correlation method showing the correlation coefficients of the seven biomarkers with each other. A correlation between variables is defined as low for values up to ±0.3, medium for values up to ±0.5 and large for values up to ±1. (D) Combinatory analysis of non-correlating biomarkers via multiple logistic regression for the identification of tumor-free men. Coupling of PEDF and FCER2 resulted in the best performing biomarker combination, with an AUC of 0.8773 and a specificity of 72.7% at 100% sensitivity. Combined biomarkers displayed a higher performance compared to the single candidates and to serum PSA (black curve, AUC=0.6020).

FIG. 4: Mass spectrometry analysis of two possible control molecules. Mass spectrometry analysis of two control molecules. Mass-spectrometry quantification of CD44 (A) and RNASE2 (B) showed no significant difference in healthy men compared to patients with PCa, making both molecules good candidates as ELISA data normalizers. A Mann-Whitney test was performed to determine significance.

FIG. 5. Validation of candidate biomarkers with ELISA for the detection of healthy men or high-grade PCa. Commercially available ELISA kits were used and results for PEDF, HPX, CD99, CANX, FCER2, HRNR, and KRT13 are represented as box-plots, where the relative concentration of the biomarkers normalized to two control molecules (CD44 and RNASE2) is compared for men with (A) no tumor to patients with any grade of PCa and (B) men with no tumor or low grade (GS=6) PCa to patients harboring a high-grade tumor (GS≥7).

Significance was assessed with a statistical Mann-Whitney test with p≤0.05 defined as statistically significant (ns p>0.05; *p≤0.05; **p≤0.01; ***p≤0.001). Results are expressed as box-plots (from the 25th to the 75th percentile and median) with whiskers representing the minimum and the maximum values. The diagnostic potential of the single biomarkers was investigated with receiver operating characteristic (ROC) analysis. All biomarkers (purple curve) showed a better performance compared to serum PSA (black curve, all grades AUC=0.6020; high-grade PCa AUC=0.5690).

FIG. 6. Multiple logistic regression analysis for the combination of biomarker levels (quantification by ELISA) with the patient's age. (A) Pearson correlation matrix showing the correlation coefficients of the seven biomarkers, age and serum PSA with each other. A correlation between variables is defined as low for values up to ±0.3, medium for values up to ±0.5 and large for values up to ±1. (B) Combinatory analysis of immunoassay validation for the detection of healthy men. The combination of PEDF and FCER2 resulted as best pair from mass spectrometry and, in addition to age, achieved a final AUC of 0.8022 and a 39.1% specificity at 100% sensitivity. ELISA results revealed that, with an AUC of 0.8196 and a specificity of 52.2%, the best performing combination of biomarker was KRT13, FCER2, and age. Combined biomarkers showed a better performance compared to the single candidates and to serum PSA (black curve, AUC=0.6020). (C) The combination of biomarkers with age can predict the presence of high-grade PCa. PEDF, FCER2, and age achieved a final AUC of 0.7523 and a 44.5% specificity at 100% sensitivity. By combining KRT13, FCER2, and age the performance reached an AUC of 0.7801 and a specificity of 48.1% (serum PSA is represented by the black curve, AUC=0.5690).

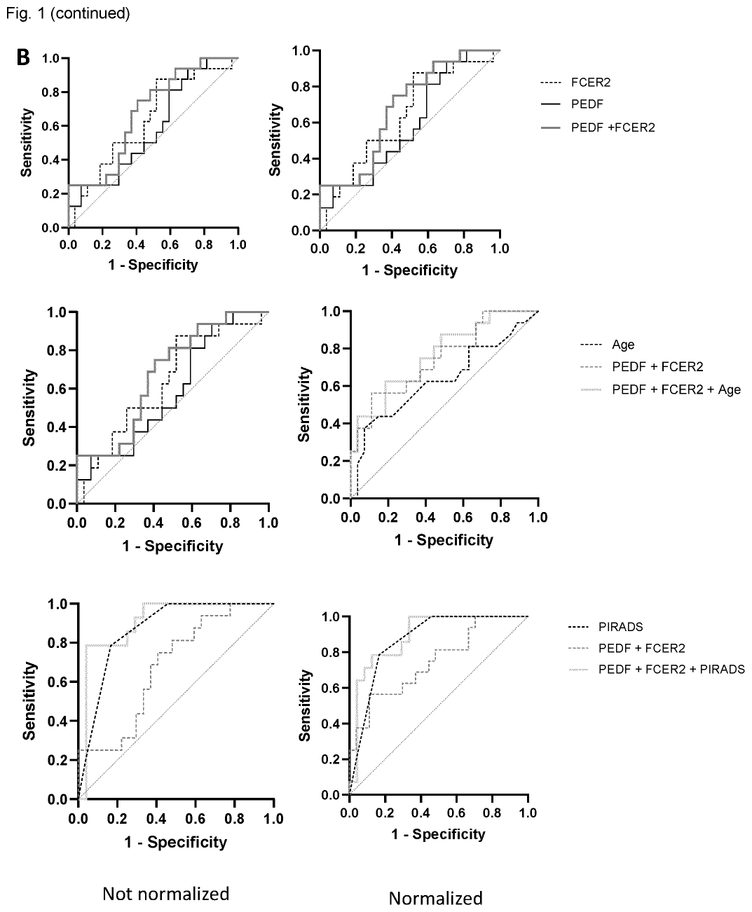

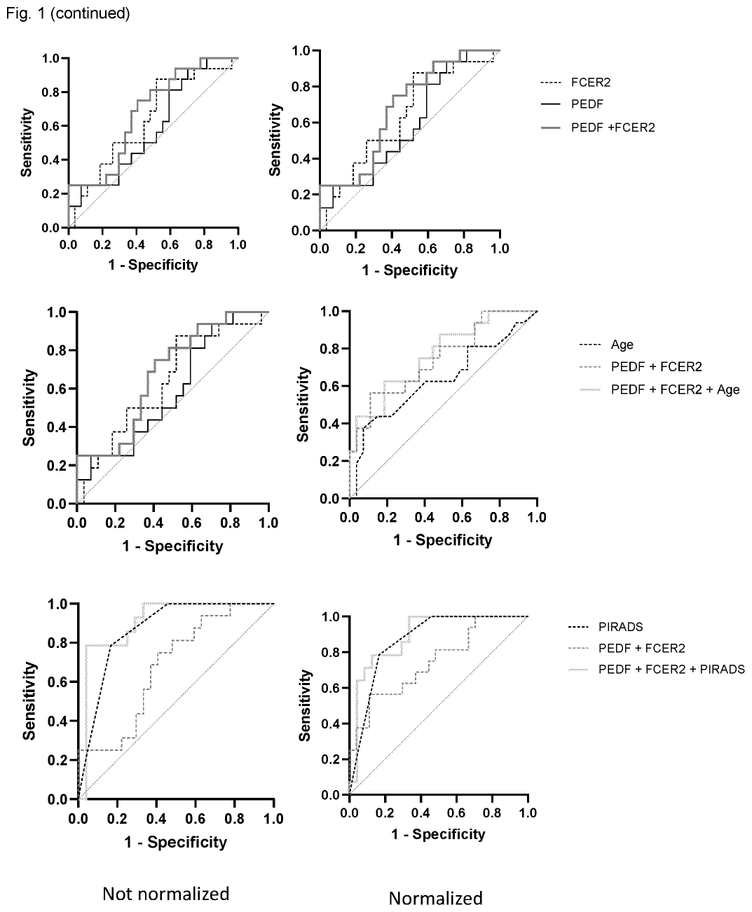

Table 1. TOP 25 candidate biomarkers and three control molecules to detect any grade of prostate cancer identified by MS screening. The Table shows gene and protein names, as well as the Uniprot ID, of the selected biomarkers and controls. Protein intensities for each protein were analyzed using a two sample Student's t-test, and p-values were corrected for overall FDR using the q-value approach. The following thresholds were applied for candidate ranking: q-value<0.05 and absolute average log 2 ratio>0.8074 (fold change>1.75). After removal of proteins that were not identified in at least 90% of the samples, a selection based on ROC analysis was performed in order to identify the final list of the best performing 25 candidates (AUC>0.670 and >10% specificity at 100% sensitivity).

Table 2. TOP 25 candidate biomarkers and three control molecules to detect high grade prostate cancer (GS≥7) identified by MS screening. The Table shows gene and protein names, as well as the Uniprot ID, of the selected biomarkers and controls. Protein intensities for each protein were analyzed using a two sample Student's t-test, and p-values were corrected for overall FDR using the q-value approach. The following thresholds were applied for candidate ranking: q-value<0.05 and absolute average log 2 ratio>0.8074 (fold change>1.75). After removal of proteins that were not identified in at least 90% of the samples, a selection based on ROC analysis was performed in order to identify the final list of the best performing 25 candidates (AUC>0.610 and >25% specificity at 100% sensitivity).

Table 3. 3.1 TOP 25 candidate biomarkers and three control molecules to detect PIRADS score (PIRADS≥3) identified by MS screening. The Table shows gene and protein names, as well as the Uniprot ID, of the selected biomarkers and controls. Protein intensities for each protein were analyzed using a two sample Student's t-test, and p-values were corrected for overall FDR using the q-value approach. The following thresholds were applied for candidate ranking: p-value<0.05 and absolute average log 2 ratio>0.8074 (fold change>1.75). After removal of proteins that were not identified in at least 90% of the samples, a selection based on ROC analysis was performed in order to identify the final list of the best performing 25 candidates (AUC>0.670 and >35% specificity at 90% sensitivity). 3.2 ELISA quantification. The Table shows the results with ELISA quantification of 3 biomarkers, normalized with the controls.

Table 4. Top 25 biomarkers identified by MS screening. The three columns show the top 25 biomarkers for the detection of all PCa grades (GS=6-9), high-grade PCa (GS=7-9) or PI-RADS score 3-5. Some biomarkers are listed in more than one case, that means that they can be used with different thresholds to detect both conditions, e.g. All tumors or high-grade tumors only.

Table 5. Summary of 60 biomarkers and 3 controls identified by MS screening. 5.1 The table shows gene name, protein name and Uniprot ID of the selected biomarkers molecules from all three conditions of Table 4. 5.2 Shows the controls.

Table 6. ROC analysis of MS results for single biomarkers from table 4 for the detection of all or high-grade prostate cancer. The Table shows gene names, protein names, Uniprot ID, statistical values generated by ROC analysis of the selected biomarkers and controls. Specificity for the identification of both, all grades and high-grade PCa, is indicated at 90% and 100% sensitivity.

Table 7. ROC and multiple logistic regression analysis examples of MS results for single or combined biomarkers with or without clinical data for the detection of all or high-grade prostate cancer. 7.1) the table shows gene names, protein names, Uniprot ID, statistical values generated by ROC analysis or multiple logistic regression of the selected biomarkers, clinical data and their combinations. Specificity for the identification of both, all grades and high-grade PCa, is indicated at 90% and 100% sensitivity. 7.2) shows the “β” variables estimates obtained with multiple logistic regression. “???” indicates coefficients that are not possible to calculate when the number of variable is too high compared to the size of the cohort.

Table 8. ROC analysis of ELISA results for single biomarkers selected from table 4 for the detection of all or high-grade prostate cancer. The table shows gene names, protein names, Uniprot ID, statistical values generated by ROC analysis of the selected biomarkers (normalized and not normalized), and controls. Specificity for the identification of both, all grades and high-grade PCa, is indicated at 90% and 100% sensitivity.

Table 9. ROC and multiple logistic regression analysis examples of ELISA results for single or combined biomarkers with or without clinical data for the detection of all or high-grade prostate cancer. 9.1) the table shows gene names, protein names, Uniprot ID, statistical values generated by ROC analysis or multiple logistic regression of the selected biomarkers, clinical data and their combinations (with normalized or not normalized data). Specificity for the identification of both, all grades and high-grade PCa, is indicated at 90% and 100% sensitivity. 9.2) shows the “β” variables estimates obtained with multiple logistic regression.

Table 10. ROC and multiple logistic regression analysis examples of ELISA results for single or combined biomarkers with or without clinical data for the prediction of PI-RADS. 10.1) the table shows gene names, protein names, Uniprot ID, statistical values generated by ROC analysis or multiple logistic regression of the selected biomarkers, clinical data and their combinations (with normalized or not normalized data). Specificity for the identification of both, all grades and high-grade PCa, is indicated at 90% and 100% sensitivity. 10.2) shows the “β” variables estimates obtained with multiple logistic regression.

Table 11: Demographic and clinical characteristics of the patients enrolled in the discovery cohort. Statistical analysis was performed using a Mann-Whitney U test, which showed age as the only variable significantly different between the “Tumor” and the “No Tumor” groups (p=0.048). * Data available for only 41 patients.

Table 12: Commercial ELISA kits used for the validation of biomarker candidates.

Table 13: Top 25 biomarkers and two control molecules resulted from mass spectrometry screening. The upper part of the table shows the top 25 biomarkers upon ranking based on mass spectrometry results, as well as diagnostic performance (AUC and specificity), while the lower part indicates the two control molecules used in the study.

Table 14: ROC curve and multiple logistic regression analysis of the mass spectrometry results. The analysis was performed on the seven biomarker candidates and their possible non-correlating combinations for the identification of healthy men.

Table 15: ROC analysis of the ELISA results for the detection of healthy men and high-grade PCa. The table shows the diagnostic performance of ELISA results obtained normalizing the concentration of the seven candidates with two control molecules (CD44 and RNASE2). The “all PCa grades” analysis identifies healthy men (reaching 100% sensitivity at a specific threshold), whereas the “high-grade (GS 7-9) PCa” analysis identifies true negatives as either healthy men or patients harboring GS 6 PCa (reaching 100% sensitivity at a specific threshold).

Table 16: ROC curve and multiple logistic regression analysis of the ELISA results for the detection of healthy men or high-grade PCa. The seven single biomarkers (not normalized) and their combinations (including patients' age as variable) were analyzed. The “all PCa grades” analysis identifies healthy men (reaching 100% sensitivity at a specific threshold), whereas the “high-grade (GS 7-9) PCa” analysis identifies true negatives as either healthy men or patients harboring GS 6 PCa (reaching 100% sensitivity at a specific threshold).

### EXAMPLES

### Materials and Methods

### Urine Collection and Processing

A total of 45 patients were enrolled in the study at the Urology Department of the University Hospital of Zurich (ZQrich, Switzerland). Samples were collected as first-morning urine from untouched men with high serum PSA levels (≥2 ng/mL) and/or abnormal digital rectal examination (DRE) results, before the performance of the prostate biopsy. After collection, urine samples were let for 30 minutes at room temperature to allow the sedimentation of existent solid debris and impurities. Only the supernatant was further processed by five freezing-thawing cycles in order to lyse cells or cellular particles potentially present. Sample aliquots were then stored at −80° C. until use. Patients' recruitment, urine sample collection and analysis were approved by the authorities of Canton Zurich.

### Mass Spectrometry Analysis

Mass spectrometry analysis was performed by Biognosys AG (Schlieren, Switzerland). All solvents were HPLC-grade from Sigma Aldrich (Switzerland) and all chemicals, if not stated otherwise, were obtained from Sigma Aldrich.

### Sample Preparation

After thawing, sample digestion was performed on single filter units (Sartorius Vivacon 500, 30'000 MWCO HY) following a modified FASP protocol (described by the Max Planck Institute of Biochemistry, Martinsried, Germany). Samples were denatured with Biognosys' Denature Buffer and reduced/alkylated using Biognosys' Reduction/Alkylation Solution for 1 h at 37° C. Subsequently, digestion to peptides was carried out using 1 μg trypsin (Promega) per sample, overnight at 37° C.

### Clean-Up for Mass Spectrometry

Peptides were desalted using C18 UltraMicroSpin columns (The Nest Group) according to the manufacturer's instructions and dried down using a SpeedVac system. Peptides were resuspended in 17 μl LC solvent A (1% acetonitrile, 0.1% formic acid (FA)) and spiked with Biognosys' iRT kit calibration peptides. Peptide concentrations were determined using a UV/VIS Spectrometer (SPECTROstar Nano, BMG Labtech).

### HPRP Fractionation

For HPRP fractionation of peptides, digested samples were pooled. Ammonium hydroxide was added to a pH value>10. The fractionation was performed using a Dionex UltiMate 3000 RS pump (Thermo Scientific™) on an Acquity UPLC CSH C18 1.7 μm, 2.1×150 mm column (Waters). The gradient was 1% to 40% solvent B in 30 min, solvents were A: 20 mM ammonium formatein water, B: acetonitrile. Fractions were taken every 30 seconds and sequentially pooled to 12 fraction pools. These were dried down and resolved in 15 μl solvent A. Prior to mass spectrometric analyses, they were spiked with Biognosys' iRTkit calibration peptides. Peptide concentrations were determined using a UV/VIS Spectrometer (SPECTROstar Nano, BMG Labtech).

### Shotgun LC-MS/MS for Spectral Library Generation

For shotgun LC-MS/MS measurements, 2 μg of peptides per fraction were injected to an in-house packed C18 column (Dr. Maisch ReproSilPur, 1.9 μm particle size, 120 A pore size; 75 μm inner diameter, 50 cm length, New Objective) on a Thermo Scientific Easy nLC 1200 nano-liquid chromatography system connected to a Thermo Scientific™ Q Exactive™ HF mass spectrometer equipped with a standard nano-electrospray source. LC solvents were A: 1% acetonitrile in water with 0.1% FA; B: 15% water in acetonitrile with 0.1% FA. The nonlinear LC gradient was 1-52% solvent B in 60 minutes followed by 52-90% B in 10 seconds, 90% B for 10 minutes, 90%-1% B in 10 seconds and 1% B for 5 minutes. A modified TOP15 method from Kelstrup was used [1]. Full MS covered the m/z range of 350-1650 with a resolution of 60'000 (AGC target value was 3e6) and was followed by 15 data dependent MS2 scans with a resolution of 15'000 (AGC target value was 2e5). MS2 acquisition precursor isolation width was 1.6 m/z, while normalized collision energy was centered at 27 (10% stepped collision energy) and the default charge state was 2+.

### HRM Mass Spectrometry Acquisition

For DIA LC-MS/MS measurements, 2 μg of peptides and 1 IE of PQ500 reference peptides were injected per sample. For samples with less than 2 μg total peptide available, the amount of reference peptides was adjusted accordingly. Peptides were injected into an in-house packed C18 column (Dr. Maisch ReproSil Pur, 1.9 μm particle size, 120 A pore size; 75 μm inner diameter, 50 cm length, New Objective) on a Thermo Scientific Easy nLC1200 nano-liquid chromatography system connected to a Thermo Scientific Q Exactive HF mass spectrometer equipped with a standard nano-electrospray source. LC solvents were A: 1% acetonitrile in water with 0.1% FA; B: 15% water in acetonitrile with 0.1% FA. The nonlinear LC gradient was 1-55% solvent B in 120 minutes followed by 55-90% B in 10 seconds, 90% B for 10 minutes, 90%-1% B in 10 seconds and 1% B for 5 minutes. A DIA method with one full range survey scan and 22 DIA windows was used.

### Database Search of Shotgun LC-MS/MS Data and Spectral Library Generation

The shotgun mass spectrometric data were analyzed using Biognosys' search engine SpectroMine™, the false discovery rate on peptide and protein level was set to 1%. A human UniProt .fasta database (Homo sapiens, 2019-07-01) was used for the search engine, allowing for 2 missed cleavages and variable modifications (N-term acetylation, methionine oxidation, deamidation (NQ), carbamylation (KR)). The results were used for generation of a sample-specific spectral library.

### HRM Data Analysis

HRM mass spectrometric data were analyzed using Spectronaut™ 14 software (Biognosys). The false discovery rate (FDR) on peptide and protein level was set to 1% and data was filtered using row-based extraction. The spectral library generated in this study was used for the analysis. The HRM measurements analyzed with Spectronaut™ were normalized using global normalization.

### Data Analysis

For testing of differential protein abundance, MS1 and MS2 protein intensity information was used [2]. Protein intensities for each protein were analyzed using a two sample Student's t-test, and p-values were corrected for overall FDR using the q-value approach [3]. The following thresholds were applied for candidate ranking: q-value<0.05 and absolute average log 2 ratio>0.8074 (fold change>1.75). After removal of proteins that were not identified in at least 90% of the samples, a selection based on ROC analysis was performed in order to identify the final list of the best performing 25 candidates (AUC>0.670 and >10% specificity at 100% sensitivity).

### ELISA Validation

Validation of mass spectrometry results was performed using commercially available ELISA kits and following the manufacturers' protocols (Table 12). Before use, urine sample aliquots were equilibrated to room temperature. Measurements were conducted using the Epoch 2 microplate reader (BioTek, Switzerland) and data were analyzed with the Gen5 software (version 2.09, BioTek, Switzerland).

### Statistics and Data Analysis

All statistical analyses (except for mass spectrometry data) were performed with the GraphPad prism software, version 9. Continuous variables were expressed as box-plots (from the 25th to the 75th percentile and median), with whiskers representing the minimum and the maximum values. Statistical significance was calculated with the unpaired non-parametric Mann-Whitney U test.

For the characterization of single biomarkers, ROC curve analysis was performed applying the Wilson/Brown method, whereas for combinatorial analysis of non-correlated proteins, a multiple logistic regression was applied. The correlation matrix was assessed with the Pearson correlation method.

An online tool was used to draw volcano plots (VolcaNoseR, https://huygens.science.uva.nl/VolcaNoseR/).

### Example 1: Patient Characteristics of the Discovery Cohort

A total of 45 consecutive men with suspected PCa were enrolled in this study and underwent a prostate biopsy after urine sample collection. Their demographic and clinical characteristics are summarized in Table 11, including age, serum PSA and prostate volume. Biopsy results are classified according to the Gleason score (GS) and evaluated for diagnostic purposes by genitourinary pathologists at the University Hospital Zurich. PCa was detected in 46.7% (21/45) and clinically significant PCa (GS 7-9) in 37.8% of the patients. More precisely, 8.9% of the patients were diagnosed with GS 6, 17.8% with GS 7a/b, and 20.0% harbored a GS 8 or GS 9 tumor. Gleason score follow-up at repeated biopsies or upon prostatectomy showed that only one patient was upgraded.

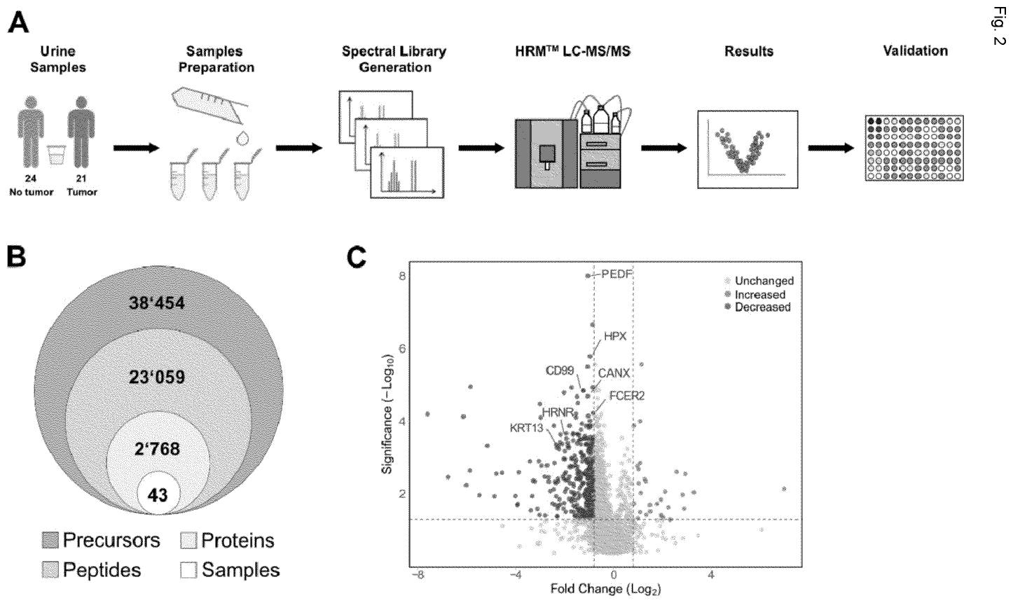

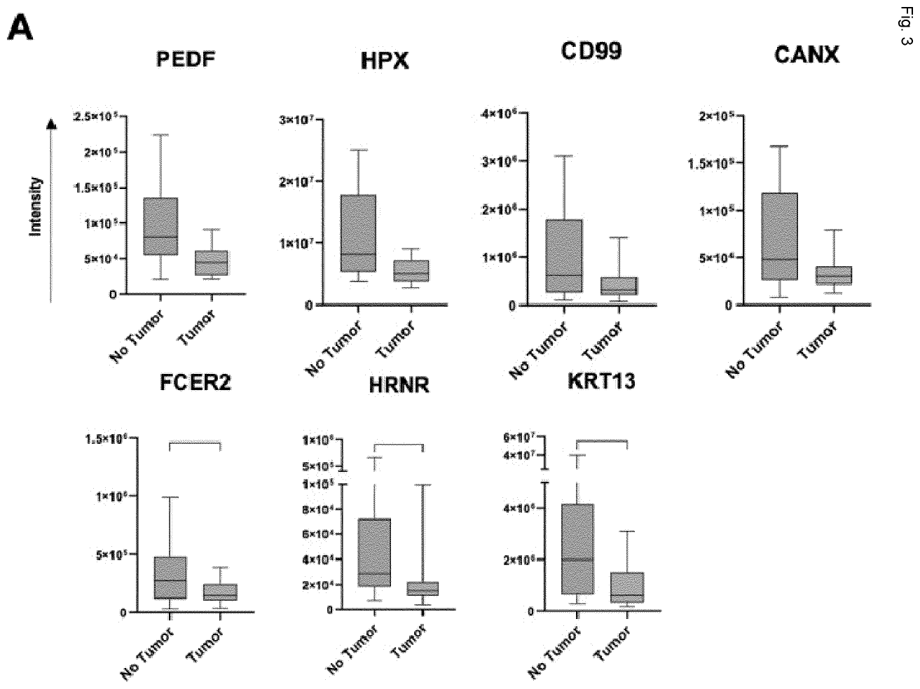

Collected urine samples were then screened by MS and potential novel biomarkers analyzed by ELISA (FIG. 2A).

### Mass Spectrometry Screening and Selection of Urine Biomarkers for PCa Detection

For mass-spectrometry, a spectral peptide library was generated by shotgun LC-MS/MS of high-pH reversed-phase chromatography (HPRP) fractions from all 45 urine samples. Two samples showed a significant contamination with albumin, which led to the suppression of other peptide signals, and were therefore excluded from further analysis (data not shown). We identified a total of 38.454 precursors (peptides including different charges and modifications), corresponding to 23.059 unique peptides and 2.768 proteins across all 43 urine samples by using a false discovery rate of 1% (FIG. 2B).

For the identification of candidate biomarkers to detect healthy men, we compared the abundance of 2.768 proteins in samples from patients not affected by tumor and those with PCa. Significantly dysregulated proteins were identified by setting the q-value below 0.05, at an average fold change of more than 1.75, resulting in 351 biomarker candidates (FIG. 2C).

Strikingly, most of the candidates (321) displayed decreased levels in the urine of PCa patients compared to healthy men. In contrast, only 30 candidate biomarker candidates were found to have increased levels in the “tumor” group.

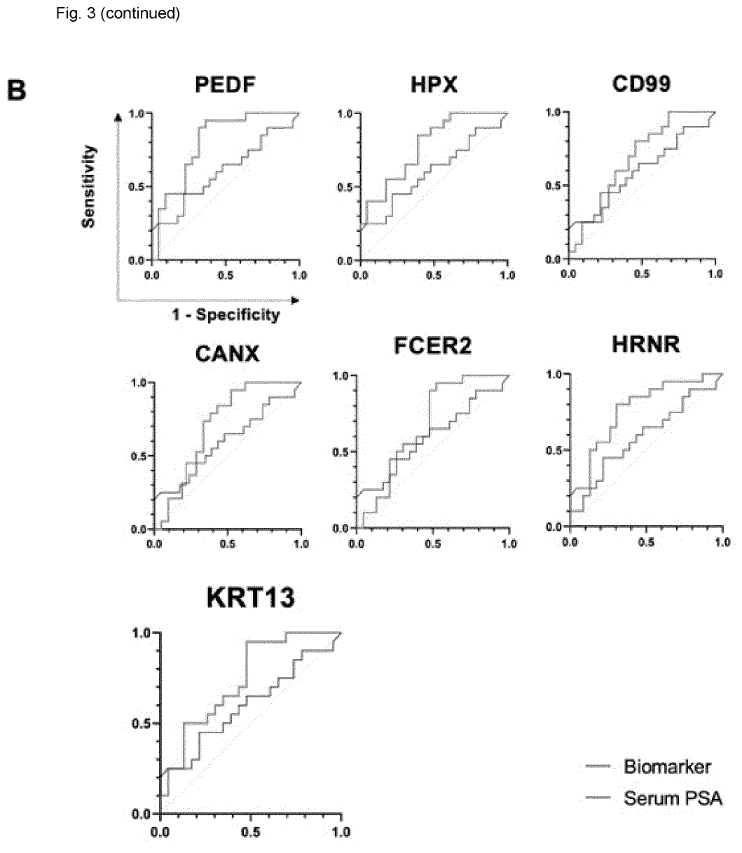

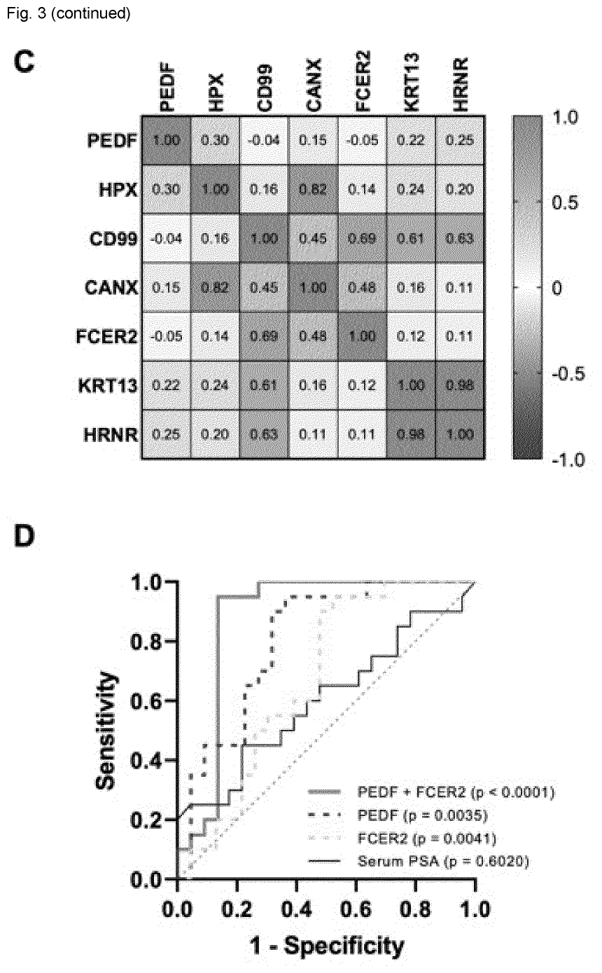

A key selection criterion for the best target molecules from the screening was the ability to discriminate healthy patients (with high specificity and accuracy), achieving a negligible number of false negatives (sensitivity>90%). For this reason, all proteins that were not detected in more than three samples were excluded from further analysis. Additionally, proteins with low diagnostic performances, displaying a receiver operating characteristic (ROC) area under the curve (AUC) smaller than 0.670 and a specificity of less than 10% at 100% sensitivity, were removed. This ranking resulted in 43 biomarkers, with the top 25 candidates listed in Table 13. Among them, pigment epithelium-derived factor (PEDF), hemopexin (HPX), cluster of differentiation 99 (CD99), calnexin precursor (CANX), FCER2 (CD23, Fc fragment Of IgE receptor II), hornerin (HRNR), and keratin 13 (KRT13) showed remarkable diagnostic performance (FIG. 3A,B; Table 14) and were selected for further validation by means of commercially available ELISA kits. Notably, all these biomarkers showed decreased levels in patients harboring prostate cancer.

The illustrated box plots in FIG. 3A show the intensities of the biomarkers in patients with and without PCa as quantified by MS. All biomarkers identify true negative patients that could be spared from performing an unnecessary prostate biopsy, although the p value was a borderline result in terms of statistical significance for two biomarkers. The ROC plots (FIG. 3B) show the ability of the single biomarkers to detect all PCa (GS 6-9, red curves) in comparison to the current standard of care, which is serum PSA (black curves). Each of the seven biomarkers had a superior performance compared to PSA and was able to correctly classify 100% of patients with PCa, while detecting tumor free men at varying specificities (Table 14).

Taken together, these data demonstrate that urine is a reliable proteomic source of biomarkers for the early detection of PCa and that the seven selected biomarker candidates are capable of sparing a relevant number of men from unnecessary prostate biopsy while avoiding misdiagnosis of patients bearing a prostate tumor.

### Example 2: Increase of PCa Detection Performance Through Combinatory Analysis of Biomarkers

To assess potential biomarker combinations via multiple logistic regression, we first performed a Pearson correlation analysis among biomarker levels in the patient cohort (FIG. 3C). In fact, the combination of variables can improve the performance of a predictive model only if the variables are not correlated to each other. In our analysis, we therefore combined biomarkers with a correlation coefficient of up to 0.3. Since the size of the cohort is limited to 43 patients, combinations of a maximum of two biomarkers were taken into consideration, in order to prevent the generation of overfitted models. All possible 14 combinations of biomarkers revealed a significantly larger AUC compared to the null hypothesis of AUC=0.5 (Table 14). Moreover, any combination of two proteins led to a superior diagnostic performance, with increased AUC and higher specificity at 90% and 100% sensitivity compared to the single biomarkers. As an example, FIG. 3D illustrates the multiple logistic regression curve of the PEDF and FCER2 combination (red line), which reached the best specificity of 72.7% at 100% sensitivity. This indicates that potentially 72.7% of healthy men could be spared from performing an unnecessary biopsy.

Our data show that the combination of biomarkers markedly improves the diagnostic power of the model and leads to the superior detection of healthy patients who could be spared from a prostate biopsy.

### Example 3: Validation of Biomarker Performance by ELISA

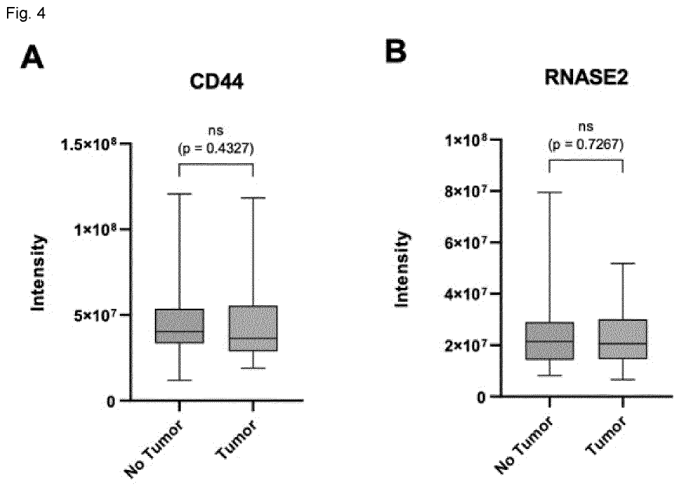

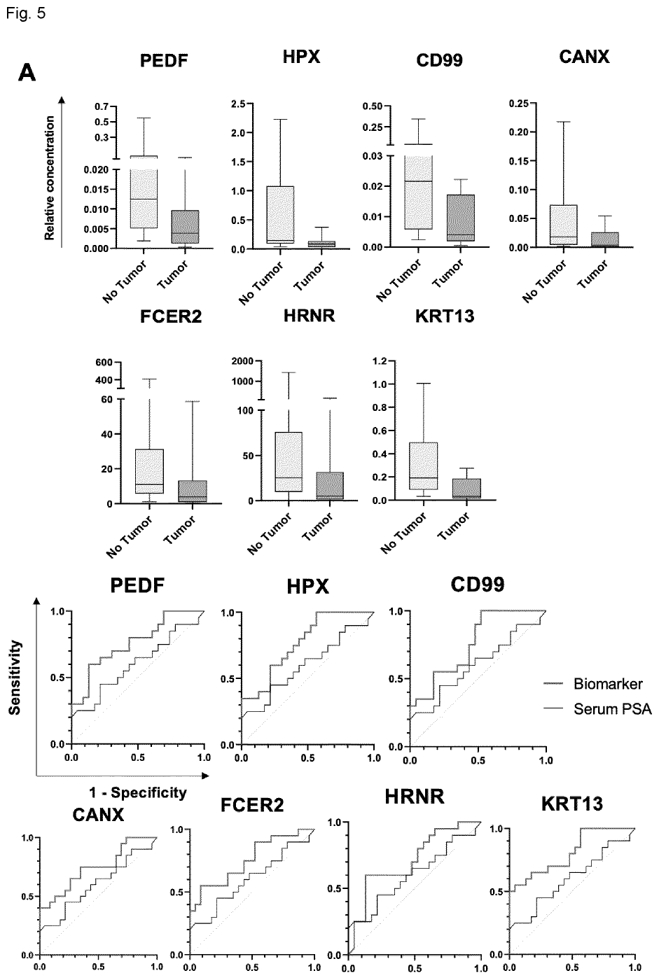

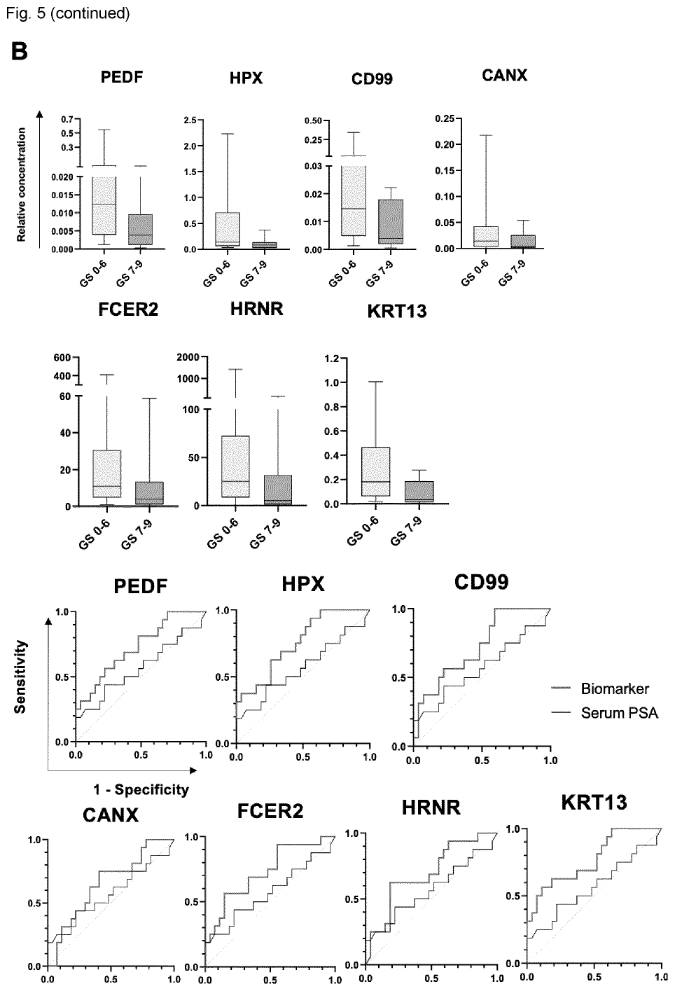

The validation of the candidate proteins selected from the MS analysis was performed by ELISA. Conversely to MS, immunoassays are standardized techniques that can be easily performed in any laboratory and allow for easy comparison among cohorts. For the MS measurements, the different urine samples were normalized according to their total peptide concentration and a defined amount of 2 μg was injected for each run. This approach cannot be applied to ELISA. Nevertheless, normalization is necessary to compensate for variations due to diet, time of collection and physiological characteristics of patients. Therefore, we have chosen non-dysregulated molecules from the mass-spectrometry analysis, i.e., cluster of differentiation 44 (CD44) and ribonuclease A family member 2 (RNASE2) and used them as controls for ELISA quantification of the single biomarkers (FIG. 4). Consistent with the corresponding MS data, Mann-Whitney U analysis of the normalized ELISA data for each analyte showed a significant difference between patients diagnosed with PCa and healthy individuals (FIG. 5A). Furthermore, ROC curve analysis is concurrent with each MS dataset, demonstrating that all biomarkers have the diagnostic potential to detect healthy men at 100% sensitivity (Table 15).

Detection of high grade PCa has a relevant clinical impact, as it allows differentiation between patients who would benefit from active surveillance and those who need active treatments. We therefore also tested the potential of our biomarkers to discriminate also PCa GS≥7. The quantitative analysis by ELISA shows that the seven biomarkers can detect high-grade PCa with high performance (FIG. 5B, Table 15).

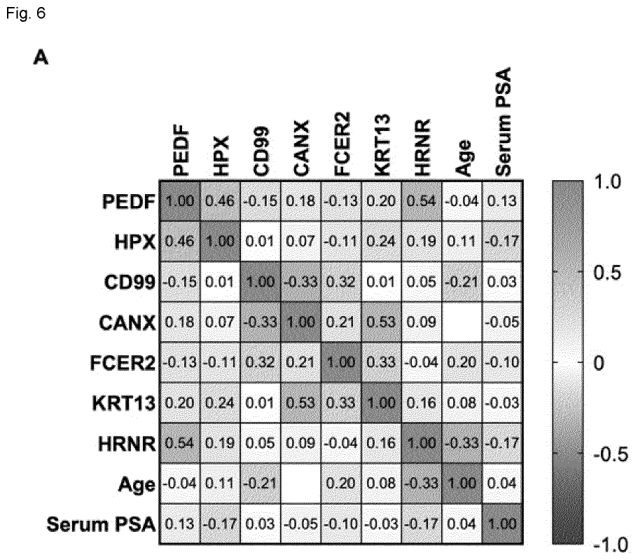

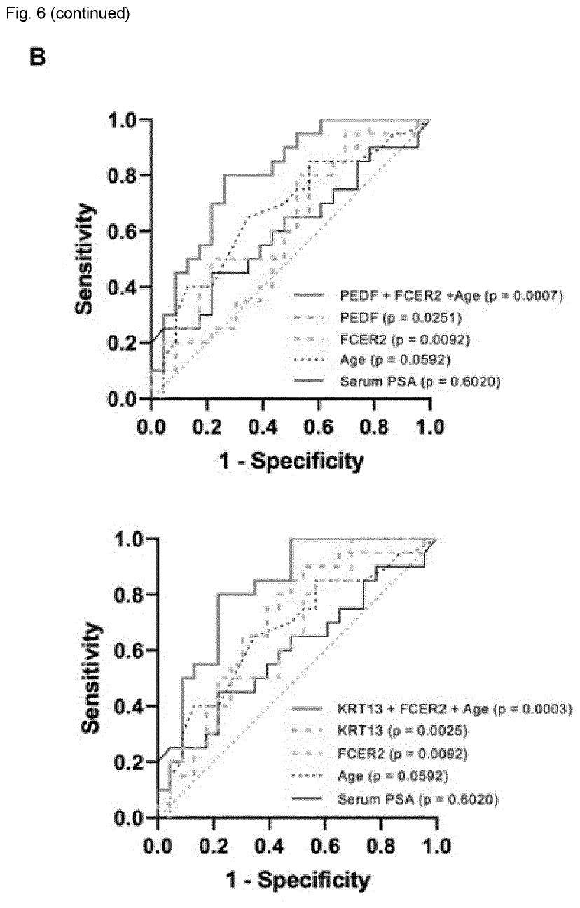

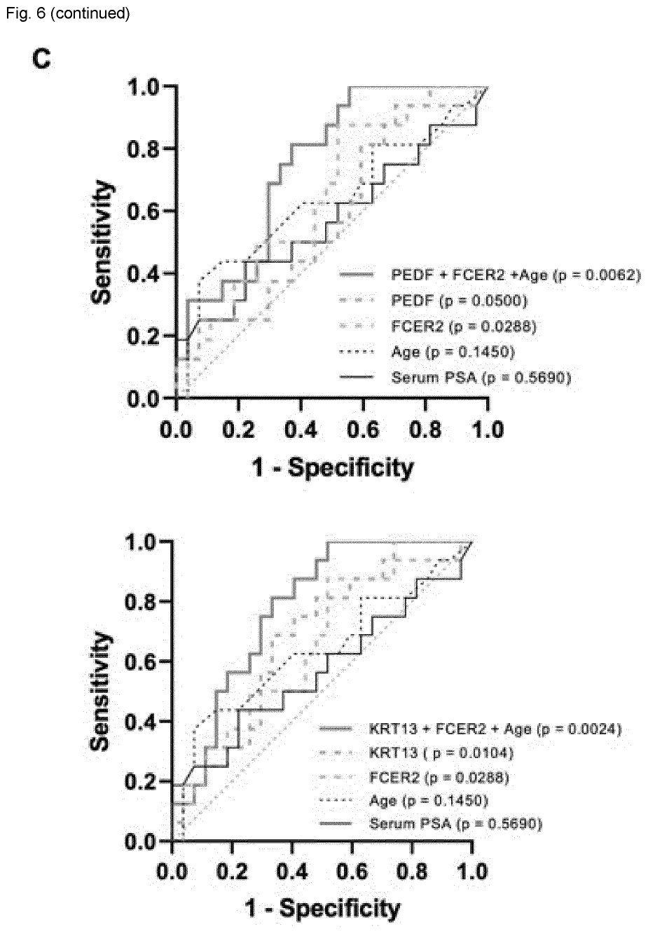

When different biomarkers are normalized by the same controls, as in this study, their combinatory power is hampered by a highly correlated dataset (data not shown), driven by the identical normalization strategy. Hence, combinatorial analysis was performed by multiple logistic regression with non-normalized ELISA data. In this study, we excluded from the nomogram any clinical and demographic information with potentially high variability among individual clinics and cohorts. Prostate volume and digital rectal examination (DRE), for example, are known to be affected by the type of instrument used or by personnel expertise. We therefore included only the age of the patients as clinical variable to improve the predictive models. The Pearson correlation analysis of all variables is shown in FIG. 6A. All combinations, including age, resulted in a significantly higher AUC compared to the null hypothesis and were able to detect all grades of PCa with 100% sensitivity (Table 16). As an example, the ROC curve of two of the best performing combinations, PEDF+FCER2+age and KRT13+FCER2+age showed a specificity of 39.1% and 52.2% at 100% sensitivity, respectively (FIG. 6B). Moreover, for the detection of high-grade tumors, the combination of uncorrelated analytes increased the overall performance of the single biomarkers. As model example, the ELISA quantification of KRT13, FCER2+age showed a striking AUC of 0.7801 with a specificity of 48.1% at 100% sensitivity (FIG. 6C).

Taken together, our data demonstrate that ELISA quantification of the biomarker candidates selected by MS is feasible and confirms the high diagnostic performance of the analytes, both as single and in combination for the detection of all PCa grades and clinically significant tumors (GS≥7).

### DISCUSSION

Despite continuous improvements in the reduction of overdiagnosis and overtreatment of men suspected of having PCa, the number of healthy men that are subject to invasive procedures remains high [Van Poppel, BJU Int.-Br. J. Urol. 2021; Loeb, S Eur. Urol. 2014]. This trend is concordant with our cohort. For this study, patients were selected for prostate biopsy only due to abnormal DRE results and/or elevated PSA levels. Approximately half (53.3%) of patients resulted having no tumor and should have been spared from performing the biopsy (Table 11).

Thus, the aim of this study was to identify novel urine biomarkers to improve the eligibility criteria for prostate biopsy and to more specifically discriminate PCa at an early stage, reducing the number of unnecessary biopsies. Here, we demonstrated the feasibility of diagnostic tests for the screening of PCa relying on urine biomarkers that can be routinely quantified by standardized laboratory methods such as ELISAs.

Urine samples were collected from patients before performing the biopsy and subjected to proteomic screening by mass-spectrometry (MS) to select biomarker candidates that are dysregulated when a prostate tumor is present. Although MS results showed promising results, the application of mass-spectrometry for urine analysis as routine diagnostic test is not feasible, due to the lack of a standard method to compare different batches of samples. A more practical approach is the implementation of quantitative immune-assays such as ELISA, which represents the gold standard for biomarker assessment and validation [Jedinak, A Oncotarget 2018]. Consequently, among the 25 most performant candidates, seven proteins (PEDF, HPX, CD99, FCER2 (CD23), CANX, HRNR, and KRT13) were subsequently quantified in the same urine samples by quantitative ELISA. Additionally, their performance for the diagnosis of PCa and prediction of high-grade tumors was assessed. Although the translation of targeted MS assays into the clinical diagnostic setting appears to be difficult due to high costs and specific expertise requirements [Khoo, A Nat. Rev. Urol. 2021], the validation by ELISA demonstrates the feasibility of a clinical implementation through standard techniques. MS results of the 25 top ranked biomarkers in this study showed a significant decrease in signal intensity when a prostate tumor is present and can identify PCa patients with better performance compared to the standard PSA test (Table 14).

PEDF showed the best performance as a single biomarker, with AUC of 0.8023 and specificity of 36.4% at 100% sensitivity (FIG. 3A,B). On the other hand, as an example of the many possible options (FIG. 3D), the best performing combination of PEDF and FCER2 markedly increase the AUC in predicting PCa compared to each individual marker and also to PSA. Specifically, with this combination 72.7% of unnecessary biopsies could be avoided, without missing any patient with PCa (100% sensitivity).

The proteomic content of urine is affected by many factors, such as individual life-style, diet and time of sampling. For this reason, absolute biomarker data need to be normalized with a different strategy compared to MS, in which normalization is based on the overall cohort protein content. FIG. 5A shows normalized ELISA results of the biomarkers panel, where each single molecule shows a strong diagnostic performance, in concurrence with the MS data. By combining KRT13 and FCER2 with age, we reached an AUC of 0.8196 and a specificity 52.2% at 100% sensitivity (FIG. 6B). Besides the early detection of PCa, risk stratification of patients to better select clinically significant tumors is important to support optimal treatment options. For this reason, we have assessed the ability of the seven biomarkers to also detect tumors with GS≥7 as well. FIG. 5B shows that all candidates can predict the presence of high-grade PCa more precisely than serum PSA. The combination of KRT13 and FCER2 with age for the detection of high-grade PCa reached an AUC of 0.7801 and a specificity of 48.1% at 100% sensitivity (FIG. 4C), thus potentially reducing the number of unnecessary biopsies almost by half, without missing any patient with clinically relevant PCa. Depending on the clinic, region and patients' characteristics (e.g., age and expectation of life), men with low grade PCa (GS 6) will either be monitored or treated by local therapy options. In both cases, the novel biomarker panel can be applied to reduce unnecessary biopsies and monitor patients continuously and non-invasively. Therefore, by combining different biomarkers, we observed a relevant reduction of unnecessary biopsies, either performed on healthy individuals or on patients affected by clinically indolent tumors.

A relevant portion of the proteins identified in our study has already been described in other mass-spectrometry analyses of urine and to a lesser extent, in urinary extracellular vesicles, plasma or prostate tissue of patients. The seven biomarkers validated in our study were chosen exclusively based on their ability to predict PCa prior to biopsy and not considering their biological function. Nevertheless, some of them have been reported to be related to cancer. Although signal reduction in case of tumor progression as described for the seven biomarkers might be surprising, both literature and tissue analysis performed in this study support these findings. Hornerin (HRNR), a member of the fused-type S100 protein family, was shown to be expressed and to play a role in different tumor types [Gutknecht, M. F Nat. Commun. 2017; Choi, J J. Breast Cancer 2016; Fu, S. J. BMC Cancer 2018]. Other members of the same protein family were examined in prostate tissue of PCa patients, demonstrating that the loss of S100A2 and increased expression of S100A4 are hallmarks of PCa progression [Gupta, S.; J. Clin. Oncol. 2003]. Similarly, the prostate tissue analysis of the pigment epithelium-derived factor (PEDF), a natural angiogenesis inhibitor in prostate and pancreas [Doll, J.A Nat. Med. 2003; Halin, S. Cancer Res. 2004], showed minimal expression in high grade PCa (GS 7-10), in contrast to healthy prostate tissue, where the staining shows high intensity [Doll, J.A Nat. Med. 2003]. The downregulation of CD99 was already shown to be essential for tumorigenesis. This has been described for several tumors [Kim, S.H Blood 2000; Manara, M.C. Mol. Biol. Cell 2006; Jung, K.C. J. Korean Med. Sci. 2002], including prostate cancer [Scotlandi, K Oncogene 2007]. In fact, the overexpression of CD99 in prostate cancer cells inhibited their migration and metastatic potential in both in vitro and in vivo experiments [Manara, M.C. Mol. Biol. Cell 2006]. Hemopexin (HPX) has been described to be downregulated in urine from PCa patients compared to tumor free men, an observation that is in concordance with our findings [Davalieva, K Proteomes 2018]. Moreover, a bioinformatics analysis of multiple urinary and tissue proteomes revealed HPX downregulation in high-grade PCa compared to healthy tissue [Lima, T.; Med. Oncol. 2021]. In contrast to our results, elevated levels in cancer have been reported for the remaining molecules. Increased levels of the Fc fragment of IgE receptor II (FCER2) have been implicated in different hematological malignancies and sarcomas [Sarfati, M.; Blood 1988; Caligaris-Cappio, F Best Pr. Res. Clin. Haematol. 2007; Barna, G Hematol. Oncol. 2008; Schlette, E Am. J. Clin. Pathol. 2003; Walters, M. Br. J. Haematol. 2010; Soriano, A. O Am. J. Hematol. 2007]. In addition, FCER2 is expressed in subsets of B cells and in particular depicts follicular dendritic cell networks [PeterRieber, E Springer US: New York, NY, USA, 1993], whereas expression changes in urine could reflect an altered immune microenvironment in prostate adenocarcinoma patients. Keratin 13 (KRT13) belongs to the type I keratin family and its reduced expression has been associated with oral squamous cell carcinoma lesions [/da-Yonemochi, H Mod. Pathol. 2012; Sakamoto, K.; Histopathology 2011; Naganuma, K, BMC Cancer 2014] and bladder cancer [Marsit, C. J PLoS ONE 2010]. In contrast to our results, a study in 2016 revealed a correlation between KRT13 tissue expression and prostate cancer metastasis [Li, Q. Oncotarget 2016]. However, as we could show expression of KRT13 in the basal cells of benign glands, and since the loss of basal cells is one hallmark of prostate adenocarcinoma [Rüschoff, J.H Pathol. Res. Pract. 2021], lower expression levels in urine could also be explained by increased tumoral occupation of the gland. The endoplasmic reticulum chaperone calnexin (CANX) is associated with newly synthesized glycoproteins and involved in correct protein folding [Schrag, J.D Mol. Cell 2001]. So far, CANX has not been described in PCa but its altered expression has been associated with other cancers [Dissemond, J. Cancer Lett. 2004; Ryan, D J. Transl. Med. 2016]. To the best of our knowledge, this is the first study to suggest a putative role in PCa for the above-described biomarkers in PCa, demonstrating their dysregulation at such an early stage (prior to biopsy) and the feasibility of their quantitative assessment in urine.

To investigate the possible origin of the biomarkers and their route to the urine, we performed a sequence-based analysis, predicting secretion pathways of proteins with the SecretomeP 2.0 server (http://www.cbs.dtu.dk/services/SecretomeP/). PEDF, HPX, CD99, and CANX are expressed with signal peptides and potentially traffic through the classical pathway (Golgi apparatus), whereas membrane protein FCER2 was predicted to traffic through a non-classical pathway. Conversely, KRT13 and HRNR do not appear to be secreted. This suggests that the proteins detected may be present in urine due to either the presence of cellular debris or particles deriving directly from the prostate or through blood filtration.

The present study has some limitations. First, it is a retrospective and single institution based study. Second, it relies on a small sample size, combining data of 43 patients for biomarker identification and validation. This became particularly evident when performing the multiple logistic regression analysis, as the cohort size determines the number of variables that can be combined to improve the model. To avoid false associations and large standard errors, a minimum number of five to ten events per predictor variable (EPV) has to be considered [Vittinghoff, E Am. J. Epidemiol. 2006]. Since our cohort comprises 23 healthy men, we included no more than two to four predictor variables. Future studies investigating larger cohort sizes will allow the inclusion of higher numbers of variables and thereby improve their diagnostic performance. Nevertheless, for an explorative analysis of the biomarker candidates, the cohort provided a sufficient sample size and the combination of two to three variables yielded robust prediction models. Although it was currently not possible to validate the biomarkers in an independent cohort, their performance in this study was proved by use of two different and independent quantitative technologies, and the concordance of the findings underscores the importance of further validation of the targets.

### CONCLUSIONS

In conclusion, here, the inventors demonstrated that an upfront urine test based solely on the quantification of novel biomarkers is a feasible approach to improve eligibility criteria for a prostate biopsy and to detect the presence of high-grade PCa, independent of serum PSA, digital rectal examination, and clinical variables. The clinical implementation of a simple urine test represents one possible and safe way to reduce the overdiagnosis and overtreatment of PCa. Furthermore, since it is completely non-invasive, it could potentially be used for disease monitoring and active surveillance.

TABLE 1

| | | | | Student's t-test | | | Mann- | ROC Analysis | | | | | |
|---|---|---|---|---|---|---|---|---|---|---|---|---|---|
| | | | | | | | Whit- | | | | | Speci- | Speci- |
| | | | | Ave- | | | ney | | | 95% | | ficity | ficity |
| | | | | rage | | | U test | | | Con- | | at 90% | at 100% |
| | | | Uniprot | Log2 | p- | q- | p- | | Std. | fidence | p- | Sensi- | Sensi- |
| | Genes | Protein Name | ID | Ratio | value | value | value | AUC | Error | interval | value | tivity | tivity |
| Bio- | SERPINF1 | Pigment | P36955 | −1.039 | 8.60E− | 9.01E− | 0.0006 | 0.8023 | 0.0696 | 0.6659 | 0.0008 | 68.2 | 36.4 |
| marker | | epithelium- | | | 12 | 09 | | | | to | | | |
| | | derived factor | | | | | | | | 0.9386 | | | |
| | CALR | Calreticulin | P27797 | −0.860 | 3.86E− | 2.16E− | 0.0004 | 0.0043 | 0.0686 | 0.6699 | 0.0007 | 47.8 | 34.8 |
| | | | | | 10 | 07 | | | | to | | | |
| | | | | | | | | | | 0.9388 | | | |
| | HPX | Hemopexin | P02790 | −0.952 | 4.25E− | 1.58E− | 0.0016 | 0.7761 | 0.0696 | 0.6396 | 0.0020 | 52.2 | 39.1 |
| | | | | | 09 | 06 | | | | to | | | |
| | | | | | | | | | | 0.9125 | | | |
| | PNP | Purine | P00491 | −5.845 | 6.87E− | 1.10E− | 0.0522 | 0.6739 | 0.0823 | 0.5126 | 0.0514 | 34.8 | 30.4 |
| | | nucleoside | | | 08 | 05 | | | | to | | | |
| | | phosphorylase | | | | | | | | 0.8352 | | | |
| | APDA4 | Apolipo- | P06727 | −0.855 | 1.03E− | 1.15E− | 0.0143 | 0.7174 | 0.0786 | 0.5634 | 0.0149 | 52.2 | 26.1 |
| | | protein | | | 07 | 05 | | | | to | | | |
| | | A-IV | | | | | | | | 0.8714 | | | |
| | CD99 | CD99 antigen | P14209 | −1.231 | 1.66F− | 1.40F− | 0.0534 | 0.6750 | 0.0835 | 0.5114 | 0.0525 | 36.4 | 31.8 |
| | | | | | 07 | 05 | | | | to | | | |
| | | | | | | | | | | 0.8386 | | | |
| | APOA1 | Apolipo- | P02647 | −1.214 | 1.72E− | 1.40E− | 0.0057 | 0.7435 | 0.0768 | 0.5930 | 0.0064 | 52.2 | 17.4 |
| | | protein | | | 07 | 05 | | | | to | | | |
| | | A-IV | | | | | | | | 0.8939 | | | |
| | CANX | Calnexin | P27824 | −1.045 | 2.84E− | 1.99E− | 0.0271 | 0.7043 | 0.0850 | 0.5377 | 0.0273 | 47.6 | 38.1 |
| | | | | | 07 | 05 | | | | to | | | |
| | | | | | | | | | | 0.8708 | | | |
| | SCUBE3 | Signal | Q8IX30 | −1.485 | 3.09E− | 2.03E− | 0.0574 | 0.6746 | 0.0863 | 0.5055 | 0.0563 | 50.0 | 22.7 |
| | | peptide, | | | 07 | 05 | | | | to | | | |
| | | CUB and | | | | | | | | 0.8438 | | | |
| | | EGF-like | | | | | | | | | | | |
| | | domain- | | | | | | | | | | | |
| | | containing | | | | | | | | | | | |
| | | protein 3 | | | | | | | | | | | |
| | VIPR1 | Vasoactive | P32241 | −1.452 | 5.21E− | 3.06E− | 0.0004 | 0.8170 | 0.0668 | 0.6861 | 0.0006 | 42.9 | 38.1 |
| | | intestinal | | | 07 | 05 | | | | to | | | |
| | | polypeptide | | | | | | | | 0.9480 | | | |
| | | receptor 1 | | | | | | | | | | | |
| | FCER2 | Low affinity | P06734 | −0.834 | 1.14E− | 5.705− | 0.0554 | 0.6717 | 0.0838 | 0.5075 | 0.0544 | 522 | 30.4 |
| | | immuno- | | | 06 | 05 | | | | to | | | |
| | | globulin | | | | | | | | 0.0360 | | | |
| | | epsilon [symbol] c | | | | | | | | | | | |
| | | receptor | | | | | | | | | | | |
| | VAT1 | Synaptic | Q99536 | −0.951 | 2.88E− | 9.46E− | 0.0554 | 0.6717 | 0.0837 | 0.5077 | 0.0544 | 52.2 | 17.4 |
| | | vesicle | | | 06 | 05 | | | | to | | | |
| | | membrane | | | | | | | | 0.8358 | | | |
| | | protoin | | | | | | | | | | | |
| | | VAT-1 | | | | | | | | | | | |
| | | homolog | | | | | | | | | | | |
| | GPR180 | Integral | Q86V85 | −0.998 | 4.53E− | 1.28E− | 0.0064 | 0.7432 | 0.0790 | 0.5883 | 0.0070 | 27.3 | 13.6 |
| | | membrane | | | 06 | 04 | | | | to | | | |
| | | protein | | | | | | | | 0.8980 | | | |
| | | GPR180 | | | | | | | | | | | |
| | MXRA8 | Matrix | Q9BRK3 | −0.904 | 5.10E− | 1.29E− | 0.0342 | 0.6891 | 0.0805 | 0.5314 | 0.0341 | 34.8 | 21.7 |
| | | remodeling- | | | 06 | 04 | | | | to | | | |
| | | associated | | | | | | | | 0.8469 | | | |
| | | protein 8 | | | | | | | | | | | |
| | LRRC15 | Leucine-rich | Q8TF66 | −0.983 | 7.13E− | 1.48E− | 0.0080 | 0.7425 | 0.0777 | 0.5902 | 0.0087 | 40.0 | 20.0 |
| | | repeat-con- | | | 06 | 04 | | | | to | | | |
| | | taining | | | | | | | | 0.8948 | | | |
| | | protein 15 | | | | | | | | | | | |
| | DCD | Dermcidin | P81605 | −1.915 | 1.11E− | 2.13E− | 0.0265 | 0.6978 | 0.0818 | 0.5375 | 0.0267 | 52.2 | 21.7 |
| | | | | | 05 | 04 | | | | to | | | |
| | | | | | | | | | | 0.8581 | | | |
| | ATP5F1A | ATP | P25705 | −2.155 | 1.29E− | 2.26E− | 0.0218 | 0.7071 | 0.0808 | 0.5487 | 0.0222 | 43.5 | 21.7 |
| | | synthase | | | 05 | 04 | | | | to | | | |
| | | subunit alpha, | | | | | | | | 0.8655 | | | |
| | | mito- | | | | | | | | | | | |
| | | chondrial | | | | | | | | | | | |
| | B2M | Beta-2- | P61769 | −1.395 | 1.36E− | 2.33E− | 0.0001 | 0.8261 | 0.0639 | 0.7008 | 0.0003 | 65.2 | 13.0 |
| | | micro- | | | 05 | 04 | | | | to | | | |
| | | globulin | | | | | | | | 0.9514 | | | |
| | HRNR | Hornerin | Q86YZ3 | −1.912 | 1.96E− | 2.91E− | 0.0041 | 0.7522 | 0.0759 | 0.6033 | 0.0047 | 47.8 | 13.0 |
| | | | | | 05 | 04 | | | | to | | | |
| | | | | | | | | | | 0.9010 | | | |
| | SCGB1A1 | Uteroglobin | P11684 | −1.412 | 2.51E− | 3.30E− | 0.0364 | 0.6870 | 0.0814 | 0.5273 | 0.0363 | 34.8 | 17.4 |
| | | | | | 05 | 04 | | | | to | | | |
| | | | | | | | | | | 0.8466 | | | |
| | KRT2 | Keratin, | P35908 | −2.176 | 2.94E− | 3.65E− | 0.0021 | 0.7696 | 0.0738 | 0.6250 | 0.0025 | 56.5 | 13.0 |
| | | type II | | | 05 | 04 | | | | to | | | |
| | | cytoskeletal | | | | | | | | 0.9142 | | | |
| | | 2 epidermal | | | | | | | | | | | |
| | IGFALS | Insulin-like | P35858 | −1.100 | 3.61E− | 4.09E− | 0.0016 | 0.7761 | 0.0721 | 0.6348 | 0.0020 | 56.5 | 52.2 |
| | | growth factor- | | | 05 | 04 | | | | to | | | |
| | | binding | | | | | | | | 0.9174 | | | |
| | | protein | | | | | | | | | | | |
| | | complex acid | | | | | | | | | | | |
| | | labile subunit | | | | | | | | | | | |
| | RNASE1 | Ribonuclease | P07998 | −1.070 | 3.69E− | 4.09E− | 0.0364 | 0.6870 | 0.0816 | 0.5270 | 0.0363 | 26.1 | 17.4 |
| | | pancreatic | | | 05 | 04 | | | | to | | | |
| | | | | | | | | | | 0.8470 | | | |
| | KRT13 | Keratin, | P13646 | −2.235 | 4.43E− | 4.60E− | 0.0067 | 0.7391 | 0.0754 | 0.5913 | 0.0074 | 52.2 | 30.4 |
| | | type I | | | 05 | 04 | | | | to | | | |
| | | cytoskeletal | | | | | | | | 0.8869 | | | |
| | | 13 | | | | | | | | | | | |
| | JUP | Junction | P14923 | −1.848 | 4.73E− | 4.76E− | 0.0152 | 0.7185 | 0.0802 | 0.5614 | 0.0158 | 21.7 | 17.4 |
| | | plakoglobin | | | 05 | 04 | | | | to | | | |
| | | | | | | | | | | 0.8757 | | | |
| Con- | CD44 | CD44 antigen | P16070 | −0.065 | 0.6755 | 0.3210 | 0.4327 | 0.5717 | 0.0909 | 0.3936 | 0.4217 | 13.0 | 4.3 |
| trol | | | | | | | | | | to | | | |
| | | | | | | | | | | 0.7499 | | | |
| | RNASE2 | Non-secretory | P10153 | −0.098 | 0.4035 | 0.2254 | 0.7267 | 0.5326 | 0.0905 | 0.3553 | 0.7149 | 13.0 | 4.3 |
| | | ribonuclease | | | | | | | | to | | | |
| | | | | | | | | | | 0.7099 | | | |
| | WFDC2 | WAP four- | Q14508 | −0.169 | 0.1933 | 0.1344 | 0.2710 | 0.6000 | 0.0875 | 0.4285 | 0.2627 | 21.7 | 0.0 |
| | | disulfide | | | | | | | | to | | | |
| | | core domain | | | | | | | | 0.7715 | | | |
| | | protein 2 | | | | | | | | | | | |

[symbol]

indicates data missing or illegible when filed

TABLE 2

| | | | | Student's t-test | | | Mann- | ROC Analysis | | | | | |
|---|---|---|---|---|---|---|---|---|---|---|---|---|---|
| | | | | | | | Whit- | | | | | Speci- | Speci- |
| | | | | | | | ney | | | 95% | | ficity | ficity |
| | | | | Average | | | U test | | | Con- | | at 90% | at 100% |
| | | Protein | | Log2 | p- | q- | p- | | Std. | fidence | | Sensi- | Sensi- |
| | Genes | Name | Uniprot ID | Ratio | value | value | value | AUC | Error | interval | p-value | tivity | tivity |
| Bio- | HPX | Hemopexin | P02790 | −0.977 | 1.39E− | 1.74E− | 0.0001 | 0.8125 | 0.0661 | 0.6830 | 0.0007 | 48.1 | 33.3 |
| marker | | | | | 11 | 08 | | | | to | | | |
| | | | | | | | | | | 0.9420 | | | |
| | SERPINF1 | Pigment | P36955 | −0.849 | 1.86E− | 1.17E− | 0.0154 | 0.7236 | 0.0784 | 0.5699 | 0.0160 | 61.5 | 30.8 |
| | | epithelium- | | | 10 | 07 | | | | to | | | |
| | | derived | | | | | | | | 0.8773 | | | |
| | | factor | | | | | | | | | | | |
| | LCN2 | Neutrophil | P80188 | −1.145 | 3.10E− | 1.29E− | 0.0042 | 0.7593 | 0.0744 | 0.6135 | 0.0049 | 48.1 | 48.1 |
| | | gelatinase- | | | 10 | 07 | | | | to | | | |
| | | associated | | | | | | | | 0.9051 | | | |
| | | lipocalin | | | | | | | | | | | |
| | CANX | Calnexin | P27824 | −1.097 | 1.13E− | 3.41E− | 0.0163 | 0.7280 | 0.0790 | 0.5731 | 0.0169 | 44.0 | 44.0 |
| | | | | | 09 | 07 | | | | to | | | |
| | | | | | | | | | | 0.8829 | | | |
| | APOA4 | Apolipo- | P06727 | −0.836 | 2.21E− | 1.69E− | 0.0218 | 0.7106 | 0.0786 | 0.5567 | 0.0222 | 48.1 | 48.1 |
| | | protein | | | 08 | 06 | | | | to | | | |
| | | A-IV | | | | | | | | 0.8646 | | | |
| | SPARCL1 | SPARC-like | Q14515 | −1.565 | 2.20E− | 1.69E− | 0.0950 | 0.6551 | 0.0826 | 0.4931 | 0.0923 | 40.7 | 37.0 |
| | | protein 1 | | | 08 | 06 | | | | to | | | |
| | | | | | | | | | | 0.8171 | | | |
| | LCP1 | Plastin-2 | P13796 | −1.385 | 3.45E− | 2.05E− | 0.0294 | 0.7019 | 0.0797 | 0.5457 | 0.0296 | 46.2 | 42.3 |
| | | | | | 08 | 06 | | | | to | | | |
| | | | | | | | | | | 0.8581 | | | |
| | MSMB | Beta-micro- | P08118 | −1.055 | 8.28E− | 4.31E− | 0.0950 | 0.6551 | 0.0841 | 0.4903 | 0.0923 | 37.0 | 29.0 |
| | | semino- | | | 08 | 06 | | | | to | | | |
| | | protein | | | | | | | | 0.8199 | | | |
| | CD99 | CD99 | P14209 | −1.135 | 8.91E− | 4.31E− | 0.0294 | 0.7019 | 0.0827 | 0.5399 | 0.0296 | 26.9 | 26.9 |
| | | antigen | | | 08 | 06 | | | | to | | | |
| | | | | | | | | | | 0.8639 | | | |
| | SCUBE2 | Signal | Q9NQ36 | −0.968 | 3.01E− | 1.10E− | 0.0422 | 0.6875 | 0.0801 | 0.5305 | 0.0418 | 37.0 | 33.3 |
| | | peptide, | | | 07 | 05 | | | | to | | | |
| | | CUB and | | | | | | | | 0.8445 | | | |
| | | EGF-like | | | | | | | | | | | |
| | | domain- | | | | | | | | | | | |
| | | containing | | | | | | | | | | | |
| | | protein 2 | | | | | | | | | | | |
| | IGLV3-10 | Immuno- | A0A075B6K4 | −0.982 | 3.09E− | 1.10E− | 0.0422 | 0.6875 | 0.0805 | 0.5207 | 0.0418 | 37.0 | 33.3 |
| | | globulin | | | 07 | 05 | | | | to | | | |
| | | lambda | | | | | | | | 0.8453 | | | |
| | | variable 3 | | | | | | | | | | | |
| | | 10 | | | | | | | | | | | |
| | TALDO1 | Trans- | P37837 | −1.024 | 3.96E− | 1.30E− | 0.0709 | 0.6700 | 0.0844 | 0.5045 | 0.0692 | 36.0 | 28.0 |
| | | aldolase | | | 07 | 05 | | | | to | | | |
| | | | | | | | | | | 0.8355 | | | |
| | SERPINA6 | Cortico- | P08185 | −0.980 | 4.33E− | 1.39E− | 0.1171 | 0.6458 | 0.0842 | 0.4807 | 0.1134 | 37.0 | 25.9 |
| | | steroid- | | | 07 | 05 | | | | to | | | |
| | | binding | | | | | | | | 0.8109 | | | |
| | | globulin | | | | | | | | | | | |
| | TGFBR2 | TGF-beta | P37173 | −0.835 | 4.49E− | 1.40E− | 0.1361 | 0.6389 | 0.0847 | 0.4729 | 0.1317 | 48.1 | 48.1 |
| | | receptor | | | 07 | 05 | | | | to | | | |
| | | type-2 | | | | | | | | 0.8049 | | | |
| | VIPR1 | Vasoactive | P32241 | −1.305 | 5.62E− | 1.56E− | 0.0038 | 0.7682 | 0.0748 | 0.6217 | 0.0045 | 37.5 | 33.3 |
| | | inleslinal | | | 07 | 05 | | | | to | | | |
| | | polypeptide | | | | | | | | 0.9118 | | | |
| | | receptor 1 | | | | | | | | | | | |
| | ANXA3 | Annexin A3 | P12429 | −0.880 | 1.03E− | 2.43E− | 0.2023 | 0.6202 | 0.0867 | 0.4502 | 0.1953 | 38.5 | 30.8 |
| | | | | | 06 | 05 | | | | to | | | |
| | | | | | | | | | | 0.7902 | | | |
| | LYVE1 | Lymphatic | Q9Y5Y7 | −1.042 | 1.70E− | 3.53E− | 0.1893 | 0.6227 | 0.0865 | 0.4532 | 0.1830 | 40.7 | 33.3 |
| | | vessel | | | 06 | 05 | | | | to | | | |
| | | endothelial | | | | | | | | 0.7921 | | | |
| | | hyaluronic | | | | | | | | | | | |
| | | acid receptor | | | | | | | | | | | |
| | | 1 | | | | | | | | | | | |
| | PTGDS | Prosta- | P41222 | −0.958 | 3.87E− | 6.81E− | 0.1002 | 0.0528 | 0.0850 | 0.4863 | 0.0973 | 55.0 | 51.9 |
| | | glandin- | | | 06 | 05 | | | | to | | | |
| | | H2 D- | | | | | | | | 0.8193 | | | |
| | | isomerase | | | | | | | | | | | |
| | IGKV3D- | Immuno- | A0ACA0MRZ8 | −0.878 | 9.38E− | 1.23E− | 0.1573 | 0.6319 | 0.0865 | 0.4624 | 0.1521 | 48.1 | 40.7 |
| | 11 | globulin | | | 06 | 04 | | | | to | | | |
| | | kappa | | | | | | | | 0.8015 | | | |
| | | variable | | | | | | | | | | | |
| | | 3D-11 | | | | | | | | | | | |
| | TKT | Trans- | P29401 | −0.890 | 9.32E− | 1.23E− | 0.0385 | 0.6923 | 0.0852 | 0.5253 | 0.0383 | 30.8 | 26.9 |
| | | ketolase | | | 06 | 04 | | | | to | | | |
| | | | | | | | | | | 0.8593 | | | |
| | AMBP | Protein | P02760 | −0.892 | 1.23E− | 1.54E− | 0.2070 | 0.6181 | 0.0849 | 0.4517 | 0.2000 | 40.7 | 33.3 |
| | | AMBP | | | 05 | 04 | | | | to | | | |
| | | | | | | | | | | 0.7844 | | | |
| | HYOU1 | Hypoxia up- | Q9Y4L1 | −1.466 | 2.04E− | 2.22E− | 0.0546 | 0.6815 | 0.0814 | 0.5219 | 0.0537 | 51.9 | 44.4 |
| | | regulated | | | 05 | 04 | | | | to | | | |
| | | protein 1 | | | | | | | | 0.8411 | | | |
| | IGFALS | Insulin-like | P35858 | −0.923 | 3.50E− | 3.27E− | 0.0189 | 0.7153 | 0.0773 | 0.5638 | 0.0195 | 51.9 | 48.1 |
| | | growth | | | 05 | 04 | | | | to | | | |
| | | factor- | | | | | | | | 0.8667 | | | |
| | | binding | | | | | | | | | | | |
| | | protein | | | | | | | | | | | |
| | | complex | | | | | | | | | | | |
| | | acid labile | | | | | | | | | | | |
| | | subunit | | | | | | | | | | | |
| | KRT13 | Keratin. | P13646 | −1.837 | 8.11E− | 6.00E− | 0.1728 | 0.6273 | 0.0852 | 0.4603 | 0.1670 | 44.4 | 25.9 |
| | | type I cyto- | | | 05 | 04 | | | | to | | | |
| | | skeletal 13 | | | | | | | | 0.7943 | | | |
| | MASP 1 | Mannan- | P18740 | −1.261 | 1.81E− | 1.02E− | 0.0132 | 0.7284 | 0.0773 | 0.5769 | 0.0139 | 46.2 | 34.6 |
| | | binding | | | 04 | 03 | | | | to | | | |
| | | lectin serine | | | | | | | | 0.8798 | | | |
| | | protease 1 | | | | | | | | | | | |
| Con- | CD44 | CD44 | P16070 | 0.069 | 0.4767 | 0.2927 | 0.9901 | 0.5023 | 0.0933 | 0.3195 | 0.9800 | 3.7 | 3.7 |
| trol | | antigen | | | | | | | | to | | | |
| | | | | | | | | | | 0.6851 | | | |
| | RNASE2 | Non- | P10153 | 0.001 | 0.9511 | 0.4500 | 0.9901 | 0.5023 | 0.0965 | 0.3132 | 0.9800 | 3.7 | 3.7 |
| | | secretory | | | | | | | | to | | | |
| | | ribonuclease | | | | | | | | 0.6914 | | | |
| | WFUC2 | WAP four- | Q14508 | −0.088 | 0.5581 | 0.3215 | 0.4938 | 0.5648 | 0.0895 | 0.3895 | 0.4817 | 22.2 | 0.0 |
| | | disulfide | | | | | | | | to | | | |
| | | core domain | | | | | | | | 0.7401 | | | |

protein 2

TABLE 3.1

| | | | | Student's t-test | | | Mann- | ROC Analysis | | | | | |
|---|---|---|---|---|---|---|---|---|---|---|---|---|---|
| | | | | | | | Whit- | | | | | Speci- | Speci- |
| | | | | Ave- | | | ney | | | 95% | | ficity | ficity |
| | | | | rage | | | U test | | | Con- | | at 90% | at 100% |
| | | | Uniprot | Log2 | p- | q- | p- | | Std. | fidence | p- | Sensi- | Sensi- |
| | Genes | Protein Name | ID | Ratio | value | value | value | AUC | Error | interval | value | tivity | tivity |
| Bio- | CEL | Bile salt- | P19835 | 0.939 | 2.82E− | 9.83E− | 0.0352 | 0.7108 | 0.0891 | 0.5362 to | 0.0351 | 38.5 | 15.4 |
| marker | | activated lipase | | | 06 | 05 | | | | 0.8853 | | | |
| | SCUBE3 | Signal peptide, | Q8IX30 | −1.363 | 8.57E− | 1.31E− | 0.0697 | 0.6856 | 0.1003 | 0.4891 to | 0.0676 | 53.8 | 23.1 |
| | | CUB and EGF- | | | 05 | 03 | | | | 0.8821 | | | |
| | | like domain- | | | | | | | | | | | |
| | | containing | | | | | | | | | | | |
| | | protein 3 | | | | | | | | | | | |
| | LYVE1 | Lymphatic | Q9Y5Y7 | −1.435 | 3.51E− | 3.57E− | 0.0275 | 0.7200 | 0.0995 | 0.5250 to | 0.0278 | 53.8 | 23.1 |
| | | vessel | | | 04 | 03 | | | | 0.9150 | | | |
| | | endothelial | | | | | | | | | | | |
| | | hyaluronic acid | | | | | | | | | | | |
| | | receptor 1 | | | | | | | | | | | |
| | EEF2 | Elongation | P13639 | −1.112 | 3.62E− | 3.65E− | 0.0663 | 0.6900 | 0.1043 | 0.4855 to | 0.0644 | 50.0 | 50.0 |
| | | factor 2 | | | 04 | 03 | | | | 0.8945 | | | |
| | SCUBE1 | Signal peptide, | Q8IWY4 | −1.185 | 4.09E− | 4.01E− | 0.0741 | 0.6800 | 0.1065 | 0.4713 to | 0.0719 | 46.2 | 23.1 |
| | | CUB and EGF- | | | 04 | 03 | | | | 0.8887 | | | |
| | | like domain- | | | | | | | | | | | |
| | | containing | | | | | | | | | | | |
| | | protein 1 | | | | | | | | | | | |
| | C4BPA | C4b-binding | P04003 | −0.977 | 6.17E− | 5.30E− | 0.0683 | 0.6945 | 0.1022 | 0.4943 to | 0.0662 | 36.4 | 9.1 |
| | | protein alpha | | | 04 | 03 | | | | 0.8948 | | | |
| | | chain | | | | | | | | | | | |
| | BASP1 | Brain acid | P80723 | −1.168 | 8.40E− | 6.71E− | 0.0557 | 0.6923 | 0.1013 | 0.4937 to | 0.0545 | 46.2 | 38.5 |
| | | soluble protein | | | 04 | 03 | | | | 0.8909 | | | |
| | | 1 | | | | | | | | | | | |
| | SPARCL1 | SPARC-like | Q14515 | −1.691 | 8.63E− | 6.85E− | 0.0352 | 0.7108 | 0.0986 | 0.5176 to | 0.0351 | 53.8 | 23.1 |
| | | protein 1 | | | 04 | 03 | | | | 0.9040 | | | |
| | IGKV2-30 | Immuno- | P06310 | −1.135 | 1.04E− | 7.86E− | 0.0164 | 0.7465 | 0.0894 | 0.5713 to | 0.0172 | 50.0 | 33.3 |
| | | globulin | | | 03 | 03 | | | | 0.9218 | | | |
| | | kappa variable | | | | | | | | | | | |
| | | 2-30 | | | | | | | | | | | |
| | SCUBE2 | Signal peptide, | Q9NQ36 | −1.119 | 1.10E− | 8.10E− | 0.0164 | 0.7385 | 0.0894 | 0.5633 to | 0.0171 | 38.5 | 15.4 |
| | | CUB and EGF- | | | 03 | 03 | | | | 0.9136 | | | |
| | | like domain- | | | | | | | | | | | |
| | | containing | | | | | | | | | | | |
| | | protein 2 | | | | | | | | | | | |
| | AZGP1 | Zinc-alpha-2- | P25311 | −0.951 | 1.32E− | 9.07E− | 0.0113 | 0.7508 | 0.0803 | 0.5935 to | 0.0122 | 46.2 | 15.4 |
| | | glycoprotein | | | 03 | 03 | | | | 0.9081 | | | |
| | APEH | Acylamino- | P13798 | −0.955 | 1.56E− | 1.01E− | 0.0645 | 0.7040 | 0.0989 | 0.5101 to | 0.0626 | 50.0 | 10.0 |
| | | acid-releasing | | | 03 | 02 | | | | 0.8979 | | | |
| | | enzyme | | | | | | | | | | | |
| | AMBP | Protein AMBP | P02760 | −1.166 | 2.28E− | 1.32E− | 0.0849 | 0.6738 | 0.1019 | 0.4741 to | 0.0821 | 38.5 | 23.1 |
| | | | | | 03 | 02 | | | | 0.8736 | | | |
| | HYOU1 | Hypoxia up- | Q9Y4L1 | −1.591 | 2.44E− | 1.36E− | 0.0331 | 0.7147 | 0.1006 | 0.5175 to | 0.0330 | 53.8 | 15.4 |
| | | regulated | | | 03 | 02 | | | | 0.9119 | | | |
| | | protein 1 | | | | | | | | | | | |
| | CDH1 | Cadherin-1 | P12830 | −2.007 | 2.77E− | 1.48E− | 0.0062 | 0.7692 | 0.0923 | 0.5883 to | 0.0071 | 46.8 | 23.1 |
| | | | | | 03 | 02 | | | | 0.9502 | | | |
| | S100A8 | Protein S100- | P05109 | −1.665 | 2.89E− | 1.54E− | 0.1019 | 0.6700 | 0.1045 | 0.4652 to | 0.0980 | 41.7 | 33.3 |
| | | A8 | | | 03 | 02 | | | | 0.8748 | | | |
| | CD55 | Complement | P08174 | −1.193 | 3.06E− | 1.60E− | 0.0480 | 0.6985 | 0.0962 | 0.5100 to | 0.0472 | 38.5 | 23.1 |
| | | decay- | | | 03 | 02 | | | | 0.8869 | | | |
| | | accelerating | | | | | | | | | | | |
| | | factor | | | | | | | | | | | |
| | DSG1 | Desmoglein-1 | Q02413 | −1.171 | 3.80E− | 1.81E− | 0.0033 | 0.7877 | 0.0788 | 0.6332 to | 0.0040 | 38.5 | 15.4 |
| | | | | | 03 | 02 | | | | 0.9422 | | | |
| | PTGFRN | Prostaglandin | Q9P2B2 | −1.033 | 4.21E− | 1.92E− | 0.0741 | 0.6800 | 0.0946 | 0.4947 to | 0.0719 | 38.5 | 15.4 |
| | | F2 receptor | | | 03 | 02 | | | | 0.8653 | | | |
| | | negative | | | | | | | | | | | |
| | | regulator | | | | | | | | | | | |
| | EMCN | Endomucin | Q9ULC0 | −1.075 | 5.37E− | 2.28E− | 0.0557 | 0.6923 | 0.1019 | 0.4925 to | 0.0545 | 46.2 | 15.4 |
| | | | | | 03 | 02 | | | | 0.8921 | | | |
| | IL15RA | Interleukin-15 | Q13261 | −1.129 | 1.14E− | 3.60E− | 0.0247 | 0.7300 | 0.0885 | 0.5565 to | 0.0252 | 41.7 | 8.3 |
| | | receptor | | | 02 | 02 | | | | 0.9035 | | | |
| | | subunit alpha | | | | | | | | | | | |
| | FCER1A | High affinity | P12319 | −1.207 | 2.20E− | 5.10E− | 0.0415 | 0.7164 | 0.1020 | 0.5165 to | 0.0410 | 45.5 | 18.2 |
| | | immuno- | | | 02 | 02 | | | | 0.9102 | | | |
| | | globulin | | | | | | | | | | | |
| | | epsilon receptor | | | | | | | | | | | |
| | | subunit alpha | | | | | | | | | | | |
| | NELL1 | Protein kinase | Q92832 | −1.010 | 2.95E− | 6.17E− | 0.0275 | 0.7200 | 0.0995 | 0.5250 to | 0.0278 | 53.8 | 23.1 |
| | | C-binding | | | 02 | 02 | | | | 0.9150 | | | |
| | | protein NELL1 | | | | | | | | | | | |
| | TALDO1 | Iransaldolase | P37837 | −1.038 | 3.40E− | 6.706− | 0.0042 | 0.7964 | 0.0979 | 0.6046 to | 0.0051 | 63.6 | 0.0 |
| | | | | | 02 | 02 | | | | 0.9881 | | | |
| | GPR37L1 | G-protein | O60883 | −3.118 | 3.73E− | 7 04E− | 0.0226 | 0.7333 | 0.0945 | 0.5481 to | 0.0231 | 41.7 | 33.3 |
| | | coupled | | | 02 | 02 | | | | 0.9185 | | | |
| | | receptor 37-like | | | | | | | | | | | |
| | | 1 | | | | | | | | | | | |
| Con- | CD44 | CD44 antigen | P16070 | −0.097 | 0.5284 | 0.3111 | 0.8794 | 0.5169 | 0.1039 | 0.3132 to | 0.8656 | 15.4 | 7.7 |
| trol | | | | | | | | | | 0.7207 | | | |
| | RNASE2 | Non-secretory | P10153 | −0.100 | 0.6209 | 0.3456 | 0.8794 | 0.5169 | 0.1029 | 0.3152 to | 0.8656 | 7.7 | 0.0 |
| | | ribonuclease | | | | | | | | 0.7187 | | | |
| | WFDC2 | WAP four- | Q14508 | 0.031 | 0.9131 | 0.4381 | 0.8317 | 0.5231 | 0.1009 | 0.3136 to | 0.8175 | 7.7 | 7.7 |
| | | disulfide core | | | | | | | | 0.7320 | | | |
| | | domain protein | | | | | | | | | | | |
| | | 2 | | | | | | | | | | | |

TABLE 3.2

| | | | | Mann- | ROC Analysis | | | | | |
|---|---|---|---|---|---|---|---|---|---|---|
| | | | | Whitney | | | 95% | | Specificity | Specificity |
| | | | Uniprot | U test | | Std. | Confidence | p- | at 90% | at 100% |
| | Genes | Protein Name | ID | p-value | AUC | Error | interval | value | Sensitivity | Sensitivity |
| Biomarker | LYVE1 | Lymphatic vessel | Q9Y5Y7 | <0.0001 | 0.5491 | 0.1015 | 0.3501 to | 0.6147 | 23.1 | 0.0 |
| | | endothelial | | | | | 0.7481 | | | |
| | | hyaluronic acid | | | | | | | | |
| | | receptor 1 | | | | | | | | |
| | SPARCL1 | SPARC-like | Q14515 | <0.0001 | 0.6382 | 0.0998 | 0.4425 to | 0.1614 | 46.1 | 0.0 |
| | | protein 1 | | | | | 0.8338 | | | |
| | AMBP | Protein AMBP | P02760 | <0.0001 | 0.6048 | 0.0941 | 0.4204 to | 0.2825 | 30.8 | 0.0 |
| | | | | | | | 0.7891 | | | |
| Biomarker | LYVE1 | Lymphatic vessel | Q9Y5Y7 | 0.3937 | 0.6724 | 0.0922 | 0.4918 to | 0.0770 | 38.5 | 0.0 |
| normalized | | endothelial | | | | | 0.8530 | | | |
| with CD44 | | hyaluronic acid | | | | | | | | |
| and | | receptor 1 | | | | | | | | |
| RNASE2 | SPARCL1 | SPARC-like | Q14515 | 0.0009 | 0.7294 | 0.0809 | 0.5709 to | 0.0186 | 23.1 | 7.7 |
| | | protein 1 | | | | | 0.8880 | | | |
| | AMBP | Protein AMBP | P02760 | 0.0009 | 0.7493 | 0.0822 | 0.5881 to | 0.0105 | 23.1 | 23.1 |
| | | | | | | | 0.9105 | | | |
| Control | CD44 | CD44 antigen | P16070 | <0.0001 | 0.5623 | 0.1088 | 0.3490 to | 0.5226 | 15.5 | 7.7 |
| | | | | | | | 0.7756 | | | |
| | RNASE2 | Non-secretory | P10153 | <0.0001 | 0.7082 | 0.0827 | 0.5462 to | 0.0327 | 23.1 | 7.7 |
| | | ribonuclease | | | | | 0.8703 | | | |
| | WFDC2 | WAP four- | Q14508 | <0.0001 | 0.7636 | 0.0801 | 0.6066 to | 0.0128 | 18.2 | 0.0 |
| | | disulfide core | | | | | 0.9207 | | | |
| | | domain protein 2 | | | | | | | | |

TABLE 4

| Column 1 | Column 2 | Column 3 |
|---|---|---|
| APOA1 | AMBP | AMBP |
| APOA4 | ANXA3 | APEH |
| ATP5F1A | APOA4 | AZGP1 |
| B2M | CANX | BASP1 |
| CALR | CD99 | CD55 |
| CANX | HPX | CDH1 |
| CD99 | HYOU1 | CEL |
| DCD | IGFALS | DSG1 |
| FCER2 | IGKV3D-11 | EEF2 |
| GPR180 | IGLV3-10 | EMCN |
| HPX | KRT13 | HYOU1 |
| HRNR | LCN2 | IGKV2-30 |
| IGFALS | LCP1 | IL15RA |
| JUP | LYVE1 | LYVE1 |
| KRT13 | MASP1 | PTGFRN |
| KRT2 | MSMB | S100A8 |
| LRRC15 | PTGDS | SCUBE1 |
| MXRA8 | SCUBE2 | SCUBE2 |
| PNP | SERPINA6 | SCUBE3 |
| RNASE1 | PEDF | SPARCL1 |
| SCGB1A1 | SPARCL1 | C4BPA |
| SCUBE3 | TALDO1 | FCER1A |
| PEDF | TGFBR2 | NELL1 |
| VAT1 | TKT | TALDO1 |
| VIPR1 | VIPR1 | GPR37L1 |

TABLE 5.1

| | Genes | Protein Name | Uniprot ID |
|---|---|---|---|
| Biomarker | SERPINF1 | Pigment epithelium-derived factor | P36955 |
| | CALR | Calreticulin | P27797 |
| | HPX | Hemopexin | P02790 |
| | PNP | Purine nucleoside phosphorylase | P00491 |
| | APOA4 | Apolipoprotein A-IV | P06727 |
| | CD99 | CD99 antigen | P14209 |
| | APOA1 | Apolipoprotein A-I | P02647 |
| | CANX | Calnexin | P27824 |
| | SCUBE3 | Signal peptide, CUB and EGF-like domain-containing protein 3 | Q8IX30 |
| | VIPR1 | Vasoactive intestinal polypeptide receptor 1 | P32241 |
| | FCER2 | Low affinity immunoglobulin epsilon Fc receptor | P06734 |
| | VAT1 | Synaptic vesicle membrane protein VAT-1 homolog | Q99536 |
| | GPR180 | Integral membrane protein GPR180 | Q86V85 |
| | MXRA8 | Matrix remodeling-associated protein 8 | Q9BRK3 |
| | LRRC15 | Leucine-rich repeat-containing protein 15 | Q8TF66 |
| | DCD | Dermcidin | P81605 |
| | ATP5F1A | ATP synthase subunit alpha, mitochondrial | P25705 |
| | B2M | Beta-2-microglobulin | P61769 |
| | HRNR | Hornerin | Q86YZ3 |
| | SCGB1A1 | Uteroglobin | P11684 |
| | KRT2 | Keratin, type II cytoskeletal 2 epidermal | P35908 |
| | IGFALS | Insulin-like growth factor-binding protein complex acid labile | P35858 |
| | | subunit | |
| | RNASE1 | Ribonuclease pancreatic | P07998 |
| | KRT13 | Keratin, type I cytoskeletal 13 | P13646 |
| | JUP | Junction plakoglobin | P14923 |
| | LCN2 | Neutrophil gelatinase-associated lipocalin | P80188 |
| | SPARCL1 | SPARC-like protein 1 | Q14515 |
| | LCP1 | Plastin-2 | P13796 |
| | MSMB | Beta-microseminoprotein | P08118 |
| | SCUBE2 | Signal peptide, CUB and EGF-like domain-containing protein 2 | Q9NQ36 |
| | IGLV3-10 | Immunoglobulin lambda variable 3-10 | A0A075B6K4 |
| | TALDO1 | Transaldolase | P37837 |
| | SERPINA6 | Corticosteroid-binding globulin | P08185 |
| | TGFBR2 | TGF-beta receptor type-2 | P37173 |
| | ANXA3 | Annexin A3 | P12429 |
| | LYVE1 | Lymphatic vessel endothelial hyaluronic acid receptor 1 | Q9Y5Y7 |
| | PTGDS | Prostaglandin-H2 D-isomerase | P41222 |
| | IGKV3D-11 | Immunoglobulin kappa variable 3D-11 | A0A0A0MRZ8 |
| | TKT | Transketolase | P29401 |
| | AMBP | Protein AMBP | P02760 |
| | HYOU1 | Hypoxia up-regulated protein 1 | Q9Y4L1 |
| | MASP1 | Mannan-binding lectin serine protease 1 | P48740 |
| | CEL | Bile salt-activated lipase | P19835 |
| | EEF2 | Elongation factor 2 | P13639 |
| | SCUBE1 | Signal peptide, CUB and EGF-like domain-containing protein 1 | Q8IWY4 |
| | C4BPA | C4b-binding protein alpha chain | P04003 |
| | BASP1 | Brain acid soluble protein 1 | P80723 |
| | IGKV2-30 | Immunoglobulin kappa variable 2-30 | P06310 |
| | AZGP1 | Zinc-alpha-2-glycoprotein | P25311 |
| | APEH | Acylamino-acid-releasing enzyme | P13798 |
| | CDH1 | Cadherin-1 | P12830 |
| | S100A8 | Protein S100-A8 | P05109 |
| | CD55 | Complement decay-accelerating factor | P08174 |
| | DSG1 | Desmoglein-1 | Q02413 |
| | PTGFRN | Prostaglandin F2 receptor negative regulator | Q9P2B2 |
| | EMCN | Endomucin | Q9ULC0 |
| | IL15RA | Interleukin-15 receptor subunit alpha | Q13261 |
| | FCER1A | High affinity immunoglobulin epsilon receptor subunit alpha | P12319 |
| | NELL1 | Protein kinase C-binding protein NELL1 | Q92832 |
| | GPR37L1 | G-protein coupled receptor 37-like 1 | O60883 |

TABLE 5.2

| | | | |
|---|---|---|---|
| Control | CD44 | CD44 antigen | P16070 |
| | RNASE2 | Non-secretory ribonuclease | P10153 |
| | WFDC2 | WAP four-disulfide core domain protein 2 | Q14508 |

TABLE 6

| | | | | Detect Pca (any grade) | | | | | | | Detect High-grade Pca (GS ≥ 7) | | | | | | |
|---|---|---|---|---|---|---|---|---|---|---|---|---|---|---|---|---|---|
| | | | | | | 95% | | | | | | | 95% | | | | |
| | | | | | | Con- | | Specificity | Specificity | | | | Con- | | Specificity | Specificity | |
| | | Protein | Uniprot | | Std. | fidence | p- | at 90% | at 100% | FIG. | | Std. | fidence | p- | at 90% | at 100% | FIG. |
| | Genes | Name | ID | AUC | Error | interval | value | Sensitivity | Sensitivity | Number | AUC | Error | interval | value | Sensitivity | Sensitivity | Number |
| Bio- | SERPINF1 | Pigment | P36955 | 0.8023 | 0.0696 | 0.6659 | 0.0008 | 68.2 | 36.4 | 1.1A | 0.7236 | 0.0784 | 0.5699 | 0.0160 | 61.5 | 30.8 | 1.1B |
| marker | | epithelium- | | | | to | | | | | | | to | | | | |
| | | derived | | | | 0.9386 | | | | | | | 0.8773 | | | | |
| | | factor | | | | | | | | | | | | | | | |
| | CALR | Calreticulin | P27797 | 0.8043 | 0.0686 | 0.6699 | 0.0007 | 47.8 | 34.8 | 1.2A | 0.7593 | 0.0758 | 0.6107 | 0.0049 | 29.6 | 29.6 | 1.2B |
| | | | | | | to | | | | | | | to | | | | |
| | | | | | | 0.9388 | | | | | | | 0.9078 | | | | |
| | IPX | Hemopexin | P02790 | 0.7761 | 0.0696 | 0.6396 | 0.0020 | 52.2 | 39.1 | 1.3A | 0.8125 | 0.0661 | 0.6830 | 0.0007 | 48.1 | 33.3 | 1.3B |
| | | | | | | to | | | | | | | to | | | | |
| | | | | | | 0.9125 | | | | | | | 0.9420 | | | | |
| | PNP | Purine | P00491 | 0.6739 | 0.0823 | 0.5126 | 0.0514 | 34.8 | 30.4 | 1.4A | 0.5972 | 0.0891 | 0.4225 | 0.2913 | 29.6 | 25.9 | 1.4B |
| | | nucleoside | | | | to | | | | | | | to | | | | |
| | | phosphorylase | | | | 0.8352 | | | | | | | 0.7719 | | | | |
| | APOA4 | Apolipoprotein | P06727 | 0.7174 | 0.0786 | 0.5634 | 0.0149 | 52.2 | 26.1 | 1.5A | 0.7106 | 0.0786 | 0.5567 | 0.0222 | 48.1 | 48.1 | 1.5B |
| | | A-IV | | | | to | | | | | | | to | | | | |
| | | | | | | 0.8714 | | | | | | | 0.8646 | | | | |
| | CD99 | CD99 antigen | P14209 | 0.0750 | 0.0835 | 0.5114 | 0.0525 | 36.4 | 31.8 | 1.6A | 0.7019 | 0.0827 | 0.5399 | 0.0296 | 26.9 | 26.9 | 1.6B |
| | | | | | | to | | | | | | | to | | | | |
| | | | | | | 0.8386 | | | | | | | 0.8639 | | | | |
| | APOA1 | Apolipoprotein | P02647 | 0.7435 | 0.0768 | 0.5930 | 0.0064 | 52.2 | 17.4 | 1.7A | 0.7407 | 0.0791 | 0.5857 | 0.0090 | 48.1 | 14.8 | 1.7B |
| | | A-IV | | | | to | | | | | | | to | | | | |
| | | | | | | 0.8939 | | | | | | | 0.8958 | | | | |
| | CANX | Calnexin | P27824 | 0.7043 | 0.0850 | 0.5377 | 0.0273 | 47.6 | 38.1 | 1.8A | 0.7280 | 0.0790 | 0.5731 | 0.0169 | 44.0 | 44.0 | 1.8B |
| | | | | | | to | | | | | | | to | | | | |
| | | | | | | 0.8708 | | | | | | | 0.8829 | | | | |
| | SCURE3 | Signal peptide, | Q81X30 | 0.6746 | 0.0863 | 0.5055 | 0.0563 | 50.0 | 22.7 | 1.9A | 0.6949 | 0.0827 | 0.5328 | 0.0307 | 46.1 | 19.2 | 1.9B |
| | | CUB and [symbol] - | | | | to | | | | | | | to | | | | |
| | | like domain- | | | | 0.8438 | | | | | | | 0.8570 | | | | |
| | | containing protein | | | | | | | | | | | | | | | |
| | | 3 | | | | | | | | | | | | | | | |
| | VIPR1 | Vasoactive | P32241 | 0.8170 | 0.0668 | 0.6861 | 0.0006 | 42.9 | 38.1 | 1.10A | 0.7682 | 0.0748 | 0.6217 | 0.0045 | 37.5 | 33.3 | 1.10B |
| | | intestinal | | | | to | | | | | | | to | | | | |
| | | polypeptide | | | | 0.9480 | | | | | | | 0.9148 | | | | |
| | | receptor 1 | | | | | | | | | | | | | | | |
| | FCER2 | Low affinity | P06734 | 0.6717 | 0.0838 | 0.5075 | 0.0544 | 52.2 | 30.4 | 1.11A | 0.6412 | 0.0834 | 0.4777 | 0.1254 | 40.7 | 25.1 | 1.11B |
| | | immunoglobulin | | | | to | | | | | | | to | | | | |
| | | epsilon Fc | | | | 0.8360 | | | | | | | 0.8047 | | | | |
| | | receptor | | | | | | | | | | | | | | | |
| | VAT1 | Synaptic vesicle | Q99536 | 0.6717 | 0.0837 | 0.5077 | 0.0544 | 52.2 | 17.4 | 1.12A | 0.6204 | 0.0867 | 0.4504 | 0.1914 | 44.4 | 14.8 | 1.12B |
| | | membrane | | | | to | | | | | | | to | | | | |
| | | protein VAT-1 | | | | 0.8350 | | | | | | | 0.7903 | | | | |
| | | homolog | | | | | | | | | | | | | | | |
| | GPR180 | Integral | Q86V85 | 0.7432 | 0.0790 | 0.5883 | 0.0070 | 27.3 | 13.6 | 1.13A | 0.6490 | 0.0912 | 0.4703 | 0.1083 | 23.1 | 11.5 | 1.13B |
| | | membrane | | | | to | | | | | | | to | | | | |
| | | protein GPR180 | | | | 0.8980 | | | | | | | 0.8278 | | | | |
| | MXRAB | Matrix | Q9BRK3 | 0.6891 | 0.0805 | 0.5314 | 0.0341 | 34.8 | 21.7 | 1.14A | 0.5625 | 0.0883 | 0.3895 | 0.4975 | 25.9 | 18.5 | 1.14B |
| | | remodeling- | | | | to | | | | | | | to | | | | |
| | | associated | | | | 0.8409 | | | | | | | 0.7355 | | | | |
| | | protein 8 | | | | | | | | | | | | | | | |
| | LRRC15 | Leucine-rich | Q8TF66 | 0.7425 | 0.0777 | 0.5902 | 0.0087 | 40.0 | 20.0 | 1.15A | 0.6354 | 0.0911 | 0.4570 | 0.1511 | 20.8 | 16.7 | 1.15B |
| | | repeat | | | | to | | | | | | | to | | | | |
| | | containing | | | | 0.8948 | | | | | | | 0.8130 | | | | |
| | | protein 15 | | | | | | | | | | | | | | | |
| | DCD | Dermcidin | P81605 | 0.6978 | 0.0818 | 0.5375 | 0.0267 | 52.2 | 21.7 | 1.16A | 0.6019 | 0.0857 | 0.4339 | 0.2689 | 37.0 | 18.5 | 1.16B |
| | | | | | | to | | | | | | | to | | | | |
| | | | | | | 0.8581 | | | | | | | 0.7698 | | | | |
| | ATP5F1A | ATP synthase | P25705 | 0.7071 | 0.0808 | 0.5437 | 0.0222 | 43.5 | 21.7 | 1.17A | 0.6019 | 0.0857 | 0.4339 | 0.2689 | 37.0 | 18.5 | 1.17B |
| | | subunil alpha | | | | to | | | | | | | to | | | | |
| | | mitochondrial | | | | 0.8655 | | | | | | | 0.7698 | | | | |
| | B2M | Beta-2- | P61769 | 0.8261 | 0.0630 | 0.7008 | 0.0003 | 65.2 | 13.0 | 1.18A | 0.7546 | 0.0774 | 0.0029 | 0.0057 | 44.4 | 11.1 | 1.18B |
| | | microglobulin | | | | to | | | | | | | to | | | | |
| | | | | | | 0.9514 | | | | | | | 0.9064 | | | | |
| | HRNR | Hornerin | Q86YZ3 | 0.7522 | 0.0759 | 0.6033 | 0.0048 | 47.8 | 13.0 | 1.19A | 0.6458 | 0.0852 | 0.4789 | 0.1134 | 33.3 | 11.1 | 1.19B |
| | | | | | | to | | | | | | | to | | | | |
| | | | | | | 0.9010 | | | | | | | 0.8127 | | | | |
| | SCGB1A1 | Uteroglobin | P11684 | 0.6870 | 0.0814 | 0.5273 | 0.0363 | 34.8 | 17.4 | 1.20A | 0.6968 | 0.0848 | 0.5306 | 0.0327 | 22.2 | 14.8 | 1.20B |
| | | | | | | to | | | | | | | to | | | | |
| | | | | | | 0.8466 | | | | | | | 0.8629 | | | | |
| | KRT2 | Keratin, type II | P35908 | 0.7696 | 0.0738 | 0.6250 | 0.0025 | 56.5 | 13.0 | 1.21A | 0.6736 | 0.0834 | 0.5101 | 0.0595 | 48.1 | 11.1 | 1.21B |
| | | cytoskeletal 2 | | | | to | | | | | | | to | | | | |
| | | epidermal | | | | 0.9142 | | | | | | | 0.8371 | | | | |
| | IGFALS | Insulin-like growth | P35858 | 0.7761 | 0.0721 | 0.6348 | 0.0020 | 56.5 | 52.2 | 1.22A | 0.7153 | 0.0773 | 0.5638 | 0.0195 | 51.9 | 48.1 | 1.22B |
| | | factor-binding | | | | to | | | | | | | to | | | | |
| | | protein complex | | | | 0.9174 | | | | | | | 0.8667 | | | | |
| | | acid labile subunit | | | | | | | | | | | | | | | |
| | RNASC1 | Ribonuclease | P07990 | 0.6870 | 0.0816 | 0.5270 | 0.0363 | 26.1 | 17.4 | 1.23A | 0.6157 | 0.0890 | 0.4412 | 0.2090 | 16.5 | 14.8 | 1.23B |
| | | pancreatic | | | | to | | | | | | | to | | | | |
| | | | | | | 0.0470 | | | | | | | 0.7902 | | | | |
| | KRT13 | Keratin. type I | P13646 | 0.7391 | 0.0754 | 0.5913 | 0.0074 | 52.2 | 30.4 | 1.24A | 0.6273 | 0.0852 | 0.4603 | 0.1670 | 44.4 | 25.9 | 1.24B |
| | | cytoskeletal 13 | | | | to | | | | | | | to | | | | |
| | | | | | | 0.8869 | | | | | | | 0.7943 | | | | |
| | JUP | Junction | P14023 | 0.7185 | 0.0802 | 0.5614 | 0.0158 | 21.7 | 17.4 | 1.25A | 0.6370 | 0.0881 | 0.4643 | 0.1390 | 19.2 | 15.4 | 1.25B |
| | | plakoglobin | | | | to | | | | | | | to | | | | |
| | | | | | | 0.8757 | | | | | | | 0.8097 | | | | |
| | LCN2 | Neutrophil | P80188 | 0.6370 | 0.0868 | 0.4668 | 0.1250 | 34.8 | 8.7 | 1.26A | 0.7593 | 0.0744 | 0.6135 | 0.0049 | 48.1 | 48.1 | 1.26B |
| | | golatinaso | | | | to | | | | | | | to | | | | |
| | | associated | | | | 0.8071 | | | | | | | 0.9051 | | | | |
| | | lipocalin | | | | | | | | | | | | | | | |
| | SPARCL1 | SPARC-like | Q14515 | 0.5913 | 0.0901 | 0.4148 | 0.3065 | 43.5 | 34.8 | 1.27A | 0.6551 | 0.0826 | 0.4931 | 0.0923 | 40.7 | 37.0 | 1.27B |
| | | prolein 1 | | | | to | | | | | | | to | | | | |
| | | | | | | 0.7678 | | | | | | | 0.8171 | | | | |
| | LCP1 | Plastin−2 | P13796 | 0.5818 | 0.0892 | 0.4070 | 0.3646 | 27.3 | 9.1 | 1.28A | 0.7019 | 0.0797 | 0.5457 | 0.0296 | 46.2 | 42.3 | 1.28B |
| | | | | | | to | | | | | | | to | | | | |
| | | | | | | 0.7567 | | | | | | | 0.8581 | | | | |
| | MSMB | Beta-micro- | P08118 | 0.6130 | 0.0865 | 0.4435 | 0.2055 | 34.4 | 26.1 | 1.29A | 0.6551 | 0.0841 | 0.4903 | 0.0923 | 37.0 | 29.6 | 1.29B |
| | | seminoprotein | | | | to | | | | | | | to | | | | |
| | | | | | | 0.7626 | | | | | | | 0.8199 | | | | |
| | SCUBE2 | Signal peptide. | Q9NQ36 | 0.6652 | 0.0847 | 0.4992 | 0.0642 | 39.1 | 30.4 | 1.30A | 0.6875 | 0.0801 | 0.5305 | 0.0418 | 37.0 | 33.3 | 1.30B |
| | | CUB and EGF- | | | | to | | | | | | | to | | | | |
| | | like domain- | | | | 0.8312 | | | | | | | 0.8445 | | | | |
| | | containing | | | | | | | | | | | | | | | |
| | | protein 2 | | | | | | | | | | | | | | | |
| | IG [symbol] V3-10 | Immunoglobulin | A0A075B6K4 | 0.6478 | 0.0857 | 0.4799 | 0.0978 | 39.1 | 8.7 | 1.31A | 0.6875 | 0.0805 | 0.5297 | 0.0418 | 37.0 | 33.3 | 1.31B |
| | | lambda variable | | | | to | | | | | | | to | | | | |
| | | 3-10 | | | | 0.8157 | | | | | | | 0.8453 | | | | |
| | TA [symbol] DO1 | Transaldolase | P37837 | 0.6548 | 0.0872 | 0.4838 | 0.0000 | 38.1 | 0.0 | 1.32A | 0.6700 | 0.0844 | 0.5045 | 0.0692 | 36.0 | 28.0 | 1.32B |
| | | | | | | to | | | | | | | to | | | | |
| | | | | | | 0.8257 | | | | | | | 0.8355 | | | | |
| | SERPINA6 | Corticosteroid- | P08185 | 0.6283 | 0.0865 | 0.4587 | 0.1508 | 39.1 | 30.4 | 1.33A | 0.6458 | 0.0842 | 0.4807 | 0.1134 | 37.0 | 25.9 | 1.33B |
| | | binding globulin | | | | to | | | | | | | to | | | | |
| | | | | | | 0.7978 | | | | | | | 0.8109 | | | | |
| | TGFBR2 | TCF-beta | P37173 | 0.5630 | 0.0938 | 0.3793 | 0.4801 | 47.8 | 30.4 | 1.34A | 0.6389 | 0.0847 | 0.4729 | 0.1317 | 48.1 | 48.1 | 1.34B |
| | | receptor type-2 | | | | to | | | | | | | to | | | | |
| | | | | | | 0.7468 | | | | | | | 0.8049 | | | | |
| | ANXA3 | Annexin A3 | P12429 | 0.5068 | 0.0933 | 0.3239 | 0.9398 | 27.3 | 0.0 | 1.35A | 0.6202 | 0.0067 | 0.4502 | 0.1953 | 38.5 | 30.8 | 1 35B |
| | | | | | | to | | | | | | | to | | | | |
| | | | | | | 0.6897 | | | | | | | 0.7902 | | | | |
| | LYVE1 | Lymphatic | Q9Y5Y7 | 0.5717 | 0.0935 | 0.3885 | 0.4217 | 43.5 | 34.8 | 1.36A | 0.6227 | 0.0865 | 0.4532 | 0.1830 | 40.7 | 33.3 | 1.36B |
| | | vessel | | | | to | | | | | | | to | | | | |
| | | endothelial | | | | 0.7550 | | | | | | | 0.7921 | | | | |
| | | hyaluronic acid | | | | | | | | | | | | | | | |
| | | receptor 1 | | | | | | | | | | | | | | | |
| | PTGDS | Prostaglandin- | P41222 | 0.5652 | 0.0930 | 0.3829 | 0.4651 | 43.5 | 8.7 | 1.37A | 0.6528 | 0.0850 | 0.4863 | 0.0973 | 55.6 | 51.9 | 1.37B |
| | | H2 D-isomerase | | | | to | | | | | | | to | | | | |
| | | | | | | 0.7475 | | | | | | | 0.8193 | | | | |
| | IGKV3D-11 | Immunoglobulin | A0A0A0MRZ8 | 0.6239 | 0.0913 | 0.4451 | 0.1652 | 47.8 | 34.8 | 1.38A | 0.6319 | 0.0865 | 0.4624 | 0.1521 | 48.1 | 40.7 | 1.38B |
| | | kappa variable | | | | to | | | | | | | to | | | | |
| | | 3D-11 | | | | 0.8028 | | | | | | | 0.8015 | | | | |
| | TKT | Transkelolase | P29401 | 0.6773 | 0.0828 | 0.5151 | 0.0495 | 31.8 | 31.8 | 1.39A | 0.6923 | 0.0852 | 0.5253 | 0.0383 | 30.8 | 26.9 | 1.39B |
| | | | | | | to | | | | | | | to | | | | |
| | | | | | | 0.8395 | | | | | | | 0.8593 | | | | |
| | AMBP | Protoin AMBP | P02760 | 0.6326 | 0.0885 | 0.4592 | 0.1375 | 47.8 | 30.1 | 1.40A | 0.6181 | 0.0840 | 0.4517 | 0.2000 | 40.7 | 33.3 | 1.40B |
| | | | | | | to | | | | | | | to | | | | |
| | | | | | | 0.8060 | | | | | | | 0.7844 | | | | |
| | HYOU1 | Hypoxia up- | Q9Y4L1 | 0.6613 | 0.0887 | 0.4874 | 0.0743 | 47.8 | 43.5 | 1.41A | 0.6815 | 0.0814 | 0.5219 | 0.0537 | 51.9 | 44.4 | 1.41B |
| | | regulated | | | | to | | | | | | | to | | | | |
| | | protein 1 | | | | 0.8352 | | | | | | | 0.8411 | | | | |
| | MASP1 | Mannan-binding | P48740 | 0.7705 | 0.0744 | 0.6247 | 0.0027 | 63.6 | 40.9 | 1.42A | 0.7284 | 0.0773 | 0.5769 | 0.0139 | 46.2 | 34.6 | 1.42B |
| | | lectin serine | | | | to | | | | | | | to | | | | |
| | | protease 1 | | | | 0.9162 | | | | | | | 0.8798 | | | | |
| | CEL | Bile salt- | P19835 | 0.5326 | 0.0895 | 0.3572 | 0.7149 | 26.1 | 21.7 | 1.43A | 0.5231 | 0.0906 | 0.3456 | 0.8016 | 25.9 | 25.9 | 1.43B |
| | | activaled lipase | | | | to | | | | | | | to | | | | |
| | | | | | | 0.7080 | | | | | | | 0.7007 | | | | |
| | [symbol] 2 | Elongation | P13639 | 0.6523 | 0.0852 | 0.4853 | 0.0915 | 36.4 | 36.4 | 1.44A | 0.6010 | 0.0085 | 0.4275 | 0.2767 | 34.6 | 34.6 | 1.44B |
| | | factor 2 | | | | to | | | | | | | to | | | | |
| | | | | | | 0.0192 | | | | | | | 0.7745 | | | | |
| | SCUBE1 | Signal peptide, | Q8IWY1 | 0.6326 | 0.0912 | 0.4539 | 0.1375 | 52.2 | 21.7 | 1.15A | 0.6736 | 0.0838 | 0.5094 | 0.0595 | 51.8 | 18.5 | 1.45B |
| | | CUB and EGF- | | | | to | | | | | | | to | | | | |
| | | like domain- | | | | 0.8113 | | | | | | | 0.8379 | | | | |
| | | containing | | | | | | | | | | | | | | | |
| | | protein 1 | | | | | | | | | | | | | | | |
| | C4BPA | C4b-binding | P04003 | 0.6357 | 0.0877 | 0.4638 | 0.1371 | 33.3 | 23.8 | 1.46A | 0.6125 | 0.0880 | 0.4400 | 0.2291 | 32.0 | 24.0 | 1.46B |
| | | protein alpha | | | | to | | | | | | | to | | | | |
| | | chain | | | | 0.8076 | | | | | | | 0.7850 | | | | |
| | BASP1 | Brain anid | P80723 | 0.6304 | 0.0856 | 0.4627 | 0.1440 | 30.4 | 21.7 | 1.47A | 0.6829 | 0.0828 | 0.5205 | 0.0472 | 22.2 | 18.5 | 1.47B |
| | | soluble protein | | | | to | | | | | | | to | | | | |
| | | 1 | | | | 0.7981 | | | | | | | 0.8452 | | | | |
| | IGKV2 30 | Immunoglobulin | P06310 | 0.6704 | 0.0841 | 0.5145 | 0.0499 | 40.9 | 40.9 | 1.48A | 0.5974 | 0.0888 | 0.4234 | 0.3037 | 34.6 | 34.6 | 1.48B |
| | | kappa variable | | | | to | | | | | | | to | | | | |
| | | 2 30 | | | | 0.8443 | | | | | | | 0.7715 | | | | |
| | AZGP1 | Zinc alpha 2 | P25311 | 0.6609 | 0.0832 | 0.4978 | 0.0716 | 34.8 | 13.0 | 1.49A | 0.7546 | 0.0768 | 0.6041 | 0.0057 | 43.1 | 11.1 | 1.49B |
| | | glycoprotein | | | | to | | | | | | | to | | | | |
| | | | | | | 0.8240 | | | | | | | 0.9052 | | | | |
| | APEH | Acylamino-acid- | P13798 | 0.5600 | 0.0928 | 0.3781 | 0.5162 | 35.0 | 5.0 | 1.50A | 0.5517 | 0.0935 | 0.3714 | 0.5621 | 33.3 | 1.1 | 1.50B |
| | | releasing | | | | to | | | | | | | to | | | | |
| | | enzyme | | | | 0.7419 | | | | | | | 0.7379 | | | | |
| | CDH1 | Cadherin-1 | P12830 | 0.5478 | 0.0904 | 0.3706 | 0.5922 | 34.8 | 17.4 | 1.51A | 0.5556 | 0.0875 | 0.3841 | 0.5465 | 22.2 | 14.8 | 1.51B |
| | | | | | | to | | | | | | | to | | | | |
| | | | | | | 0.7251 | | | | | | | 0.7270 | | | | |
| | S100A8 | Protein S100- | P05109 | 0.5818 | 0.0891 | 0.1072 | 0.3646 | 27.3 | 18.2 | 1.52A | 0.6755 | 0.0833 | 0.5121 | 0.0587 | 30.8 | 23.1 | 1.52B |
| | | A8 | | | | to | | | | | | | to | | | | |
| | | | | | | 0.7565 | | | | | | | 0.8388 | | | | |
| | CD55 | Complement | P08174 | 0.5565 | 0.0918 | 0.3766 | 0.5267 | 34.8 | 26.1 | 1 53A | 0.5741 | 0.0864 | 0.4048 | 0.4214 | 33.3 | 29.6 | 1.53B |
| | | decay- | | | | to | | | | | | | to | | | | |
| | | accelerating | | | | 0.7364 | | | | | | | 0.7434 | | | | |
| | | factor | | | | | | | | | | | | | | | |
| | DSG1 | Desmoglein-1 | Q02413 | 0.0891 | 0.0851 | 0.5223 | 0.0341 | 47.6 | 34.8 | 1.54A | 0.6551 | 0.0828 | 0.4929 | 0.0923 | 40.7 | 29.6 | 1.54B |
| | | | | | | to | | | | | | | to | | | | |
| | | | | | | 0.8559 | | | | | | | 0.8173 | | | | |
| | PTGERN | Pmstaglancin | Q9P2B2 | 0.6565 | 0.0843 | 0.4912 | 0.0796 | 39.1 | 8.7 | 1.55A | 0.6181 | 0.0876 | 0.4464 | 0.2000 | 37.1 | 22.2 | 1.55B |
| | | [symbol] 2 receptor | | | | to | | | | | | | to | | | | |
| | | negative | | | | 0.8218 | | | | | | | 0.7897 | | | | |
| | | regulator | | | | | | | | | | | | | | | |
| | EMCN | Endomucin | Q9ULC0 | 0.6304 | 0.0871 | 0.4591 | 0.1440 | 39.1 | 8.7 | 1.56A | 0.5741 | 0.0878 | 0.4019 | 0.4211 | 33.3 | 7.4 | 1.56B |
| | | | | | | to | | | | | | | to | | | | |
| | | | | | | 0.8018 | | | | | | | 0.7462 | | | | |
| | IL15RA | Interleukin-15 | Q13261 | 0.7977 | 0.0749 | 0.6510 | 0.0010 | 40.9 | 4.5 | 1.57A | 0.6611 | 0.0903 | 0.4041 | 0.0827 | 11.5 | 3.8 | 1.57B |
| | | receptor | | | | to | | | | | | | to | | | | |
| | | subunit alpha | | | | 0.9445 | | | | | | | 0.8380 | | | | |
| | FCER1A | High affinity | P12319 | 0.7024 | 0.0835 | 0.5388 | 0.0266 | 52.4 | 19.4 | 1.58A | 0.6175 | 0.0885 | 0.4440 | 0.2091 | 48.0 | 16.0 | 1.58B |
| | | immunoglobulin | | | | to | | | | | | | to | | | | |
| | | epsilon | | | | 0.8660 | | | | | | | 0.7910 | | | | |
| | | receptor | | | | | | | | | | | | | | | |
| | | subunit alpha | | | | | | | | | | | | | | | |
| | NELL1 | Protein kinase | Q92832 | 0.6194 | 0.0930 | 0.4366 | 0.2087 | 20.0 | 5.0 | 1.59A | 0.7024 | 0.0897 | 0.5266 | 0.0396 | 33.3 | 4.2 | 1.59B |
| | | C-binding | | | | to | | | | | | | to | | | | |
| | | protein NELL1 | | | | 0.8023 | | | | | | | 0.8782 | | | | |
| | GPR37L1 | G-protein | O60883 | 0.7091 | 0.0819 | 0.5486 | 0.0205 | 31.8 | 18.2 | 1.60A | 0.7019 | 0.0876 | 0.5303 | 0.0296 | 19.2 | 15.4 | 1.60B |
| | | coupled | | | | to | | | | | | | to | | | | |
| | | receptor 37-like 1 | | | | 0.8696 | | | | | | | 0.8736 | | | | |
| Control | CD44 | CD44 antigen | P16070 | 0.5717 | 0.0909 | 0.3936 | 0.427 | 13.0 | 4.3 | 1.61A | 0.5023 | 0.0933 | 0.3195 | 0.9800 | 3.7 | 3.7 | 1.61B |
| | | | | | | to | | | | | | | to | | | | |
| | | | | | | 0.7499 | | | | | | | 0.6851 | | | | |
| | RNASE2 | Non-secretory | P10153 | 0.5326 | 0.0905 | 0.3553 | 0.7149 | 13.0 | 4.3 | 1.62A | 0.5023 | 0.0965 | 0.3132 | 0.9800 | 3.7 | 3.7 | 1.62B |
| | | ribonuclease | | | | to | | | | | | | to | | | | |
| | | | | | | 0.7099 | | | | | | | 0.6914 | | | | |

[symbol]

indicates data missing or illegible when filed

TABLE 7.1

| | Detect PCa (any grade) | | | | | | Detect High-grade PCa (GS ≥ 7) | | | | | |
|---|---|---|---|---|---|---|---|---|---|---|---|---|
| | | | | | Speci- | Speci- | | | | | Speci- | Speci- |
| | | | 95% | | ficity | ficity | | | 95% | | ficity | ficity |
| | | | Con- | | at 90% | at 100% | | | Con- | | at 90% | at 100% |
| | | Std. | fidence | p- | Sensi- | Sensi- | | Std. | fidence | p- | Sensi- | Sensi- |
| | AUC | Error | interval | value | tivity | tivity | AUC | Error | interval | value | tivity | tivity |
| PEDF | 0.8023 | 0.06956 | 0.6659 to | 0.0008 | 68.2 | 36.4 | 0.7236 | 0.07842 | 0.5699 to | 0.016 | 61.5 | 30.8 |
| | | | 0.9386 | | | | | | 0.8773 | | | |
| FCFR2 | 0.6717 | 0.08378 | 0.5075 to | 0.0544 | 52.2 | 30.4 | 0.6412 | 0.0834 | 0.4777 to | 0.1254 | 40.7 | 44464.0 |
| | | | 0.8360 | | | | | | 0.8047 | | | |
| CANX | 0.7043 | 0.08496 | 0.5377 to | 0.0273 | 17.6 | 38.1 | 0.728 | 0.07902 | 0.5731 to | 0.0169 | 44.0 | 44.0 |
| | | | 0.8708 | | | | | | 0.8829 | | | |
| KRT13 | 0.7391 | 0.0754 | 0.5913 to | 0.0074 | 52.2 | 30.4 | 0.6273 | 0.08522 | 0.4603 to | 0.167 | 44.4 | 25.9 |
| | | | 0.0869 | | | | | | 0.7943 | | | |
| HPX | 0.7761 | 0.06961 | 0.6396 to | 0.002 | 52.2 | 39.1 | 0.8125 | 0.06605 | 0.6830 to | 0.0007 | 48.1 | 33.3 |
| | | | 0.9125 | | | | | | 0.9420 | | | |
| HRNR | 0.7522 | 0.0759 | 0.6033 to | 0.0047 | 47.8 | 13.0 | 0.6458 | 0.0852 | 0.4789 to | 0.1134 | 33.3 | 11.2 |
| | | | 0.9010 | | | | | | 0.8127 | | | |
| CD99 | 0.6750 | 0.0835 | 0.5114 to | 0.0525 | 36.4 | 31.8 | 0.7019 | 0.0827 | 0.5399 to | 0.0296 | 26.9 | 26.9 |
| | | | 0.8386 | | | | | | 0.8639 | | | |
| Age | 0.6685 | 0.08401 | 0.5038 to | 0.0592 | 17.4 | 0.0 | 0.6343 | 0.09306 | 0.4519 to | 0.145 | 11.1 | 0.0 |
| | | | 0.8331 | | | | | | 0.8167 | | | |
| PI-RADS | 0.0403 | 0.06679 | 0.7094 to | 0.0003 | 60.0 | 0.0 | 0.869 | 0.05767 | 0.7560 to | 0.0002 | 54.2 | 54.2 |
| | | | 0.9712 | | | | | | 0.9821 | | | |
| PEDF + Age | 0.8432 | 0.0631 | 0.7195 to | 0.0001 | 72.7 | 54.5 | 0.7716 | 0.07212 | 0.6303 to | 0.0034 | 65.4 | 46.1 |
| | | | 0.9669 | | | | | | 0.9130 | | | |
| FCER2 + Age | 0.7217 | 0.0787 | 0.5675 to | 0.013 | 52.2 | 47.8 | 0.6898 | 0.07995 | 0.5331 to | 0.0394 | 40.7 | 37.0 |
| | | | 0.8760 | | | | | | 0.8465 | | | |
| CANX + Age | 0.782 | 0.07375 | 0.6374 to | 0.0023 | 52.4 | 42.9 | 0.784 | 0.07197 | 0.6420 to | 0.0029 | 68.0 | 52.0 |
| | | | 0.9265 | | | | | | 0.9251 | | | |
| KRT13 + Age | 0.7348 | 0.07628 | 0.5853 to | 0.0085 | 52.2 | 30.4 | 0.6505 | 0.0848 | 0.4843 to | 0.1024 | 40.7 | 33.3 |
| | | | 0.8843 | | | | | | 0.8167 | | | |
| HPX + Age | 0.8326 | 0.06063 | 0.7138 to | 0.0002 | 65.2 | 56.5 | 0.838 | 0.05956 | 0.7212 to | 0.0002 | 55.6 | 55.6 |
| | | | 0.0515 | | | | | | 0.9547 | | | |
| HRNR + Age | 0.8 | 0.1 | 0.6281 to | 0.0 | 43.5 | 17.4 | 0.6852 | 0.0845 | 0.5196 to | 0.0444 | 25.9 | 14.8 |
| | | | 0.9154 | | | | | | 0.8508 | | | |
| CD99 + Age | 0.7 | 0.1 | 0.5469 to | 0.0 | 45.5 | 31.8 | 0.6851 | 0.0834 | 0.5217 to | 0.0461 | 26.9 | 26.9 |
| | | | 0.8668 | | | | | | 0.8485 | | | |
| PEDF + PI-RADS | 0.9211 | 0.04833 | 0.8352 to | <0.0001 | 63.2 | 47.4 | 0.9161 | 0.04976 | 0.8186 to | <0.0001 | 73.9 | 69.6 |
| | | | 1.000 | | | | | | 1.000 | | | |
| FCER2 + PI-RADS | 0.8722 | 0.05981 | 0.7550 to | <0.0001 | 75.0 | 15.0 | 0.8869 | 0.05488 | 0.7793 to | <0.0001 | 66.6 | 66.6 |
| | | | 0.9894 | | | | | | 0.9945 | | | |
| CANX + PI-RADS | 0.8562 | 0.06444 | 0.7200 to | 0.0003 | 72.2 | 33.3 | 0.0126 | 0.04662 | 0.8212 to | <0.0001 | 72.7 | 72.7 |
| | | | 0.9825 | | | | | | 1.000 | | | |
| HPX + PI-RADS | 0.8667 | 0.05827 | 0.7525 to | 0.0001 | 75.0 | 35.0 | 0.8958 | 0.04924 | 0.7993 to | <0.0001 | 70.8 | 70.8 |
| | | | 0.9800 | | | | | | 0.0923 | | | |
| KRT13 + PI-RADS | 0.8778 | 0.05658 | 0.7669 to | <0.0001 | 75.0 | 45.0 | 0.8631 | 0.05988 | 0.7457 to | 0.0002 | 66.6 | 66.6 |
| | | | 0.9887 | | | | | | 0.9805 | | | |
| HRNR + PI-RADS | 0.9 | 0.1 | 0.7768 to | <0.0001 | 55.0 | 25.0 | 0.8929 | 0.0521 | 0.7007 to | <0.0001 | 70.8 | 66.7 |
| | | | 0.9954 | | | | | | 0.9950 | | | |
| CD99 + PI-RADS | 0.9 | 0.1 | 0.7553 to | <0.0001 | 70.0 | 25.0 | 0.8958 | 0.0515 | 0.7949 to | <0.0001 | 70.2 | 70.2 |
| | | | 0.9891 | | | | | | 0.0068 | | | |
| PEDF + FCER2 | 0.8773 | 0.06342 | 0.7530 to | <0.0001 | 84.4 | 72.7 | 0.7957 | 0.07154 | 0.6555 to | 0.0014 | 73.1 | 61.5 |
| | | | 1.000 | | | | | | 0.9359 | | | |
| PEDF + FCER2 + | 0.8727 | 0.06661 | 0.7422 to | <0.0001 | 86.4 | 81.8 | 0.7981 | 0.07198 | 0.6570 to | 0.0012 | 73.1 | 69.2 |
| Age | | | 1.000 | | | | | | 0.9392 | | | |
| PEDF + FCER2 + | 0.9444 | 0.03595 | 0.8740 to | <0.0001 | 84.2 | 73.7 | 0.9255 | 0.04778 | 0.8318 to | <0.0001 | 02.6 | 69.6 |
| PI-RADS | | | 1.000 | | | | | | 1.000 | | | |
| PEDF + FCER2 + | 0.9444 | 0.03595 | 0.8740 to | <0.0001 | 84.2 | 13.7 | 0.9255 | 0.04778 | 0.8318 to | <0.0001 | 82.6 | 69.6 |
| Age + PI-RADS | | | 1.000 | | | | | | 1.000 | | | |
| PEDF + CANX | 0.9105 | 0.053 | 0.8067 to | <0.0001 | 85.0 | 70.0 | 0.8472 | 0.06278 | 0.7242 to | 0.0003 | 66.6 | 51.2 |
| | | | 1.000 | | | | | | 0.9703 | | | |
| PEDF + CANX + | 0.9184 | 0.04897 | 0.8224 to | <0.0001 | 80.0 | 80.0 | 0.8583 | 0.06158 | 0.7376 to | 0.0002 | 75.0 | 75.0 |
| Age | | | 1.000 | | | | | | 0.9790 | | | |
| PEDF + CANX + | 0.9273 | 0.04539 | 0.8384 to | <0.0001 | 76.5 | 76.5 | 0.9231 | 0.04335 | 0.8381 to | <0.0001 | 76.2 | 76.2 |
| PI-RADS | | | 1.000 | | | | | | 1.000 | | | |
| PEDF + CANX + | 0.9446 | 0.04111 | 0.8641 to | <0.0001 | 82.3 | 76.5 | 0.9267 | 0.04251 | 0.8434 to | <0.0001 | 80.9 | 76.2 |
| Age + PI-RADS | | | 1.000 | | | | | | 1.000 | | | |
| HPX + KRT13 | 0.8413 | 0.06133 | 0.7211 to | 0.0001 | 60.9 | 56.5 | 0.838 | 0.0609 | 0.7186 to | 0.0002 | 59.3 | 40.7 |
| | | | 0.9615 | | | | | | 0.9573 | | | |
| HPX + KKT13 + | 0.8522 | 0.05917 | 0.7352 to | <0.0001 | 78.3 | 60.9 | 0.8333 | 0.05998 | 0.7158 to | 0.0003 | 62.9 | 55.6 |
| Age | | | 0.9692 | | | | | | 0.9509 | | | |
| HPX + KRT13 + | 0.8778 | 0.05592 | 0.7682 to | <0.0001 | 75.0 | 60.0 | 0.8958 | 0.04908 | 0.7990 to | <0.0001 | 70.8 | 66.6 |
| PI-RADS | | | 0.9874 | | | | | | 0.9920 | | | |
| HPX + KRT13 + | 0.8889 | 0.05243 | 0.7861 to | <0.0001 | 75.0 | 70.0 | 0.8929 | 0.05009 | 0.7935 to | <0.0001 | 70.8 | 70.8 |
| Age + PI-RADS | | | 0.9917 | | | | | | 0.9922 | | | |
| PEDF + FCFR2 + | 0.9079 | 0.05658 | 0.7970 to | <0.0001 | 90.0 | 70.0 | 0.8361 | 0.06457 | 0.7096 to | 0.0005 | 66.7 | 54.2 |
| CANX | | | 1.000 | | | | | | 0.9627 | | | |
| PEDF + FCER2 + | 0.9211 | 0.05304 | 0.8171 to | <0.0001 | 90.0 | 85.0 | 0.8556 | 0.06394 | 0.7302 to | 0.0002 | 79.2 | 70.8 |
| CANX + Age | | | 1.000 | | | | | | 0.9809 | | | |
| PEDF + FCCR2 | 0.9308 | 0.04463 | 0.8433 to | <0.0001 | 76.5 | 76.5 | 0.9341 | 0.03940 | 0.0560 to | <0.0001 | 76.2 | 76.2 |
| CANX + PI-RADS | | | 1.000 | | | | | | 1.000 | | | |
| PEDF + FCER2 + | 0.0377 | 0.04109 | 0.8572 to | <0.0001 | 76.5 | 76.5 | 0.0341 | 0.04000 | 0.8557 to | <0.0001 | 80.6 | 80.6 |
| CANX + Age + | | | 1.000 | | | | | | 1.000 | | | |
| PI-RADS | | | | | | | | | | | | |
| PEDF + FCER2 + | 0.9105 | 0.05624 | 0.8003 to | <0.0001 | 90.0 | 75.0 | 0.0389 | 0.06398 | 0.7135 to | 0.0004 | 66.7 | 58.3 |
| CANX + KRT13 | | | 1.000 | | | | | | 0.9643 | | | |
| PEDF + FCER2 + | 0.9211 | 0.05267 | 0.8178 to | <0.0001 | 90.0 | 85.0 | 0.8630 | 0.06328 | 0.7399 to | 0.0002 | 83.3 | 70.8 |
| CANX + KRT13 + | | | 1.000 | | | | | | 0.0870 | | | |
| Age | | | | | | | | | | | | |
| PEDF + FCER2 + | 0.9273 | 0.04539 | 0.8384 to | <0.0001 | 76.5 | 76.5 | 0.9414 | 0.03686 | 0.8691 to | <0.0001 | 81.0 | 76.2 |
| CANX + KRT13 + | | | 1.000 | | | | | | 1.000 | | | |
| PI-RADS | | | | | | | | | | | | |
| PEDF + FCER2 + | 0.9343 | 0.04144 | 0.8530 to | <0.0001 | 76.5 | 76.5 | 0.0377 | 0.03830 | 0.8627 to | <0.0001 | 81.0 | 81.0 |
| CANX + KRT13 + | | | 1.000 | | | | | | 1.000 | | | |
| Age + PI-RADS | | | | | | | | | | | | |
| PEDF + FCER2 + | 0.9368 | 0.04104 | 0.8564 to | <0.0001 | 85.0 | 75.0 | 0.8694 | 0.05733 | 0.7571 to | 0.0001 | 58.3 | 50.0 |
| CANX + KRT13 + | | | 1.000 | | | | | | 0.9818 | | | |
| HPX | | | | | | | | | | | | |
| PEDF + FCER2 + | 0.9474 | 0.03465 | 0.8795 to | <0.0001 | 85.0 | 80.0 | 0.8861 | 0.05311 | 0.7920 to | <0.0001 | 58 3 | 54.2 |
| CANX + KRT13 + | | | 1.000 | | | | | | 0.9902 | | | |
| HDX + Age | | | | | | | | | | | | |
| PEDF + FCER2 + | 0.9343 | 0.04117 | 0.8536 to | <0.0001 | 82.4 | 76.5 | 0.9414 | 0.03686 | 0.8691 to | <0.0001 | 81.0 | 76.2 |
| CANX + KRT13 + | | | 1.000 | | | | | | 1.000 | | | |
| HPX + PI-RADS | | | | | | | | | | | | |
| PEDF + FCER2 + | 0.9516 | 0.03755 | 0.8780 to | <0.0001 | 82.4 | 82.4 | 0.9377 | 0.03860 | 0.8621 to | <0.0001 | 81.0 | 81.0 |
| CANX + KRT13 + | | | 1.000 | | | | | | 1.000 | | | |
| HPX + Age + | | | | | | | | | | | | |
| PI-RADS | | | | | | | | | | | | |
| PEDF + FCER2 + | 0.9658 | 0.02588 | 0.9151 to | <0.0001 | 90.0 | 60.0 | 0.9194 | 0.04667 | 0.8280 to | <0.0001 | 87.5 | 41.7 |
| CANX + KRT13 + | | | 1.000 | | | | | | 1.000 | | | |
| HPX + HRNR | | | | | | | | | | | | |
| PEDF + FCER2 + | 0.9737 | 0.02320 | 0.9282 to | <0.0001 | 05.0 | 85.0 | 0.9111 | 0.04667 | 0.8196 to | <0.0001 | 87.5 | 62.5 |
| CANX + KRT13 + | | | 1.000 | | | | | | 1.000 | | | |
| HPX + HRNR + | | | | | | | | | | | | |
| Age | | | | | | | | | | | | |
| PEDF + CLR2 + | 0.9689 | 0.02461 | 0.9206 to | <0.0001 | 82.4 | 82.4 | Overfitted | | | | | |
| CANX + KRT13 + | | | 1.000 | | | | | | | | | |
| HPX + HRNR + | | | | | | | | | | | | |
| PI-RADS | | | | | | | | | | | | |
| PEDF + FCER2 + | 0.9654 | 0.02590 | 0.9126 to | <0.0001 | 82.4 | 82.4 | Overfitted | | | | | |
| CANX + KRT13 + | | | 1.000 | | | | | | | | | |
| HPX + HKNR + | | | | | | | | | | | | |
| Age + PI-RADS | | | | | | | | | | | | |
| PEDF + FCER2 + | 0.9723 | 0.02186 | 0.9295 to | <0.0001 | 89.5 | 68.4 | 0.9188 | 0.04473 | 0.8312 to | <0.0001 | 73.9 | 47.8 |
| CANX + KRT13 + | | | 1.000 | | | | | | 1.000 | | | |
| HPX + HRNR + | | | | | | | | | | | | |
| CD99 | | | | | | | | | | | | |
| PEDF + FCER2 + | 0.9834 | 0.01536 | 0.9533 to | <0.0001 | 84.2 | 84.2 | 0.9275 | 0.04045 | 0.8483 to | <0.0001 | 65.2 | 65.2 |
| CANX + KRT13 + | | | 1.000 | | | | | | 1.000 | | | |
| HPX + HRNR+ | | | | | | | | | | | | |
| CD99 + Age | | | | | | | | | | | | |

| | | | | | | | |
|---|---|---|---|---|---|---|---|
| PEDF + FCER2 + | 0.9758 | 0.02055 | 0.9355 to | <0.0001 | 88.2 | 82.4 | Overfitted |
| CANX + KRT13 + | | | 1.000 | | | | |
| HPX + HRNR+ | | | | | | | |
| CD99 + PI-RADS | | | | | | | |
| PEDF + FCER2 + | 0.9689 | 0.02461 | 0.9206 to | <0.0001 | 82.4 | 82.4 | Overtitted |
| CANX + KRT13 + | | | 1.000 | | | | |
| HPX + HRNR+ | | | | | | | |
| CD99 + Age + | | | | | | | |
| PI-RADS | | | | | | | |

TABLE 7.2
Formulas
Tumor~β0 + (β1*x1) + (β2*x2) + (βn*xn); x = biomarker concentration or clinical variable (Age, PI-RADS, etc.)

| | Detection of all PCa grades | | | | | | Detection of high-grade PCa | | | | | |
|---|---|---|---|---|---|---|---|---|---|---|---|---|
| | Parameter | Variable | | Standard | 95% CI | | Parameter | Variable | | Standard | 95% CI | |
| | estimates | (x) | Estimate | error | (profile likelihood) | | estimates | (x) | Estimate | error | (profile likelihood) | |
| PEDF + Age | β0 | Intercept | 3.073 | 3.744 | 10.03 to | 4.141 | β0 | Intercept | 2.685 | 3.412 | 0.805 to | 3.886 |
| | β1 | PEDF | −0.0000417 | 0.00001481 | −7.608e−005 to | −1.734e−005 | β1 | PEDF | −0.00002853 | 0.00001239 | −5.703e−005 to | −7.852e−006 |
| | β2 | Age | 0.0873 | 0.05898 | −0.02141 to | 0.2153 | β2 | Age | 0.06127 | 0.05188 | −0.03664 to | 0.1714 |
| FCER2 + Age | β0 | Intercept | −3.504 | 3.313 | −10.48 to | 2.768 | β0 | Intercept | −3.307 | 3.281 | −10.18 to | 2.944 |
| | β1 | FCER2 | −0.000005195 | 0.000002518 | −1.088e−005 to | −8.613e−007 | β1 | FCER2 | −4.554E−06 | 0.000002603 | −1.052e−005 to | −1.435e−007 |
| | β2 | Age | 0.07049 | 0.05079 | −0.02428 to | 0.1791 | β2 | Age | 0.05847 | 0.04966 | 0.03570 to | 0.1634 |
| CANX + Age | β0 | Intercept | −6.327 | 3.984 | −15.00 to | 1.063 | β0 | Intercept | −5.884 | 4.079 | −14.66 to | 1.729 |
| | β1 | CANX | −0.00003489 | 0.00001427 | −7.083e−005 to | −1.201e−005 | β1 | CANX | −0.00004341 | 0.00002058 | −9.484e−005 to | −1.326e−005 |
| | β2 | Age | 0.1217 | 0.06392 | 0.006029 to | 0.2637 | β2 | Age | 0.1109 | 0.06469 | −0.007790 to | 0.2519 |
| KRT13 + Age | β0 | Intercept | −1.643 | 3.418 | −8.675 to | 5.052 | β0 | Intercept | −2.4 | 3.333 | −9.316 to | 4.049 |
| | β1 | KRT13 | −7.318E−07 | 3.494E−07 | −1.541e−006 to | −1.429e−007 | β1 | KRT13 | −4.125E−07 | 3.058E−07 | −1.114e−006 to | 9.540e−009 |
| | β2 | Age | 0.03965 | 0.05119 | 0.05917 to | 0.1463 | β2 | Age | 0.03855 | 0.0495 | −0.05719 to | 0.1414 |
| HPX + Age | β0 | Intercept | −6.343 | 4.249 | −15.60 to | 1.534 | β0 | Intercept | −4.707 | 4.226 | −13.75 to | 3.329 |
| | β1 | HPX | −3.837E−07 | 0.000000164 | −7.716e−007 to | −1.444e−007 | β1 | HPX | −5.187E−07 | 2.155E−07 | −1.011e−006 to | −1.764e−007 |
| | β2 | Age | 0.139 | 0.06728 | 0.01849 to | 0.2898 | β2 | Age | 0.1169 | 0.06631 | −0.004229 to | 0.2632 |
| HRNR + Age | β0 | Intercept | −2.174 | 3.329 | −9.080 to | 4.262 | β0 | Intercept | −2.721 | 3.289 | −9.585 to | 3.584 |
| | β1 | HRNR | −0.00003055 | 0.00001709 | −7.182e−005 to | −3.542e−006 | β1 | HRNR | −0.0000171 | 0.00001471 | −5.254e−005 to | 1.261e−006 |
| | β2 | Age | 0.04616 | 0.04991 | −0.04910 to | 0.1508 | β2 | Age | 0.04248 | 0.04883 | −0.05121 to | 0.1444 |
| CD99 + Age | β0 | Intercept | 3.161 | 3.524 | 10.58 to | 3.563 | β0 | Intercept | 2.839 | 3.481 | 10.12 to | 3.839 |
| | β1 | CD99 | −0.000001203 | 6.703E−07 | −2.833e−006 to | −1.051e−007 | β1 | CD99 | −1.135E−06 | 7.219E−07 | −2.935e−006 to | 2.163e−008 |
| | β2 | Age | 0.06025 | 0.05322 | −0.04018 to | 0.1733 | β2 | Age | 0.0476 | 0.05195 | −0.05175 to | 0.1567 |
| PEDF + | β0 | Intercept | 0.05892 | 1.523 | −3.313 to | 2.969 | β0 | Intercept | −3.604 | 2.244 | −8.817 to | 0.08144 |
| PI-RADS | β1 | PEDF | −0.00007082 | 0.00002977 | −0.0001449 to | −2.436e−005 | β1 | PEDF | −0.0000411 | 0.00002246 | −9.265e−005 to | −3.495e−006 |
| | β2 | PI-RADS | 1.305 | 0.4735 | 0.5272 to | 2.466 | β2 | PI-RADS | 1.621 | 0.6143 | 0.6582 to | 3.126 |
| FCER2 + | β0 | Intercept | −2.44 | 1.49 | −5.785 to | 0.1735 | β0 | Intercept | −5.232 | 2.187 | −10.36 to | −1.602 |
| PI-RADS | β1 | FCER2 | −0.000004565 | 0.000003098 | −1.146e−005 to | 7.648e−007 | β1 | FCER2 | −3.799E−06 | 0.000003476 | −1.162e−005 to | 2.277e−006 |
| | β2 | PI-RADS | 1.103 | 0.429 | 0.3953 to | 2.115 | β2 | PI-RADS | 1.675 | 0.6077 | 0.6938 to | 3.124 |
| CANX + | β0 | Intercept | −1.649 | 1.511 | −4.983 to | 1.104 | β0 | Intercept | −5.032 | 2.643 | −11.41 to | −0.7036 |
| PI-RADS | β1 | CANX | −0.00002805 | 0.00001688 | −6.907e−005 to | −1.377e−006 | β1 | CANX | −0.00006604 | 0.00003347 | −0.0001443 to | −1.171e−005 |
| | β2 | PI-RADS | 0.9309 | 0.4027 | 0.2459 to | 1.865 | β2 | PI-RADS | 2.021 | 0.8514 | 0.7290 to | 4.176 |
| KRT13 + | β0 | Intercept | −2.138 | 1.409 | −5.396 to | 0.2773 | β0 | Intercept | −5.558 | 2.257 | −10.78 to | −1.890 |
| PI-RADS | β1 | KRT13 | −6.907E−07 | 4.245E−07 | −1.692e−006 to | −1.447e−008 | β1 | KRT13 | −1.293E−07 | 3.667E−07 | −1.002e−006 to | 1.207e−007 |
| | β2 | PI-RADS | 1.002 | 0.4084 | 0.3255 to | 1.977 | β2 | PI-RADS | 1.59 | 0.5912 | 0.6343 to | 2.979 |
| HPX + | β0 | Intercept | −1.076 | 1.755 | −4.824 to | 2.348 | β0 | Intercept | −3.064 | 2.498 | −8.627 to | 1.427 |
| PI-RADS | β1 | HPX | −2.265E−07 | 1.505E−07 | −6.091e−007 to | −4.806e−009 | β1 | HPX | −3.496E−07 | 2.517E−07 | −9.322e−007 to | 9.561e−009 |
| | β2 | PI-RADS | 0.8699 | 0.3792 | 0.2339 to | 1.771 | β2 | PI-RADS | 1.435 | 0.5899 | 0.4927 to | 2.841 |
| HRNR + | β0 | Intercept | −2.439 | 1.438 | −5.787 to | −0.001338 | β0 | Intercept | −5.486 | 2.186 | −10.63 to | −1.931 |
| PI-RADS | β1 | HRNR | −0.00003137 | 0.00001794 | −7.427e−005 to | −1.186e−006 | β1 | HRNR | −0.00001357 | 0.00001717 | −5.184e−005 to | 6.225e−006 |
| | β2 | PI-RADS | 1.093 | 0.4301 | 0.3877 to | 2.127 | β2 | PI-RADS | 1.634 | 0.6042 | 0.6649 to | 3.074 |
| CD99 + | β0 | Intercept | 2.28 | 1.442 | 5.565 to | 0.2149 | β0 | Intercept | 4.937 | 2.179 | 10.06 to | 1.353 |
| PI-RADS | β1 | CD99 | −0.000001527 | 9.912E−07 | −3.935e−006 to | 4.814e−008 | β1 | CD99 | −1.519E−06 | 0.000001218 | −4.449e−006 to | 4.257e−007 |
| | β2 | PI-RADS | 1.024 | 0.4129 | 0.3364 to | 1.994 | β2 | PI-RADS | 1.595 | 0.6016 | 0.6265 to | 3.033 |
| PEDF + | β0 | Intercept | 5.075 | 1.561 | 2.457 to | 8.727 | β0 | Intercept | 2.852 | 1.152 | 0.8289 to | 5.432 |
| FCER2 | β1 | PEDF | −0.00005188 | 0.00001701 | −9.222e−005 to | −2.386e−005 | β1 | PEDF | −0.00003347 | 0.00001335 | −6.427e−005 to | −1.103e−005 |
| | β2 | FCER2 | −0.000008438 | 0.000003702 | −1.741e−005 to | −2.454e−006 | β2 | FCER2 | −5.878E−06 | 0.000003154 | −1.337e−005 to | −7.866e−007 |
| PEDF + | β0 | Intercept | 2.033 | 4.661 | −6.977 to | 11.97 | β0 | Intercept | 0.3041 | 3.865 | −7.457 to | 8.132 |
| FCER2 + | β1 | PEDF | −0.00005013 | 0.00001686 | −9.012e−005 to | −2.230e−005 | β1 | PEDF | −0.00003234 | 0.00001333 | −6.301e−005 to | −9.853e−006 |
| Age | β2 | FCER2 | −0.000007722 | 0.000003759 | −1.682e−005 to | −1.714e−006 | β2 | FCER2 | −0.00000552 | 0.000003215 | −1.313e−005 to | −3.273e−007 |
| | β3 | Age | 0.04306 | 0.06429 | −0.08191 to | 0.1776 | β3 | Age | 0.037 | 0.05443 | −0.06811 to | 0.1506 |
| PEDF + | β0 | Intercept | 2.007 | 1.948 | −1.933 to | 6.139 | β0 | Intercept | −2.512 | 2.437 | −8.005 to | 1.783 |
| FCER2 + | β1 | PEDF | −0.00006842 | 0.00002808 | −0.0001411 to | −2.526e−005 | β1 | PEDF | −0.00003906 | 0.00002136 | −8.871e−005 to | −3.539e−006 |
| PI-RADS | β2 | FCER2 | −0.00000633 | 0.000004277 | −1.729e−005 to | 6.664e−008 | β2 | FCER2 | −4.415E−06 | 0.000004171 | −1.435e−005 to | 2.333e−006 |
| | β3 | PI-RADS | 1.13 | 0.4595 | 0.3576 to | 2.260 | β3 | PI-RADS | 1.551 | 0.6306 | 0.5584 to | 3.078 |
| PEDF + | β0 | Intercept | −0.7865 | 5.876 | −12.73 to | 11.40 | β0 | Intercept | −4.353 | 5.575 | −16.67 to | 6.467 |
| FCER2 + | β1 | PEDF | −0.0000685 | 0.00002888 | −0.0001440 to | −2.445e−005 | β1 | PEDF | −0.0000394 | 0.00002168 | −9.019e−005 to | −3.495e−006 |
| Age + | β2 | FCER2 | −0.000005882 | 0.000004343 | −1.689e−005 to | 7.600e−007 | β2 | FCER2 | −4.416E−06 | 0.000004205 | −1.441e−005 to | 2.424e−006 |
| PI-RADS | β3 | Age | 0.04022 | 0.0802 | −0.1255 to | 0.2061 | β3 | Age | 0.0303 | 0.08145 | −0.1363 to | 0.1991 |
| | β4 | PI-RADS | 1.144 | 0.4624 | 0.3612 to | 2.270 | β4 | PI-RADS | 1.514 | 0.629 | 0.5421 to | 3.049 |
| PEDF + | β0 | Intercept | 5.15 | 1.689 | 2.414 to | 9.241 | β0 | Intercept | 2.964 | 1.177 | 0.9379 to | 5.674 |
| CANX | β1 | PEDF | −0.00005525 | 0.00002071 | −0.0001056 to | −2.203e−005 | β1 | PEDF | −0.00003086 | 0.00001454 | −6.429e−005 to | −6.802e−006 |
| | β2 | CANX | −0.00003392 | 0.00001503 | −7.213e−005 to | −9.670e−006 | β2 | CANX | −0.00003452 | 0.0000176 | −7.850e−005 to | −7.689e−006 |
| PEDF + | β0 | Intercept | −2.554 | 5.05 | −13.37 to | 7.335 | β0 | Intercept | −3.295 | 4.45 | −12.63 to | 5.393 |
| CANX + Age | β1 | PEDF | −0.00005634 | 0.0000211 | −0.0001076 to | −2.215e−005 | β1 | PEDF | −0.00003096 | 0.00001488 | −6.496e−005 to | −5.744e−006 |
| | β2 | CANX | −0.00004195 | 0.00001791 | −8.840e−005 to | −1.361e−005 | β2 | CANX | −0.0000416 | 0.00002094 | −9.627e−005 to | −1.103e−005 |
| | β3 | Age | 0.1248 | 0.08409 | −0.02236 to | 0.3231 | β3 | Age | 0.09998 | 0.07147 | −0.03152 to | 0.2569 |
| PEDF + | β0 | Intercept | 2.198 | 2.021 | −1.869 to | 6.498 | β0 | Intercept | −3.196 | 2.896 | −9.857 to | 1.883 |
| CANX + | β1 | PEDF | −0.00006167 | 0.00002791 | −0.0001342 to | −1.813e−005 | β1 | PEDF | −0.00002961 | 0.00002114 | −7.796e−005 to | 7.509e−006 |
| PI-RADS | β2 | CANX | −0.00002659 | 0.00001865 | −7.588e−005 to | 3.565e−006 | β2 | CANX | −0.00006724 | 0.00003789 | −0.0001563 to | −6.321e−006 |
| | β3 | PI-RADS | 0.8909 | 0.4703 | 0.06581 to | 2.031 | β3 | PI-RADS | 1.96 | 0.9116 | 0.5830 to | 4.268 |
| PEDF + | β0 | Intercept | 4.909 | 6.393 | −19.28 to | 7.864 | β0 | Intercept | −8.198 | 6.8 | −25.07 to | 4.185 |
| CANX + | β1 | PEDF | −0.00006678 | 0.00003106 | −0.0001487 to | −1.930e−005 | β1 | PEDF | −0.00003093 | 0.00002268 | −8.428e−005 to | 8.912e−006 |
| Age + | β2 | CANX | −0.00003097 | 0.00002114 | −8.975e−005 to | 1.755e−006 | β2 | CANX | −0.00007557 | 0.00003967 | −0.0001688 to | −9.686e−006 |
| PI-RADS | β3 | Age | 0.1201 | 0.1071 | −0.07937 to | 0.3747 | β3 | Age | 0.08135 | 0.09586 | −0.1040 to | 0.2967 |
| | β4 | PI-RADS | 0.8082 | 0.4701 | −0.01673 to | 1.948 | β4 | PI-RADS | 1.95 | 0.9395 | 0.5344 to | 4.356 |
| HPX + | β0 | Intercept | 3.237 | 1.138 | 1.357 to | 5.899 | β0 | Intercept | 2.834 | 1.229 | 0.7377 to | 5.543 |
| KRT13 | β1 | HPX | −3.028E−07 | 1.484E−07 | −6.620e−007 to | −9.109e−008 | β1 | HPX | −4.162E−07 | 0.000000195 | −8.586e−007 to | −1.154e−007 |
| | β2 | KRT13 | 0.000000815 | 3.669E−07 | −1.671e−006 to | −1.958e−007 | β2 | KRT13 | 4.133E−07 | 3.241E−07 | −1.157e−006 to | 8.010e−008 |
| HPX + | β0 | Intercept | −3.945 | 4.686 | −13.97 to | 5.113 | β0 | Intercept | −3.559 | 4.422 | −12.89 to | 5.019 |
| KRT13 + | β1 | HPX | −3.487E−07 | 1.592E−07 | −7.415e−007 to | −1.191e−007 | β1 | HPX | −4.692E−07 | 2.137E−07 | −9.619e−007 to | −1.428e−007 |
| Age | β2 | KRT13 | 7.504E−07 | 3.895E−07 | −1.664e−006 to | −9.368e−008 | β2 | KRT13 | 3.178E−07 | 3.421E−07 | −1.096e−006 to | 1.865e−007 |
| | β3 | Age | 0.1145 | 0.07544 | −0.02365 to | 0.2819 | β3 | Age | 0.1016 | 0.06933 | −0.02772 to | 0.2522 |
| HPX + | β0 | Intercept | 0.1997 | 1.906 | −3.794 to | 4.053 | β0 | Intercept | −3.107 | 2.562 | −8.710 to | 1.648 |
| KRT13 + | β1 | HPX | −0.000000202 | 1.454E−07 | −5.789e−007 to | 1.403e−008 | β1 | HPX | −3.556E−07 | 2.674E−07 | −9.740e−007 to | 1.797e−008 |
| PI-RADS | β2 | KRT13 | −0.000000643 | 4.059E−07 | −1.605e−006 to | 2.504e−008 | β2 | KRT13 | 2.262E−08 | 3.111E−07 | −8.4696−007 to | 3.505e−007 |
| | β3 | PI-RADS | 0.7168 | 0.3894 | 0.05160 to | 1.653 | β3 | PI-RADS | 1.448 | 0.6199 | 0.4390 to | 2.893 |
| HPX + | β0 | Intercept | −5.908 | 5.504 | −17.98 to | 4.538 | β0 | Intercept | −6.848 | 5.646 | −19.66 to | 3.583 |
| KRT13 + | β1 | HPX | −2.555E−07 | 1.799E−07 | −7.087e−007 to | −2.521e−009 | β1 | HPX | −4.317E−07 | 2.999E−07 | −1.117e−006 to | 1.188e−009 |
| Age + | β2 | KRT13 | 6.368E−07 | 0.000000417 | −1.626e−006 to | 4.930e−008 | β2 | KRT13 | 4.257E−08 | 3.373E−07 | −8.502e−007 to | 3.986e−007 |
| PI-RADS | β3 | Age | 0.1074 | 0.09191 | −0.06200 to | 0.3123 | β3 | Age | 0.068 | 0.08866 | −0.1039 to | 0.2602 |
| | β4 | PI-RADS | 0.5646 | 0.4005 | −0.1422 to | 1.511 | β4 | PI-RADS | 1.377 | 0.6389 | 0.3241 to | 2.843 |
| PEDF + | β0 | Intercept | 5.775 | 1.878 | 2.760 to | 10.39 | β0 | Intercept | 3.379 | 1.399 | 1.056 to | 6.771 |
| FCER2 + | β1 | PEDF | −0.00005542 | 0.0000202 | −0.0001045 to | −2.278e−005 | β1 | PEDF | −0.00003178 | 0.00001464 | −6.528e−005 to | −7.460e−006 |
| CANX | β2 | CANX | −0.0000268 | 0.00001502 | 6.6020e−005 to | 1.138e−006 | β2 | CANX | 0.00003057 | 0.00001854 | 7.611e−05 to | 2.288e−006 |
| | β3 | FCER2 | −0.000004613 | 0.000004626 | −1.565e−005 to | 2.489e−006 | β3 | FCER2 | −2.652E−06 | 0.000003966 | −1.154e−005 to | 4.139e−006 |
| PEDF + | β0 | Intercept | −1.258 | 5.474 | −12.61 to | 9.757 | β0 | Intercept | −2.818 | 4.575 | −12.32 to | 6.208 |
| FCER2 + | β1 | PCDI | −0.00005504 | 0.00002051 | −0.0001052 to | −2.166e−005 | β1 | PCDI | −0.00003183 | 0.0000151 | −6.628e−005 to | −6.228e−006 |
| CANX + | β2 | CANX | −0.00003781 | 0.00001917 | 8.635e−005 to | 6.110e−006 | β2 | CANX | 0.00003958 | 0.00002158 | 9.539e−005 to | 7.108e−006 |
| Age | β3 | FCER2 | −0.000003335 | 0.000004985 | −1504e−005 to | 4.687e−006 | β3 | FCER2 | −2.572E−06 | 0.000004387 | −1.243e−005 to | 5.104e−006 |
| | β4 | Age | 0.1102 | 0.08805 | −0.04467 to | 0.3142 | β4 | Age | 0.09938 | 0.07334 | −0.03500 to | 0.2608 |
| PEDF + | β0 | Intercept | 2.646 | 2.164 | 1.582 to | 7.478 | β0 | Intercept | 2.441 | 3.115 | 9.427 to | 3.378 |
| FCER2 + | β1 | PEDF | −0.000063 | 0.00002783 | −0.0001353 to | −1.931e−005 | β1 | PEDF | −0.00003357 | 0.00002304 | −8.682e−005 to | 5.965e−006 |
| CANX + | β2 | CANX | −0.00001542 | 0.00002029 | −6.883e−005 to | 1.862e−005 | β2 | CANX | −0.00007684 | 0.00004809 | −0.0001981 to | −3.146e−006 |
| PI-RADS | β3 | FCER2 | −0.000004331 | 0.000004868 | 1.617e−005 to | 3.665e−006 | β3 | FCER2 | −4.314E−06 | 0.000007324 | 2.152e−005 to | 7.183e−006 |
| | β4 | PI-RADS | 0.9137 | 0.4864 | 0.06833 to | 2.094 | β4 | PI-RADS | 2.11 | 1.014 | 0.6163 to | 4.753 |
| PEDF + | β0 | Intercept | −3.29 | 6.866 | −18.31 to | 10.34 | β0 | Intercept | −8.748 | 7.25 | −27.21 to | 4.253 |
| FCER2 + | β1 | PEDF | −0.00006406 | 0.00002958 | −0.0001433 to | −1.839e−005 | β1 | PEDF | −0.00003719 | 0.00002544 | −9.733e−005 to | 6.370e−006 |
| CANX + | β2 | CANX | −0.00002393 | 0.00002362 | −8.592e−005 to | 1.495e−005 | β2 | CANX | −0.00009765 | 0.00005786 | −0.0002461 to | −1.043e−005 |
| Age + | β3 | FCER2 | −0.000003163 | 0.000005066 | −1.523e−005 to | 5.590e−006 | β3 | FCER2 | −8.741E−06 | 0.000008495 | −2.636e−005 to | 6.474e−006 |
| PI-RADS | β4 | Age | 0.0968 | 0.111 | −0.1054 to | 0.3581 | β4 | Age | 0.106 | 0.1052 | 0.09018 to | 0.3533 |
| | β5 | PI-RADS | 0.834 | 0.4827 | −0.004864 to | 2.006 | β5 | PI-RADS | 2.284 | 1.151 | 0.6165 to | 5.408 |
| PEDF + | β0 | Intercept | 5.683 | 1.897 | 2.656 to | 10.38 | β0 | Intercept | 3.343 | 1.404 | 1.008 to | 6.712 |
| FCER2 + | β1 | PEDF | −0.00005037 | 0.00002017 | −9.931e−005 to | −1.739e−005 | β1 | PEDF | −0.00003036 | 0.00001524 | −6.522e−005 to | −4.661e−006 |
| CANX + | β2 | CANX | −0.00002638 | 0.0000149 | −6.509e−005 to | −6.760e−007 | β2 | CANX | −0.00003009 | 0.00001853 | −7.571e−005 to | −1.874e−006 |
| KRT13 | β3 | FCER2 | −0.000003585 | 0.000005002 | −1.503e−005 to | 4.808e−006 | β3 | FCER2 | −2.422E−06 | 0.000004084 | −1.142e−005 to | 4.845e−006 |
| | β4 | KRT13 | −3.891E−07 | 4.675E−07 | −1.608e−006 to | 1.874e−007 | β4 | KRT13 | −9.893E−08 | 3.858E−07 | −1.047e−006 to | 1.816e−007 |
| PEDF + | β0 | Intercept | −1.353 | 5.655 | −13.23 to | 9.875 | β0 | Intercept | −2.778 | 4.595 | −12.32 to | 6.297 |
| FCER2 + | β1 | PEDF | −0.00005078 | 0.00002037 | −0.0001007 to | −1.741e−005 | β1 | PEDF | −0.00003123 | 0.00001565 | −6.695e−005 to | −4.467e−006 |
| CANX + | β2 | CANX | −0.00003777 | 0.00001989 | −8.903e−005 to | −5.478e−008 | β2 | CANX | −0.00003937 | 0.0000217 | −9.543e−005 to | −6.678e−006 |
| KRT13 + | β3 | FCER2 | 0.000002677 | 0.000005366 | −1.489e−005 to | 6.739e−006 | β3 | FCER2 | 2.501E−06 | 0.000004428 | −1.240e−005 to | 5.386e−006 |
| Age | β4 | KRT13 | −4.142E−07 | 5.273E−07 | −1.823e−006 to | 2.288e−007 | β4 | KRT13 | −4.825E−08 | 3.858E−07 | −1.050e−006 to | 2.242e−007 |
| | β5 | Age | 0.1126 | 0.09355 | −0.04786 to | 0.3327 | β5 | Age | 0.0987 | 0.07392 | −0.03710 to | 0.2615 |
| PEDF + | β0 | Intercept | 2.566 | 2.189 | −1.686 to | 7.470 | β0 | Intercept | −2.646 | 3.283 | −10.08 to | 3.452 |
| FCER2 + | β1 | PEDF | −0.00006059 | 0.0000285 | −0.0001341 to | −1.569e−005 | β1 | PEDF | −0.0000385 | 0.00002506 | −9.716e−005 to | 3.781e−006 |
| CANX + | β2 | CANX | −0.00001442 | 0.00002027 | 6.797e−005 to | 2.019e−005 | β2 | CANX | 0.00008829 | 0.00005384 | 0.0002226 to | 7.105e−006 |
| KRT13 + | β3 | FCER2 | −0.000003824 | 0.000005214 | −1.598e−005 to | 5.639e−006 | β3 | FCER2 | −5.575E−06 | 0.000008195 | −2.477e−005 to | 6.814e−006 |
| PI-RADS | β4 | KRT13 | −1.817E−07 | 0.154E−07 | −1.835e−006 to | 3.055e−007 | β4 | KRT13 | 2.105E−07 | 1.919E−07 | −7.301e−007 to | 6.066e−007 |
| | β5 | PI-RADS | 0.9105 | 0.4937 | 0.05143 to | 2.128 | β5 | PI-RADS | 2.345 | 1.126 | 0.6949 to | 5.283 |
| PEDF + | β0 | Intercept | −3.443 | 6.766 | −18.41 to | 10.05 | β0 | Intercept | −9.824 | 7.775 | −29.90 to | 3.848 |
| FCER2 + | β1 | PEDF | −0.00006088 | 0.00003009 | −0.0001414 to | −1.461e−005 | β1 | PEDF | −0.00004357 | 0.00002814 | −0.0001112 to | 3.567e−006 |
| CANX + | β2 | CANX | −0.00002317 | 0.00002396 | −8.573e−005 to | 1.637e−005 | β2 | CANX | −0.000115 | 0.00006695 | −0.0002928 to | −1.639e−005 |
| KRT13 + | β3 | FCER2 | −0.000002548 | 0.000005441 | −1.497e−005 to | 7.831e−006 | β3 | FCER2 | −8.887E−06 | 0.000009716 | −3.200e−005 to | 5.889e−006 |
| Age + | β4 | KRT13 | 2.491E−07 | 0.000000677 | 2.091e−006 to | 3.627e−007 | β4 | KRT13 | 2.803E−07 | 2.228E−07 | 7.188e−007 to | 7.894e−007 |
| PI-RADS | β5 | Age | 0.09848 | 0.1103 | −0.1015 to | 0.3598 | β5 | Age | 0.1187 | 0.1101 | −0.08458 to | 0.3786 |
| | β6 | PI-RADS | 0.8249 | 0.4939 | −0.03663 to | 2.049 | β6 | PI-RADS | 2.632 | 1.339 | 0.7278 to | 6.398 |
| PEDF + | β0 | Intercept | 7.311 | 2.456 | 3.517 to | 13.75 | β0 | Intercept | 5.224 | 2.091 | 1.878 to | 10.26 |
| FCER2 + | β1 | PEDF | 0.0000524 | 0.00002156 | −0.0001050 to | −1.697e−005 | β1 | PEDF | −0.00003167 | 0.0000169 | −7.030e−005 to | −3.052e−006 |
| CANX + | β2 | HPX | −4.406E−07 | 0.00000027 | −1.112e−006 to | 2.008e−008 | β2 | HPX | −4.296E−07 | 2.647E−07 | −1.029e−006 to | 2.878e−008 |
| KRT13 + | β3 | CANX | 0.00002544 | 0.00003301 | −3.902e−005 to | 9.772e−005 | β3 | CANX | −2.648E−06 | 0.00002555 | −5.675e−005 to | 4.692e−005 |
| HPX | β4 | FCER2 | −0.000006105 | 0.000005257 | −1.858e−005 to | 2.909e−006 | β4 | FCER2 | −3.989E−06 | 0.000004428 | −1.401e−005 to | 3.793e−006 |
| | β5 | KRT13 | −5.046E−07 | 0.000000554 | −1.944e−006 to | 2.841e−007 | β5 | KRT13 | −4.417E−08 | 4.291E−07 | −1.070e−006 to | 3.586e−007 |
| PEDF + | β0 | Intercept | −2.259 | 6.541 | −16.10 to | 11.09 | β0 | Intercept | −1.753 | 4.969 | −11.90 to | 8.433 |
| FCER2 + | β1 | PEDF | −0.00005739 | 0.00002467 | −0.0001230 to | −1.844e−005 | β1 | PEDF | −0.0000337 | 0.0000174 | −7.332e−005 to | −3.635e−006 |
| CANX + | β2 | HPX | −6.054E−07 | 3.606E−07 | −1.515e−006 to | −1.841e−008 | β2 | HPX | −4.91E−07 | 2.928E−07 | −1.17e−006 to | 1.301e−008 |
| KRT13 + | β3 | CANX | 0.00002542 | 0.00004007 | −5.199e−005 to | 0.0001172 | β3 | CANX | 9.289E−06 | 0.00002954 | −7.489e−005 to | 4.609e−005 |
| HPX + Age | β4 | FCER2 | −0.000006467 | 0.000006039 | −2.060e−005 to | 4.081e−006 | β4 | FCER2 | −4.779E−06 | 0.000004997 | −1.655e−005 to | 3.924e−006 |
| | β5 | KRT13 | −5.233E−07 | 6.355E−07 | −2.255e−006 to | 3.778e−007 | β5 | KRT13 | 2.822E−08 | 4.475E−07 | −1.050e−006 to | 4.334e−007 |
| | β6 | Age | 0.1697 | 0.1224 | −0.04034 to | 0.4634 | β6 | Age | 0.1186 | 0.08347 | −0.03387 to | 0.3078 |
| PEDF + | β0 | Intercept | 4.211 | 3.014 | −1.315 to | 11.66 | β0 | Intercept | −2.768 | 3.932 | −11.92 to | 4.729 |
| FCER2 + | β1 | PEDF | −0.00006197 | 0.00002926 | −0.0001388 to | −1.601e−005 | β1 | PEDF | −0.00003871 | 0.00002548 | −0.0001000 to | 3.794e−006 |
| CANX + | β2 | HPX | −2.645E−07 | 3.149E−07 | −1.040e−006 to | 2.890e−007 | β2 | HPX | 2.28E−08 | 0.000000401 | −8.181e−007 to | 8.722e−007 |
| KRT13 + | β3 | CANX | 0.00001559 | 0.00004083 | −0.512e−005 to | 0.0001073 | β3 | CANX | −0.00009072 | 0.00006949 | −0.0002767 to | 9.231e−006 |
| HPX + | β4 | FCER2 | −0.000005887 | 0.000005931 | −1.988e−005 to | 4.599e−006 | β4 | FCER2 | −5.612E−06 | 0.000008268 | −2.496e−005 to | 7.053e−006 |
| PI-RADS | β5 | KRT13 | −3.485E−07 | 7.023E−07 | 2.141e−006 to | 3.945e−007 | β5 | KRT13 | 2.079E−07 | 1.974E−07 | 7.395e−007 to | 6.083e−007 |
| | β6 | PI-RADS | 0.8054 | 0.5164 | −0.1280 to | 2.054 | β6 | PI-RADS | 2.369 | 1.214 | 0.6371 to | 5.809 |
| PEDF + | β0 | Intercept | −4.174 | 7.458 | −21.15 to | 11.04 | β0 | Intercept | −10.42 | 8.039 | −31.20 to | 3.541 |
| FCER2 + | β1 | PEDF | −0.00006775 | 0.00003569 | −0.0001780 to | −1.611e−005 | β1 | PEDF | −0.00004364 | 0.00002746 | −0.0001099 to | 2.843e−006 |
| CANX + | β2 | HPX | −4.281E−07 | 3.977E−07 | −1.408e−006 to | 2.465e−007 | β2 | HPX | −2.545E−07 | 4.924E−07 | −1.477e−006 to | 6.895e−007 |
| KRT13 + | β3 | CANX | 0.0000164 | 0.00004487 | −7.016e−005 to | 0.0001202 | β3 | CANX | −0.00009837 | 0.00006908 | −0.0002879 to | 4.516e−006 |
| HPX + | β4 | FCER2 | 0.000005616 | 0.000006418 | −2.030e−005 to | 6.029e−006 | β4 | FCER2 | 0.527E−06 | 0.000009978 | −3.365e−005 to | 5.465e−006 |
| Age + | β5 | KRT13 | 4.396E−07 | 7.739E−07 | 2.443e−006 to | 5.077e−007 | β5 | KRT13 | 3.359E−07 | 2.562E−07 | 6.908e−007 to | 9.4626−007 |
| PI-RADS | β6 | Age | 0.1563 | 0.1384 | −0.08638 to | 0.5046 | β6 | Age | 0.149 | 0.1273 | 0.08230 to | 0.4623 |
| | β7 | PI-RADS | 0.6748 | 0.5306 | −0.3174 to | 1.952 | β7 | PI-RADS | 2.535 | 1.291 | 0.6463 to | 6.215 |
| PEDF + | β0 | Intercept | 11.57 | 5.038 | 4.999 to | 25.10 | β0 | Intercept | 9.686 | 4.006 | 3.536 to | 19.74 |
| FCER2 + | β1 | PEDF | −0.00005693 | 0.00002465 | −0.0001198 to | −1.746e−005 | β1 | PEDF | −0.00003728 | 0.00002035 | −8.619e−005 to | −3.035e−006 |
| CANX + | β2 | HPX | −7.711E−07 | 0.000000489 | −1.986e−006 to | −1.002e−007 | β2 | HPX | −8.305E−07 | 4.123E−07 | −1.862e−006 to | −1.594e−007 |
| KRT13 + | β3 | CANX | 0.00002616 | 0.00003623 | −4.173e−005 to | 0.0001094 | β3 | CANX | 2.82E−07 | 0.00002623 | −5.557e−005 to | 5.203e−005 |
| HPX + | β4 | FCER2 | −0.000007272 | 0.000005321 | −2.030e−005 to | 2.004e−006 | β4 | FCER2 | −5.686E−06 | 0.000004621 | −1.718e−005 to | 2.459 e−006 |
| HRNR | β5 | KRT13 | 0.000001366 | 0.000001444 | −1.012e−006 to | 4.889e−006 | β5 | KRT13 | 0.00000175 | 0.000001157 | −2.341e−007 to | 4.461e−006 |
| | β6 | HRNR | −0.0001921 | 0.0001282 | 0.0005267 to | 1.047e−005 | β6 | HRNR | −0.0001742 | 0.000102 | 0.0004177 to | 9.287e−006 |
| PEDF + | β0 | Intercept | −0.01394 | 7.22 | −15.88 to | 16.03 | β0 | Intercept | 2.852 | 6.072 | −8.636 to | 16.62 |
| FCER2 + | β1 | PEDF | −0.00007543 | 0.00003688 | −0.0001865 to | −2.407e−005 | β1 | PEDF | −0.0000364 | 0.00001966 | −8.427e−005 to | −2.965e−006 |
| CANX + | β2 | HPX | −0.000001498 | 8.831E−07 | −4.006e−006 to | −2.956e−007 | β2 | HPX | −8.981E−07 | 4.378E−07 | −1.988e−006 to | −1.865e−007 |
| KRT13 + | β3 | CANX | 0.00004611 | 0.00005812 | −5.582e−005 to | 0.0001878 | β3 | CANX | −0.00000348 | 0.00003097 | −7.495e−005 to | 5.566e−005 |
| HPX + | β4 | FCER2 | −0.00001101 | 0.000009382 | −3.684e−005 to | 3.258e−006 | β4 | FCER2 | −6.044E−06 | 0.000005645 | −2.147e−005 to | 3.125e−006 |
| HRNR + Age | β5 | KRT13 | 0.000002857 | 0.000002554 | −7.837e−007 to | 1.007e−005 | β5 | KRT13 | 1.833E−06 | 0.0000012 | −2.413e−007 to | 4.650e−006 |
| | β6 | HRNR | −0.000344 | 0.0002421 | 0.001063 to | 4.009e−005 | β6 | HRNR | −0.0001783 | 0.0001081 | 0.0004355 to | 1.827e−006 |
| | β7 | Age | 0.2958 | 0.1919 | 0.002446 to | 0.8275 | β7 | Age | 0.1141 | 0.09069 | −0.0517810 to | 0.3274 |
| PEDF + | β0 | Intercept | 7.558 | 4.65 | 0.3054 to | 20.27 | | | | | | |
| FCER2 + | β1 | PEDF | −0.00005772 | 0.00002729 | −0.0001302 to | −1.338e−005 | | | | | | |
| CANX + | β2 | HPX | −5.381E−07 | 4.641E−07 | −1.692e−006 to | 1.746e−007 | | | | | | |
| KRT13 + | β3 | CANX | 0.00002366 | 0.00005144 | −7.990e−005 to | 0.0001355 | | | | | | |
| HPX + | β4 | FCER2 | −0.000006743 | 0.000006404 | −2.202e−005 to | 5.135e−006 | | | | | | |
| HRNR + | β5 | KRT13 | 0.000001417 | 0.000001471 | −1.183e−006 to | 5.105e−006 | | | | | | |
| PI-RADS | β6 | HRNR | −0.0001867 | 0.0001239 | −0.0005227 to | −5.679e−006 | | | | | | |
| | β7 | PI-RADS | 0.7906 | 0.5511 | −0.1659 to | 2.254 | | | | | | |
| PEDF + | β0 | Intercept | −1.764 | 8.334 | −20.64 to | 14.98 | | | | | | |
| FCER2 + | β1 | PEDF | −0.000077 | 0.00004179 | −0.0002190 to | −1.923e−005 | | | | | | |
| CANX + | β2 | HPX | 0.000001117 | 8.286E−07 | −3.692e−006 to | 2.202e−008 | | | | | | |
| KRT13 + | β3 | CANX | 0.00003441 | 0.00006973 | −9.143e−005 to | 0.0002183 | | | | | | |
| HPX + | β4 | FCER2 | −0.00000829 | 0.000009806 | −3.670e−005 to | 7.449e−006 | | | | | | |
| HRNR + | β5 | KRT13 | 0.000002324 | 0.000002342 | −1.144e−006 to | 9.525e−006 | | | | | | |
| Age + | β6 | HRNR | −0.0002849 | 0.0002161 | −0.001002 to | −1.951e−005 | | | | | | |
| PI-RADS | β7 | Age | 0.2466 | 0.1917 | −0.07003 to | 0.7781 | | | | | | |
| | β8 | PI-RADS | 0.5456 | 0.5696 | −0.4919 to | 2.067 | | | | | | |
| PEDF + | β0 | Intercept | 13.78 | 5.906 | 5.979 to | 31.02 | β0 | Intercept | 10.03 | 4.336 | 3.567 to | 21.25 |
| FCER2 + | β1 | PEDF | −0.00007429 | 0.00003313 | −0.0001744 to | −2.550e−005 | β1 | PEDF | −0.00004274 | 0.00002195 | −9.788e−005 to | −6.665e−006 |
| CANX + | β2 | HPX | −9.878E−07 | 5.414E−07 | −2.443e−006 to | −2.401e−007 | β2 | HPX | −8.906E−07 | 4.573E−07 | −2.055e−006 to | −1.781e−007 |
| KRT13 + | β3 | CD99 | −0.000004352 | 0.000002813 | −1.106e−005 to | 8.497e−007 | β3 | CD99 | −2.419E−06 | 0.00000211 | −7.008e−006 to | 1.567e−006 |
| HPX + | β4 | CANX | 0.00006045 | 0.00004582 | −2.037e−005 to | 0.0001723 | β4 | CANX | 0.00001494 | 0.00002975 | −4.576e−005 to | 7.612e−005 |
| HRNR+ | β5 | FCER2 | −0.000001829 | 0.000006796 | −1.764e−005 to | 1.173e−005 | β5 | FCER2 | −1.713E−06 | 0.000005732 | −1.475e−005 to | 9.159e−006 |
| CD99 | β6 | KRT13 | 0.000001503 | 0.000001554 | −9.090e−007 to | 5.761e−006 | β6 | KRT13 | 1.624E−06 | 0.000001164 | −3.752e−007 to | 4.385e−006 |
| | β7 | HRNR | −0.0001965 | 0.0001411 | −0.0005982 to | −2.772e−006 | β7 | HRNR | −0.0001557 | 0.0001059 | −0.0004086 to | 1.143e−005 |
| PEDF + | β0 | Intercept | 3.89 | 8.418 | −13.00 to | 23.68 | β0 | Intercept | 3.413 | 6.348 | −8.506 to | 18.10 |
| FCER2 + | β1 | PEDF | −0.00008603 | 0.00004055 | −0.0002042 to | −2.956e−005 | β1 | PEDF | −0.00004380 | 0.00002247 | −9.992e−005 to | −6.933e−006 |
| CANX + | β2 | HPX | −0.000001356 | 7.467E−07 | −3.614e−006 to | −3.444e−007 | β2 | HPX | −9.672E−07 | 4.673E−07 | −2.111e−006 to | −2.150e−007 |
| KRT13 + | β3 | CD99 | −0.000004093 | 0.000003274 | −1.177e−005 to | 2.710e−006 | β3 | CD99 | −2.483E−06 | 0.000002322 | −7.716e−006 to | 1.961e−006 |
| HPX + | β4 | CANX | 0.00004023 | 0.00005808 | −6.271e−005 to | 0.0001794 | β4 | CANX | 7.251E−06 | 0.00003448 | −6.888e−005 to | 7.461e−005 |
| HRNR+ | β5 | FCER2 | −0.000003182 | 0.00001076 | −3.060e−005 to | 1.862e−005 | β5 | FCER2 | −2.482E−06 | 0.000007251 | −1.987e−005 to | 1.013e−005 |
| CD99 + Age | β6 | KRT13 | 0.000003004 | 0.000002614 | −6.812e−007 to | 1.035e−005 | β6 | KRT13 | 1.777E−06 | 0.000001234 | −4.199e−007 to | 4.650e−006 |
| | β7 | HRNR | −0.0003448 | 0.0002421 | −0.001061 to | −3.680e−005 | β7 | HRNR | −0.0001716 | 0.0001124 | −0.0004368 to | 1.493e−005 |
| | β8 | Age | 0.2362 | 0.1786 | −0.03731 to | 0.7500 | β8 | Age | 0.1187 | 0.1033 | −0.06168 to | 0.3711 |
| PEDF + | β0 | Intercept | 11.82 | 6.788 | 2.328 to | 32.48 | | | | | | |
| FCER2 + | β1 | PEDF | −0.0000975 | 0.00005551 | −0.0003097 to | −2.709e−005 | | | | | | |
| CANX + | β2 | HPX | −7.028E−07 | 5.577E−07 | −2.221e−006 to | 1.227e−007 | | | | | | |
| KRT13 + | β3 | CD99 | −0.000007309 | 0.000004664 | −2.205e−005 to | 2.161e−007 | | | | | | |
| HPX + | β4 | CANX | 0.00002931 | 0.00007218 | −0.0001079 to | 0.0002229 | | | | | | |
| HRNR+ | β5 | FCER2 | 0.000004781 | 0.00001125 | −1.798e−005 to | 3.446e−005 | | | | | | |
| CD99 + | β6 | KRT13 | 0.000002535 | 0.000002148 | −7.789e−007 to | 8.9226−006 | | | | | | |
| PI-RADS | β7 | HRNR | −0.0002633 | 0.0001858 | −0.0008360 to | −6.300e−006 | | | | | | |
| | β8 | PI-RADS | 0.9 | 0.6658 | −0.2572 to | 2.810 | | | | | | |
| PEDF + | β0 | Intercept | 1.384 | 12.43 | ??? to | 27.71 | | | | | | |
| FCER2 + | β1 | PEDF | −0.0001426 | 0.0001212 | ??? to | −3.212e−005 | | | | | | |
| CANX + | β2 | HPX | −0.000001126 | 9.007E−07 | ??? to | 3.522e−008 | | | | | | |
| KRT13 + | β3 | CD99 | −0.000007906 | 0.000007323 | ??? to | 1.449e−006 | | | | | | |
| HPX + | β4 | CANX | 0.000005735 | 0.0001106 | ??? | | | | | | | |
| HRNR+ | β5 | FCER2 | 0.000007713 | 0.0000195 | −2.615e−005 to | ??? | | | | | | |
| CD99 + | β6 | KRT13 | 0.000004002 | 0.000004552 | 1.222e−006 to | ??? | | | | | | |
| Age + | β7 | HRNR | −0.0004415 | 0.0004388 | ??? to | −3.086e−005 | | | | | | |
| PI-RADS | β8 | Age | 0.2745 | 0.3337 | −0.1777 to | ??? | | | | | | |
| | β9 | PI-RADS | 1.103 | 1.163 | −0.3292 to | ??? | | | | | | |

TABLE 8

| | | | Detect PCa (any grade) | | | | | | Detect High-grade PCa (GS ≥ 7) | | | | | |
|---|---|---|---|---|---|---|---|---|---|---|---|---|---|---|
| | | | | | 95% | | Specificity | Specificity | | | 95% | | Specificity | Specificity |
| | | | | | Confidence | | at 90% | at 100% | | | Confidence | | at 90% | at 100% |
| | Protein Name | Uniprot ID | AUC | Std. Error | interval | p-value | Sensitivity | Sensitivity | AUC | Std. Error | interval | p-value | Sensitivity | Sensitivity |
| Not | Pigment | P36955 | 0.5848 | 0.08781 | 0.4127 to 0.7569 | 0.3423 | 30.4 | 21.7 | 0.5880 | 0.08888 | 0.4138 to 0.7622 | 0.340 | 29.6 | 18.5 |
| normalized | epithelium- | | | | | | | | | | | | | |
| | derived factor | | | | | | | | | | | | | |
| | Homopoxin | P02790 | 0.5848 | 0.08812 | 0.4121 to 0.7575 | 0.3423 | 26.1 | 8.7 | 0.6319 | 0.09101 | 0.4536 to 0.8103 | 0.1521 | 14.8 | 7.4 |
| | CD99 antigen | P14209 | 0.6304 | 0.08512 | 0.4636 to 0.7973 | 0.144 | 39.1 | 13.0 | 0.6667 | 0.08637 | 0.4974 to 0.8359 | 0.070 | 25.1 | 11.1 |
| | Calnexin | P27824 | 0.5804 | 0.08888 | 0.4062 to 0.7546 | 0.3676 | 21.0 | 17.4 | 0.5208 | 0.08985 | 0.3447 to 0.6969 | 0.821 | 22.2 | 14.8 |
| | Low affinity | P06734 | 0.6478 | 0.0847 | 0.4818 to 0.8138 | 0.0978 | 30.4 | 4.3 | 0.6389 | 0.08706 | 0.4683 to 0.8095 | 0.132 | 25.9 | 3.8 |
| | immunoglobulin | | | | | | | | | | | | | |
| | epsilon | | | | | | | | | | | | | |
| | Fc receptor | | | | | | | | | | | | | |
| | Hornerin | Q86YZ3 | 0.6533 | 0.08453 | 0.4876 to 0.8189 | 0.086 | 30.4 | 13.0 | 0.6609 | 0.08716 | 0.4900 to 0.8317 | 0.081 | 25.9 | 11.1 |
| | Keratin, type 1 | P13646 | 0.7043 | 0.07986 | 0.5478 to 0.8609 | 0.0221 | 47.8 | 30.4 | 0.6852 | 0.08149 | 0.5255 to 0.8449 | 0.044 | 29.6 | 25.0 |
| | cytoskeletal 13 | | | | | | | | | | | | | |
| Normalized | Pigment | P36955 | 0.7609 | 0.0730 | 0.6176 to 0.9041 | 0.0035 | 34.8 | 30.4 | 0.7292 | 0.0790 | 0.5752 to 0.8831 | 0.0129 | 33.3 | 29.6 |
| | epithelium- | | | | | | | | | | | | | |
| | derived factor | | | | | | | | | | | | | |
| | Hemopexin | P02790 | 0.7696 | 0.07049 | 0.6314 to 0.9077 | 0.0025 | 47.8 | 43.5 | 0.7708 | 0.07278 | 0.6282 to 0.9135 | 0.0033 | 44.4 | 37.0 |
| | CD99 antigen | P14209 | 0.7565 | 0.0730 | 0.6136 to 0.8994 | 0.0041 | 52.2 | 47.8 | 0.7222 | 0.0780 | 0.5688 to 0.8756 | 0.0159 | 40.7 | 40.7 |
| | Calnexin | P27824 | 0.7457 | 0.0760 | 0.5971 to 0.8942 | 0.0059 | 30.4 | 26.1 | 0.6528 | 0.0860 | 0.4849 to 0.8207 | 0.0973 | 25.9 | 22.1 |
| | Low affinity | P06734 | 0.7565 | 0.0740 | 0.6114 to 0.9017 | 0.0041 | 47.8 | 13.0 | 0.7269 | 0.0810 | 0.5690 to 0.8847 | 0.0138 | 44.4 | 11.2 |
| | immunoglobulin | | | | | | | | | | | | | |
| | epsilon | | | | | | | | | | | | | |
| | Fc receptor | | | | | | | | | | | | | |
| Normalized | Hornerin | Q86YZ3 | 0.7120 | 0.0800 | 0.5553 to 0.8686 | 0.0176 | 39.1 | 17.4 | 0.6956 | 0.0830 | 0.5321 to 0.8591 | 0.0337 | 37.0 | 14.8 |
| | Keratin, type I | P13646 | 0.8087 | 0.0660 | 0.6797 to 0.9377 | 0.0005 | 43.5 | 43.5 | 0.7708 | 0.0750 | 0.6247 to 0.9170 | 0.0033 | 40.7 | 37.1 |
| | cytoskeletal 13 | | | | | | | | | | | | | |
| | CD44 antigen | P16070 | 0.6957 | 0.08213 | 0.5347 to 0.8566 | 0.0284 | 47.8 | 30.4 | 0.09 | 0.08 | 0.5318 to 0.8478 | 0.04 | 44.4 | 25.9 |
| | Non-secretory | P10153 | 0.6783 | 0.08282 | 0.5159 to 0.8406 | 0.0459 | 39.1 | 8.7 | 0.63 | 0.09 | 0.4506 to 0.8086 | 0.16 | 18.5 | 7.4 |
| | ribonuclease | | | | | | | | | | | | | |
| | WAP | Q14508 | 0.7 | 0.08304 | 0.5373 to 0.8627 | 0.0251 | 43.5 | 0.0 | 0.60 | 0.09 | 0.4184 to 0.7806 | 0.28 | 3.7 | 0.0 |
| | four-disulfide | | | | | | | | | | | | | |
| | core domain | | | | | | | | | | | | | |
| | protein 2 | | | | | | | | | | | | | |
| | Serum Prostate | | 0.6022 | 0.0884 | 0.4289 to 0.7755 | 0.2525 | 21.7 | 0.0 | 0.5694 | 0.0960 | 0.3812 to 0.7576 | 0.4510 | 3.7 | 0.0 |
| | Specific Antigen | | | | | | | | | | | | | |

TABLE 9.1

| | | Detect Pca (any grade) | | | | | | Detect High-grade Pca (GS ≥ 7) | | | | | |
|---|---|---|---|---|---|---|---|---|---|---|---|---|---|
| | | | | 95% | | Specificity | Specificity | | | 95% | | Specificity | Specificity |
| | | | | Confidence | | at 90% | at 100% | | | Confidence | | at 90% | at 100% |
| | | AUC | Std. Error | interval | p-value | Sensitivity | Sensitivity | AUC | Std. Error | interval | p-value | Sensitivity | Sensitivity |
| Not | PEDF | 0.5848 | 0.08781 | 0.4127 to 0.7569 | 0.3423 | 30.4 | 21.7 | 0.5880 | 0.08888 | 0.4138 to 0.7622 | 0.3397 | 29.6 | 18.5 |
| normalized | FCER2 | 0.6478 | 0.0847 | 0.4818 to 0.8138 | 0.0978 | 30.4 | 4.3 | 0.6389 | 0.08706 | 0.4683 to 0.8095 | 0.1317 | 25.9 | 3.8 |
| | CANX | 0.5804 | 0.08888 | 0.4062 to 0.7546 | 0.3676 | 21.0 | 17.4 | 0.5208 | 0.08985 | 0.3447 to 0.6969 | 0.8211 | 22.2 | 14.8 |
| | KRT13 | 0.7043 | 0.07986 | 0.5478 to 0.8609 | 0.0221 | 47.8 | 30.4 | 0.6852 | 0.08149 | 0.5255 to 0.8449 | 0.0444 | 29.6 | 25.9 |
| | HPX | 0.5848 | 0.08812 | 0.4121 to 0.7575 | 0.3423 | 26.1 | 8.7 | 0.6319 | 0.09101 | 0.4536 to 0.8103 | 0.1521 | 14.8 | 7.4 |
| | HRNR | 0.6533 | 0.08453 | 0.4876 to 0.8189 | 0.086 | 30.4 | 13.4 | 0.6609 | 0.08716 | 0.4900 to 0.8317 | 0.0808 | 25.9 | 11.1 |
| | CD99 | 0.6304 | 0.08512 | 0.4636 to 0.7973 | 0.144 | 39.1 | 13.0 | 0.6667 | 0.08637 | 0.4974 to 0.8359 | 0.0704 | 25.9 | 11.1 |
| | Age | 0.6685 | 0.0840 | 0.5038 to 0.8331 | 0.0592 | 17.4 | 0.0 | 0.6343 | 0.09306 | 0.4519 to 0.8167 | 0.1450 | 11.1 | 0.0 |
| | PI-RADS | 0.8403 | 0.0668 | 0.7094 to 0.9712 | 0.0003 | 60.0 | 0.0 | 0.8690 | 0.05767 | 0.7560 to 0.9821 | 0.0002 | 54.2 | 54.2 |
| | PEDF + Age | 0.7000 | 0.0814 | 0.5404 to 0.8596 | 0.0251 | 30.4 | 30.4 | 0.6806 | 0.08492 | 0.5141 to 0.8470 | 0.0500 | 33.3 | 18.5 |
| | FCER2 + Age | 0.7326 | 0.0772 | 0.5813 to 0.8839 | 0.0092 | 52.2 | 39.1 | 0.7014 | 0.07886 | 0.5468 to 0.8560 | 0.0288 | 44.4 | 33.3 |
| | CANX + Age | 0.7043 | 0.0798 | 0.5479 to 0.8608 | 0.0221 | 30.4 | 17.4 | 0.6574 | 0.08506 | 0.4907 to 0.8241 | 0.0875 | 22.2 | 14.8 |
| | KRT13 + Age | 0.7696 | 0.0713 | 0.6298 to 0.9093 | 0.0025 | 52.2 | 30.4 | 0.7361 | 0.07689 | 0.5854 to 0.8868 | 0.0104 | 40.7 | 25.9 |
| | HPX + Age | 0.6978 | 0.08122 | 0.5386 to 0.8570 | 0.0267 | 39.1 | 8.7 | 0.6968 | 0.08701 | 0.5262 to 0.8673 | 0.0327 | 7.4 | 7.4 |
| | HRNR + Age | 0.7413 | 0.07898 | 0.5865 to 0.8961 | 0.0069 | 52.2 | 8.7 | 0.7199 | 0.08407 | 0.5551 to 0.8847 | 0.017 | 14.8 | 7.4 |
| | CD99 + Age | 0.6652 | 0.08268 | 0.5032 to 0.8273 | 0.0642 | 34.8 | 21.7 | 0.6644 | 0.08555 | 0.4967 to 0.8320 | 0.0744 | 29.6 | 18.5 |
| | PEDF + PI-RADS | 0.8750 | 0.0618 | 0.7539 to 0.9961 | <0.0001 | 65.0 | 5.0 | 0.8810 | 0.05661 | 0.7700 to 0.9919 | 0.0001 | 58.3 | 58.3 |
| | FCER2 + PI-RADS | 0.8722 | 0.0618 | 0.7512 to 0.9933 | <0.0001 | 70.0 | 5.0 | 0.8929 | 0.05469 | 0.7857 to 1.000 | <0.0001 | 66.6 | 66.6 |
| | CANX + PI-RADS | 0.8750 | 0.0654 | 0.7469 to 1.000 | <0.0001 | 75.0 | 0.0 | 0.8780 | 0.05806 | 0.7642 to 0.9918 | 0.0001 | 62.5 | 62.5 |
| | HPX + PI-RADS | 0.8472 | 0.06786 | 0.7142 to 0.9802 | 0.0003 | 70.0 | 5.6 | 0.875 | 0.05582 | 0.7656 to 0.9844 | 0.0001 | 75.0 | 62.5 |
| | KRT13 + PI-RADS | 0.8944 | 0.0559 | 0.7848 to 1.000 | <0.0001 | 60.0 | 15.0 | 0.9077 | 0.05037 | 0.8090 to 1.000 | <0.0001 | 75.0 | 58.3 |
| | HRNR + PI-RADS | 0.8361 | 0.06825 | 0.7023 to 0.9699 | 0.0004 | 60.0 | 5.0 | 0.881 | 0.0575 | 0.7683 to 0.9936 | 0.0001 | 79.2 | 79.2 |
| | CD99 + PI-RADS | 0.8444 | 0.06595 | 0.7152 to 0.9737 | 0.0003 | 70.0 | 15.0 | 0.878 | 0.05574 | 0.7687 to 0.9872 | 0.0001 | 66.7 | 62.5 |
| | PEDF + FCER2 | 0.7152 | 0.0782 | 0.5619 to 0.8685 | 0.0159 | 39.1 | 26.1 | 0.6806 | 0.08239 | 0.5191 to 0.8420 | 0.0500 | 37.0 | 22.2 |
| | PEDF + FCER2 + Age | 0.8022 | 0.0667 | 0.6714 to 0.9329 | 0.0007 | 52.2 | 39.1 | 0.7523 | 0.07313 | 0.6090 to 0.8956 | 0.0062 | 48.1 | 44.4 |
| | PEDF + FCER2 + | 0.9056 | 0.0583 | 0.7912 to 1.000 | <0.0001 | 70.0 | 0.0 | 0.9048 | 0.05163 | 0.8036 to 1.000 | <0.0001 | 70.8 | 66.6 |
| | PI-RADS | | | | | | | | | | | | |
| | PEDF + FCFR2 + Age + | 0.9167 | 0.0559 | 0.8072 to 1.000 | <0.0001 | 60.0 | 5.0 | 0.9048 | 0.05087 | 0.8051 to 1.000 | <0.0001 | 70.8 | 62.5 |
| | PI-RADS | | | | | | | | | | | | |
| | PEDF + CANX | 0.6500 | 0.0853 | 0.4829 to 0.8171 | 0.0929 | 43.5 | 39.1 | 0.6157 | 0.08496 | 0.4492 to 0.7823 | 0.2090 | 44.4 | 33.3 |
| | PEDF + CANX + Age | 0.7348 | 0.0768 | 0.5844 to 0.8852 | 0.0085 | 47.8 | 26.1 | 0.6898 | 0.08137 | 0.5303 to 0.8493 | 0.0394 | 44.4 | 18.5 |
| | PEDF + CANX + | 0.8944 | 0.0627 | 0.7715 to 1.000 | <0.0001 | 80.0 | 0.0 | 0.8929 | 0.05400 | 0.7870 to 0.9987 | <0.0001 | 66.6 | 66.6 |
| | PI-RADS | | | | | | | | | | | | |
| | PEDF + CANX + Age + | 0.8944 | 0.0611 | 0.7748 to 1.000 | <0.0001 | 60.0 | 5.0 | 0.8899 | 0.05494 | 0.7822 to 0.9976 | <0.0001 | 66.6 | 66.6 |
| | PI-RADS | | | | | | | | | | | | |
| | HPX + KRT13 | 0.7196 | 0.07855 | 0.5656 to 0.8735 | 0.0139 | 43.5 | 34.8 | 0.7222 | 0.07796 | 0.5694 to 0.8750 | 0.0159 | 40.7 | 29.6 |
| | HPX + KRT13 + Age | 0.7826 | 0.06959 | 0.6462 to 0.9190 | 0.0015 | 52.2 | 30.4 | 0.787 | 0.07337 | 0.6432 to 0.9308 | 0.0018 | 33.3 | 18.5 |
| | HPX + KRT13 + | 0.8833 | 0.05959 | 0.7665 to 1.000 | <0.0001 | 40.0 | 15.0 | 0.9048 | 0.05276 | 0.8013 to 1.000 | <0.0001 | 79.2 | 54.2 |
| | PI-RADS | | | | | | | | | | | | |
| | HPX + KRT13 + Age + | 0.9056 | 0.05469 | 0.7984 to 1.000 | <0.0001 | 75.0 | 10.0 | 0.9137 | 0.04929 | 0.8171 to 1.000 | <0.0001 | 75.0 | 66.7 |
| | PI-RADS | | | | | | | | | | | | |
| | PEDF + FCER2 + | 0.7457 | 0.07522 | 0.5982 to 0.8931 | 0.0059 | 30.4 | 26.1 | 0.6991 | 0.08067 | 0.5410 to 0.8572 | 0.0307 | 37.0 | 22.2 |
| | CANX | | | | | | | | | | | | |
| | PEDF + FCER2 + | 0.8087 | 0.06766 | 0.6761 to 0.9413 | 0.0005 | 56.5 | 26.1 | 0.7639 | 0.07196 | 0.6228 to 0.9049 | 0.0042 | 51.8 | 40.7 |
| | CANX + Age | | | | | | | | | | | | |
| | PEDF + FCER2 + | 0.9222 | 0.05577 | 0.8129 to 1.000 | <0.0001 | 80.0 | 0.0 | 0.9107 | 0.05034 | 0.8120 to 1.000 | <0.0001 | 79.2 | 62.5 |
| | CANX + PI-RADS | | | | | | | | | | | | |
| | PEDF + FCER2 + | 0.9194 | 0.05661 | 0.8085 to 1.000 | <0.0010 | 70.0 | 0.0 | 0.9107 | 0.04921 | 0.8143 to 1.000 | <0.0001 | 75.0 | 62.5 |
| | CANX + Age + | | | | | | | | | | | | |
| | PI-RADS | | | | | | | | | | | | |
| | PEDF + FCER2 + | 0.7783 | 0.07149 | 0.6381 to 0.9184 | 0.0018 | 52.2 | 26.1 | 0.7685 | 0.07395 | 0.6236 to 0.9135 | 0.0036 | 40.7 | 22.2 |
| | CANX + KRT13 | | | | | | | | | | | | |
| | PEDF + FCER2 + | 0.8413 | 0.06077 | 0.7222 to 0.9604 | 0.0001 | 69.6 | 34.8 | 0.7963 | 0.06668 | 0.6656 to 0.9270 | 0.0013 | 55.6 | 48.1 |
| | CANX + KRT13 + Age | | | | | | | | | | | | |
| | PEDF + FCER2 + | 0.9194 | 0.05853 | 0.8047 to 1.000 | <0.0001 | 55.0 | 0.0 | 0.9196 | 0.05061 | 0.8205 to 1.000 | <0.0001 | 83.3 | 54.2 |
| | CANX + KRT13 + | | | | | | | | | | | | |
| | PI-RADS | | | | | | | | | | | | |
| | PEDF + FCER2 + | 0.9278 | 0.05137 | 0.8271 to 1.000 | <0.0001 | 75.0 | 10.0 | 0.9167 | 0.04936 | 0.8199 to 1.000 | <0.0001 | 66.7 | 62.5 |
| | CANX + KRT13 + | | | | | | | | | | | | |
| | Age + PI-RADS | | | | | | | | | | | | |
| | PEDF + FCER2 + | 0.7826 | 0.07214 | 0.6412 to 0.9240 | 0.0015 | 47.8 | 26.1 | 0.7523 | 0.07802 | 0.5994 to 0.9052 | 0.0062 | 25.9 | 18.5 |
| | CANX + KRT13 + HPX | | | | | | | | | | | | |
| | PEDF + FCER2 + | 0.8457 | 0.05896 | 0.7301 to 0.9612 | 0.0001 | 56.5 | 39.1 | 0.8171 | 0.06688 | 0.6860 to 0.9482 | 0.0006 | 40.7 | 40.7 |
| | CANX + KRT13 + | | | | | | | | | | | | |
| | HPX + Age | | | | | | | | | | | | |
| | PEDF + FCER2 + | 0.9194 | 0.05853 | 0.8047 to 1.000 | <0.0001 | 55.0 | 0.0 | 0.9167 | 0.05215 | 0.8144 to 1.000 | <0.0001 | 83.3 | 50.0 |
| | CANX + KRT13 + | | | | | | | | | | | | |
| | HPX + PI-RADS | | | | | | | | | | | | |
| | PEDF + FCER2 + | 0.9278 | 0.05137 | 0.8271 to 1.000 | <0.0001 | 75.0 | 10.0 | 0.9167 | 0.04936 | 0.8199 to 1.000 | <0.0001 | 66.7 | 62.5 |
| | CANX + KRT13 + | | | | | | | | | | | | |
| | HPX + Age + PI-RADS | | | | | | | | | | | | |
| | PEDF + FCER2 + | 0.7891 | 0.06932 | 0.6533 to 0.9250 | 0.0012 | 52.2 | 30.4 | 0.7708 | 0.07308 | 0.6276 to 0.9141 | 0.0033 | 37.0 | 25.9 |
| | CANX + KRT13 + | | | | | | | | | | | | |
| | HPX + HRNR | | | | | | | | | | | | |
| | PEDF + FCER2 + | 0.8435 | 0.05932 | 0.7272 to 0.9598 | 0.0001 | 52.2 | 39.1 | 0.8264 | 0.06498 | 0.6990 to 0.9537 | 0.0004 | 40.7 | 40.7 |
| | CANX + KRT13 + | | | | | | | | | | | | |
| | HPX + HRNR + Age | | | | | | | | | | | | |
| | PEDF + FCER2 + | 0.9167 | 0.05956 | 0.7999 to 1.000 | <0.0001 | 50.0 | 0.0 | 0.9256 | 0.05145 | 0.8248 to 1.000 | <0.0001 | 95.8 | 50.0 |
| | CANX + KRT13 + | | | | | | | | | | | | |
| | HPX + HRNR + | | | | | | | | | | | | |
| | PI-RADS | | | | | | | | | | | | |
| | PEDF + FCER2 + | 0.9278 | 0.05522 | 0.8195 to 1.000 | <0.0001 | 85.0 | 0.0 | 0.9494 | 0.03794 | 0.8751 to 1.000 | <0.0001 | 91.7 | 87.5 |
| | CANX + KRT13 + | | | | | | | | | | | | |
| | HPX + HRNK + Age + | | | | | | | | | | | | |
| | PI-RADS | | | | | | | | | | | | |
| | PEDF + FCER2 + | 0.8261 | 0.06502 | 0.6987 to 0.9535 | 0.0003 | 34.8 | 26.1 | 0.7986 | 0.07307 | 0.6554 to 0.9418 | 0.0012 | 33.3 | 29.6 |
| | CANX + KRT13 + | | | | | | | | | | | | |
| | HPX + HRNR + CD99 | | | | | | | | | | | | |
| | PEDF + FCER2 + | 0.8478 | 0.05855 | 0.7331 to 0.9626 | <0.0001 | 47.8 | 34.8 | 0.8184 | 0.06848 | 0.6806 to 0.0490 | 0.0006 | 37.0 | 37.0 |
| | CANX + KRT13 + | | | | | | | | | | | | |
| | HPX + HRNR + | | | | | | | | | | | | |
| | CD99 + Age | | | | | | | | | | | | |
| | PEDF + FCER2 + | 0.9111 | 0.0607 | 0.7922 to 1.000 | <0.0001 | 45.0 | 0.0 | 0.9256 | 0.04967 | 0.8283 to 1.000 | <0.0001 | 91.7 | 50.0 |
| | CANX + KRT13 + | | | | | | | | | | | | |
| | HPX + HRNR + CD99 + | | | | | | | | | | | | |
| | PI-RADS | | | | | | | | | | | | |
| | PEDF + FCER2 + | 0.9278 | 0.05522 | 0.8195 to 1.000 | <0.0001 | 80.0 | 0.0 | 0.9464 | 0.03690 | 0.8739 to 1.000 | <0.0001 | 91.7 | 79.2 |
| | CANX + KRT13 + | | | | | | | | | | | | |
| | HPX + HRNR + CD99 + | | | | | | | | | | | | |
| | Age + PI-RADS | | | | | | | | | | | | |
| Normalized | PEDF | 0.7609 | 0.0730 | 0.6176 to 0.9041 | 0.0035 | 34.8 | 30.4 | 0.7292 | 0.079 | 0.5752 to 0.8831 | 0.0129 | 33.3 | 29.6 |
| | FCER22 | 0.7565 | 0.0740 | 0.6114 to 0.9017 | 0.0041 | 47.8 | 13.0 | 0.7269 | 0.081 | 0.5690 ti 0.8847 | 0.0138 | 44.4 | 11.2 |
| | CANX | 0.7457 | 0.0760 | 0.5971 to 0.8942 | 0.0059 | 30.4 | 26.1 | 0.6528 | 0.086 | 0.4849 to 0.8207 | 0.0973 | 26.9 | 23.1 |
| | KRT13 | 0.8087 | 0.0660 | 0.6797 to 0.9377 | 0.0005 | 43.5 | 43.5 | 0.7708 | 0.075 | 0.6247 to 0.9170 | 0.0033 | 40.7 | 37.1 |
| | HPX | 0.7696 | 0.07049 | 0.6314 to 0.9077 | 0.0025 | 47.8 | 43.5 | 0.7546 | 0.07407 | 0.6094 to 0.8998 | 0.0057 | 44.4 | 37.0 |
| | HRNR | 0.712 | 0.07991 | 0.5553 to 0.8686 | 0.0176 | 39.1 | 17.4 | 0.6956 | 0.0834 | 0.5321 to 0.8591 | 0.0337 | 37.0 | 14.8 |
| | CD99 | 0.7565 | 0.0729 | 0.6136 to 0.8994 | 0.0041 | 52.2 | 47.8 | 0.7222 | 0.07826 | 0.5688 to 0.8756 | 0.0159 | 40.7 | 40.7 |
| | Age | 0.6685 | 0.0840 | 0.5038 to 0.8331 | 0.0592 | 17.4 | 0.0 | 0.6343 | 0.093 | 0.4519 to 0.8167 | 0.145 | 11.1 | 0.0 |
| | PI-RADS | 0.8403 | 0.0668 | 0.7094 to 0.9712 | 0.0003 | 60.0 | 0.0 | 0.9 | 0.1 | 0.7560 to 0.9821 | 0.0 | 54.2 | 54.2 |
| | PEDF + Age | 0.8022 | 0.0691 | 0.6668 to 0.9375 | 0.0007 | 43.5 | 26.1 | 0.7731 | 0.07295 | 0.6302 to 0.9161 | 0.003 | 40.8 | 25.9 |
| | FCER2 + Age | 0.7848 | 0.0698 | 0.6480 to 0.9215 | 0.0014 | 43.5 | 30.4 | 0.7292 | 0.08007 | 0.5722 to 0.8861 | 0.0129 | 25.9 | 25.9 |
| | CANX + Age | 0.7522 | 0.0738 | 0.6075 to 0.8969 | 0.0047 | 34.8 | 21.7 | 0.6991 | 0.08233 | 0.5377 to 0.8604 | 0.0307 | 25.9 | 18.5 |
| | KRT13 + Age | 0.8652 | 0.056 | 0.7555 to 0.9750 | <0.0001 | 43.5 | 34.8 | 0.7986 | 0.0692 | 0.6630 to 0.9342 | 0.0012 | 40.7 | 29.6 |
| | HPX + Age | 0.8283 | 0.06179 | 0.7072 to 0.9494 | 0.0002 | 56.5 | 30.4 | 0.787 | 0.06999 | 0.6499 to 0.9242 | 0.0018 | 51.8 | 29.6 |
| | HRNR + Age | 0.7348 | 0.07783 | 0.5822 to 0.8873 | 0.0085 | 39.1 | 17.4 | 0.7014 | 0.08364 | 0.5375 to 0.8653 | 0.0288 | 33.3 | 14.8 |
| | CD99 + Age | 0.7935 | 0.06777 | 0.6607 to 0.9263 | 0.001 | 52.2 | 26.1 | 0.75 | 0.07602 | 0.6010 to 0.8990 | 0.0067 | 33.3 | 22.2 |
| | PEDF + PI-RADS | 0.8889 | 0.0582 | 0.7748 to 1.000 | <0.0001 | 75.0 | 10.0 | 0.8899 | 0.05316 | 0.7857 to 0.9941 | <0.0001 | 70.8 | 66.6 |
| | FCER2+ PI-RADS | 0.8833 | 0.0564 | 0.7728 to 0.9939 | <0.0001 | 75.0 | 20.0 | 0.8869 | 0.05501 | 0.7791 to 0.9947 | <0.0001 | 62.5 | 62.5 |
| | CANX + PI-RADS | 0.8972 | 0.0553 | 0.7888 to 1.000 | <0.0001 | 75.0 | 25.0 | 0.8929 | 0.05334 | 0.7883 to 0.9974 | <0.0001 | 79.2 | 66.6 |
| | HPX + PI-RADS | 0.875 | 0.0596 | 0.7582 to 0.9918 | <0.0001 | 75.0 | 20.0 | 0.875 | 0.05574 | 0.7658 to 0.9842 | 0.0001 | 66.7 | 66.7 |
| | KRT13 + PI-RADS | 0.9000 | 0.0520 | 0.7981 to 1.000 | <0.0001 | 65.0 | 25.0 | 0.9137 | 0.04657 | 0.8224 to 1.000 | <0.0001 | 75.0 | 66.6 |
| | HRNR + PI-RADS | 0.8528 | 0.06453 | 0.7263 to 0.9793 | 0.0002 | 55.0 | 10.0 | 0.872 | 0.05802 | 0.7583 to 0.9857 | 0.0002 | 62.5 | 62.5 |
| | CD99 + PI-RADS | 0.8778 | 0.05661 | 0.7668 to 0.9887 | <0.0001 | 65.0 | 30.0 | 0.878 | 0.05551 | 0.7692 to 0.9868 | 0.0001 | 66.7 | 62.5 |
| | PEDF + FCER2 | 0.7848 | 0.0707 | 0.6463 to 0.9233 | 0.0014 | 34.8 | 30.5 | 0.7477 | 0.07807 | 0.5947 to 0.9007 | 0.0072 | 33.3 | 29.6 |
| | PEDF + FCER2 + Age | 0.8022 | 0.0668 | 0.6712 to 0.9332 | 0.0007 | 39.1 | 30.4 | 0.7662 | 0.07473 | 0.6197 to 0.9127 | 0.0039 | 33.3 | 25.9 |
| | PEDF + FCER2 + | 0.9000 | 0.0516 | 0.7989 to 1.000 | <0.0001 | 75.0 | 25.0 | 0.8929 | 0.05284 | 0.7893 to 0.9964 | <0.0001 | 66.6 | 66.6 |
| | PI-RADS | | | | | | | | | | | | |
| | PEDF + FCER2 + Age | 0.8917 | 0.0566 | 0.7807 to 1.000 | <0.0001 | 55.0 | 20.0 | 0.8958 | 0.05172 | 0.7945 to 0.9972 | <0.0001 | 79.2 | 66.6 |
| | PI-RADS | | | | | | | | | | | | |
| | PEDF + CANX | 0.7609 | 0.0726 | 0.6186 to 0.9031 | 0.0035 | 34.8 | 34.8 | 0.7199 | 0.07878 | 0.5655 to 0.8743 | 0.017 | 33.3 | 33.3 |
| | PEDF + CANX + Age | 0.7870 | 0.0692 | 0.6513 to 0.9226 | 0.0013 | 47.8 | 30.4 | 0.7685 | 0.07278 | 0.6259 to 0.9112 | 0.0036 | 44.4 | 25.9 |
| | PEDF + CANX + | 0.8917 | 0.0586 | 0.7767 to 1.000 | <0.0001 | 75.0 | 15.0 | 0.8929 | 0.05214 | 0.7907 to 0.9950 | <0.0001 | 70.8 | 66.6 |
| | PI-RADS | | | | | | | | | | | | |
| | PEDF + CANX + Age + | 0.8861 | 0.0583 | 0.7718 to 1.000 | <0.0001 | 55.0 | 20.0 | 0.8929 | 0.05272 | 0.7805 to 0.9962 | <0.0001 | 79.2 | 66.6 |
| | PI-RADS | | | | | | | | | | | | |
| | HPX + KRT13 | 0.8022 | 0.06614 | 0.6725 to 0.9318 | 0.0007 | 43.5 | 43.5 | 0.7477 | 0.07661 | 0.5975 to 0.8978 | 0.0072 | 44.4 | 40.7 |
| | HPX + KRT13 + Age | 0.8739 | 0.05402 | 0.7680 to 0.9798 | <0.0001 | 52.2 | 34.8 | 0.8148 | 0.06571 | 0.6860 to 0.9436 | 0.0006 | 51.9 | 33.3 |
| | HPX + KRT13 + | 0.9 | 0.05199 | 0.7981 to 1.000 | <0.0001 | 65.0 | 25.0 | 0.9137 | 0.04778 | 0.8200 to 1.000 | <0.0001 | 75.0 | 66.7 |
| | PI-RADS | | | | | | | | | | | | |
| | HPX + KRT13 + Age + | 0.8972 | 0.05441 | 0.7906 to 1.000 | <0.0001 | 50.0 | 25.0 | 0.9167 | 0.046 | 0.8265 to 1.000 | <0.0001 | 75.0 | 66.7 |
| | PI-RADS | | | | | | | | | | | | |
| | PEDF + FCER2 + | 0.7848 | 0.0697 | 0.6482 to 0.9214 | 0.0014 | 34.8 | 34.8 | 0.7431 | 0.07843 | 0.5893 to 0.8968 | 0.0083 | 33.3 | 25.3 |
| | CANX | | | | | | | | | | | | |
| | PEDF + FCFR2 + | 0.8043 | 0.06665 | 0.6737 to 0.9350 | 0.0007 | 34.8 | 30.4 | 0.7708 | 0.07462 | 0.6246 to 0.9171 | 0.0033 | 37.0 | 22.2 |
| | CANX + Age | | | | | | | | | | | | |
| | PEDF + FCER2 + | 0.8944 | 0.05694 | 0.7828 to 1.000 | <0.0001 | 75.0 | 20.0 | 0.8929 | 0.05233 | 0.7903 to 0.9954 | <0.0001 | 66.7 | 66.7 |
| | CANX + PI-RADS | | | | | | | | | | | | |
| | PEDF + FCER2 + | 0.8833 | 0.05993 | 0.7659 to 1.000 | <0.0001 | 45.0 | 15.0 | 0.8958 | 0.05172 | 0.7945 to 0.9972 | <0.0001 | 79.2 | 66.7 |
| | CANX + Age + | | | | | | | | | | | | |
| | PI-RADS | | | | | | | | | | | | |
| | PEDF + FCER2 + | 0.8152 | 0.06445 | 0.6889 to 0.9415 | 0.0004 | 47.8 | 43.5 | 0.7963 | 0.06892 | 0.6612 to 0.9314 | 0.0013 | 48.1 | 37.0 |
| | CANX + KRT13 | | | | | | | | | | | | |
| | PEDF + FCER2 + | 0.8826 | 0.05261 | 0.7795 to 0.9857 | <0.0001 | 65.2 | 34.8 | 0.8472 | 0.06097 | 0.7277 to 0.9667 | 0.0002 | 55.6 | 29.6 |
| | CANX + KRT13 + Age | | | | | | | | | | | | |
| | PEDF + FCER2 + | 0.9 | 0.05199 | 0.7981 to 1.000 | <0.0001 | 70.0 | 25.0 | 0.9107 | 0.04711 | 0.8184 to 1.000 | <0.0001 | 70.8 | 70.8 |
| | CANX + KRT13 + | | | | | | | | | | | | |
| | PI-RADS | | | | | | | | | | | | |
| | PEDF + FCER2 + | 0.9028 | 0.05175 | 0.8013 to 1.000 | <0.0001 | 55.0 | 30.0 | 0.9107 | 0.04832 | 0.8160 to 1.000 | <0.0001 | 75.0 | 66.7 |
| | CANX + KRT13 + | | | | | | | | | | | | |
| | Age + PI-RADS | | | | | | | | | | | | |
| | PEDF + FCER2 + | 0.8109 | 0.06448 | 0.6845 to 0.9373 | 0.0005 | 52.2 | 43.5 | 0.794 | 0.06949 | 0.6578 to 0.9302 | 0.0014 | 44.4 | 37.0 |
| | CANX + KRT13 + | | | | | | | | | | | | |
| | HPX | | | | | | | | | | | | |
| | PEDF + FCER2 + | 0.8826 | 0.05261 | 0.7795 to 0.9857 | <0.0001 | 65.2 | 34.8 | 0.8449 | 0.0612 | 0.7250 to 0.9649 | 0.0002 | 55.6 | 29.6 |
| | CANX + KRT13 + | | | | | | | | | | | | |
| | HPX + Age | | | | | | | | | | | | |
| | PEDF + FCER2 | 0.9083 | 0.05037 | 0.8096 to 1.000 | <0.0001 | 65.0 | 25.0 | 0.9167 | 0.0491 | 0.8204 to 1.000 | <0.0001 | 79.2 | 62.5 |
| | CANX + KRT13 + | | | | | | | | | | | | |
| | HPX + PI-RADS | | | | | | | | | | | | |
| | PEDF + FCER2 + | 0.9194 | 0.04791 | 0.8256 to 1.000 | <0.0001 | 65.0 | 25.0 | 0.9256 | 0.04534 | 0.8367 to 1.000 | <0.0001 | 75.0 | 70.8 |
| | CANX + KRT13 + | | | | | | | | | | | | |
| | HPX + Age + PI-RADS | | | | | | | | | | | | |
| | PEDF + FCER2 + | 0.8043 | 0.06496 | 0.6770 to 0.9317 | 0.0007 | 47.8 | 43.5 | 0.7963 | 0.06747 | 0.6641 to 0.9285 | 0.0013 | 48.1 | 44.5 |
| | CANX + KRT13 + | | | | | | | | | | | | |
| | HPX + HRNR | | | | | | | | | | | | |
| | PEDF + FCER2 + | 0.8848 | 0.05235 | 0.7822 to 0.9874 | <0.0001 | 65.2 | 39.1 | 0.8519 | 0.05708 | 0.7400 to 0.9637 | 0.0001 | 66.7 | 66.7 |
| | CANX + KRT13 + | | | | | | | | | | | | |
| | HPX + HRNR + Age | | | | | | | | | | | | |
| | PEDF + FCER2 + | 0.9083 | 0.05095 | 0.8085 to 1.000 | <0.0001 | 65.0 | 25.0 | 0.9256 | 0.04493 | 0.8375 to 1.000 | <0.0001 | 87.5000 | 70.8000 |
| | CANX + KRT13 + | | | | | | | | | | | | |
| | HPX + HRNR + | | | | | | | | | | | | |
| | PI-RADS | | | | | | | | | | | | |
| | PEDF + FCER2 + | 0.9306 | 0.04385 | 0.8446 to 1.000 | <0.0001 | 75.0 | 30.0 | 0.9256 | 0.04454 | 0.8383 to 1.000 | <0.0001 | 83.3000 | 79.2000 |
| | CANX + KRT13 + | | | | | | | | | | | | |
| | HPX + HRNR + Age + | | | | | | | | | | | | |
| | PI-RADS | | | | | | | | | | | | |
| | PEDF + FCER2 | 0.8217 | 0.06491 | 0.6945 to 0.9490 | 0.0003 | 52.2 | 47.8 | 0.7986 | 0.06669 | 0.6679 to 0.9293 | 0.0012 | 51.8 | 45.5 |
| | CANX + KRT13 + | | | | | | | | | | | | |
| | HPX + HRNR + CD99 | | | | | | | | | | | | |
| | PEDF + FCER2 | 0.8804 | 0.05249 | 0.7776 to 0.9833 | <0.0001 | 69.6 | 43.5 | 0.8565 | 0.05617 | 0.7464 to 0.9666 | 0.0001 | 66.7 | 66.7 |
| | CANX + KRT13 + | | | | | | | | | | | | |
| | HPX + HRNR + | | | | | | | | | | | | |
| | CD99 + Age | | | | | | | | | | | | |
| | PEDF + FCER2 + | 0.9167 | 0.04763 | 0.8233 to 1.000 | <0.0001 | 650 | 30.0 | 0.9345 | 0.04244 | 0.8513 to 1.000 | <0.0001 | 91.7000 | 66.7000 |
| | CANX + KRT13 + | | | | | | | | | | | | |
| | HPX + HRNR + | | | | | | | | | | | | |
| | CD99 + PI-RADS | | | | | | | | | | | | |
| | PEDF + FCER2 + | 0.9278 | 0.04463 | 0.8403 to 1.000 | <0.0001 | 85.0 | 30.0 | 0.9375 | 0.04238 | 0.8544 to 1.000 | <0.0001 | 83.3000 | 75.0000 |
| | CANX + KRT13 + | | | | | | | | | | | | |
| | HPX + HRNR + CD99 + | | | | | | | | | | | | |
| | Age + PI-RADS | | | | | | | | | | | | |

TABLE 9.2

| | | Detection of all PCa grades | | | | | Detection of high-grade PCa | | | | |
|---|---|---|---|---|---|---|---|---|---|---|---|
| | | Pa- | | | | | Pa- | | | | |
| | | ram- | | | | | ram- | | | | |
| | | eter | Vari- | | | 95% CI | eter | Vari- | | | 95% CI |
| | | esti- | able | | Standard | (profile | esti- | able | | Standard | (profile |
| | | mates | (x) | Estimate | error | likelihood) | mates | (x) | Estimate | error | likelihood) |
| Not | PEDF + | β0 | Inter- | −4.523 | 3.222 | −11.37 to | β0 | Inter- | −4.126 | 3.203 | −10.86 to 1.933 |
| nor- | Age | | cept | | | 1.507 | | cept | | | |
| mal- | | β1 | PEDF | −0.6064 | 0.4002 | −1.505 to | β1 | PEDF | −0.5899 | 0.429 | −1.561 to |
| ized | | | | | | 0.1031 | | | | | 0.1569 |
| | | β2 | Age | 0.07817 | 0.04981 | −0.01418 to | β2 | Age | 0.06513 | 0.04892 | 0.02720 to |
| | | | | | | 0.1851 | | | | | 0.1685 |
| | FCER2 + | β0 | Inter- | −5.594 | 3.226 | −12.47 to | β0 | Inter- | −5.174 | 3.234 | −12.01 to |
| | Age | | cept | | | 0.4321 | | cept | | | 0.9225 |
| | | β1 | FCER2 | −0.00185 | 0.0008583 | −0.003754 to | β1 | FCER2 | −0.001656 | 0.0008712 | −0.003588 to |
| | | | | | | −0.0003275 | | | | | −0.0001113 |
| | | β2 | Age | 0.1081 | 0.05246 | 0.01126 to | β2 | Age | 0.09226 | 0.05185 | −0.004868 to |
| | | | | | | 0.2210 | | | | | 0.2025 |
| | CANX + | β0 | Inter- | −4.583 | 3.101 | −11.14 to | β0 | Inter- | −4.364 | 3.108 | −10.90 to 1.534 |
| | Age | | cept | | | 1.268 | | cept | | | |
| | | β1 | CANX | −0.7502 | 0.4741 | −1.861 to | β1 | CANX | −0.4466 | 0.4329 | −1.450 to |
| | | | | | | 0.05198 | | | | | 0.3091 |
| | | β2 | Age | 0.08089 | 0.04834 | −0.009829 to | β2 | Age | 0.06647 | 0.04763 | −0.02424 to |
| | | | | | | 0.1835 | | | | | 0.1663 |
| | KRT13 + | β0 | Inter- | −5.288 | 3.334 | −12.43 to | β0 | Inter- | −4.939 | 3.323 | −12.00 to 1.317 |
| | Age | | cept | | | 0.9259 | | cept | | | |
| | | β1 | KRT13 | −0.1492 | 0.06022 | −0.2847 to | β1 | KRT13 | −0.1342 | 0.06145 | −0.2731 to |
| | | | | | | −0.04420 | | | | | −0.02739 |
| | | β2 | Age | 0.1034 | 0.05334 | 0.005433 to | β2 | Age | 0.08852 | 0.05259 | −0.009640 to |
| | | | | | | 0.2194 | | | | | 0.2012 |
| | HPX + | β0 | Inter- | −4.935 | 3.194 | −11.77 to | β0 | Inter- | −4.521 | 3.245 | −11.37 to 1.637 |
| | Age | | cept | | | 1.034 | | cept | | | |
| | | β1 | HPX | −0.06832 | 0.04806 | −0.1741 to | β1 | HPX | −0.08748 | 0.05335 | −0.2030 to |
| | | | | | | 0.01545 | | | | | 0.005068 |
| | | β2 | Age | 0.08932 | 0.0507 | −0.004101 to | β2 | Age | 0.08017 | 0.05104 | −0.01535 to |
| | | | | | | 0.1994 | | | | | 0.1894 |
| | HRNR + | β0 | Inter- | −3.31 | 3.356 | −10.31 to | β0 | Inter- | −3.149 | 3.356 | −10.10 to 3.319 |
| | Age | | cept | | | 3.111 | | cept | | | |
| | | β1 | Age | 0.05767 | 0.05055 | −0.03827 to | β1 | Age | 0.04819 | 0.05013 | −0.04842 to |
| | | | | | | 0.1641 | | | | | 0.1523 |
| | | β2 | HRNR | −0.0002662 | 0.000184 | −0.0006816 to | β2 | HRNR | −0.0002495 | 0.0001957 | −0.0006991 to |
| | | | | | | 6.125e−005 | | | | | 9.365e−005 |
| | CD99 + | β0 | Inter- | −2.4 | 3.577 | −9.740 to | β0 | Inter- | −1.774 | 3.668 | −9.188 to 5.485 |
| | Age | | cept | | | 4.566 | | cept | | | |
| | | β1 | Age | 0.06657 | 0.04959 | −0.02678 to | β1 | Age | 0.0566 | 0.05065 | −0.04019 to |
| | | | | | | 0.1718 | | | | | 0.1632 |
| | | β2 | CD99 | −2.28 | 1.607 | −5.806 to | β2 | CD99 | −2.752 | 1.782 | −6.675 to |
| | | | | | | 0.5257 | | | | | 0.3309 |
| | PEDF + | β0 | Inter- | −2.914 | 1.386 | −6.109 to | β0 | Inter- | −5.457 | 2.108 | −10.47 to |
| | PI- | | cept | | | −0.5738 | | cept | | | −2.040 |
| | RADS | β1 | PEDF | −0.612 | 0.5151 | −1.784 to | β1 | PEDF | −0.6691 | 0.6252 | −2. 106 to |
| | | | | | | 0.2785 | | | | | 0.4026 |
| | | β2 | PI- | 1.143 | 0.4101 | 0.4652 to | β2 | PI- | 1.705 | 0.5946 | 0.7468 to 3.123 |
| | | | RADS | | | 2.109 | | RADS | | | |
| | FCER2 + | β0 | Inter- | −2.414 | 1.454 | −5.708 to | β0 | Inter- | −5.123 | 2.16 | −10.20 to |
| | PI- | | cept | | | 0.1083 | | cept | | | −1.553 |
| | RADS | β1 | FCER2 | −0.001439 | 0.0009008 | −0.003440 to | β1 | FCER2 | −0.001314 | 0.0009963 | −0.003528 to |
| | | | | | | 0.0001834 | | | | | 0.0004731 |
| | | β2 | PI- | 1.164 | 0.4239 | 0.4655 to | β2 | PI- | 1.721 | 0.612 | 0.7391 to 3.191 |
| | | | RADS | | | 2.164 | | RADS | | | |
| | CANX + | β0 | Inter- | −2.586 | 1.387 | −5.793 to | β0 | Inter- | −5.379 | 2.122 | −10.41 to |
| | PI- | | cept | | | −0.2265 | | cept | | | −1.948 |
| | RADS | β1 | CANX | −0.9188 | 0.552 | −2.330 to | β1 | CANX | −0.5095 | 0.5296 | −1.814 to |
| | | | | | | −0.01020 | | | | | 0.4086 |
| | | β2 | PI- | 1.151 | 0.4104 | 0.4743 to | β2 | PI- | 1.643 | 0.5814 | 0.7106 to 3.030 |
| | | | RADS | | | 2.124 | | RADS | | | |
| | KRT13 + | β0 | Inter- | −2.245 | 1.444 | −5.555 to | β0 | Inter- | −4.913 | 2.198 | −10.11 to |
| | PI- | | cept | | | 0.2349 | | cept | | | −1.330 |
| | RADS | β1 | KRT13 | −0.1225 | 0.06018 | −0.2598 to | β1 | KRT13 | −0.1074 | 0.06678 | −0.2652 to |
| | | | | | | −0.01559 | | | | | 0.009924 |
| | | β2 | PI- | 1.143 | 0.4269 | 0.4437 to | β2 | PI- | 1.668 | 0.6159 | 0.6899 to 3.153 |
| | | | RADS | | | 2.161 | | RADS | | | |
| | HPX + | β0 | Inter- | −3.329 | 1.663 | −7.181 to | β0 | Inter- | −5.728 | 2.538 | −11.73 to |
| | PI- | | cept | | | −0.4925 | | cept | | | −1.605 |
| | RADS | β1 | HPX | −0.009635 | 0.05489 | −0.1392 to | β1 | HPX | −0.01346 | 0.07264 | −0.1739 to |
| | | | | | | 0.08961 | | | | | 0.1113 |
| | | β2 | PI- | 1.111 | 0.4026 | 0.4460 to | β2 | PI- | 1.639 | 0.5888 | 0.6959 to 3.038 |
| | | | RADS | | | 2.058 | | RADS | | | |
| | HRNR + | β0 | Inter- | −3.346 | 1.638 | −7.182 to | β0 | Inter- | −7.258 | 2.825 | −14.05 to |
| | PI- | | cept | | | −0.6751 | | cept | | | −2.733 |
| | RADS | β1 | PI- | 1.098 | 0.4241 | 0.4037 to | β1 | PI- | 1.901 | 0.6967 | 0.7880 to |
| | | | RADS | | | 2.096 | | RADS | | | 3.582 |
| | | β2 | HRNR | 0.00003972 | 0.0002368 | −0.0005369 to | β2 | HRNR | 0.0002158 | 0.0002877 | −0.0003664 to |
| | | | | | | 0.0004333 | | | | | 0.0008172 |
| | CD99 + | β0 | Inter- | −1.868 | 2.351 | −6.695 to | β0 | Inter- | −3.642 | 3.039 | −10.28 to 1.992 |
| | PI- | | cept | | | 2.776 | | cept | | | |
| | RADS | β1 | PI- | 1.027 | 0.4085 | 0.3398 to | β1 | PI- | 1.573 | 0.5892 | 0.6099 to 2.961 |
| | | | RADS | | | 1.974 | | RADS | | | |
| | | β2 | CD99 | −1.531 | 1.936 | −5.894 to | β2 | CD99 | −2.442 | 2.568 | −8.086 to 1.940 |
| | | | | | | 1.741 | | | | | |
| | PEDF + | β0 | Inter- | 1.931 | 0.8802 | 0.3074 to | β0 | Inter- | 1.303 | 0.8521 | −0.3026 to |
| | FCER2 | | cept | | | 3.812 | | cept | | | 3.097 |
| | | β1 | PEDF | −0.6994 | 0.3884 | −1.568 to | β1 | PEDF | −0.6482 | 0.4083 | −1.579 to |
| | | | | | | 0.001910 | | | | | 0.07436 |
| | | β2 | FCER2 | −0.001579 | 0.0007836 | −0.003299 to | β2 | FCER2 | −0.001421 | 0.0008116 | −0.003218 to |
| | | | | | | −0.0001636 | | | | | 3.477e−005 |
| | PEDF + | β0 | Inter- | −5.021 | 3.384 | −12.24 to | β0 | Inter- | −4.554 | 3.335 | −11.59 to 1.753 |
| | FCER2 + | | cept | | | 1.304 | | cept | | | |
| | Age | β1 | PEDF | −0.7518 | 0.4054 | −1.669 to | β1 | PEDF | −0.6768 | 0.42 | −1.647 to |
| | | | | | | −0.02106 | | | | | 0.06473 |
| | | β2 | FCER2 | −0.00217 | 0.0009432 | −0.004321 to | β2 | FCER2 | −0.001888 | 0.0009344 | −0.004008 to |
| | | | | | | −0.0005330 | | | | | −0.0002596 |
| | | β3 | Age | 0.1162 | 0.05581 | 0.01449 to | β3 | Age | 0.09662 | 0.05383 | −0.003454 to |
| | | | | | | 0.2381 | | | | | 0.2121 |
| | PEDF + | β0 | Inter- | −1.394 | 1.602 | −4.903 to | β0 | Inter- | −4.359 | 2.253 | −9.570 to |
| | FCER2 + | | cept | | | 1.523 | | cept | | | −0.5328 |
| | PI- | β1 | PEDF | −0.7194 | 0.5073 | −1.901 to | β1 | PEDF | −0.6095 | 0.5989 | −2.048 to |
| | RADS | | | | | 0.1690 | | | | | 0.4150 |
| | | β2 | FCER2 | −0.001704 | 0.0009751 | −0.003909 to | β2 | FCER2 | −0.001309 | 0.001024 | −0.003609 to |
| | | | | | | 2.445e−005 | | | | | 0.0005278 |
| | | β3 | PI- | 1.186 | 0.4431 | 0.4623 to | β3 | PI- | 1.7 | 0.6159 | 0.7151 to 3.191 |
| | | | RADS | | | 2.246 | | RADS | | | |
| | PEDF + | β0 | Inter- | −8.778 | 4.839 | −19.70 to | β0 | Inter- | −8.404 | 5.662 | −21.24 to 1.901 |
| | FCER2 + | | cept | | | 0.07848 | | cept | | | |
| | Age + | β1 | PEDF | −0.875 | 0.5345 | −2.122 to | β1 | PEDF | −0.6475 | 0.5862 | −2.055 to |
| | PI- | | | | | 0.05408 | | | | | 0.3694 |
| | RADS | β2 | FCER2 | −0.002254 | 0.001165 | −0.004983 to | β2 | FCER2 | −0.001561 | 0.001129 | −0.004167 to |
| | | | | | | −0.0002781 | | | | | 0.0004058 |
| | | β3 | PI- | 1.017 | 0.421 | 0.3140 to | β3 | PI- | 1.553 | 0.6348 | 0.5597 to 3.080 |
| | | | RADS | | | 2.044 | | RADS | | | |
| | | β4 | Age | 0.1329 | 0.07759 | −0.01734 to | β4 | Age | 0.07394 | 0.0912 | −0.1056 to |
| | | | | | | 0.3005 | | | | | 0.2648 |
| | PEDF + | β0 | Inter- | 0.9844 | 0.6199 | −0.1855 to | β0 | Inter- | 0.3308 | 0.6036 | −0.8434 to |
| | CANX | | cept | | | 2.281 | | cept | | | 1.557 |
| | | β1 | PEDF | −0.4962 | 0.3862 | −1.358 to | β1 | PEDF | −0.5213 | 0.4195 | −1.473 to |
| | | | | | | 0.2027 | | | | | 0.2148 |
| | | β2 | CANX | −0.5566 | 0.4134 | −1.503 to | β2 | CANX | −0.2973 | 0.3967 | −1.194 to |
| | | | | | | 0.1810 | | | | | 0.4287 |
| | PEDF + | β0 | Inter- | −4.096 | 3.191 | −10.83 to | β0 | Inter- | −3.884 | 3.186 | −10.57 to 2.173 |
| | CANX + | | cept | | | 1.931 | | cept | | | |
| | Age | β1 | PEDF | −0.4775 | 0.3926 | −1.359 to | β1 | PEDF | −0.5119 | 0.4273 | −1.486 to |
| | | | | | | 0.2339 | | | | | 0.2365 |
| | | β2 | CANX | −0.6224 | 0.4646 | −1.716 to | β2 | CANX | −0.3241 | 0.4269 | −1.311 to |
| | | | | | | 0.1754 | | | | | 0.4368 |
| | | β3 | Age | 0.07944 | 0.0493 | −0.01266 to | β3 | Age | 0.06536 | 0.04852 | −0.02669 to |
| | | | | | | 0.1846 | | | | | 0.1674 |
| | PEDF + | β0 | Inter- | −2.32 | 1.424 | −5.582 to | β0 | Inter- | −5.1 | 2.146 | −10.18 to |
| | CANX + | | cept | | | 0.1318 | | cept | | | −1.604 |
| | PI- | | | | | | | | | | −2.012 to |
| | RADS | β1 | PEDF | −0.3514 | 0.4894 | −1.484 to | β1 | PEDF | −0.4972 | 0.6415 | 0.5847 |
| | | | | | | 0.5309 | | | | | |
| | | β2 | CANX | −0.7838 | 0.5458 | −2.201 to | β2 | CANX | −0.332 | 0.5344 | −1.665 to |
| | | | | | | 0.1179 | | | | | 0.6049 |
| | | β3 | PI- | 1.148 | 0.4141 | 0.4666 to | β3 | PI- | 1.66 | 0.5894 | 0.7148 to |
| | | | RADS | | | 2.133 | | RADS | | | 3.068 |
| | PEDF + | β0 | Inter- | −6.17 | 4.772 | −16.58 to | β0 | Inter- | −6.434 | 5.888 | −19.74 to 4.302 |
| | CANX + | | cept | | | 2.852 | | cept | | | |
| | Age + | β1 | PEDF | −0.4496 | 0.5122 | −1.643 to | β1 | PEDF | −0.5323 | 0.6584 | −2.074 to |
| | PI- | | | | | 0.4644 | | | | | 0.5875 |
| | RADS | β2 | CANX | −0.6914 | 0.5541 | −2.110 to | β2 | CANX | −0.2843 | 0.5646 | −1.676 to |
| | | | | | | 0.2438 | | | | | 0.7305 |
| | | β3 | PI- | 1.052 | 0.4066 | 0.3823 to | β3 | PI- | 1.619 | 0.6092 | 0.6483 to 3.060 |
| | | | RADS | | | 2.036 | | RADS | | | |
| | | β4 | Age | 0.065 | 0.07391 | −0.08583 to | β4 | Age | 0.02258 | 0.09143 | −0.1607 to |
| | | | | | | 0.2141 | | | | | 0.2096 |
| | HPX + | β0 | Inter- | 1.457 | 0.791 | −0.004835 to | β0 | Inter- | 1.112 | 0.8098 | −0.3912 to |
| | KRT13 | | cept | | | 3.164 | | cept | | | 2.846 |
| | | β1 | KRT13 | −0.1147 | 0.05351 | −0.2329 to | β1 | KRT13 | −0.09635 | 0.05488 | −0.2181 to |
| | | | | | | −0.01879 | | | | | 0.002047 |
| | | β2 | HPX | −0.02825 | 0.04422 | −0.1270 to | β2 | HPX | −0.04897 | 0.05102 | −0.1619 to |
| | | | | | | 0.05269 | | | | | 0.03967 |
| | HPX + | β0 | Inter- | −5.336 | 3.404 | −12.66 to | β0 | Inter- | −5.116 | 3.477 | −12.57 to 1.408 |
| | KRT13 + | | cept | | | 1.003 | | cept | | | |
| | Age | β1 | KRT13 | −0.1384 | 0.0605 | −0.2740 to | β1 | KRT13 | −0.1187 | 0.0614 | −0.2566 to |
| | | | | | | −0.03208 | | | | | −0.01065 |
| | | β2 | HPX | −0.04363 | 0.04937 | −0.1529 to | β2 | HPX | −0.06571 | 0.05496 | −0.1854 to |
| | | | | | | 0.04364 | | | | | 0.02954 |
| | | β3 | Age | 0.112 | 0.05572 | 0.01066 to | β3 | Age | 0.1027 | 0.05701 | −0.001401 to |
| | | | | | | 0.2346 | | | | | 0.2283 |
| | HPX + | β0 | Inter- | −2.556 | 1.697 | −6.509 to | β0 | Inter- | −5.196 | 2.616 | −11.47 to |
| | KRT13 + | | cept | | | 0.3353 | | cept | | | −0.9861 |
| | PI- | β1 | KRT13 | −0.1284 | 0.063 | −0.2748 to | β1 | KRT13 | −0.1105 | 0.06873 | −0.2752 to |
| | RADS | | | | | −0.01739 | | | | | 0.01001 |
| | | β2 | HPX | 0.02083 | 0.05285 | −0.1037 to | β2 | HPX | 0.01547 | 0.07111 | −0.1445 to |
| | | | | | | 0.1236 | | | | | 0.1414 |
| | | β3 | PI- | 1.164 | 0.4375 | 0.4522 to | β3 | PI- | 1.694 | 0.6347 | 0.6931 to 3.231 |
| | | | RADS | | | 2.211 | | RADS | | | |
| | HPX + | β0 | Inter- | −7.296 | 4.826 | −17.89 to | β0 | Inter- | −6.447 | 5.707 | −19.40 to 4.090 |
| | KRT13 + | | cept | | | 1.789 | | cept | | | |
| | Age + | β1 | KRT13 | −0.1336 | 0.06565 | −0.2853 to | β1 | KRT13 | −0.1098 | 0.06866 | −0.2739 to |
| | PI- | | | | | −0.01849 | | | | | 0.01082 |
| | RADS | β2 | HPX | 0.01633 | 0.05354 | −0.1095 to | β2 | HPX | 0.01445 | 0.07092 | −0.1450 to |
| | | | | | | 0.1208 | | | | | 0.1404 |
| | | β3 | PI- | 1.052 | 0.4369 | 0.3407 to | β3 | PI- | 1.652 | 0.6562 | 0.6073 to 3.218 |
| | | | RADS | | | 2.107 | | RADS | | | |
| | | β4 | Age | 0.08144 | 0.07439 | −0.06882 to | β4 | Age | 0.02176 | 0.08693 | −0.1541 to |
| | | | | | | 0.2332 | | | | | 0.1999 |
| | PEDF + | β0 | Inter- | 2.191 | 0.9405 | 0.4755 to | β0 | Inter- | 1.394 | 0.8863 | −0.2689 to |
| | FCER2 + | | cept | | | 4.232 | | cept | | | 3.271 |
| | CANX | β1 | PEDF | −0.6338 | 0.3925 | −1.503 to | β1 | PEDF | −0.6187 | 0.4119 | −1.556 to |
| | | | | | | 0.08485 | | | | | 0.1155 |
| | | β2 | FCER2 | −0.001443 | 0.000795 | −0.003183 to | β2 | FCER2 | −0.001359 | 0.0008221 | −0.003176 to |
| | | | | | | 2.496e−006 | | | | | 0.0001196 |
| | | β3 | CANX | −0.4249 | 0.4205 | −1.354 to | β3 | CANX | −0.1656 | 0.409 | −1.061 to |
| | | | | | | 0.3575 | | | | | 0.6084 |
| | PEDF + | β0 | Inter- | −4.704 | 3.365 | −11.86 to | β0 | Inter- | −4.423 | 3.339 | −11.47 to 1.899 |
| | FCER2 + | | cept | | | 1.618 | | cept | | | |
| | CANX + | β1 | PEDF | −0.6826 | 0.4081 | −1.595 to | β1 | PEDF | −0.651 | 0.4235 | −1.629 to |
| | Age | | | | | 0.06436 | | | | | 0.1016 |
| | | β2 | FCER2 | −0.002048 | 0.0009561 | −0.004226 to | β2 | FCER2 | −0.001831 | 0.0009453 | −0.003971 to |
| | | | | | | −0.0003815 | | | | | −0.0001793 |
| | | β3 | CANX | −0.4642 | 0.4701 | −1.536 to | β3 | CANX | −0.1541 | 0.4422 | −1.137 to |
| | | | | | | 0.3906 | | | | | 0.6734 |
| | | β4 | Age | 0.116 | 0.0556 | 0.01433 to | β4 | Age | 0.09594 | 0.0537 | −0.004024 to |
| | | | | | | 0.2372 | | | | | 0.2111 |
| | PEDF + | β0 | Inter- | −0.9563 | 1.631 | −4.516 to | β0 | Inter- | −4.142 | 2.281 | −9.397 to |
| | FCER2 + | | cept | | | 2.069 | | cept | | | −0.2560 |
| | CANX + | β1 | PEDF | −0.5347 | 0.5034 | −1.687 to | β1 | PEDF | −0.5065 | 0.6068 | −1.989 to |
| | PI- | | | | | 0.3794 | | | | | 0.5458 |
| | RADS | β2 | FCER2 | −0.001617 | 0.001018 | −0.003927 to | β2 | FCER2 | −0.001263 | 0.00103 | −0.003576 to |
| | | | | | | 0.0001854 | | | | | 0.0005871 |
| | | β3 | CANX | −0.6977 | 0.5295 | −2.022 to | β3 | CANX | −0.2657 | 0.5309 | −1.567 to |
| | | | | | | 0.2184 | | | | | 0.6844 |
| | | β4 | PI- | 1.21 | 0.4435 | 0.4842 to | β4 | PI- | 1.681 | 0.616 | 0.7012 to 3.177 |
| | | | RADS | | | 2.281 | | RADS | | | |
| | PEDF + | β0 | Inter- | −7.211 | 4.912 | −18.19 to | β0 | Inter- | −7.813 | 6.175 | −21.95 to 3.346 |
| | FCER2 + | | cept | | | 2.044 | | cept | | | |
| | CANX + | β1 | PEDF | −0.7267 | 0.5436 | −1.977 to | β1 | PEDF | −0.5964 | 0.6205 | −2.099 to |
| | Age + | | | | | 0.2406 | | | | | 0.4895 |
| | PI- | β2 | FCER2 | −0.002115 | 0.001183 | −0.004876 to | β2 | FCER2 | −0.00151 | 0.001144 | −0.004159 to |
| | RADS | | | | | −9.797e−005 | | | | | 0.0004846 |
| | | β3 | CANX | −0.5386 | 0.5472 | −1.863 to | β3 | CANX | −0.1251 | 0.5662 | −1.471 to |
| | | | | | | 0.4482 | | | | | 0.9402 |
| | | β4 | PI- | 1.005 | 0.4235 | 0.3088 to | β4 | PI- | 1.555 | 0.6377 | 0.5562 to 3.088 |
| | | | RADS | | | 2.059 | | RADS | | | |
| | | β5 | Age | 0.1144 | 0.08067 | −0.04690 to | β5 | Age | 0.0654 | 0.09856 | −0.1283 to |
| | | | | | | 0.2839 | | | | | 0.2722 |
| | PEDF + | β0 | Inter- | 2.555 | 1.02 | 0.7199 to | β0 | Inter- | 1.778 | 0.9648 | −0.0006037 to |
| | FCER2 + | | cept | | | 4.795 | | cept | | | 3.856 |
| | CANX + | β1 | PEDF | −0.5768 | 0.4032 | −1.457 to | β1 | PEDF | −0.5677 | 0.4202 | −1.512 to |
| | KRT13 | | | | | 0.1709 | | | | | 0.1915 |
| | | β2 | FCER2 | −0.001217 | 0.0007926 | −0.002945 to | β2 | FCER2 | −0.00115 | 0.0008189 | −0.002964 to |
| | | | | | | 0.0002712 | | | | | 0.0003611 |
| | | β3 | CANX | −0.06775 | 0.4841 | −1.092 to | β3 | CANX | 0.2563 | 0.4963 | −0.7652 to |
| | | | | | | 0.8764 | | | | | 1.256 |
| | | β4 | KRT13 | −0.09582 | 0.06538 | −0.2365 to | β4 | KRT13 | −0.1068 | 0.07046 | −0.2606 to |
| | | | | | | 0.02674 | | | | | 0.02276 |
| | PEDF + | β0 | Inter- | −5.38 | 3.544 | −12.95 to | β0 | Inter- | −5.132 | 3.55 | −12.70 to 1.544 |
| | FCER2 + | | cept | | | 1.246 | | cept | | | |
| | CANX + | β1 | PEDF | −0.6292 | 0.4273 | −1.565 to | β1 | PEDF | −0.6028 | 0.4364 | −1.590 to |
| | KRT13 + | | | | | 0.1615 | | | | | 0.1858 |
| | Age | β2 | FCER2 | −0.001935 | 0.001003 | −0.004268 to | β2 | FCER2 | −0.001733 | 0.0009939 | −0.004026 to |
| | | | | | | −0.0001864 | | | | | −1.991e−006 |
| | | β3 | CANX | −0.01459 | 0.5398 | −1.187 to | β3 | CANX | 0.3642 | 0.5368 | −0.7516 to |
| | | | | | | 1.020 | | | | | 1.440 |
| | | β4 | KRT13 | −0.1291 | 0.07444 | −0.2933 to | β4 | KRT13 | −0.1373 | 0.07956 | −0.3159 to |
| | | | | | | 0.006453 | | | | | 0.004931 |
| | | β5 | Age | 0.137 | 0.06062 | 0.02756 to | β5 | Age | 0.1169 | 0.05916 | 0.008696 to |
| | | | | | | 0.2703 | | | | | 0.2461 |
| | PEDF + | β0 | Inter- | −0.6605 | 1.682 | −4.311 to | β0 | Inter- | −3.748 | 2.346 | −9.134 to |
| | FCER2 + | | cept | | | 2.500 | | cept | | | 0.2758 |
| | CANX + | β1 | PEDF | −0.4232 | 0.525 | −1.587 to | β1 | PEDF | −0.4194 | 0.6133 | −1.923 to |
| | KRT13 + | | | | | 0.5695 | | | | | 0.6833 |
| | PI- | | | | | | | | | | 9.2 |
| | RADS | β2 | FCER2 | −0.001426 | 0.0009949 | −0.003693 to | β2 | FCER2 | −0.0009995 | 0.001017 | −0.003329 to |
| | | | | | | 0.0003813 | | | | | 0.0008444 |
| | | β3 | CANX | −0.5266 | 0.5692 | −1.915 to | β3 | CANX | −0.1145 | 0.5856 | −1.488 to |
| | | | | | | 0.5053 | | | | | 0.9957 |
| | | β4 | KRT13 | −0.084 | 0.07028 | −0.2389 to | β4 | KRT13 | −0.08166 | 0.07543 | −0.2557 to |
| | | | | | | 0.04763 | | | | | 0.05614 |
| | | β5 | PI- | 1.242 | 0.4618 | 0.4889 to | β5 | PI- | 1.669 | 0.6265 | 0.6772 to 3.188 |
| | | | RADS | | | 2.367 | | RADS | | | |
| | PEDF + | β0 | Inter- | −7.874 | 5.148 | −19.54 to | β0 | Inter- | −7.468 | 6.272 | −21.68 to 4.016 |
| | FCER2 + | | cept | | | 1.829 | | cept | | | |
| | CANX + | β1 | PEDF | −0.6921 | 0.5912 | −2.014 to | β1 | PEDF | −0.5413 | 0.6408 | −2.085 to |
| | KRT13 + | | | | | 0.3767 | | | | | 0.6189 |
| | Age + | β2 | FCER2 | −0.001966 | 0.00117 | −0.004808 to | β2 | FCER2 | −0.001252 | 0.001145 | −0.004031 to |
| | PI- | | | | | 4.785e−005 | | | | | 0.0007385 |
| | RADS | β3 | CANX | −0.2977 | 0.5948 | −1.698 to | β3 | CANX | 0.04424 | 0.6237 | −1.390 to 1.265 |
| | | | | | | 0.8274 | | | | | |
| | | β4 | KRT13 | −0.1036 | 0.07854 | −0.2806 to | β4 | KRT13 | −0.08315 | 0.07703 | −0.2611 to |
| | | | | | | 0.03988 | | | | | 0.05733 |
| | | β5 | PI- | 1.016 | 0.449 | 0.2825 to | β5 | PI- | 1.527 | 0.6525 | 0.5019 to 3.089 |
| | | | RADS | | | 2.138 | | RADS | | | |
| | | β6 | Age | 0.1346 | 0.08663 | −0.03741 to | β6 | Age | 0.0678 | 0.1027 | −0.1345 to |
| | | | | | | 0.3202 | | | | | 0.2829 |
| | PEDF + | β0 | Inter- | 2.812 | 1.18 | 0.7100 to | β0 | Inter- | 2.27 | 1.168 | 0.1603 to 4.826 |
| | FCER2 + | | cept | | | 5.439 | | cept | | | |
| | CANX + | β1 | PEDF | −0.5195 | 0.4299 | −1.430 to | β1 | PEDF | −0.485 | 0.4488 | −1.464 to |
| | KRT13 + | | | | | 0.3098 | | | | | 0.3619 |
| | HPX | β2 | FCER2 | −0.001255 | 0.0007991 | −0.002998 to | β2 | FCER2 | −0.001224 | 0.0008365 | −0.003082 to |
| | | | | | | 0.0002416 | | | | | 0.0003064 |
| | | β3 | CANX | −0.1042 | 0.4858 | −1.131 to | β3 | CANX | 0.1817 | 0.4944 | −0.8370 to |
| | | | | | | 0.8487 | | | | | 1.182 |
| | | β4 | KRT13 | −0.08629 | 0.06704 | −0.2327 to | β4 | KRT13 | −0.08597 | 0.07165 | −0.2445 to |
| | | | | | | 0.03858 | | | | | 0.04566 |
| | | β5 | HPX | −0.0246 | 0.05279 | −0.1362 to | β5 | HPX | −0.04729 | 0.05773 | −0.1714 to |
| | | | | | | 0.07605 | | | | | 0.05930 |
| | PEDF + | β0 | Inter- | −5.441 | 3.595 | −13.13 to | β0 | Inter- | −5.483 | 3.709 | −13.44 to 1.469 |
| | FCER2 + | | cept | | | 1.287 | | cept | | | |
| | CANX + | β1 | PEDF | −0.512 | 0.4636 | −1.490 to | β1 | PEDF | −0.4843 | 0.468 | −1.515 to |
| | KRT13 + | | | | | 0.3899 | | | | | 0.4003 |
| | HPX + | β2 | FCER2 | −0.002077 | 0.001062 | −0.004581 to | β2 | FCER2 | −0.001999 | 0.001116 | −0.004635 to |
| | Age | | | | | −0.0002637 | | | | | −0.0001270 |
| | | β3 | CANX | −0.0837 | 0.5452 | −1.281 to | β3 | CANX | 0.2542 | 0.5422 | −0.8939 to |
| | | | | | | 0.9573 | | | | | 1.331 |
| | | β4 | KRT13 | −0.11 | 0.07522 | −0.2759 to | β4 | KRT13 | −0.106 | 0.07949 | −0.2838 to |
| | | | | | | 0.02827 | | | | | 0.03837 |
| | | β5 | HPX | −0.05185 | 0.05937 | −0.1755 to | β5 | HPX | −0.07673 | 0.06135 | −0.2065 to |
| | | | | | | 0.06034 | | | | | 0.03822 |
| | | β6 | Age | 0.1468 | 0.06279 | 0.03408 to | β6 | Age | 0.1365 | 0.06461 | 0.02077 to |
| | | | | | | 0.2859 | | | | | 0.2804 |
| | PEDF + | β0 | Inter- | −1.095 | 1.98 | −5.593 to | β0 | Inter- | −4.012 | 2.85 | −10.71 to |
| | FCER2 + | | cept | | | 2.485 | | cept | | | 0.7659 |
| | CANX + | β1 | PEDF | −0.5587 | 0.594 | −1.837 to | β1 | PEDF | −0.4533 | 0.6412 | −1.979 to |
| | KRT13 + | | | | | 0.5997 | | | | | 0.7505 |
| | HPX + | β2 | FCER2 | −0.001343 | 0.0009932 | −0.003616 to | β2 | FCER2 | −0.0009547 | 0.00104 | −0.003376 to |
| | PI- | | | | | 0.0004869 | | | | | 0.0009301 |
| | RADS | β3 | CANX | −0.4772 | 0.5764 | −1.872 to | β3 | CANX | −0.089 | 0.603 | −1.495 to 1.071 |
| | | | | | | 0.5846 | | | | | |
| | | β4 | KRT13 | −0.09341 | 0.07411 | −0.2633 to | β4 | KRT13 | −0.08563 | 0.07925 | −0.2747 to |
| | | | | | | 0.04384 | | | | | 0.06032 |
| | | β5 | HPX | 0.03548 | 0.07426 | −0. 1218 to | β5 | HPX | 0.01511 | 0.08816 | −0.1679 to |
| | | | | | | 0.1866 | | | | | 0.1880 |
| | | β6 | PI- | 1.264 | 0.4765 | 0.4946 to | β6 | PI- | 1.687 | 0.64 | 0.6794 to 3.242 |
| | | | RADS | | | 2.431 | | RADS | | | |
| | PEDF + | β0 | Inter- | −8.431 | 5.393 | −20.97 to | β0 | Inter- | −7.789 | 6.58 | −23.08 to 4.068 |
| | FCER2 + | | cept | | | 1.548 | | cept | | | |
| | CANX + | β1 | PEDF | −0.8269 | 0.6403 | −2.206 to | β1 | PEDF | −0.5755 | 0.6633 | −2.125 to |
| | KRT13 + | | | | | 0.3943 | | | | | 0.6772 |
| | HPX + | β2 | FCER2 | −0.001866 | 0.001163 | −0.004708 to | β2 | FCER2 | −0.001203 | 0.001166 | −0.004040 to |
| | Age + | | | | | 0.0001626 | | | | | 0.0008219 |
| | PI- | β3 | CANX | −0.2554 | 0.6047 | −1.665 to | β3 | CANX | 0.07179 | 0.6416 | −1.394 to 1.346 |
| | RADS | | | | | 0.9059 | | | | | |
| | | β4 | KRT13 | −0.1162 | 0.08469 | −0.3166 to | β4 | KRT13 | −0.08765 | 0.08146 | −0.2854 to |
| | | | | | | 0.03564 | | | | | 0.06141 |
| | | β5 | HPX | 0.03908 | 0.07875 | −0.1262 to | β5 | HPX | 0.0161 | 0.08884 | −0.1669 to |
| | | | | | | 0.2002 | | | | | 0.1899 |
| | | β6 | PI- | 1.061 | 0.4699 | 0.2998 to | β6 | PI- | 1.545 | 0.6628 | 0.5032 to 3.138 |
| | | | RADS | | | 2.225 | | RADS | | | |
| | | β7 | Age | 0.1349 | 0.08626 | −0.03722 to | β7 | Age | 0.06852 | 0.1033 | −0.1354 to |
| | | | | | | 0.3208 | | | | | 0.2846 |
| | PEDF + | β0 | Inter- | 3.075 | 1.221 | 0.9070 to | β0 | Inter- | 2.373 | 1.162 | 0.2708 to 4.932 |
| | FCER2 + | | cept | | | 5.812 | | cept | | | |
| | CANX + | β1 | PEDF | −0.1792 | 0.4981 | −1.220 to | β1 | PEDF | −0.2038 | 0.5307 | −1.333 to |
| | KRT13 + | | | | | 0.7880 | | | | | 0.8108 |
| | HPX + | β2 | FCER2 | −0.001331 | 0.000824 | −0.003139 to | β2 | FCER2 | −0.001265 | 0.0008533 | −0.003165 to |
| | HRNR | | | | | 0.0001961 | | | | | 0.0002863 |
| | | β3 | CANX | −0.1042 | 0.4794 | −1.110 to | β3 | CANX | 0.1797 | 0.4911 | −0.8230 to |
| | | | | | | 0.8531 | | | | | 1.189 |
| | | β4 | KRT13 | −0.08677 | 0.06747 | −0.2343 to | β4 | KRT13 | −0.0855 | 0.07176 | −0.2445 to |
| | | | | | | 0.03868 | | | | | 0.04607 |
| | | β5 | HPX | −0.02648 | 0.05035 | −0.1329 to | β5 | HPX | −0.04383 | 0.05511 | −0.1644 to |
| | | | | | | 0.07156 | | | | | 0.05861 |
| | | β6 | HRNR | −0.0002486 | 0.000212 | −0.0007137 to | β6 | HRNR | −0.0001993 | 0.0002213 | −0.0006869 to |
| | | | | | | 0.0001395 | | | | | 0.0002081 |
| | PEDF + | β0 | Inter- | −5.028 | 3.835 | −13.08 to | β0 | Inter- | −5.319 | 3.892 | −13.52 to 2.089 |
| | FCER2 + | | cept | | | 2.282 | | cept | | | |
| | CANX + | β1 | PEDF | −0.3978 | 0.5784 | −1.643 to | β1 | PEDF | −0.4317 | 0.595 | −1.710 to |
| | KRT13 + | | | | | 0.6960 | | | | | 0.6904 |
| | HPX + | β2 | FCER2 | −0.002082 | 0.00107 | −0.004603 to | β2 | FCER2 | −0.001996 | 0.001117 | −0.004630 to |
| | HRNR + | | | | | −0.0002583 | | | | | −0.0001210 |
| | Age | β3 | CANX | −0.0755 | 0.5359 | −1.263 to | β3 | CANX | 0.2543 | 0.5389 | −0.8915 to |
| | | | | | | 0.9557 | | | | | 1.329 |
| | | β4 | KRT13 | −0.1088 | 0.07495 | −0.2743 to | β4 | KRT13 | −0.1052 | 0.07948 | −0.2833 to |
| | | | | | | 0.02892 | | | | | 0.03891 |
| | | β5 | HPX | −0.05101 | 0.0584 | −0.1740 to | β5 | HPX | −0.07567 | 0.06155 | −0.2065 to |
| | | | | | | 0.05956 | | | | | 0.03873 |
| | | β6 | Age | 0.1406 | 0.06575 | 0.02064 to | β6 | Age | 0.1339 | 0.06727 | 0.01125 to |
| | | | | | | 0.2842 | | | | | 0.2814 |
| | | β7 | HRNR | −0.0000783 | 0.0002476 | −0.0005960 to | β7 | HRNR | 0.00003636 | 0.0002568 | −0.0005786 to |
| | | | | | | 0.0004103 | | | | | 0.0004652 |
| | PEDF + | β0 | Inter- | −1.333 | 2.298 | −6.666 to | β0 | Inter- | −6.596 | 4.014 | −16.24 to |
| | FCER2 + | | cept | | | 2.687 | | cept | | | −0.1325 |
| | CANX + | β1 | PEDF | −0.6233 | 0.6662 | −2.065 to | β1 | PEDF | −0.9147 | 0.7948 | −2.822 to |
| | KRT13 + | | | | | 0.6616 | | | | | 0.5098 |
| | HPX + | β2 | FCER2 | −0.001305 | 0.001002 | −0.003611 to | β2 | FCER2 | −0.0007221 | 0.001074 | −0.003221 to |
| | HRNR + | | | | | 0.0005468 | | | | | 0.001280 |
| | PI- | β3 | CANX | −0.4829 | 0.5799 | −1.901 to | β3 | CANX | −0.04338 | 0.6328 | −1.561 to 1.151 |
| | RADS | | | | | 0.5797 | | | | | |
| | | β4 | KRT13 | −0.09157 | 0.07415 | −0.2620 to | β4 | KRT13 | −0.07899 | 0.07851 | −0.2633 to |
| | | | | | | 0.04592 | | | | | 0.06904 |
| | | β5 | HPX | 0.03737 | 0.07569 | −0.1222 to | β5 | HPX | 0.01986 | 0.09726 | −0.1766 to |
| | | | | | | 0.1928 | | | | | 0.2180 |
| | | β6 | PI- | 1.296 | 0.5051 | 0.4853 to | β6 | PI- | 2.207 | 0.8835 | 0.8510 to 4.416 |
| | | | RADS | | | 2.538 | | RADS | | | |
| | | β7 | HRNR | 0.00006685 | 0.0003059 | −0.0005419 to | β7 | HRNR | 0.0004537 | 0.0003964 | −0.0002636 to |
| | | | | | | 0.0007242 | | | | | 0.001374 |
| | PEDF + | β0 | Inter- | −10.64 | 6.608 | −26.57 to | β0 | Inter- | −22.24 | 13.64 | −58.48 to − |
| | FCER2 + | | cept | | | 0.8929 | | cept | | | 1.703 |
| | CANX + | β1 | PEDF | −1.099 | 0.7786 | −2.895 to | β1 | PEDF | −1.711 | 1.045 | −4.372 to |
| | KRT13 + | | | | | 0.3173 | | | | | 0.08005 |
| | HPX + | β2 | FCER2 | −0.001756 | 0.001152 | −0.004580 to | β2 | FCER2 | −0.001055 | 0.001321 | −0.004394 to |
| | HRNR + | | | | | 0.0002813 | | | | | 0.001251 |
| | Age + | β3 | CANX | −0.2592 | 0.6372 | −1.804 to | β3 | CANX | 0.5307 | 0.8104 | −1.162 to 2.351 |
| | PI- | | | | | 0.9460 | | | | | |
| | RADS | β4 | KRT13 | −0.1144 | 0.08542 | −0.3170 to | β4 | KRT13 | −0.08764 | 0.08563 | −0.2922 to |
| | | | | | | 0.03928 | | | | | 0.07357 |
| | | β5 | HPX | 0.04604 | 0.08573 | −0.1320 to | β5 | HPX | 0.0175 | 0.1053 | −0.1896 to |
| | | | | | | 0.2244 | | | | | 0.2435 |
| | | β6 | PI- | 1.174 | 0.532 | 0.3420 to | β6 | PI- | 2.767 | 1.341 | 0.8958 to 6.492 |
| | | | RADS | | | 2.517 | | RADS | | | |
| | | β7 | Age | 0.1573 | 0.09437 | −0.02520 to | β7 | Age | 0.2062 | 0.1471 | −0.05272 to |
| | | | | | | 0.3706 | | | | | 0.5560 |
| | | β8 | HRNR | 0.0002352 | 0.0003315 | −0.0004166 to | β8 | HRNR | 0.0008922 | 0.0005673 | −4.752e−005 to |
| | | | | | | 0.0009565 | | | | | 0.002365 |
| | PEDF + | β0 | Inter- | 5.557 | 2.22 | 1.814 to | β0 | Inter- | 4.729 | 2.23 | 0.9516 to 9.822 |
| | FCER2 + | | cept | | | 10.68 | | cept | | | |
| | CANX + | β1 | PEDF | −0.3014 | 0.5341 | −1.447 to | β1 | PEDF | −0.3048 | 0.5664 | −1.536 to |
| | KRT13 + | | | | | 0.7196 | | | | | 0.7618 |
| | HPX + | β2 | FCER2 | −0.0008146 | 0.0009307 | −0.002787 to | β2 | FCER2 | −0.0008432 | 0.000965 | −0.002904 to |
| | HRNR+ | | | | | 0.0009584 | | | | | 0.0009757 |
| | CD99 | β3 | CANX | −0.5304 | 0.5595 | −1.745 to | β3 | CANX | −0.2261 | 0.5695 | −1.430 to |
| | | | | | | 0.5480 | | | | | 0.9048 |
| | | β4 | KRT13 | −0.05938 | 0.06873 | −0.2069 to | β4 | KRT13 | −0.05296 | 0.07414 | −0.2141 to |
| | | | | | | 0.07300 | | | | | 0.08826 |
| | | β5 | HPX | −0.02132 | 0.05312 | −0.1328 to | β5 | HPX | −0.04517 | 0.05733 | −0.1702 to |
| | | | | | | 0.08266 | | | | | 0.06146 |
| | | β6 | HRNR | −0.0001882 | 0.0002188 | −0.0006655 | β6 | HRNR | −0.0001499 | 0.00023 | −0.0006541 to |
| | | | | | | to 0.0002196 | | | | | 0.0002776 |
| | | β7 | CD99 | −3.094 | 2.151 | −7.966 to | β7 | CD99 | −2.895 | 2.198 | −7.894 to |
| | | | | | | 0.5299 | | | | | 0.8321 |
| | PEDF + | β0 | Inter- | −2.474 | 4.811 | −12.44 to | β0 | Inter- | −2.933 | 4.8 | −12.88 to 6.750 |
| | FCER2 + | | cept | | | 7.228 | | cept | | | |
| | CANX + | β1 | PEDF | −0.4573 | 0.5953 | −1.753 to | β1 | PEDF | −0.4823 | 0.6086 | −1.804 to |
| | KRT13 + | | | | | 0.6581 | | | | | 0.6594 |
| | HPX + | β2 | FCER2 | −0.001716 | 0.001184 | −0.004394 to | β2 | FCER2 | −0.00171 | 0.001229 | −0.004499 to |
| | HRNR + | | | | | 0.0003883 | | | | | 0.0004554 |
| | CD99 + | β3 | CANX | −0.3113 | 0.5929 | −1.587 to | β3 | CANX | 0.01108 | 0.6048 | −1.250 to 1.228 |
| | Age | | | | | 0.8545 | | | | | |
| | | β4 | KRT13 | −0.08599 | 0.07835 | −0.2573 to | β4 | KRT13 | −0.0778 | 0.08453 | −0.2647 to |
| | | | | | | 0.06163 | | | | | 0.08009 |
| | | β5 | HPX | −0.04822 | 0.06015 | −0.1743 to | β5 | HPX | −0.07677 | 0.06302 | 0.2113 to |
| | | | | | | 0.06603 | | | | | 0.04033 |
| | | β6 | Age | 0.1218 | 0.06868 | −0.007501 to | β6 | Age | 0.1189 | 0.06981 | −0.01263 to |
| | | | | | | 0.2700 | | | | | 0.2696 |
| | | β7 | HRNR | 0.00006268 | 0.0002505 | −0.0005859 to | β7 | HRNR | 0.00002476 | 0.0002594 | −0.0005729 to |
| | | | | | | 0.0004336 | | | | | 0.0004818 |
| | | β8 | CD99 | −1.797 | 2.202 | −6.653 to | β8 | CD99 | −1.822 | 2.292 | −6.885 to 2.306 |
| | | | | | | 2.173 | | | | | |
| | PEDF + | β0 | Inter- | 2.224 | 4.106 | −5.440 to | β0 | Inter- | −3.45 | 5.188 | −15.08 to 6.651 |
| | FCER2 + | | cept | | | 11.34 | | cept | | | |
| | CANX + | β1 | PEDF | −0.7482 | 0.6977 | −2.299 to | β1 | PEDF | −1.007 | 0.8263 | −3.097 to |
| | KRT13 + | | | | | 0.5678 | | | | | 0.4485 |
| | HPX + | β2 | FCER2 | −0.0005235 | 0.001207 | −0.003115 to | β2 | FCER2 | −0.0001127 | 0.001372 | −0.002947 to |
| | HRNR+ | | | | | 0.001892 | | | | | 0.002803 |
| | CD99 + | β3 | CANX | −0.9033 | 0.7256 | −2.702 to | β3 | CANX | −0.3604 | 0.7402 | −2.131 to 1.011 |
| | PI- | | | | | 0.3706 | | | | | |
| | RADS | β4 | KRT13 | −0.07749 | 0.07536 | −0.2464 to | β4 | KRT13 | −0.06608 | 0.08404 | 0.2524 to |
| | | | | | | 0.07185 | | | | | 0.1072 |
| | | β5 | HPX | 0.06107 | 0.08245 | −0.1055 to | β5 | HPX | 0.03794 | 0.1038 | −0.1673 to |
| | | | | | | 0.2429 | | | | | 0.2629 |
| | | β6 | PI- | 1.007 | 0.5274 | 0.1544 to | β6 | PI- | 2.04 | 0.8798 | 0.6553 to 4.222 |
| | | | RADS | | | 2.289 | | RADS | | | |
| | | β7 | HRNR | 0.00007854 | 0.0003025 | −0.0005286 to | β7 | HRNR | 0.0004391 | 0.0003799 | −0.0002664 to |
| | | | | | | 0.0007226 | | | | | 0.001312 |
| | | β8 | CD99 | −3.656 | 3.683 | −12.71 to | β8 | CD99 | −3.589 | 4.372 | −14.31 to 3.179 |
| | | | | | | 1.807 | | | | | |
| | PEDF + | β0 | Inter- | −7.499 | 8.526 | −26.19 to | β0 | Inter- | −19.66 | 14.47 | −56.74 to 4.153 |
| | FCER2 + | | cept | | | 9.177 | | cept | | | |
| | CANX + | β1 | PEDF | −1.097 | 0.7829 | −2.897 to | β1 | PEDF | −1.812 | 1.104 | −4.670 to |
| | KRT13 + | | | | | 0.3265 | | | | | 0.05408 |
| | HPX + | β2 | FCER2 | −0.001215 | 0.001441 | −0.004538 to | β2 | FCER2 | −0.0006391 | 0.001598 | −0.004309 to |
| | HRNR+ | | | | | 0.001489 | | | | | 0.002463 |
| | CD99 + | β3 | CANX | −0.5445 | 0.8147 | −2.508 to | β3 | CANX | 0.2547 | 0.9152 | −1.709 to 2.210 |
| | Age + | | | | | 0.9825 | | | | | |
| | PI- | β4 | KRT13 | −0.09791 | 0.08618 | −0.3058 to | β4 | KRT13 | −0.0663 | 0.09348 | −0.2749 to |
| | RADS | | | | | 0.06572 | | | | | 0.1311 |
| | | β5 | HPX | 0.05792 | 0.08914 | −0.1239 to | β5 | HPX | 0.02794 | 0.1088 | −0.1852 to |
| | | | | | | 0.2510 | | | | | 0.2670 |
| | | β6 | PI- | 1.096 | 0.5468 | 0.2176 to | β6 | PI- | 2.735 | 1.36 | 0.8295 to 6.505 |
| | | | RADS | | | 2.452 | | RADS | | | |
| | | β7 | Age | 0.133 | 0.1011 | −0.06419 to | β7 | Age | 0.2042 | 0.1543 | −0.07172 to |
| | | | | | | 0.3594 | | | | | 0.5727 |
| | | β8 | HRNR | 0.0002379 | 0.0003351 | −0.0004177 | β8 | HRNR | 0.0009099 | 0.0005806 | −5.972e−005 to |
| | | | | | | to 0.0009719 | | | | | 0.002400 |
| | | β9 | CD99 | −2.069 | 3.863 | −11.59 to | β9 | CD99 | −3.209 | 4.722 | −14.38 to 4.950 |
| | | | | | | 3.802 | | | | | |
| | PEDF + | β0 | Inter- | −4.258 | 3.292 | −11.23 to | β0 | Inter- | −4.273 | 3.401 | −11.52 to 2.169 |
| | Age | | cept | | | 1.977 | | cept | | | |
| | | β1 | nPEDF | −45.3 | 27.91 | −117.4 to | β1 | nPEDF | −51.1 | 34.04 | −137.5 to −6.147 |
| | | | | | | −7.545 | | | | | |
| | | β2 | Age | 0.07475 | 0.05112 | −0.02122 to | β2 | Age | 0.06864 | 0.05291 | −0.03086 to |
| | | | | | | 0.1842 | | | | | 0.1828 |
| | FCER2 + | β0 | Inter- | −6.375 | 3.549 | −14.09 to | β0 | Inter- | −5.781 | 3.557 | −13.47 to |
| | Age | | cept | | | 0.1754 | | cept | | | 0.8287 |
| | | β1 | nFCER2 | −0.05597 | 0.02632 | −0.1182 to | β1 | nFCER2 | −0.04845 | 0.02744 | −0.1154 to |
| | | | | | | −0.01041 | | | | | −0.004202 |
| | | β2 | Age | 0.1092 | 0.05615 | 0.006544 to | β2 | Age | 0.09153 | 0.05554 | 0.01125 to |
| | | | | | | 0.2320 | | | | | 0.2118 |
| | CANX + | β0 | Inter- | −5.118 | 3.363 | −12.35 to | β0 | Inter- | −4.631 | 3.312 | −11.69 to 1.620 |
| | Age | | cept | | | 1.160 | | cept | | | |
| | | β1 | nCANX | −34.51 | 19.33 | −78.83 to | β1 | nCANX | −25.44 | 17.16 | −67.52 to −1.952 |
| | | | | | | −6.816 | | | | | |
| | | β2 | Age | 0.0881 | 0.05293 | −0.009740 to | β2 | Age | 0.07153 | 0.05129 | −0.02511 to |
| | | | | | | 0.2030 | | | | | 0.1814 |
| | KRT13 + | β0 | Inter- | −8.254 | 4.229 | −17.46 to | β0 | Inter- | −6.862 | 4.026 | −15.57 to |
| | Age | | cept | | | −0.5295 | | cept | | | 0.5603 |
| | | β1 | nKRT13 | −10.26 | 3.944 | −19.54 to | β1 | nKRT13 | −8.229 | 3.623 | −16.77 to −2.565 |
| | | | | | | −4.020 | | | | | |
| | | β2 | Age | 0.1497 | 0.06859 | 0.02599 to | β2 | Age | 0.1161 | 0.06387 | −0.0009410 to |
| | | | | | | 0.3001 | | | | | 0.2547 |
| | HPX + | β0 | Inter- | −6.32 | 3.896 | −14.77 to | β0 | Inter- | −5.292 | 3.797 | −13.36 to 1.873 |
| | Age | | cept | | | 0.9202 | | cept | | | |
| | | β1 | nHPX | −4.448 | 1.915 | −9.530 to | β1 | nHPX | −4.453 | 2.355 | −11.01 to |
| | | | | | | −1.508 | | | | | −1.164 |
| | | β2 | Age | 0.1117 | 0.06147 | −0.0006677 | β2 | Age | 0.0875 | 0.05862 | −0.02210 to |
| | | | | | | to 0.2468 | | | | | 0.2131 |
| | HRNR + | β0 | Inter- | −3.615 | 3.219 | −10.40 to | β0 | Inter- | −3.499 | 3.216 | −10.22 to 2.661 |
| | Age | | cept | | | 2.499 | | cept | | | |
| | | β1 | Age | 0.06015 | 0.04919 | −0.03285 to | β1 | Age | 0.05115 | 0.04879 | −0.04254 to |
| | | | | | | 0.1645 | | | | | 0.1530 |
| | | β2 | nHRNR | −0.00934 | 0.007248 | −0.02963 to | β2 | nHRNR | −0.00806 | 0.00747 | −0.02947 to |
| | | | | | | −0.0002995 | | | | | 0.0003749 |
| | CD99 + | β0 | Inter- | −4.681 | 3.587 | −12.26 to | β0 | Inter- | −4.388 | 3.59 | −11.97 to 2.447 |
| | Age | | cept | | | 2.126 | | cept | | | |
| | | β1 | Age | 0.08732 | 0.05644 | −0.01814 to | β1 | Age | 0.07334 | 0.05585 | −0.03203 to |
| | | | | | | 0.2080 | | | | | 0.1922 |
| | | β2 | nCD99 | −77.28 | 33.59 | −153.6 to | β2 | nCD99 | −65.29 | 33.87 | −143.5 to −9.663 |
| | | | | | | −19.68 | | | | | |
| | PEDF + | β0 | Inter- | −2.666 | 1.366 | −5.837 to | β0 | Inter- | −5.181 | 2.1 | −10.19 to −1.793 |
| | PI- | | cept | | | −0.3437 | | cept | | | |
| | RADS | β1 | nPEDF | −26.38 | 17.19 | −76.84 to | β1 | nPEDF | −20.92 | 17.96 | −80.25 to 4.066 |
| | | | | | | −1.864 | | | | | |
| | | β2 | PI- | 1.041 | 0.4055 | 0.3639 to | β2 | PI- | 1.544 | 0.5779 | 0.6141 to 2.921 |
| | | | RADS | | | 1.996 | | RADS | | | |
| | FCER2 + | β0 | Inter- | −2.736 | 1.366 | −5.883 to | β0 | Inter- | −5.413 | 2.092 | −10.39 to |
| | PI- | | cept | | | −0.3886 | | cept | | | −2.017 |
| | RADS | β1 | nFCER2 | −0.033 | 0.02665 | −0.09011 to | β1 | nFCER2 | −0.02695 | 0.02908 | −0.08837 to |
| | | | | | | 0.0007824 | | | | | 0.004564 |
| | | β2 | PI- | 1.047 | 0.4032 | 0.3719 to | β2 | PI- | 1.6 | 0.5825 | 0.6582 to 2.978 |
| | | | RADS | | | 1.992 | | RADS | | | |
| | CANX + | β0 | Inter- | −2.705 | 1.376 | −5.917 to | β0 | Inter- | −5.365 | 2.113 | −10.41 to |
| | PI- | | cept | | | −0.3793 | | cept | | | −1.976 |
| | RADS | β1 | nCANX | −25.6 | 17.66 | −74.36 to | β1 | nCANX | −17.19 | 14.76 | −62.09 to 3.866 |
| | | | | | | −2.830 | | | | | |
| | | β2 | PI- | 1.07 | 0.4124 | 0.3841 to | β2 | PI- | 1.596 | 0.5863 | 0.6528 to 2.993 |
| | | | RADS | | | 2.046 | | RADS | | | |
| | KRT13 + | β0 | Inter- | −1.724 | 1.372 | −4.885 to | β0 | Inter- | −4.355 | 2.106 | −9.398 to |
| | PI- | | cept | | | 0.6708 | | cept | | | −0.9666 |
| | RADS | β1 | nKRT13 | −7.639 | 3.744 | −16.84 to | β1 | nKRT13 | −5.711 | 3.868 | −15.26 to |
| | | | | | | −1.678 | | | | | 0.5800 |
| | | β2 | PI- | 0.9365 | 0.4029 | 0.2566 to | β2 | PI- | 1.416 | 0.5675 | 0.5045 to 2.774 |
| | | | RADS | | | 1.886 | | RADS | | | |
| | HPX + | β0 | Inter- | −2.02 | 1.387 | −5.222 to | β0 | Inter- | −4.645 | 2.19 | −9.845 to |
| | PI- | | cept | | | 0.3904 | | cept | | | −1.117 |
| | RADS | β1 | nHPX | −3.124 | 2.187 | −8.505 to | β1 | nHPX | −2.08 | 2.271 | −8.179 to |
| | | | | | | −0.1576 | | | | | 0.7480 |
| | | β2 | PI- | 0.8902 | 0.3887 | 0.2285 to | β2 | PI- | 1.4 | 0.5753 | 0.4795 to 2.773 |
| | | | RADS | | | 1.800 | | RADS | | | |
| | HRNR + | β0 | Inter- | −2.732 | 1.333 | −5.873 to | β0 | Inter- | −5.508 | 2.204 | −10.87 to −2.041 |
| | PI- | | cept | | | −0.5099 | | cept | | | |
| | RADS | β1 | PI- | 0.9861 | 0.3874 | 0.3399 to | β1 | PI- | 1.556 | 0.5927 | 0.6141 to 2.986 |
| | | | RADS | | | 1.899 | | RADS | | | |
| | | β2 | nHRNR | 0.006452 | 0.005968 | −0.02281 to | β2 | nHRNR | −0.002466 | 0.005537 | −0.01880 to |
| | | | | | | 0.001421 | | | | | 0.004568 |
| | CD99 + | β0 | Inter- | −1.986 | 1.448 | −5.279 to | β0 | Inter- | −4.898 | 2.202 | −10.07 to |
| | PI-RADS | | cept | | | 0.5700 | | cept | | | −1.299 |
| | | β1 | PI- | 0.8923 | 0.3989 | 0.2092 to | β1 | PI- | 1.461 | 0.5775 | 0.5237 to 2.830 |
| | | | RADS | | | 1.817 | | RADS | | | |
| | | β2 | nCD99 | −52.57 | 36.96 | −136.1 to | β2 | nCD99 | −29.48 | 39.78 | −120.7 to 7.414 |
| | | | | | | −1.127 | | | | | |
| | PEDF + | β0 | Inter- | 0.6089 | 0.4488 | −0.2221 to | β0 | Inter- | 0.1495 | 0.4475 | −0.6904 to |
| | FCER2 | | cept | | | 1.540 | | cept | | | 1.066 |
| | | β1 | nPEDF | −23.36 | 26.45 | −92.05 to | β1 | nPEDF | −31.14 | 34.39 | −114.5 to 2.430 |
| | | | | | | 1.902 | | | | | |
| | | β2 | nFCER2 | −0.02091 | 0.02992 | −0.08810 to | β2 | nFCER2 | −0.01268 | 0.03 | −0.08584 to |
| | | | | | | 0.01342 | | | | | 0.01955 |
| | PEDF + | β0 | Inter- | −5.848 | 3.662 | −13.78 to | β0 | Inter- | −5.184 | 3.666 | −13.06 to 1.648 |
| | FCER2 + | | cept | | | 0.9249 | | cept | | | |
| | | β1 | nPEDF | −17.84 | 22.12 | −94.01 to | β1 | nPEDF | −26.59 | 37.51 | −126.7 to 4.331 |
| | | | | | | 3.998 | | | | | |
| | Age | β2 | nFCER2 | −0.04092 | 0.02977 | −0.1058 to | β2 | nFCER2 | −0.02867 | 0.03338 | −0.1004 to |
| | | | | | | 0.009981 | | | | | 0.01992 |
| | | β3 | Age | 0.1025 | 0.05812 | −0.004016 to | β3 | Age | 0.08405 | 0.05749 | −0.02254 to |
| | | | | | | 0.2292 | | | | | 0.2080 |
| | PEDF + | β0 | Inter- | −2.562 | 1.362 | −5.713 to | β0 | Inter- | −5.096 | 2.082 | −10.08 to |
| | FCER2 + | | cept | | | −0.2146 | | cept | | | −1.740 |
| | PI- | β1 | nPEDF | −18.54 | 18.13 | −74.00 to | β1 | nPEDF | −16.16 | 19.72 | −82.53 to 10.79 |
| | RADS | | | | | 5.449 | | | | | |
| | | β2 | nFCER2 | −0.01031 | 0.02395 | −0.07607 to | β2 | nFCER2 | −0.006832 | 0.02284 | −0.07924 to |
| | | | | | | 0.01135 | | | | | 0.01549 |
| | | β3 | PI- | 1.021 | 0.4019 | 0.3465 to | β3 | PI- | 1.527 | 0.5729 | 0.6048 to 2.896 |
| | | | RADS | | | 1.966 | | RADS | | | |
| | PEDF + | β0 | Inter- | −7.435 | 4.9 | −18.32 to | β0 | Inter- | −6.781 | 5.334 | −18.55 to 3.124 |
| | FCER2 + | | cept | | | 1.600 | | cept | | | |
| | Age + | β1 | nPEDF | −16.83 | 17.52 | −74.20 to | β1 | nPEDF | −15.75 | 19.68 | −83.17 to 10.92 |
| | PI- | | | | | 5.946 | | | | | |
| | RADS | β2 | nFCER2 | −0.01502 | 0.03342 | −0.08615 to | β2 | nFCER2 | 0.007518 | 0.02506 | −0.08425 to |
| | | | | | | 0.01108 | | | | | 0.01549 |
| | | β3 | PI- | 0.9144 | 0.4057 | 0.2224 to | β3 | PI- | 1.474 | 0.5895 | 0.5255 to 2.871 |
| | | | RADS | | | 1.864 | | RADS | | | |
| | | β4 | Age | 0.08153 | 0.07686 | −0.06606 to | β4 | Age | 0.02887 | 0.08283 | −0.1381 to |
| | | | | | | 0.2489 | | | | | 0.1991 |
| | PEDF + | β0 | Inter- | 0.563 | 0.4219 | −0.2284 to | β0 | Inter- | 0.1246 | 0.4277 | −0.6982 to |
| | CANX | | cept | | | 1.446 | | cept | | | 0.9932 |
| | | β1 | nPEDF | −21.91 | 29.18 | −98.07 to | β1 | nPEDF | −40.3 | 41.09 | −138.0 to 4.097 |
| | | | | | | 5.267 | | | | | |
| | | β2 | nCANX | −13.81 | 18.63 | −57.52 to | β2 | nCANX | −2.024 | 19.57 | −46.67 to 35.62 |
| | | | | | | 20.27 | | | | | |
| | PEDF + | β0 | Inter- | −4.743 | 3.403 | −12.03 to | β0 | Inter- | −4.317 | 3.417 | −11.63 to 2.145 |
| | CANX + | | cept | | | 1.640 | | cept | | | |
| | Age | β1 | nPEDF | −19.07 | 28.99 | −106.9 to | β1 | nPEDF | −44.53 | 48.65 | −158.7 to 5.829 |
| | | | | | | 8.008 | | | | | |
| | | β2 | nCANX | −21.53 | 22.99 | −71.52 to | β2 | nCANX | −4.25 | 23.35 | −55.30 to 39.25 |
| | | | | | | 19.67 | | | | | |
| | | β3 | Age | 0.08312 | 0.05341 | −0.01582 to | β3 | Age | 0.06932 | 0.0532 | −0.03049 to |
| | | | | | | 0.1988 | | | | | 0.1847 |
| | PEDF + | β0 | Inter- | −2.575 | 1.369 | −5.777 to | β0 | Inter- | −5.153 | 2.11 | −10.21 to −1.771 |
| | CANX + | | cept | | | −0.2622 | | cept | | | |
| | PI- | β1 | nPEDF | −9.191 | 16.57 | −68.71 to | β1 | nPEDF | −10.13 | 23.23 | 92.71 to 16.26 |
| | RADS | | | | | 12.43 | | | | | |
| | | β2 | nCANX | −18.14 | 19.56 | −71.76 to | β2 | nCANX | −9.954 | 20.54 | −61.33 to 33.20 |
| | | | | | | 16.17 | | | | | |
| | | β3 | PI- | 1.042 | 0.4096 | 0.3606 to | β3 | PI- | 1.546 | 0.5822 | 0.6128 to 2.940 |
| | | | RADS | | | 2.013 | | RADS | | | |
| | PEDF + | β0 | Inter- | −6.723 | 4.901 | −17.42 to | β0 | Inter- | −6.331 | 5.415 | −18.36 to 3.608 |
| | CANX + | | cept | | | 2.370 | | cept | | | |
| | Age + | β1 | nPEDF | −10.77 | 16.68 | −73.64 to | β1 | nPEDF | −10.97 | 24.01 | −97.73 to 16.15 |
| | PI- | | | | | 11.60 | | | | | |
| | RADS | β2 | nCANX | −16.11 | 19.05 | −69.08 to | β2 | nCANX | −9.001 | 20.94 | −60.85 to 35.98 |
| | | | | | | 18.96 | | | | | |
| | | β3 | PI- | 0.9455 | 0.4066 | 0.2676 to | β3 | PI- | 1.507 | 0.6009 | 0.5470 to 2.935 |
| | | | RADS | | | 1.918 | | RADS | | | |
| | | β4 | Age | 0.06917 | 0.07605 | −0.08020 to | β4 | Age | 0.02017 | 0.08431 | −0.1486 to |
| | | | | | | 0.2251 | | | | | 0.1928 |
| | HPX + | β0 | Inter- | 1.038 | 0.5068 | 0.1056 to | β0 | Inter- | 0.4789 | 0.493 | −0.4455 to |
| | KRT13 | | cept | | | 2.124 | | cept | | | 1.523 |
| | | β1 | nKRT13 | −5.039 | 4.041 | −13.91 to | β1 | nKRT13 | −3.256 | 4.15 | −12.28 to 4.426 |
| | | | | | | 2.159 | | | | | |
| | | β2 | nHPX | −1.631 | 2.435 | −8.039 to | β2 | nHPX | −2.611 | 3.18 | −10.72 to 1.671 |
| | | | | | | 1.899 | | | | | |
| | HPX + | β0 | Inter- | −8.054 | 4.265 | −17.32 to | β0 | Inter- | −6.476 | 4.064 | −15.24 to 1.053 |
| | KRT13 + | | cept | | | −0.2522 | | cept | | | |
| | Age | β1 | nKRT13 | −8.632 | 5.212 | −20.26 to | β1 | nKRT13 | −5.754 | 4.866 | −16.29 to 3.028 |
| | | | | | | 0.4811 | | | | | |
| | | β2 | nHPX | −0.9857 | 2.245 | −6.706 to | β2 | nHPX | −1.801 | 2.705 | −9.190 to 2.222 |
| | | | | | | 2.698 | | | | | |
| | | β3 | Age | 0.1462 | 0.06911 | 0.02146 to | β3 | Age | 0.11 | 0.06431 | −0.007940 to |
| | | | | | | 0.2974 | | | | | 0.2494 |
| | HPX + | β0 | Inter- | −1.724 | 1.378 | −4.910 to | β0 | Inter- | −4.384 | 2.154 | −9.577 to |
| | KRT13 + | | cept | | | 0.6794 | | cept | | | −0.9310 |
| | PI- | β1 | nKRT13 | −7.623 | 5.167 | −19.59 to | β1 | nKRT13 | −5.962 | 5.282 | −18.06 to 3.305 |
| | RADS | | | | | 1.143 | | | | | |
| | | β2 | nHPX | −0.009089 | 2.015 | −5.922 to | β2 | nHPX | 0.1525 | 2.135 | −6.583 to |
| | | | | | | 3.319 | | | | | 3.643 |
| | | β3 | PI- | 0.9363 | 0.4074 | 0.2512 to | β3 | PI- | 1.425 | 0.5836 | 0.4947 to 2.832 |
| | | | RADS | | | 1.901 | | RADS | | | |
| | HPX + | β0 | Inter- | −6.734 | 5.311 | −18.41 to | β0 | Inter- | −5.687 | 5.582 | −17.90 to 4.784 |
| | KRT13 + | | cept | | | 3.147 | | cept | | | |
| | Age + | β1 | nKRT13 | −8.05 | 5.402 | −20.60 to | β1 | nKRT13 | −5.975 | 5.298 | −18.11 to 3.325 |
| | PI- | | | | | 1.150 | | | | | |
| | RADS | β2 | nHPX | 0.09808 | 1.992 | −5.589 to | β2 | nHPX | 0.1644 | 2.118 | −6.514 to 3.649 |
| | | | | | | 3.482 | | | | | |
| | | β3 | PI- | 0.8053 | 0.427 | 0.07364 to | β3 | PI- | 1.386 | 0.6006 | 0.4139 to 2.822 |
| | | | RADS | | | 1.799 | | RADS | | | |
| | | β4 | Age | 0.08481 | 0.08564 | −0.07820 to | β4 | Age | 0.02209 | 0.08646 | −0.1519 to |
| | | | | | | 0.2694 | | | | | 0.1987 |
| | PEDF + | β0 | Inter- | 0.6094 | 0.4467 | −0.2140 to | β0 | Inter- | 0.1526 | 0.4499 | −0.6935 to |
| | FCER2 + | | cept | | | 1.537 | | cept | | | 1.073 |
| | CANX | β1 | nPEDF | −19.26 | 26.35 | −94.87 to | β1 | nPEDF | −34.55 | 40.59 | −133.6 to 3.973 |
| | | | | | | 4.909 | | | | | |
| | | β2 | nFCER2 | −0.01544 | 0.03325 | −0.09099 to | β2 | nFCER2 | −0.015 | 0.03336 | −0.09698 to |
| | | | | | | 0.02330 | | | | | 0.02297 |
| | | β3 | nCANX | −6.881 | 20.97 | −59.45 to | β3 | nCANX | 3.719 | 21.08 | −46.93 to 44.24 |
| | | | | | | 31.35 | | | | | |
| | PEDF + | β0 | Inter- | −5.779 | 3.682 | −13.79 to | β0 | Inter- | −5.357 | 3.744 | −13.48 to 1.570 |
| | FCER2 + | | cept | | | 1.009 | | cept | | | |
| | CANX + | β1 | nPEDF | −16.62 | 22.66 | −99.09 to | β1 | nPEDF | −33.36 | 46.26 | −147.9 to 5.252 |
| | Age | | | | | 6.672 | | | | | |
| | | β2 | nFCER2 | −0.03769 | 0.03729 | −0.1177 to | β2 | nFCER2 | −0.03507 | 0.03886 | −0.1220 to |
| | | | | | | 0.02322 | | | | | 0.02358 |
| | | β3 | nCANX | −3.323 | 23.78 | −65.09 to | β3 | nCANX | 8.242 | 23.61 | −49.31 to 53.40 |
| | | | | | | 38.23 | | | | | |
| | | β4 | Age | 0.1014 | 0.05842 | −0.005332 to | β4 | Age | 0.08689 | 0.05876 | −0.02129 to |
| | | | | | | 0.2292 | | | | | 0.2148 |
| | PEDF + | β0 | Inter- | −2.569 | 1.373 | −5.782 to | β0 | Inter- | −5.12 | 2.104 | −10.18 to |
| | FCER2 + | | cept | | | −0.2317 | | cept | | | −1.744 |
| | CANX + | β1 | nPEDF | −9.29 | 16.66 | −68.70 to | β1 | nPEDF | −10.68 | 23.8 | −92.27 to 16.28 |
| | PI- | | | | | 12.93 | | | | | |
| | RADS | β2 | nFCER2 | −0.001053 | 0.0193 | −0.07168 to | β2 | nFCER2 | −0.003527 | 0.02209 | −0.08889 to |
| | | | | | | 0.03112 | | | | | 0.02751 |
| | | β3 | nCANX | −17.26 | 24.89 | −86.04 to | β3 | nCANX | −7.268 | 25.21 | −71.44 to 43.93 |
| | | | | | | 24.44 | | | | | |
| | | β4 | PI- | 1.041 | 0.4103 | 0.3572 to | β4 | PI- | 1.538 | 0.5805 | 0.6079 to 2.933 |
| | | | RADS | | | 2.015 | | RADS | | | |
| | PEDF + | β0 | Inter- | −6.859 | 4.95 | −17.83 to | β0 | Inter- | −6.515 | 5.467 | −18.65 to 3.556 |
| | FCER2 + | | cept | | | 2.325 | | cept | | | |
| | CANX + | β1 | nPEDF | −11.2 | 16.78 | −73.29 to | β1 | nPEDF | −11.89 | 24.98 | −97.32 to 16.15 |
| | Age + | | | | | 11.72 | | | | | |
| | PI- | β2 | nFCER2 | −0.00456 | 0.02511 | −0.09206 to | β2 | nFCER2 | 0.004823 | 0.02481 | −0.1042 to |
| | RADS | | | | | 0.02872 | | | | | 0.02723 |
| | | β3 | nCANX | −12.68 | 24.37 | −79.79 to | β3 | nCANX | 5.258 | 26.35 | −70.96 to 50.22 |
| | | | | | | 32.12 | | | | | |
| | | β4 | PI- | 0.9347 | 0.4091 | 0.2381 to | β4 | PI- | 1.489 | 0.6008 | 0.5294 to 2.923 |
| | | | RADS | | | 1.911 | | RADS | | | |
| | | β5 | Age | 0.07191 | 0.07741 | −0.07966 to | β5 | Age | 0.02408 | 0.08595 | −0.1481 to |
| | | | | | | 0.2415 | | | | | 0.2045 |
| | PEDF + | β0 | Inter- | 1.097 | 0.5258 | 0.1407 to | β0 | Inter- | 0.6311 | 0.5162 | −0.3359 to |
| | FCER2 + | | cept | | | 2.234 | | cept | | | 1.724 |
| | CANX + | β1 | nPEDF | −16.18 | 30.97 | −94.67 to | β1 | nPEDF | −48.23 | 43.66 | −143.7 to 10.37 |
| | KRT13 | | | | | 13.48 | | | | | |
| | | β2 | nFCER2 | −0.009471 | 0.01855 | −0.07953 to | β2 | nFCER2 | −0.01505 | 0.02079 | −0.08707 to |
| | | | | | | 0.01474 | | | | | 0.01207 |
| | | β3 | nCANX | 25.55 | 30.23 | −29.73 to | β3 | nCANX | 55.4 | 42.48 | −16.21 to 152.5 |
| | | | | | | 100.6 | | | | | |
| | | β4 | nKRT13 | −8.41 | 4.777 | −19.77 to | β4 | nKRT13 | −9.284 | 5.56 | −23.11 to |
| | | | | | | −0.8028 | | | | | −0.5903 |
| | PEDF + | β0 | Inter- | −9.266 | 4.523 | −19.32 to | β0 | Inter- | −8.079 | 4.473 | −18.12 to |
| | FCER2 + | | cept | | | −1.136 | | cept | | | −0.06979 |
| | CANX + | β1 | nPEDF | −17.85 | 37.36 | −110.4 to | β1 | nPEDF | −57.26 | 47.81 | −160.2 to 12.23 |
| | KRT13 + | | | | | 18.73 | | | | | |
| | Age | β2 | nFCER2 | −0.0152 | 0.03721 | −0.1079 to | β2 | nFCER2 | −0.02261 | 0.04063 | −0.1167 to |
| | | | | | | 0.01465 | | | | | 0.01165 |
| | | β3 | nCANX | 41.24 | 36.56 | −20.27 to | β3 | nCANX | 73.38 | 45.55 | −6.129 to 173.8 |
| | | | | | | 128.1 | | | | | |
| | | β4 | nKRT13 | −14.05 | 6.489 | −28.92 to | β4 | nKRT13 | −13.1 | 6.653 | −28.78 to −2.648 |
| | | | | | | −3.507 | | | | | |
| | | β5 | Age | 0.1688 | 0.07431 | 0.03694 to | β5 | Age | 0.1397 | 0.07194 | 0.01223 to |
| | | | | | | 0.3352 | | | | | 0.3021 |
| | PEDF + | β0 | Inter- | −1.795 | 1.44 | −5.113 to | β0 | Inter- | −4.4 | 2.212 | −9.759 to − |
| | FCER2 + | | cept | | | 0.7443 | | cept | | | |
| | CANX + | β1 | nPEDF | −2.816 | 16.87 | −62.43 to | β1 | nPEDF | −7.226 | 25.34 | 0.8324 |
| | KRT13 + | | | | | 22.49 | | | | | −94.87 to 24.19 |
| | PI- | β2 | nFCER2 | −0.001306 | 0.01618 | −0.06949 to | β2 | nFCER2 | −0.003276 | 0.02 | −0.08680 to |
| | RADS | | | | | 0.02732 | | | | | 0.02573 |
| | | β3 | nCANX | −1.581 | 25.76 | −70.36 to | β3 | nCANX | 2.34 | 29.33 | −65.75 to 87.21 |
| | | | | | | 57.20 | | | | | |
| | | β4 | nKRT13 | −6.448 | 6.032 | −21.71 to | β4 | nKRT13 | −4.263 | 6.594 | −21.14 to 5.774 |
| | | | | | | 2.812 | | | | | |
| | | β5 | PI- | 0.9436 | 0.4129 | 0.2458 to | β5 | PI- | 1.414 | 0.5836 | 0.4794 to 2.824 |
| | | | RADS | | | 1.919 | | RADS | | | |
| | PEDF + | β0 | Inter- | −7.168 | 5.48 | −19.29 to | β0 | Inter- | −6.077 | 5.653 | −18.50 to 4.556 |
| | FCER2 + | | cept | | | 2.957 | | cept | | | |
| | CANX + | β1 | nPEDF | −6.139 | 18.64 | −75.56 to | β1 | nPEDF | −8.907 | 27.5 | −104.6 to 23.83 |
| | KRT13 + | | | | | 21.42 | | | | | |
| | Age + | β2 | nFCER2 | −0.004333 | 0.01918 | −0.09148 to | β2 | nFCER2 | 0.004708 | 0.02274 | −0.1042 to |
| | PI- | | | | | 0.02429 | | | | | 0.02531 |
| | RADS | β3 | nCANX | 6.441 | 26.8 | −61.45 to | β3 | nCANX | 5.253 | 31.5 | −64.78 to 99.19 |
| | | | | | | 75.82 | | | | | |
| | | β4 | nKRT13 | −7.265 | 6.412 | −23.12 to | β4 | nKRT13 | −4.424 | 6.678 | −21.32 to 5.737 |
| | | | | | | 2.515 | | | | | |
| | | β5 | PI- | 0.7753 | 0.4363 | 0.01580 to | β5 | PI- | 1.35 | 0.6101 | 0.3530 to 2.806 |
| | | | RADS | | | 1.785 | | RADS | | | |
| | | β6 | Age | 0.09259 | 0.08976 | −0.07700 to | β6 | Age | 0.02935 | 0.09006 | −0.1504 to |
| | | | | | | 0.2872 | | | | | 0.2202 |
| | PEDF + | β0 | Inter- | 1.101 | 0.5287 | 0.1431 to | β0 | Inter- | 0.643 | 0.5258 | −0.3317 to |
| | FCER2 + | | cept | | | 2.258 | | cept | | | 1.791 |
| | CANX + | β1 | nPEDF | −10.97 | 34.03 | −93.14 to | β1 | nPEDF | −40.43 | 46.05 | −140.4 to 33.12 |
| | KRT13 + | | | | | 33.18 | | | | | |
| | HPX | β2 | nFCER2 | −0.004042 | 0.02473 | −0.07759 to | β2 | nFCER2 | 0.007635 | 0.02848 | −0.08429 to |
| | | | | | | 0.04195 | | | | | 0.04457 |
| | | β3 | nCANX | 20.97 | 32.08 | −37.25 to | β3 | nCANX | 48.94 | 43.64 | −25.22 to 148.8 |
| | | | | | | 99.08 | | | | | |
| | | β4 | nKRT13 | −7.33 | 5.426 | −19.68 to | β4 | nKRT13 | −7.807 | 6.292 | −22.60 to 2.757 |
| | | | | | | 1.781 | | | | | |
| | | β5 | nHPX | −1.236 | 3.175 | −9.172 to | β5 | nHPX | −1.771 | 3.786 | −11.19 to 4.302 |
| | | | | | | 4.047 | | | | | |
| | PEDF + | β0 | Inter- | −9.298 | 4.558 | −19.38 to | β0 | Inter- | −8.024 | 4.513 | −18.11 to |
| | FCER2 + | | cept | | | −1.062 | | cept | | | 0.1344 |
| | CANX + | β1 | nPEDF | −18.4 | 38.55 | −112.1 to | β1 | nPEDF | −55.91 | 49.74 | −162.8 to 25.93 |
| | KRT13 + | | | | | 30.02 | | | | | |
| | HPX + | β2 | nFCER2 | −0.0158 | 0.03813 | −0.1088 to | β2 | nFCER2 | −0.02173 | 0.04284 | −0.1166 to |
| | Age | | | | | 0.03075 | | | | | 0.03406 |
| | | β3 | nCANX | 41.92 | 38.55 | −24.63 to | β3 | nCANX | 72.07 | 47.41 | −11.87 to 177.0 |
| | | | | | | 131.7 | | | | | |
| | | β4 | nKRT13 | −14.27 | 7.634 | −31.18 to | β4 | nKRT13 | −12.74 | 7.625 | −29.94 to |
| | | | | | | −1.221 | | | | | 0.08316 |
| | | β5 | nHPX | 0.1529 | 2.731 | −6.849 to | β5 | nHPX | −0.2988 | 3.121 | −9.033 to 5.152 |
| | | | | | | 5.351 | | | | | |
| | | β6 | Age | 0.1694 | 0.07505 | 0.03569 to | β6 | Age | 0.1388 | 0.07257 | 0.009148 to |
| | | | | | | 0.3367 | | | | | 0.3021 |
| | PEDF + | β0 | Inter- | −2.055 | 1.6 | −5.845 to | β0 | Inter- | −5.7 | 2.934 | −13.08 to −1.189 |
| | FCER2 + | | cept | | | 0.6822 | | cept | | | |
| | CANX + | β1 | nPEDF | −14.46 | 29.6 | −85.07 to | β1 | nPEDF | −31.56 | 35.29 | −126.0 to 30.62 |
| | KRT13 + | | | | | 37.46 | | | | | |
| | HPX + | β2 | nFCER2 | −0.01273 | 0.0282 | −0.07683 to | β2 | nFCER2 | −0.02733 | 0.03037 | −0.09816 to |
| | PI- | | | | | 0.04431 | | | | | 0.03343 |
| | RADS | β3 | nCANX | 5.473 | 30.01 | −67.99 to | β3 | nCANX | 16.07 | 32.61 | −56.76 to 102.6 |
| | | | | | | 69.73 | | | | | |
| | | β4 | nKRT13 | −7.462 | 6.325 | −23.02 to | β4 | nKRT13 | −6.073 | 6.44 | −22.50 to 4.746 |
| | | | | | | 2.788 | | | | | |
| | | β5 | nHPX | 2.415 | 5.077 | −8.016 to | β5 | nHPX | 5.22 | 5.64 | −6.150 to 17.10 |
| | | | | | | 12.74 | | | | | |
| | | β6 | PI- | 1.002 | 0.4469 | 0.2641 to | β6 | PI- | 1.727 | 0.7563 | 0.5698 to 3.619 |
| | | | RADS | | | 2.077 | | RADS | | | |
| | PEDF + | β0 | Inter- | −10.65 | 6.145 | −24.37 to | β0 | Inter- | −11.43 | 7.283 | −27.45 to 2.208 |
| | FCER2 + | | cept | | | 1.132 | | cept | | | |
| | CANX + | β1 | nPEDF | −37.57 | 31.12 | −114.4 to | β1 | nPEDF | −47.54 | 39.15 | −150.7 to 24.89 |
| | KRT13 + | | | | | 25.62 | | | | | |
| | HPX + | β2 | nFCER2 | −0.03717 | 0.03228 | −0.1185 to | β2 | nFCER2 | −0.04362 | 0.03576 | −0.1357 to |
| | Age + | | | | | 0.02820 | | | | | 0.02675 |
| | PI- | β3 | nCANX | 31.17 | 34.97 | −46.76 to | β3 | nCANX | 31.38 | 37.26 | −47.45 to 128.3 |
| | RADS | | | | | 109.3 | | | | | |
| | | β4 | nKRT13 | −12.24 | 7.972 | −30.82 to | β4 | nKRT13 | −8.096 | 6.91 | −24.65 to 3.840 |
| | | | | | | 1.022 | | | | | |
| | | β5 | nHPX | 6.711 | 5.83 | −5.193 to | β5 | nHPX | 7.996 | 6.47 | −4.867 to 21.77 |
| | | | | | | 19.21 | | | | | |
| | | β6 | PI- | 0.8949 | 0.4956 | 0.06541 to | β6 | PI- | 1.716 | 0.784 | 0.4900 to 3.652 |
| | | | RADS | | | 2.083 | | RADS | | | |
| | | β7 | Age | 0.1404 | 0.09538 | −0.04675 to | β7 | Age | 0.08822 | 0.09907 | −0.1127 to |
| | | | | | | 0.3486 | | | | | 0.2946 |
| | PEDF + | β0 | Inter- | 1.165 | 0.5427 | 0.1876 to | β0 | Inter- | 0.8184 | 0.5944 | −0.2407 to |
| | FCER2 + | | cept | | | 2.364 | | cept | | | 2.135 |
| | CANX + | β1 | nPEDF | −14.06 | 25.09 | −90.55 to | β1 | nPEDF | −31.65 | 46.25 | −132.2 to 25.59 |
| | KRT13 + | | | | | 32.97 | | | | | |
| | HPX + | β2 | nFCER2 | −0.0123 | 0.03238 | −0.08114 to | β2 | nFCER2 | −0.02374 | 0.034 | −0.09587 to |
| | HRNR | | | | | 0.04189 | | | | | 0.04197 |
| | | β3 | nCANX | 24.51 | 29.53 | −37.00 to | β3 | nCANX | 50.51 | 45.18 | −24.82 to 153.0 |
| | | | | | | 99.50 | | | | | |
| | | β4 | nKRT13 | −7.471 | 5.676 | −20.31 to | β4 | nKRT13 | −7.672 | 6.625 | −23.28 to 3.646 |
| | | | | | | 2.216 | | | | | |
| | | β5 | nHPX | −2.308 | 4.085 | −12.15 to | β5 | nHPX | −5.439 | 6.331 | −19.54 to 3.561 |
| | | | | | | 3.774 | | | | | |
| | | β6 | nHRNR | 0.006958 | 0.008637 | −0.01309 to | β6 | nHRNR | 0.01298 | 0.01338 | −0.01125 to |
| | | | | | | 0.02608 | | | | | 0.04094 |
| | PEDF + | β0 | Inter- | −12.56 | 5.38 | −24.80 to | β0 | Inter- | −11.42 | 5.535 | −24.20 to −1.797 |
| | FCER2 + | | cept | | | −3.116 | | cept | | | |
| | CANX + | β1 | nPEDF | −40.75 | 28.3 | −124.3 to | β1 | nPEDF | −56.49 | 41.98 | −164.0 to 3.574 |
| | KRT13 + | | | | | 10.62 | | | | | |
| | HPX + | β2 | nFCER2 | −0.05732 | 0.04335 | −0.1509 to | β2 | nFCER2 | −0.07533 | 0.04799 | −0.1803 to |
| | HRNR + | | | | | 0.01825 | | | | | 0.01373 |
| | Age | β3 | nCANX | 69.45 | 40.52 | −11.30 to | β3 | nCANX | 96.52 | 54.31 | 2.850 to 220.2 |
| | | | | | | 163.6 | | | | | |
| | | β4 | nKRT13 | −17.84 | 8.939 | −38.02 to | β4 | nKRT13 | −16.16 | 9.08 | −36.88 to −1.223 |
| | | | | | | −2.759 | | | | | |
| | | β5 | nHPX | −0.3202 | 3.331 | −10.33 to | β5 | nHPX | −4.851 | 7.322 | −20.49 to 4.203 |
| | | | | | | 5.280 | | | | | |
| | | β6 | Age | 0.2249 | 0.08937 | 0.06994 to | β6 | Age | 0.1981 | 0.09036 | 0.04304 to |
| | | | | | | 0.4295 | | | | | 0.4078 |
| | | β7 | nHRNR | 0.01955 | 0.011 | −0.002660 to | β7 | nHRNR | 0.02828 | 0.01864 | −0.001940 to |
| | | | | | | 0.04593 | | | | | 0.06819 |
| | PEDF + | β0 | Inter- | −1.971 | 1.597 | −5.759 to | β0 | Inter- | −5.475 | 2.942 | −12.93 to − |
| | FCER2 + | | cept | | | 0.7884 | | cept | | | 0.8456 |
| | CANX + | β1 | nPEDF | −14.91 | 25.05 | −81.18 to | β1 | nPEDF | −27.55 | 27.73 | −116.1 to 23.53 |
| | KRT13 + | | | | | 35.71 | | | | | |
| | HPX + | β2 | nFCER2 | −0.01076 | 0.03016 | −0.08073 to | β2 | nFCER2 | −0.03436 | 0.04165 | −0.1203 to |
| | HRNR + | | | | | 0.04639 | | | | | 0.03753 |
| | PI- | β3 | nCANX | 4.566 | 31.93 | −79.86 to | β3 | nCANX | 18.66 | 36.8 | −71.41 to 109.5 |
| | | | | | | 68.71 | | | | | |
| | RADS | β4 | nKRT13 | −7.604 | 6.844 | −24.57 to | β4 | nKRT13 | −6.309 | 8.07 | −27.21 to 7.049 |
| | | | | | | 3.771 | | | | | |
| | | β5 | nHPX | 0.7556 | 5.265 | −9.897 to | β5 | nHPX | 1.234 | 6.334 | −13.17 to 15.37 |
| | | | | | | 11.96 | | | | | |
| | | β6 | PI- | 0.9999 | 0.4459 | 0.2590 to | β6 | PI- | 1.726 | 0.7611 | 0.5559 to 3.640 |
| | | | RADS | | | 2.070 | | RADS | | | |
| | | β7 | nHRNR | 0.006387 | 0.007846 | −0.01612 to | β7 | nHRNR | 0.01206 | 0.01218 | −0.01200 to |
| | | | | | | 0.02547 | | | | | 0.04084 |
| | PEDF + | β0 | Inter- | −12.98 | 7.335 | −29.83 to | β0 | Inter- | −18.38 | 10.77 | 45.04 to |
| | FCER2 + | | cept | | | 0.07233 | | cept | | | −0.6707 |
| | CANX + | β1 | nPEDF | −45.45 | 34.75 | −126.4 to | β1 | nPEDF | −65.46 | 41.68 | −178.7 to 6.972 |
| | KRT13 + | | | | | 16.66 | | | | | |
| | HPX + | β2 | nFCER2 | −0.05362 | 0.05106 | −0.1640 to | β2 | nFCER2 | −0.1032 | 0.07073 | −0.2760 to |
| | HRNR + | | | | | 0.02733 | | | | | 0.01655 |
| | Age + | β3 | nCANX | 40.04 | 41.94 | −55.28 to | β3 | nCANX | 67.76 | 57.78 | −42.55 to 214.7 |
| | PI- | | | | | 129.7 | | | | | |
| | RADS | β4 | nKRT13 | −12.31 | 8.803 | −33.18 to | β4 | nKRT13 | −10.03 | 8.727 | −33.28 to 4.630 |
| | | | | | | 1.882 | | | | | |
| | | β5 | nHPX | 4.715 | 7.258 | −7.819 to | β5 | nHPX | 3.184 | 6.798 | −13.10 to 18.96 |
| | | | | | | 18.72 | | | | | |
| | | β6 | PI- | 0.8461 | 0.5118 | −0.02322 to | β6 | PI- | 1.886 | 0.9074 | 0.4880 to |
| | | | RADS | | | 2.067 | | RADS | | | 4.168 |
| | | β7 | Age | 0.1806 | 0.1143 | −0.02826 to | β7 | Age | 0.192 | 0.1445 | −0.06372 to |
| | | | | | | 0.4360 | | | | | 0.5392 |
| | | β8 | nHRNR | 0.01189 | 0.01408 | −0.01112 to | β8 | nHRNR | 0.02653 | 0.01802 | −0.006014 to |
| | | | | | | 0.03996 | | | | | 0.07463 |
| | PEDF + | β0 | Inter- | 1.189 | 0.5372 | 0.2190 to | β0 | Inter- | 0.8101 | 0.5771 | −0.2247 to |
| | FCER2 + | | cept | | | 2.374 | | cept | | | 2.102 |
| | CANX + | β1 | nPEDF | 25.06 | 31.48 | −116.3 to | β1 | nPEDF | −49.43 | 52.76 | −173.4 to 21.39 |
| | KRT13 + | | | | | 28.62 | | | | | |
| | HPX + | β2 | nFCER2 | 0.01472 | 0.03579 | −0.07481 to | β2 | nFCER2 | 0.006014 | 0.05187 | −0.09923 to |
| | HRNR+ | | | | | 0.08562 | | | | | 0.09716 |
| | CD99 | β3 | nCANX | 24.87 | 31.41 | 42.91 to | β3 | nCANX | 54.35 | 46.88 | −30.02 to 160.3 |
| | | | | | | 105.0 | | | | | |
| | | β4 | nKRT13 | −7.29 | 5.696 | −20.19 to | β4 | nKRT13 | −8.118 | 6.592 | −23.63 to 3.851 |
| | | | | | | 3.058 | | | | | |
| | | β5 | nHPX | −1.286 | 3.944 | −11.17 to | β5 | nHPX | −3.559 | 6.334 | −18.28 to 4.943 |
| | | | | | | 4.900 | | | | | |
| | | β6 | nHRNR | 0.01423 | 0.012 | −0.01019 to | β6 | nHRNR | 0.01733 | 0.01475 | 0.009135 to |
| | | | | | | 0.04163 | | | | | 0.05238 |
| | | β7 | nCD99 | −49.11 | 53.09 | −178.1 to | β7 | nCD99 | −46.13 | 62.42 | −184.8 to 69.45 |
| | | | | | | 54.63 | | | | | |
| | PEDF + | β0 | Inter- | −12.32 | 5.467 | −24.70 to | β0 | Inter- | −11.43 | 5.649 | −24.52 to −1.581 |
| | FCER2 + | | cept | | | −2.669 | | cept | | | |
| | CANX + | β1 | nPEDF | −46.41 | 33.58 | −148.8 to | β1 | nPEDF | −71.24 | 60.34 | −211.6 to 3.491 |
| | KRT13 + | | | | | 10.25 | | | | | |
| | HPX + | β2 | nFCER2 | −0.04131 | 0.06105 | −0.1642 to | β2 | nFCER2 | −0.05591 | 0.06424 | −0.1855 to |
| | HRNR+ | | | | | 0.06505 | | | | | 0.07655 |
| | CD99 + | β3 | nCANX | 69.26 | 41.63 | −14.55 to | β3 | nCANX | 100.4 | 57.48 | 1.367 to 228.8 |
| | Age | | | | | 167.6 | | | | | |
| | | β4 | nKRT13 | −17.52 | 8.963 | −37.74 to | β4 | nKRT13 | −15.71 | 8.853 | −36.38 to −1.005 |
| | | | | | | −2.441 | | | | | |
| | | β5 | nHPX | 0.1677 | 3.532 | −10.08 to | β5 | nHPX | −3.616 | 7.888 | −19.96 to 5.806 |
| | | | | | | 6.335 | | | | | |
| | | β6 | Age | 0.2209 | 0.09072 | 0.06280 to | β6 | Age | 0.1973 | 0.09184 | 0.03931 to |
| | | | | | | 0.4277 | | | | | 0.4115 |
| | | β7 | nHRNR | 0.02358 | 0.01579 | −0.004270 to | β7 | nHRNR | 0.03308 | 0.02237 | 0.002994 to |
| | | | | | | 0.06051 | | | | | 0.08380 |
| | | β8 | nCD99 | −27.33 | 72.71 | −192.0 to | β8 | nCD99 | −37.55 | 80.48 | −208.7 to 109.8 |
| | | | | | | 105.4 | | | | | |
| | PEDF + | β0 | Inter- | −1.942 | 1.676 | −5.826 to | β0 | Inter- | −6.116 | 3.187 | −14.10 to −1.087 |
| | FCER2 + | | cept | | | 1.030 | | cept | | | |
| | CANX + | β1 | nPEDF | −27.5 | 31.85 | 108.0 to | β1 | nPEDF | −49.37 | 36.73 | −179.7 to 15.64 |
| | KRT13 + | | | | | 31.99 | | | | | |
| | HPX + | β2 | nFCER2 | 0.01115 | 0.04219 | −0.08562 to | β2 | nFCER2 | 0.009079 | 0.05384 | −0.1109 to |
| | HRNR+ | | | | | 0.1126 | | | | | 0.1260 |
| | CD99 + | β3 | nCANX | 4.907 | 34.6 | −87.62 to | β3 | nCANX | 18.52 | 40.86 | −84.90 to 120.1 |
| | PI- | | | | | 72.16 | | | | | |
| | RADS | β4 | nKRT13 | −6.208 | 7.016 | −23.44 to | β4 | nKRT13 | −5.119 | 7.526 | 25.24 to 10.44 |
| | | | | | | 6.766 | | | | | |
| | | β5 | nHPX | 1.671 | 5.358 | −9.595 to | β5 | nHPX | 3.558 | 6.579 | −11.69 to 17.51 |
| | | | | | | 13.87 | | | | | |
| | | β6 | PI- | 0.9681 | 0.462 | 0.1876 to | β6 | PI- | 1.854 | 0.8177 | 0.5875 to 3.890 |
| | | | RADS | | | 2.054 | | RADS | | | |
| | | β7 | nHRNR | 0.01555 | 0.01572 | −0.01430 to | β7 | nHRNR | 0.02659 | 0.02024 | −0.009537 to |
| | | | | | | 0.05461 | | | | | 0.08441 |
| | | β8 | nCD99 | −53.54 | 79.33 | −255.8 to | β8 | nCD99 | −89.15 | 89.72 | −302.2 to 80.69 |
| | | | | | | 83.37 | | | | | |
| | PEDF + | β0 | Inter- | −13.23 | 7.295 | −30.78 to | β0 | Inter- | −21.64 | 11.91 | −51.48 to |
| | FCER2 + | | cept | | | −0.1950 | | cept | | | −2.431 |
| | CANX + | β1 | nPEDF | −59.19 | 38.3 | 156.2 to | β1 | nPEDF | −97.5 | 54.06 | −278.7 to |
| | KRT13 | | | | | 10.50 | | | | | −8.196 |
| | HPX + | β2 | nFCER2 | −0.01641 | 0.05876 | 0.1552 to | β2 | nFCER2 | −0.05332 | 0.08433 | −0.2396 to |
| | HRNR+ | | | | | 0.08825 | | | | | 0.1146 |
| | CD99 + | β3 | nCANX | 39.79 | 44.25 | −62.82 to | β3 | nCANX | 76.58 | 68.34 | −52.40 to 239.9 |
| | Age + | | | | | 136.3 | | | | | |
| | PI- | β4 | nKRT13 | −12.13 | 9.094 | 33.24 to | β4 | nKRT13 | 10.64 | 8.815 | −32.39 to 7.552 |
| | RADS | | | | | 3.296 | | | | | |
| | | β5 | nHPX | 6.091 | 6.67 | −6.904 to | β5 | nHPX | 6.021 | 8.356 | −11.74 to 24.47 |
| | | | | | | 19.90 | | | | | |
| | | β6 | PI- | 0.875 | 0.5287 | −0.02376 to | β6 | PI- | 2.118 | 0.9643 | 0.5961 to 4.453 |
| | | | RADS | | | 2.139 | | RADS | | | |
| | | β7 | Age | 0.1821 | 0.1118 | −0.02215 to | β7 | Age | 0.2271 | 0.159 | −0.04153 to |
| | | | | | | 0.4452 | | | | | 0.6204 |
| | | β8 | nHRNR | 0.02326 | 0.01898 | −0.01059 to | β8 | nHRNR | 0.04795 | 0.02931 | −2.822e−005 to |
| | | | | | | 0.06661 | | | | | 0.1350 |
| | | β9 | nCD99 | −69.08 | 81.67 | −255.7 to | β9 | nCD99 | −114.6 | 97.13 | −353.7 to 62.40 |
| | | | | | | 90.34 | | | | | |

TABLE 10.1

| | Detect PI-RADS (3-5) | | | | | |
|---|---|---|---|---|---|---|
| | | | | | Specificity | Specificity |
| | | Std. | 95% Confidence | | at 90% | at 100% |
| | AUC | Error | interval | p-value | Sensitivity | Sensitivity |
| LYVE1* | 0.6711 | 0.09241 | 0.4900 to 0.8522 | 0.0793 | 38.5 | 0.0 |
| SPARCL1 * | 0.7294 | 0.08090 | 0.5709 to 0.8880 | 0.0186 | 23.1 | 7.7 |
| AMBP * | 0.7427 | 0.08362 | 0.5788 to 0.9066 | 0.0128 | 23.1 | 23.1 |
| KRT13 * | 0.6419 | 0.09270 | 0.4602 to 0.8236 | 0.1455 | 30.8 | 30.8 |
| CD99 * | 0.7109 | 0.08383 | 0.5466 to 0.8752 | 0.0305 | 30.8 | 7.7 |
| HRNR* | 0.6751 | 0.09297 | 0.4929 to 0.8573 | 0.0725 | 15.4 | 15.4 |
| Age | 0.7427 | 0.0869 | 0.5724 to 0.9131 | 0.0128 | 23.1 | 0.0 |
| PSA | 0.6220 | 0.09459 | 0.4366 to 0.8074 | 0.2107 | 15.4 | 0.0 |
| AMBP + Age | 0.7401 | 0.08712 | 0.5693 to 0.9108 | 0.0138 | 38.5 | 23.1 |
| CD99 + Age | 0.7427 | 0.08692 | 0.5723 to 0.9131 | 0.0128 | 30.8 | 15.4 |
| HRNR + Age | 0.7454 | 0.08699 | 0.5749 to 0.9159 | 0.0118 | 38.5 | 15.4 |
| KRT13 + Age | 0.7613 | 0.07346 | 0.6173 to 0.9052 | 0.0074 | 23.1 | 23.1 |
| LYVE1 + Age | 0.7958 | 0.06887 | 0.6608 to 0.9307 | 0.0024 | 30.8 | 7.7 |
| SPARCL1 + Age | 0.7666 | 0.0782 | 0.6133 to 0.9198 | 0.0 | 46.2 | 7.7 |
| CD99 + HRNR | 0.7003 | 0.08470 | 0.5343 to 0.8663 | 0.0400 | 15.4 | 15.4 |
| CD99 + HRNR + Age | 0.7427 | 0.08703 | 0.5721 to 0.9133 | 0.0128 | 38.5 | 15.4 |
| CD99 + SPARCL1 | 0.7188 | 0.08133 | 0.5594 to 0.8782 | 0.0248 | 15.4 | 7.7 |
| CD99 + SPARCL 1 + Age | 0.7905 | 0.07077 | 0.6517 to 0.9292 | 0.0029 | 30.8 | 7.7 |
| HRNR + LYVE1 | 0.7454 | 0.07821 | 0.5921 to 0.8986 | 0.0118 | 30.8 | 15.4 |
| HRNR + LYVE1 + Age | 0.8011 | 0.06778 | 0.6682 to 0.9339 | 0.0020 | 30.8 | 15.4 |
| CD99 + KRT13 | 0.6499 | 0.09176 | 0.4700 to 0.8297 | 0.1242 | 30.8 | 30.8 |
| CD99 + KRT13 + Age | 0.7613 | 0.07346 | 0.6173 to 0.9052 | 0.0074 | 23.1 | 23.1 |
| AMBP + SPARCL1 | 0.7427 | 0.08428 | 0.5775 to 0.9079 | 0.0128 | 30.8 | 23.1 |
| AMBP + SPARCL1 + Age | 0.7613 | 0.08378 | 0.5971 to 0.9255 | 0.0074 | 46.2 | 23.1 |
| KRT13 + LYVE1 | 0.6764 | 0.08832 | 0.5033 to 0.8495 | 0.0704 | 30.8 | 23.1 |
| KRT13 + LYVE1 + Age | 0.7878 | 0.07002 | 0.6506 to 0.9250 | 0.0032 | 38.5 | 23.1 |
| CD99 + HRNR + AMBP | 0.7613 | 0.07809 | 0.6082 to 0.9143 | 0.0074 | 23.1 | 23.1 |
| CD99 + HRNR + AMBP + Age | 0.7560 | 0.08766 | 0.5842 to 0.9278 | 0.0087 | 46.2 | 23.1 |
| CD99 + HRNR + AMBP + KRT13 | 0.6897 | 0.09309 | 0.5072 to 0.8721 | 0.0517 | 38.5 | 30.8 |
| CD99 + HRNR + AMBP + KRT13 + Age | 0.8037 | 0.06964 | 0.6672 to 0.9402 | 0.0018 | 38.5 | 30.8 |
| CD99 + HRNR + AMBP + KR113 + LYVE1 | 0.7719 | 0.08749 | 0.6004 to 0.9434 | 0.0053 | 53.9 | 38.5 |
| CD99 + HRNR + AMBP + KRT13 + | 0.8647 | 0.05832 | 0.7504 to 0.9790 | 0.0002 | 53.9 | 30.8 |
| LYVE1 + Age | | | | | | |
| CD99 + HRNR + AMBP + KRT13 + LYVE1 + | 0.7666 | 0.08835 | 0.5934 to 0.9397 | 0.0063 | 53.9 | 38.5 |
| SPARCL1 | | | | | | |
| CD99 + HRNR + AMBP + KRT13 + LYVE1 + | 0.8647 | 0.06006 | 0.7470 to 0.9824 | 0.0002 | 61.5 | 23.1 |
| SPARCL1 + Age | | | | | | |

*Data normalized to CD44 and RNASE2

TABLE 10

| | Detection of PI-RADS (3-5) | | | | |
|---|---|---|---|---|---|
| | Parameter | Variable | | Standard | 95% CI (profile |
| | estimates | (x) | Estimate | error | likelihood) |
| AMBP + Age | β0 | Intercept | −4.649 | 3.944 | −13.19 to 2.637 |
| | β1 | Age | 0.09369 | 0.06175 | −0.01822 to 0.2295 |
| | β2 | AMBP | −21.66 | 14.61 | −57.38 to −1.169 |
| CD99 + Age | β0 | Intercept | −5.694 | 3.874 | −14.12 to 1.410 |
| | β1 | Age | 0.1055 | 0.06102 | −0.004664 to 0.2397 |
| | β2 | CD99 | −6806 | 5900 | −22515 to 3510 |
| HRNR + Age | β0 | Intercept | −5.209 | 3.914 | −13.68 to 2.017 |
| | β1 | Age | 0.09779 | 0.06151 | −0.01393 to 0.2325 |
| | β2 | HRNR | −2.483 | 2.193 | −8.821 to 0.6670 |
| KRT13 + Age | β0 | Intercept | −6.13 | 3.755 | −14.35 to 0.8083 |
| | β1 | Age | 0.1197 | 0.06015 | 0.01067 to 0.2531 |
| | β2 | KRT13 | −3311 | 1740 | −7280 to −291.6 |
| LYVE1 + Age | β0 | Intercept | −6.818 | 3.697 | −14.88 to −0.003846 |
| | β1 | Age | 0.1255 | 0.05906 | 0.01799 to 0.2554 |
| | β2 | LYVE1 | −0.3992 | 0.2263 | −0.9259 to −0.001407 |
| SPARCL1 + Age | β0 | Intercept | −5.759 | 3.733 | −13.98 to 1.065 |
| | β1 | Age | 0.1098 | 0.0591 | 0.003220 to 0.2413 |
| | β2 | SPARCL1 | −7.701 | 5.664 | −20.66 to 0.2013 |
| CD99 + HRNR | β0 | Intercept | 1.071 | 0.3841 | 0.3523 to 1.875 |
| | β1 | CD99 | −427.2 | 10218 | −21755 to 24948 |
| | β2 | HRNR | −2.875 | 3.531 | −13.00 to 1.905 |
| CD99 + | β3 | Intercept | −5.216 | 3.914 | −13.69 to 2.011 |
| HRNR + Age | β1 | Age | 0.09804 | 0.06154 | −0.01376 to 0.2329 |
| | β2 | CD99 | −1170 | 10255 | −22489 to 24227 |
| | β3 | HRNR | −2.148 | 3.561 | −12.17 to 2.716 |
| CD99 + SPARCL1 | β0 | Intercept | 1.237 | 0.4403 | 0.4413 to 2.181 |
| | β1 | SPARCL1 | −10.22 | 8.95 | −29.34 to 5.295 |
| | β2 | CD99 | 5605 | 13124 | −20801 to 33147 |
| CD99 + SPARCL1 + | β0 | Intercept | −7.177 | 3.977 | −15.90 to 0.09231 |
| Age | β1 | Age | 0.1339 | 0.06396 | 0.01877 to 0.2759 |
| | β2 | SPARCL1 | −18.49 | 11.09 | −41.99 to 1.153 |
| | β3 | CD99 | 17405 | 14927 | −11725 to 48847 |
| HRNR + LYVE1 | β0 | Intercept | 1.2 | 0.4029 | 0.4499 to 2.048 |
| | β1 | LYVE1 | −0.2144 | 0.2117 | −0.7144 to 0.2021 |
| | β2 | HRNR | −2.091 | 1.945 | −8.285 to 0.8944 |
| HRNR + LYVE1 + Age | β0 | Intercept | −6.016 | 3.878 | −14.43 to 1.179 |
| | β1 | Age | 0.1135 | 0.06144 | 0.001042 to 0.2481 |
| | β2 | LYVE1 | −0.3427 | 0.2468 | −0.8952 to 0.1252 |
| | β3 | HRNR | −1.058 | 1.919 | −7.140 to 2.099 |
| CD99 + KRT13 | β0 | Intercept | 1.417 | 0.4772 | 0.5372 to 2.435 |
| | β1 | KRT13 | −2478 | 1854 | −6740 to 977.8 |
| | β2 | CD99 | −2125 | 7292 | −19376 to 12090 |
| CD99 + KRT13 + Age | β0 | Intercept | −6.509 | 3.941 | −15.10 to 0.7429 |
| | β1 | Age | 0.1262 | 0.06368 | 0.01148 to 0.2672 |
| | β2 | KRT13 | −3900 | 2515 | −9434 to 341.8 |
| | β3 | CD99 | 2886 | 8443 | −15131 to 20064 |
| AMBP + SPARCL1 | β0 | Intercept | 1.413 | 0.4729 | 0.5434 to 2.421 |
| | β1 | SPARCL1 | −1.307 | 6.478 | −14.68 to 12.02 |
| | β2 | AMBP | −22.82 | 15.8 | −61.87 to 4.281 |
| AMBP + SPARCL1 + | β0 | Intercept | −4.752 | 3.903 | −13.22 to 2.499 |
| Age | β1 | Age | 0.0963 | 0.06131 | −0.01571 to 0.2311 |
| | β2 | SPARCL1 | −3.109 | 6.988 | −17.96 to 10.93 |
| | β3 | AMBP | −17.83 | 16.58 | −57.61 to 11.27 |
| KRT13 + LYVE1 | β0 | Intercept | 1.434 | 0.4757 | 0.5565 to 2.447 |
| | β1 | LYVE1 | −0.1478 | 0.2323 | −0.6824 to 0.3067 |
| | β2 | KRT13 | −2225 | 1669 | −5971 to 972.6 |
| KRT13 + LYVE1 + Age | β3 | Intercept | −6.478 | 3.728 | −14.55 to 0.4745 |
| | β1 | Age | 0.1254 | 0.05978 | 0.01590 to 0.2563 |
| | β2 | LYVE1 | −0.2268 | 0.2543 | −0.7969 to 0.2585 |
| | β3 | KRT13 | −2279 | 1911 | −6784 to 1135 |
| CD99 + HRNR + AMBP | β0 | Intercept | 1.34 | 0.4618 | 0.4942 to 2.331 |
| | β1 | AMBP | −27.36 | 18.76 | −74.88 to 1.442 |
| | β2 | CD99 | 8645 | 12956 | −14942 to 47577 |
| | β3 | HRNR | −1.5 | 3.756 | −12.85 to 4.133 |
| CD99 + HRNR + | β0 | Intercept | −4.6 | 3.983 | −13.21 to 2.766 |
| AMBP + Age | β1 | Age | 0.09238 | 0.06237 | −0.02086 to 0.2293 |
| | β2 | AMBP | −26.08 | 19.34 | −74.88 to 2.690 |
| | β3 | CD99 | 7458 | 12920 | −16150 to 46352 |
| | β4 | HRNR | −0.7615 | 3.696 | −12.15 to 5.000 |
| CD99 + HRNR + | β0 | Intercept | 1.634 | 0.5379 | 0.6540 to 2.791 |
| AMBP + KRT13 | β1 | AMBP | −26.79 | 18.55 | −74.47 to 1.184 |
| | β2 | KRT13 | −2227 | 1864 | −6492 to 1407 |
| | β3 | CD99 | 13357 | 15063 | −11430 to 65134 |
| | β4 | HRNR | −0.7706 | 3.391 | −11.34 to 4.782 |
| CD99 + HRNR + AMBP + | β0 | Intercept | −5.724 | 4.088 | −14.60 to 1.847 |
| KRT13 + Age | β1 | Age | 0.117 | 0.06585 | −0.002096 to 0.2628 |
| | β2 | AMBP | −25.36 | 19.47 | −75.51 to 2.525 |
| | β3 | KRT13 | −3538 | 2471 | −9241 to 638.6 |
| | β4 | CD99 | 14064 | 15736 | −11117 to 67148 |
| | β5 | HRNR | 0.5667 | 3.321 | −9.682 to 6.355 |
| CD99 + HRNR + AMBP + | β0 | Intercept | 1.843 | 0.5955 | 0.7777 to 3.155 |
| KRT13 + LYVE1 | β1 | LYVE1 | −1.502 | 0.944 | −3.651 to 0.004418 |
| | β2 | AMBP | −27.69 | 15.2 | −69.58 to −3.344 |
| | β3 | KRT13 | −2475 | 2248 | −7438 to 2370 |
| | β4 | CD99 | 75681 | 43815 | 5817 to 179474 |
| | β5 | HRNR | −7.212 | 5.876 | −24.36 to 1.649 |
| CD99 + HRNR + AMBP + | β0 | Intercept | −8.813 | 5.09 | −20.35 to 0.3129 |
| KRT13 + LYVE1 + | β1 | Age | 0.1665 | 0.08108 | 0.02456 to 0.3527 |
| Age | β2 | LYVE1 | −1.277 | 0.8804 | −3.751 to −0.1741 |
| | β3 | AMBP | −25.29 | 14.92 | −66.14 to −1.677 |
| | β4 | KRT13 | −2411 | 2319 | −8201 to 1987 |
| | β5 | CD99 | 66454 | 40693 | 11839 to 174205 |
| | β6 | HRNR | −5.284 | 5.435 | −23.92 to 2.606 |
| CD99 + HRNR + AMBP + | β0 | Intercept | 1.842 | 0.5976 | 0.7712 to 3.159 |
| KRT13 + LYVE1 + | β1 | LYVE1 | −1.506 | 0.9444 | −3.645 to 0.02790 |
| SPARCL1 | β2 | SPARCL1 | −2.233 | 15.04 | −32.76 to 28.68 |
| | β3 | AMBP | −26.73 | 16.71 | −69.47 to 3.153 |
| | β4 | KRT13 | −2272 | 2635 | −7865 to 3265 |
| | β5 | CD99 | 78593 | 48446 | −128.3 to 197427 |
| | β6 | HRNR | −7.447 | 6.028 | −25.09 to 1.936 |
| CD99 + HRNR + AMBP + | β0 | Intercept | −8.836 | 5.103 | −20.38 to 0.2909 |
| KRT13 + LYVE1 + | β1 | Age | 0.1665 | 0.0811 | 0.02486 to 0.3527 |
| SPARCL1 + Age | β2 | LYVE1 | −1.285 | 0.9136 | −3.795 to −0.1384 |
| | β3 | SPARCL1 | −3.883 | 17.93 | −40.76 to 31.92 |
| | β4 | AMBP | −23.4 | 17.57 | −65.83 to 9.344 |
| | β5 | KRT13 | −2069 | 2818 | −8493 to 3440 |
| | β6 | CD99 | 72088 | 50599 | 5052 to 209278 |
| | β7 | HRNR | −5.772 | 5.893 | −25.24 to 2.932 |

Tumor ~ β0 + (β1*x1) + (β2*x2) + (βn*xn)
x = biomarker concentration or clinical variable (Age, etc.)

TABLE 11

| | Number of | | Median | Median | Prostate |
|---|---|---|---|---|---|
| | Samples | Gleason | Age | Serum PSA | Volume |
| | (% of total) | Score | (Min-Max) | (Min-Max) | (Min-Max) * |
| No Tumor | 24 (53.3%) | 0 | 63.5 | 6.60 | 60.19 |
| | | | (52-82) | (2.00-14.97) | (18.56-203.68) |
| Tumor | 21 (46.7%) | 6-9 | 65 | 7.22 | 48.59 |
| | | | (52 76) | (2.00 38.80) | (17.00 80.63) |
| | 4 (8.9%) | 6 | 65 | 8.53 | 60.54 |
| | | | (64-70) | (4.53-17.37) | (30.90-80.63) |
| | 8 (17.8%) | 7 | 65 | 4.94 | 50.00 |
| | | | (52-73) | (2.00-11.00) | (26.45-72.54) |
| | 9 (20.0%) | 8-9 | 74 | 12.41 | 47.17 |
| | | | (58-76) | (4.86-38.80) | (17.00-60.00) |
| Total | 45 (100%) | | 65 | 6.90 | 52.00 |
| | | | (52-82) | (2.00-38.80) | (17.00-203.68) |

| | ELISA Catalogue | ELISA | |
|---|---|---|---|
| Target | Number | Producer | Notes |
| CD99 | ELH-CD99 | RayBiotech (Peachtree | — |
| | | Corners, GA, USA) | |
| HRNR | LS-F8355 | LifeSpan BioScience | — |
| | | (Seattle, WA, USA) | |
| KRT13 | LS-F36678 | LifeSpan BioScience | — |
| | | (Seattle, WA, USA) | |
| FCER2 | LS-F2751 | LifeSpan BioScience | — |
| | | (Seattle, WA, USA) | |
| PEDF | LS-F33276 | LifeSpan BioScience | — |
| | | (Seattle, WA, USA) | |
| HPX | EH238RB | Thermo Fisher | — |
| | | Scientific (Basel, | |
| | | Switzerland) | |
| CANX | ABIN6965340 | Antibodies Online | — |
| | | (Aachen, Germany) | |
| CD44 | ab45912 | Abcam | Samples diluted |
| | | (Cambridge, UK) | 1:100/200 |
| RNASE2 | 7630 | MBL Int. Corp. | Samples |
| | | (Woburn, MA, USA) | diluted 1:50 |

TABLE 13

| | | Mass Spectrometry | | | | | | | |
|---|---|---|---|---|---|---|---|---|---|
| | | Analysis | | ROC Curve Analysis | | | | | |
| | | Absolute | | | | 95% | | Specificity | Specificity |
| | | Log2 Fold | | | Std. | Confidence | | at 90% | at 100% |
| | Genes | Change | q-value | AUC | Error | Interval | p-value | Sensitivity | Sensitivity |
| BIOMARKERS | B2M | 1.395 | 2.33E−04 | 0.8261 | 0.064 | 0.7008 to | 0.0003 | 65.2 | 13 |
| | | | | | | 0.9514 | | | |
| | VIPR1 | 1.452 | 3.06E−05 | 0.817 | 0.067 | 0.6861 to | 0.0006 | 42.9 | 38.1 |
| | | | | | | 0.9480 | | | |
| | CALR | 0.86 | 2.16E−07 | 0.8043 | 0.069 | 0.6699 to | 0.0007 | 47.8 | 34.8 |
| | | | | | | 0.9388 | | | |
| | SERPINF1 | 1.039 | 9.61E−09 | 0.8023 | 0.07 | 0.6659 to | 0.0008 | 68.2 | 36.4 |
| | | | | | | 0.9386 | | | |
| | HPX | 0.952 | 1.58E−06 | 0.7761 | 0.07 | 0.6396 to | 0.0020 | 52.2 | 39.1 |
| | | | | | | 0.9125 | | | |
| | IGFALS | 1.1 | 4.09E−04 | 0.7761 | 0.072 | 0.6348 to | 0.0020 | 56.5 | 52.2 |
| | | | | | | 0.9174 | | | |
| | KRT2 | 2.176 | 3.65E−04 | 0.7696 | 0.074 | 0.6250 to | 0.0025 | 56.5 | 13 |
| | | | | | | 0.9142 | | | |
| | HRNR | 1.912 | 2.91E−04 | 0.7522 | 0.076 | 0.6033 to | 0.0047 | 47.8 | 13 |
| | | | | | | 0.9010 | | | |
| | APOA1 | 1.214 | 1.40E−05 | 0.7435 | 0.077 | 0.5930 to | 0.0064 | 52.2 | 17.4 |
| | | | | | | 0.8939 | | | |
| | GPR180 | 0.998 | 1.28E−04 | 0.7432 | 0.079 | 0.5883 to | 0.0070 | 27.3 | 13.6 |
| | | | | | | 0.8980 | | | |
| | KRT13 | 2.235 | 4.60E−04 | 0.7391 | 0.075 | 0.5913 to | 0.0074 | 52.2 | 30.4 |
| | | | | | | 0.8869 | | | |
| | LRRC15 | 0.983 | 1.48E−04 | 0.7425 | 0.078 | 0.5902 to | 0.0087 | 40 | 20 |
| | | | | | | 0.8948 | | | |
| | APOA4 | 0.855 | 1.15E−05 | 0.7174 | 0.079 | 0.5634 to | 0.0149 | 52.2 | 26.1 |
| | | | | | | 0.8714 | | | |
| | JUP | 1.848 | 4.76E−04 | 0.7185 | 0.08 | 0.5614 to | 0.0158 | 21.7 | 17.4 |
| | | | | | | 0.8757 | | | |
| | ATP5F1A | 2.155 | 2.26E−04 | 0.7071 | 0.081 | 0.5487 to | 0.0222 | 43.5 | 21.7 |
| | | | | | | 0.8655 | | | |
| | DCD | 1.915 | 2.13E−04 | 0.6978 | 0.082 | 0.5375 to | 0.0267 | 52.2 | 21.7 |
| | | | | | | 0.8581 | | | |
| | CANX | 1.045 | 1.99E−05 | 0.7043 | 0.085 | 0.5377 to | 0.0273 | 47.6 | 38.1 |
| | | | | | | 0.8708 | | | |
| | MXRA8 | 0.904 | 1.29E−04 | 0.6891 | 0.08 | 0.5314 to | 0.0341 | 34.8 | 21.7 |
| | | | | | | 0.8469 | | | |
| | SCGB1A1 | 1.412 | 3.30E−04 | 0.687 | 0.081 | 0.5273 to | 0.0363 | 34.8 | 17.4 |
| | | | | | | 0.8466 | | | |
| | RNASE1 | 1.07 | 4.09E−04 | 0.687 | 0.082 | 0.5270 to | 0.0363 | 26.1 | 17.4 |
| | | | | | | 0.8470 | | | |
| | PNP | 5.845 | 1.10E−05 | 0.6739 | 0.082 | 0.5126 to | 0.0514 | 34.8 | 30.4 |
| | | | | | | 0.8352 | | | |
| | CD99 | 1.231 | 1.40E−05 | 0.675 | 0.083 | 0.5114 to | 0.0525 | 36.4 | 31.8 |
| | | | | | | 0.8386 | | | |
| | FCER2 | 0.834 | 5.78E−05 | 0.6717 | 0.084 | 0.5075 to | 0.0544 | 52.2 | 30.4 |
| | | | | | | 0.8360 | | | |
| | VAT1 | 0.951 | 9.46E−05 | 0.6717 | 0.084 | 0.5077 to | 0.0544 | 52.2 | 17.4 |
| | | | | | | 0.8358 | | | |
| | SCUBE3 | 1.485 | 2.03E−05 | 0.6746 | 0.086 | 0.5055 to | 0.0563 | 50 | 22.7 |
| | | | | | | 0.8438 | | | |
| CONTROLS | CD44 | 0.065 | 0.32 | 0.5717 | 0.091 | 0.3936 to | 0.4217 | 13.0 | 4.3 |
| | | | | | | 0.7499 | | | |
| | RNASE2 | 0.065 | 0.32 | 0.5326 | 0.090 | 0.3553 to | 0.7149 | 13.0 | 4.3 |
| | | | | | | 0.7099 | | | |

TABLE 14

| | | | 95% Confidence | | Specificity at | Specificity at |
|---|---|---|---|---|---|---|
| Biomarker | AUC | Std. Error | Interval | p-Value | 90% Sensitivity | 100% Sensitivity |
| PEDF | 0.8023 | 0.070 | 0.6659 to 0.9386 | 0.0008 | 68.2 | 36.4 |
| HPX | 0.7761 | 0.070 | 0.6396 to 0.9125 | 0.0020 | 52.2 | 39.1 |
| HRNR | 0.7522 | 0.076 | 0.6033 to 0.9010 | 0.0047 | 47.8 | 13.0 |
| KRT13 | 0.7391 | 0.075 | 0.5913 to 0.8869 | 0.0074 | 52.2 | 30.4 |
| CANX | 0.7043 | 0.085 | 0.5377 to 0.8708 | 0.0273 | 47.6 | 38.1 |
| CD99 | 0.6750 | 0.083 | 0.5114 to 0.8386 | 0.0525 | 36.4 | 31.8 |
| FCER2 | 0.6717 | 0.084 | 0.5075 to 0.8360 | 0.0544 | 52.2 | 30.4 |
| PEDF + HPX | 0.8977 | 0.050 | 0.7999 to 0.9956 | <0.0001 | 72.7 | 50.0 |
| PEDF + CD99 | 0.8786 | 0.056 | 0.7689 to 0.9883 | <0.0001 | 76.2 | 66.7 |
| PEDF + FCER2 | 0.8773 | 0.063 | 0.7530 to 1.000 | <0.0001 | 86.4 | 72.7 |
| PEDF + KRT13 | 0.8705 | 0.055 | 0.7618 to 0.9791 | <0.0001 | 72.7 | 54.5 |
| PEDF + HRNR | 0.8568 | 0.058 | 0.7437 to 0.9699 | <0.0001 | 77.3 | 54.5 |
| PEDF + CANX | 0.9105 | 0.053 | 0.8067 to 1.000 | <0.0001 | 85.0 | 70.0 |
| HPX + HRNR | 0.8739 | 0.054 | 0.7682 to 0.9797 | <0.0001 | 73.9 | 34.8 |
| HPX + KRT13 | 0.8413 | 0.061 | 0.7211 to 0.9615 | 0.0001 | 60.9 | 56.5 |
| HRNR + CANX | 0.8496 | 0.062 | 0.7272 to 0.9720 | 0.0002 | 66.7 | 66.7 |
| HPX + FCER2 | 0.8000 | 0.068 | 0.6670 to 0.9330 | 0.0008 | 60.9 | 60.9 |
| HPX + CD99 | 0.7864 | 0.071 | 0.6462 to 0.9265 | 0.0015 | 63.6 | 54.5 |
| KRT13 + CANX | 0.7820 | 0.076 | 0.6322 to 0.9318 | 0.0023 | 61.9 | 61.9 |
| KRT13 + FCER2 | 0.7652 | 0.074 | 0.6193 to 0.9111 | 0.0030 | 60.9 | 47.8 |
| HRNR + FCER2 | 0.7457 | 0.076 | 0.5964 to 0.8949 | 0.0059 | 60.9 | 34.8 |

TABLE 15

| | | | | 95% Confidence | | Specificity at 90% | Specificity at |
|---|---|---|---|---|---|---|---|
| | Biomarker | AUC | Std. Error | Interval | p-Value | Sensitivity | 100% Sensitivity |
| All | KRT13 | 0.8087 | 0.066 | 0.6797 to 0.9377 | 0.0005 | 43.5 | 43.5 |
| PCa | HPX | 0.7696 | 0.071 | 0.6314 to 0.9077 | 0.0025 | 47.8 | 43.5 |
| grades | PEDF | 0.7609 | 0.073 | 0.6176 to 0.9041 | 0.0035 | 34.8 | 30.4 |
| | CD99 | 0.7565 | 0.073 | 0.6136 to 0.8994 | 0.0041 | 52.2 | 47.8 |
| | FCER2 | 0.7565 | 0.074 | 0.6114 to 0.9017 | 0.0041 | 47.8 | 13.0 |
| | CANX | 0.7457 | 0.076 | 0.5971 to 0.8942 | 0.0059 | 30.4 | 26.1 |
| | HRNR | 0.7120 | 0.080 | 0.5553 to 0.8686 | 0.0176 | 39.1 | 17.4 |
| High-grade | KRT13 | 0.7708 | 0.075 | 0.6247 to 0.9170 | 0.0033 | 40.7 | 37.1 |
| PCa | HPX | 0.7546 | 0.074 | 0.6094 to 0.8998 | 0.0057 | 44.4 | 37.0 |
| | PFDF | 0.7292 | 0.079 | 0.5752 to 0.8831 | 0.0129 | 33.3 | 29.6 |
| | FCER2 | 0.7269 | 0.081 | 0.5690 to 0.8847 | 0.0138 | 44.4 | 11.2 |
| | CD99 | 0.7222 | 0.078 | 0.5688 to 0.8756 | 0.0159 | 40.7 | 40.7 |
| | HRNR | 0.6956 | 0.083 | 0.5321 to 0.8591 | 0.0337 | 37.0 | 14.8 |
| | CANX | 0.6528 | 0.086 | 0.4849 to 0.8207 | 0.0973 | 25.9 | 22.1 |

TABLE 16

| | | | | 95% Confidence | | Specificity at | Specificity at 100% |
|---|---|---|---|---|---|---|---|
| | Biomarker | AUC | Std. Error | Interval | p-Value | 90% Sensitivity | Sensitivity |
| All | KRT13 | 0.7696 | 0.071 | 0.6298 to 0.9093 | 0.0025 | 52.2 | 30.4 |
| PCa | HRNR | 0.7413 | 0.079 | 0.5865 to 0.8961 | 0.0069 | 52.2 | 8.7 |
| grades | FCER2 | 0.7326 | 0.077 | 0.5813 to 0.8839 | 0.0092 | 52.2 | 39.1 |
| | CANX | 0.7043 | 0.080 | 0.5479 to 0.8608 | 0.0221 | 30.4 | 17.4 |
| | PEDF | 0.700 | 0.081 | 0.5404 to 0.8596 | 0.0251 | 30.4 | 30.4 |
| | HPX | 0.6978 | 0.081 | 0.5386 to 0.8570 | 0.0267 | 39.1 | 8.7 |
| | CD99 | 0.6652 | 0.083 | 0.5032 to 0.8273 | 0.0642 | 34.8 | 21.7 |
| | KRT13 + | 0.8196 | 0.065 | 0.6927 to 0.9464 | 0.0003 | 52.2 | 52.2 |
| | FCER2 | | | | | | |
| | HPX + FCER2 | 0.8087 | 0.067 | 0.6767 to 0.9407 | 0.0005 | 43.5 | 30.4 |
| | PEDF + FCER2 | 0.8022 | 0.067 | 0.6714 to 0.9329 | 0.0007 | 52.2 | 39.1 |
| | HPX + KRT13 | 0.7826 | 0.070 | 0.6462 to 0.9190 | 0.0015 | 52.2 | 30.4 |
| | HRNR + FCER2 | 0.7826 | 0.071 | 0.6429 to 0.9223 | 0.0015 | 56.5 | 13.0 |
| | PEDF + KRT13 | 0.7804 | 0.070 | 0.6431 to 0.9178 | 0.0017 | 52.2 | 39.1 |
| | KRT13 + CANX | 0.7609 | 0.072 | 0.6189 to 0.9028 | 0.0035 | 47.8 | 30.4 |
| | HPX + HRNR | 0.7478 | 0.078 | 0.5960 to 0.8997 | 0.0055 | 43.5 | 8.7 |
| | PEDF + CANX | 0.7348 | 0.077 | 0.5844 to 0.8852 | 0.0085 | 47.8 | 26.1 |
| | HRNR + CANX | 0.7326 | 0.079 | 0.5781 to 0.8871 | 0.0092 | 43.5 | 8.7 |
| | PEDF + CD99 | 0.7304 | 0.076 | 0.5808 to 0.8801 | 0.0099 | 43.5 | 34.8 |
| | PEDF + HRNR | 0.7283 | 0.080 | 0.5723 to 0.8842 | 0.0106 | 43.5 | 8.7 |
| | HPX + CD99 | 0.7283 | 0.078 | 0.5753 to 0.8812 | 0.0106 | 39.1 | 17.4 |
| | PEDF + HPX | 0.7000 | 0.081 | 0.5417 to 0.8583 | 0.0251 | 26.1 | 13.0 |
| High-grade | KRT13 | 0.7361 | 0.077 | 0.5854 to 0.8868 | 0.0104 | 40.7 | 25.9 |
| PCa | HRNR | 0.7199 | 0.084 | 0.5551 to 0.8847 | 0.0170 | 14.8 | 7.4 |
| | FCER2 | 0.7014 | 0.079 | 0.5468 to 0.8560 | 0.0288 | 44.4 | 33.3 |
| | HPX | 0.6968 | 0.087 | 0.5262 to 0.8673 | 0.0327 | 7.4 | 7.4 |
| | PEDF | 0.6806 | 0.085 | 0.5141 to 0.8470 | 0.0500 | 33.3 | 18.5 |
| | CD99 | 0.6644 | 0.086 | 0.4967 to 0.8320 | 0.0744 | 29.6 | 18.5 |
| | CANX | 0.6574 | 0.085 | 0.4907 to 0.8241 | 0.0875 | 22.2 | 14.8 |
| | HPX + FCER2 | 0.7894 | 0.077 | 0.6376 to 0.9411 | 0.0017 | 33.3 | 33.3 |
| | HPX + KRT13 | 0.7870 | 0.073 | 0.6432 to 0.9308 | 0.0018 | 33.3 | 18.5 |
| | KRT13 + | 0.7801 | 0.069 | 0.6447 to 0.9155 | 0.0024 | 51.8 | 48.1 |
| | FCER2 | | | | | | |
| | HPX + CD99 | 0.7662 | 0.078 | 0.6136 to 0.9188 | 0.0039 | 29.6 | 14.8 |
| | PEDF + FCER2 | 0.7523 | 0.073 | 0.6090 to 0.8956 | 0.0062 | 48.1 | 44.5 |
| | HRNR + FCER2 | 0.7523 | 0.076 | 0.6024 to 0.9022 | 0.0062 | 51.8 | 11.1 |
| | HPX + HRNR | 0.7500 | 0.084 | 0.5845 to 0.9155 | 0.0067 | 11.1 | 7.4 |
| | PEDF + KRT13 | 0.7431 | 0.075 | 0.5964 to 0.8898 | 0.0083 | 44.5 | 33.3 |
| | KRT13 + CANX | 0.7384 | 0.076 | 0.5886 to 0.8882 | 0.0097 | 40.7 | 29.6 |
| | PEDF + CD99 | 0.7176 | 0.078 | 0.5657 to 0.8695 | 0.0182 | 37.0 | 37.0 |
| | PEDF + HPX | 0.7083 | 0.083 | 0.5461 to 0.8705 | 0.0237 | 14.8 | 14.8 |
| | HRNR + CANX | 0.7014 | 0.083 | 0.5384 to 0.8644 | 0.0288 | 29.6 | 3.7 |
| | PEDF + HRNR | 0.6968 | 0.082 | 0.5358 to 0.8577 | 0.0327 | 33.3 | 11.1 |
| | PEDF + CANX | 0.6898 | 0.081 | 0.5303 to 0.8493 | 0.0394 | 44.4 | 18.5 |

1. A method for determining whether a subject has prostate cancer, said method comprising
the quantitative detection, in a sample obtained from the subject, of the concentration of a biomarker selected from the following Table (Table 5.1):

| Gene | Protein Name | Uniprot ID |
|---|---|---|
| SERPINF1 | Pigment epithelium-derived factor | P36955 |
| (PEDF) | | |
| CALR | Calreticulin | P27797 |
| HPX | Hemopexin | P02790 |
| PNP | Purine nucleoside phosphorylase | P00491 |
| APOA4 | Apolipoprotein A-IV | P06727 |
| CD99 | CD99 antigen | P14209 |
| APOA1 | Apolipoprotein A-I | P02647 |
| CANX | Calnexin | P27824 |
| SCUBE3 | Signal peptide, CUB and EGF-like domain- | Q8IX30 |
| | containing protein 3 | |
| VIPR1 | Vasoactive intestinal polypeptide receptor 1 | P32241 |
| FCER2 | Low affinity immunoglobulin epsilon Fc | P06734 |
| | receptor | |
| VAT1 | Synaptic vesicle membrane protein VAT-1 | Q99536 |
| | homolog | |
| GPR180 | Integral membrane protein GPR180 | Q86V85 |
| MXRA8 | Matrix remodeling-associated protein 8 | Q9BRK3 |
| LRRC15 | Leucine-rich repeat-containing protein 15 | Q8TF66 |
| DCD | Dermcidin | P81605 |
| ATP5F1A | ATP synthase subunit alpha, mitochondrial | P25705 |
| B2M | Beta-2-microglobulin | P61769 |
| HRNR | Hornerin | Q86YZ3 |
| SCGB1A1 | Uteroglobin | P11684 |
| KRT2 | Keratin, type II cytoskeletal 2 epidermal | P35908 |
| IGFALS | Insulin-like growth factor-binding protein | P35858 |
| | complex acid labile subunit | |
| RNASE1 | Ribonuclease pancreatic | P07998 |
| KRT13 | Keratin, type I cytoskeletal 13 | P13646 |
| JUP | Junction plakoglobin | P14923 |
| LCN2 | Neutrophil gelatinase-associated lipocalin | P80188 |
| SPARCL1 | SPARC-like protein 1 | Q14515 |
| LCP1 | Plastin-2 | P13796 |
| MSMB | Beta-microseminoprotein | P08118 |
| SCUBE2 | Signal peptide, CUB and EGF-like domain- | Q9NQ36 |
| | containing protein 2 | |
| IGLV3-10 | Immunoglobulin lambda variable 3-10 | A0A075B6K4 |
| TALDO1 | Transaldolase | P37837 |
| SERPINA6 | Corticosteroid-binding globulin | P08185 |
| TGFBR2 | TGF-beta receptor type-2 | P37173 |
| ANXA3 | Annexin A3 | P12429 |
| LYVE1 | Lymphatic vessel endothelial hyaluronic acid | Q9Y5Y7 |
| | receptor 1 | |
| PTGDS | Prostaglandin-H2 D-isomerase | P41222 |
| IGKV3D-11 | Immunoglobulin kappa variable 3D-11 | A0A0A0MRZ8 |
| TKT | Transketolase | P29401 |
| AMBP | Protein AMBP | P02760 |
| HYOU1 | Hypoxia up-regulated protein 1 | Q9Y4L1 |
| MASP1 | Mannan-binding lectin serine protease 1 | P48740 |
| CEL | Bile salt-activated lipase | P19835 |
| EEF2 | Elongation factor 2 | P13639 |
| SCUBE1 | Signal peptide, CUB and EGF-like domain- | Q8IWY4 |
| | containing protein 1 | |
| C4BPA | C4b-binding protein alpha chain | P04003 |
| BASP1 | Brain acid soluble protein 1 | P80723 |
| IGKV2-30 | Immunoglobulin kappa variable 2-30 | P06310 |
| AZGP1 | Zinc-alpha-2-glycoprotein | P25311 |
| APEH | Acylamino-acid-releasing enzyme | P13798 |
| CDH1 | Cadherin-1 | P12830 |
| S100A8 | Protein S100-A8 | P05109 |
| CD55 | Complement decay-accelerating factor | P08174 |
| DSG1 | Desmoglein-1 | Q02413 |
| PTGFRN | Prostaglandin F2 receptor negative regulator | Q9P2B2 |
| EMCN | Endomucin | Q9ULC0 |
| IL15RA | Interleukin-15 receptor subunit alpha | Q13261 |
| FCER1A | High affinity immunoglobulin epsilon receptor | P12319 |
| | subunit alpha | |
| NELL1 | Protein kinase C-binding protein NELL1 | Q92832 |
| GPR37L1 | G-protein coupled receptor 37-like 1 | O60883 |

establishing the statistical significance of the concentration of the biomarker.

2. The method according to claim 1, wherein the concentration of more than one biomarker is determined, and optionally combined with clinical data of the human subject.

3. The method according to claim 1, wherein a biomarker of at least one, two or of each column of the following Table (Table 4) is determined:

| No Tumor vs | Low vs High | |
|---|---|---|
| Tumor | Grade | PIRADS |
| GS8 vs GS6-9 | GS0-6 vs GS7-9 | 1-2 vs 3-5 |
| APOA1 | AMBP | AMBP |
| APOA4 | ANXA3 | APEH |
| ATP5F1A | APOA4 | AZGP1 |
| B2M | CANX | BASP1 |
| CALR | CD99 | CD55 |
| CANX | HPX | CDH1 |
| CD99 | HYOU1 | CEL |
| DCD | IGFALS | DSG1 |
| FCER2 | IGKV3D-11 | EEF2 |
| GPR180 | IGLV3-10 | EMCN |
| HPX | KRT13 | HYOU1 |
| HRNR | LCN2 | IGKV2-30 |
| IGFALS | LCP1 | IL15RA |
| JUP | LYVE1 | LYVE1 |
| KRT13 | MASP1 | PTGFRN |
| KRT2 | MSMB | S100A8 |
| LRRC15 | PTGDS | SCUBE1 |
| MXRA8 | SCUBE2 | SCUBE2 |
| PNP | SERPINA6 | SCUBE3 |
| RNASE1 | PEDF | SPARCL1 |
| SCGB1A1 | SPARCL1 | C4BPA |
| SCUBE3 | TALDO1 | FCER1A |
| PEDF | TGFBR2 | NELL1 |
| VAT1 | TKT | TALDO1 |
| VIPR1 | VIPR1 | GPR37L1 |

4. The method according to claim 1, wherein the sample is a urine or blood sample, particularly wherein the sample is a urine sample.

5.-7. (canceled)

8. The method according to claim 1, wherein the concentration of the following biomarkers is determined:
a. PEDF and FCER2; or
b. PEDF and CANX; or
c. HPX and KRT13; or
d. PEDF and FCER2 and CANX; or
e. PEDF and FCER2 and CANX and KRT13; or
f. PEDF and FCER2 and CANX and KRT13 and HPX; or
g. PEDF and FCER2 and CANX and KRT13 and HPX and HRNR; or
h. PEDF and FCER2 and CANX and KRT13 and HPX and HRNR and CD99
i. PEDF and FCER2 and CANX and KRT13 and HPX and HRNR and CD99 and SPARCL1
j. PEDF and FCER2 and CANX and KRT13 and HPX and HRNR and CD99 and SPARCL1 and AMBP
k. PEDF and FCER2 and CANX and KRT13 and HPX and HRNR and CD99 and SPARCL1 and AMBP and LYVE1

9. The method according to claim 8, wherein the biomarker is PEDF, and a concentration of PEDF is determined by mass spectrometry, and an intensity threshold score to detect men who should perform a prostate biopsy is below 100,000.

10. The method according to claim 1, wherein the concentration of the biomarkers is used to calculate a score value,
particularly wherein the score value is calculated by the following formula:
$$Score = {β}_{0} + {β}_{1} * {x}_{1} + {β}_{2} * {x}_{2} + … + {β}_{n} * {x}_{n}$$
wherein
Score is indicative for the probability of the subject to have prostate cancer, particularly high-grade prostate cancer and/or a PI-RADS score of 3-5;
“β” values are the regression coefficients,
“x” values are the measured concentrations of the respective proteins in urine samples or the value of clinical data, particularly age and/or PI-RADS score.
β₀ is the intercept,
index “n” represents the number of variables used.

11. The method according to claim 11, wherein the biomarker concentration is determined via mass spectrometry, and
β₀ is in the range of −10,000 to 10,000, particularly β₀ is in the range of −10 to 10, more particularly β₀ is in the range of 4 to 6, and/or
β₁ is in the range of −10,000 to 10,000, particularly β₁ is in the range of −10 to 10, particularly β₁ is in the range of −1 to 1, and/or
β₂ is in the range of −10,000 to 10,000, particularly β₂ is in the range of −10 to 10, particularly β₂ is in the range of −1 to 1, and/or
βₙ is in the range of −10,000 to 10,000, particularly βₙ is in the range of −10 to 10, particularly βₙ is in the range of −1 to 1, and/or
Score is in the range of −100 to 1000.

12. The method according to claim 11, wherein the biomarker concentration is determined via mass spectrometry, and the score value is calculated by the following formula:
$$Score = 5.075 + ( - 0.00005188 * {x}_{1} ) + ( - 0.000008438 * {x}_{2} )$$
wherein:
Score is indicative for the probability of the subject to have prostate cancer, particularly high-grade prostate cancer and/or a PI-RADS score of 3-5;
x₁=MS intensity of patient x for PEDF
x₂=MS intensity of patient x for FCER2.

13. The method according to claim 11, wherein the biomarker concentration is determined via ELISA, and
β₀ is in the range of −10,000 to 10,000, particularly β₀ is in the range of −10 to 10, more particularly β₀ is in the range of 4 to 6, and/or
β₁i is in the range of −10,000 to 10,000, particularly β₁ is in the range of −10 to 10, particularly β₁ is in the range of −1 to 1, and/or
β₂ is in the range of −10,000 to 10,000, particularly β₂ is in the range of −10 to 10, particularly β₂ is in the range of −1 to 1, and/or
βₙ is in the range of −10,000 to 10,000, particularly βₙ is in the range of −10 to 10, particularly βₙ is in the range of −1 to 1, and/or
Score is in the range of −100 to 1000.

14. The method according to claim 11, wherein the biomarker concentration is determined via ELISA, and the score value is calculated by the following formula:
$$Score = 1.931 + ( - 0.6994 * {x}_{1} ) + ( - 0.001579 * {x}_{2} )$$
wherein:
Score is indicative for the probability of the subject to have prostate cancer, particularly high-grade prostate cancer and/or a PI-RADS score of 3-5;
x₁=ELISA quantification of patient x for PEDF concentration
x₂=ELISA quantification of patient x for FCER2 concentration.

15. The method according to claim 11, wherein the biomarker concentration is determined via ELISA, and the score value is calculated by the following formula:
$$Score = 0.4349 + ( - 6.045 * {x}_{1} ) + ( - 0.001971 * {x}_{2} )$$
wherein:
Score is indicative for the probability of the subject to have prostate cancer, particularly high-grade prostate cancer and/or a PI-RADS score of 3-5;
x₁=ELISA quantification of patient x for KRT13 concentration
x₂=ELISA quantification of patient x for FCER2 concentration.

16. The method according to claim 11, wherein the biomarker concentration is determined via ELISA, and the score value is calculated by the following formula:
$$Score = 0.3256 + ( - 38.53 * {x}_{1} ) + ( - 19.75 * {x}_{2} )$$
wherein:
Score is indicative for the probability of the subject to have prostate cancer, particularly high-grade prostate cancer and/or a PI-RADS score of 3-5;
x₁=ELISA quantification of patient x for PEDF concentration
x₂=ELISA quantification of patient x for CD99 concentration.

17. The method according to claim 11, wherein the biomarker concentration is determined via ELISA, and the score value is calculated by the following formula:
$$Score = 0.4546 + ( - 0.007238 * {x}_{1} ) + ( - 6.736 * {x}_{2} )$$
wherein:
Score is indicative for the probability of the subject to have prostate cancer, particularly high-grade prostate cancer and/or a PI-RADS score of 3-5;
x₁=ELISA quantification of patient x for HPX concentration
x₂=ELISA quantification of patient x for HRNR concentration.

18. The method according to claim 11, wherein the biomarker concentration is determined via ELISA, and the score value is calculated by the following formula:
$$Score = 1.071 + ( - 427.2 * {x}_{1} ) + ( - 2.875 * {x}_{2} )$$
wherein:
Score is indicative for the probability of the subject to have prostate cancer, particularly high-grade prostate cancer and/or a PI-RADS score of 3-5;
x₁=ELISA quantification of patient x for CD99 concentration
x₂=ELISA quantification of patient x for HRNR concentration.

19. The method according to claim 11, wherein the biomarker concentration is determined via ELISA, and the score value is calculated by the following formula:
$$Score = 1.237 + ( - 10.22 * {x}_{1} ) + ( 5605 * {x}_{2} )$$
wherein:
Score is indicative for the probability of the subject to have prostate cancer, particularly high-grade prostate cancer and/or a PI-RADS score of 3-5;
x₁=ELISA quantification of patient x for CD99 concentration
x₂=ELISA quantification of patient x for SPARCL1 concentration.

20. The method according to claim 11, wherein the biomarker concentration is determined via ELISA, and the score value is calculated by the following formula:
$$Score = 1.413 + ( - 1.307 * {x}_{1} ) + ( - 22.82 * {x}_{2} )$$
wherein:
Score is indicative for the probability of the subject to have prostate cancer, particularly high-grade prostate cancer and/or a PI-RADS score of 3-5;
x₁=ELISA quantification of patient x for AMBP concentration
x₂=ELISA quantification of patient x for SPARCL1 concentration.

21. The method according to claim 11, wherein the biomarker concentration is determined via ELISA, and the score value is calculated by the following formula:
$$Score = 1.434 + ( - 0.1478 * {x}_{1} ) + ( - 222.5 * {x}_{2} )$$
wherein:
Score is indicative for the probability of the subject to have prostate cancer, particularly high-grade prostate cancer and/or a PI-RADS score of 3-5;
x₁=ELISA quantification of patient x for KRT13 concentration
x₂=ELISA quantification of patient x for LYVE1 concentration.

22. The method according to claim 11, wherein the age and/or PI-RADS of the subject contributes to the calculation of the score value.

23. The method according to claim 1, wherein collecting information about the health status comprises determining whether the subject
a. has, or is at risk of developing prostate cancer; and/or
b. has, or is at risk of having a high-grade prostate cancer;
and/or
c. has, or is at risk of biochemical recurrence; and/or
d. has, or is at risk of relapsing; and/or
e. is likely to benefit from a biopsy; and/or
f. is likely to benefit from active treatment; and/or
g. is likely to benefit from active surveillance; and/or
h. is likely to benefit from prostatectomy; and/or
i. is likely to benefit from chemotherapy or radiotherapy or hormone depletion treatment.

24.-25. (canceled)
